(19) United States
(12) Patent Application Publication
(10) Pub. No.: US 2024/0197729 A1
(43) Pub. Date: Jun. 20, 2024

(54) ALK-5 Inhibitors and Uses Thereof

(71) Applicant: Sumitomo Pharma Oncology, Inc., Marlborough, MA (US)

(72) Inventors: Bettina Franz, South Jordan, UT (US); Adam Siddiqui-Jain, South Jordan, UT (US); Steven L. Warner, Draper, UT (US)

(21) Appl. No.: 18/552,492

(22) PCT Filed: Mar. 25, 2022
(86) PCT No.: PCT/US2022/071345

§ 371 Date: Sep. 26, 2023

Related U.S. Application Data
(60) Provisional application No. 63/215,122, filed on Jun. 25, 2021.
(60) Provisional application No. 63/166,610, filed on Mar. 26, 2021.

(51) Int. Cl. A61K 31/502; A61K 31/5377; A61K 45/06; A61P 35/00; C07D 401/12; C07D 401/14; C07D 405/14

(52) U.S. Cl. CPC A61K 31/502; A61K 31/5377; A61K 45/06; A61P 35/00; C07D 401/12; C07D 401/14; C07D 405/14

(57) ABSTRACT

Provided herein are compounds (e.g., compounds of Formulae (I), (II), (III) and (IV), compounds listed in Table 1) and pharmaceutically acceptable salts thereof, pharmaceutical compositions of either of the foregoing, and kits comprising the same. The compounds provided herein are activin receptor-like kinase (e.g., ALK-5) inhibitors and are useful for treating and/or preventing diseases (e.g., proliferative diseases, e.g., cancer) in a subject, for inhibiting tumor growth in a subject, and/or for inhibiting the activity of an activin receptor-like kinase (e.g., ALK-5) in vitro or in vivo.

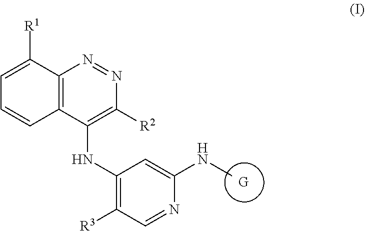

### RELATED APPLICATIONS

This application claims the benefit of U.S. Provisional Application No. 63/166,610, filed on Mar. 26, 2021, and U.S. Provisional Application No. 63/215,122, filed on Jun. 25, 2021. The entire teachings of the above applications are incorporated herein by reference.

### BACKGROUND

Activin receptor-like kinase 5 (ALK-5) (also known as TGF-β receptor type 1 (TGFβR1)) is a therapeutic target, e.g., in proliferative diseases such as cancer, because of its suggested roles in promoting tumor growth, survival, and metastasis. ALK-5 is a member of the TGF-β superfamily of receptors that has been suggested to regulate a wide array of cellular processes. Modulating TGF-β signaling is important to controlling cellular processes implicated in cellular proliferation. See, for example, Akhurst, R. J. and Hata, A., “Targeting the TGF-β Signalling Pathway in Disease”, Nat. Rev. Drug Disc., 11 pp 790-811 (2012) and Hallberg and Palmer, “The role of the ALK receptor in cancer biology”, Annals of Oncology, 2016, 27, iii4.

Generally during TGF-β signaling, a type I receptor is brought together with a type II receptor, both of which are serine/threonine kinases. To date, there are seven known type I receptors: activin receptor-like kinases 1 through 7 (ALK-1 through ALK-7). In some instances, TGF-β signals through a combination of TOR-II (a type II receptor) and ALK-5. Upon activation, the type I receptors transduce signals through various proteins, for example, activated type I receptors phosphorylate members of the receptor-regulated subfamily of SMADs, which allows them to complex with mediator SMADs. The resulting activated SMAD complexes accumulate in the nucleus, where they play a role in the transcription of target genes. Blocking this TGF-β signaling pathway through ALK inhibition (in particular, ALK-5 inhibition) is an attractive target for therapy due to the complex roles the pathway plays in cell proliferation, differentiation, adhesion, migration, and apoptosis. It has been noted that in proliferative and fibrotic diseases, cellular mutations occur wherein the normal proliferative suppression function of TGF-β signaling is conferred, thus allowing uncontrolled proliferation of the cells, see, e.g., Blobe, G. C., et al., “Role of Transforming Growth Factor β in Human Disease”, N Engl J Med (342), pp 1350-1358 (2000); Ballester, B. et al, “Idiopathic Pulmonary Fibrosis and Lung cancer: Mechanisms and Molecular Targets”, Int. J. of Molecular Sciences 20(593), doi:10.3390/ijms20030593 (2019), and Huang, J. J. and Blobe, G. C., “Dichotomous Roles of TGF-β in Human Cancer”, Biochem Soc. Trans 342(2016); 1441-1454 (https://doi.org/10.1042/BST20160065).

TGF-β is an important pathway in cancer that facilitates tumor growth and immune evasion, as well as playing a role in other cancer process such as metastasis and angiogenesis. Upregulation of the components of the TGF-β pathway, including the ligand and receptors, is observed in many types of cancer and is often associated with poor outcomes (de Reyniès, A., Javelaud, D., Elarouci, N. et al., Sci Rep 10, 14491 (2020). https://doi.org/10.1038/s41598-020-71559-w). Aberrant TGF-β signaling has been shown to be involved in the development of multiple cancer types, including triple negative breast cancer (Bhola, Neil E., et al. “TGF-β inhibition enhances chemotherapy action against triple-negative breast cancer.” The Journal of clinical investigation 123.3 (2013) https://doi.org/10.1172/JCI65416; Vishnubalaji, Radhakrishnan, and Nehad M. Alajez. “Epigenetic regulation of triple negative breast cancer (TNBC) by TGF-β signaling.” Scientific Reports 11.1 (2021) https://doi.org/10.1038/s41598-021-94514-9), pancreatic cancer (Goggins, Michael, et al “Progress in cancer genetics: lessons from pancreatic cancer.” Annals of oncology 10 (1999) https://doi.org/10.1093/annonc/10.suppl_4.S4), Truty, Mark J., and Raul Urrutia. “Basics of TGF-β and pancreatic cancer.” Pancreatology 7.5-6 (2007) https://doi.org/10.1159/000108959), and ovarian cancer (Monsivais, Diana, et al. “Activin-like kinase 5 (ALK5) inactivation in the mouse uterus results in metastatic endometrial carcinoma.” Proceedings of the National Academy of Sciences 116.9 (2.019) https://doi.org/10.1073/pnas.1806838116, Newsted, Daniel, et al. “Blockade of TGF-β signaling with novel synthetic antibodies limits imnmune exclusion and improves chemotherapy response in metastatic ovarian cancer models.” Oncoimmunology 8.2 (2019) https://doi.org/10.1080/2162402X.2018.1539613).

Signaling though this pathway begins with the liberation of the latent ligand (TGF-β) and binding specific serine/threonine residues on a specific receptor (TGF-β R2), which then binds to and phosphorylates a second receptor (TGF-β R1, also named ALK5). This complex in turn phosphorylates and activates members of the SMAD family of proteins, which translocate to the nucleus and regulate the expression of target genes of this TGF-β pathway (Weiss, Alexander, and Liliana Attisano. “The TGFbeta superfamily signaling pathway.” Wiley Interdisciplinary Reviews: Developmental Biology 2.1 (2013) https://doi.org/10.1002/wdev.86).

Activation of the TGF-β pathway can lead to immune evasion of tumor cells through epithelial-to-mesenchymal transition (EMT) (Wang, G., Xu, D., Zhang, Z. et al. The pan-cancer landscape of crosstalk between epithelial-mesenchymal transition and immune evasion relevant to prognosis and immunotherapy response. npj Precis. Onc. 5, 56 (2021). https://doi.org/10.1038/s41698-021-00200-4). It can also lead to immunosuppression through direct suppressive effects on innate and adaptive immune cells, as well as stimulation of suppressive Tregs and MDSCs (de Streel, Grégoire, and Sophie Lucas. “Targeting immunosuppression by TGF-β1 for cancer immunotherapy.” Biochemical Pharmacology (2021) https://doi.org/10.1016/j.bcp.2021.114697). TGF-β additionally potently regulates the tumor microenvironment by altering levels of ECM proteins and signaling molecules, leading to immune cell exclusion (Ghahremanifard, P.; Chanda, A.; Bonni, S.; Bose, P. TGF-β Mediated Immune Evasion in Cancer Spotlight on Cancer-Associated Fibroblasts. Cancers 2020, 12, 3650. https://doi.org/10.3390/cancers12123650).

Granulosa cell tumors (GCTs) of the ovary represent ˜5% of malignant ovarian cancers and it has recently been reported that 95-97% of adult granulosa cell tumors carry a unique somatic mutation 402C>C in the FOXL2 gene (Jamieson, S., Butzow, R., Andersson, N. et al. The FOXL2 C134W mutation is characteristic of adult granulosa cell tumors of the ovary. Mod Pathol 23, 1477-1485 (2010). https://doi.org/i0.1038/modpathol.2010.145). The 402C:>G mutation results in an amino acid substitution of typtophan for cysteine (C134W) (Shah S P, Kobel M, Senz J, Morin R D, Clarke B A, et al. (2009) Mutation of FOXL2 in granulosa-cell tumors of the ovary, N Engl J Med 360: 2719-2729) which is located in the second wing on the surface of the forkhead domain. Computer modelling suggests this alteration does not disrupt the folding of the FOXL2 forkhead domain or its interactions with DNA. In addition, it has been shown that mutation does not affect the localisation of the FOXL2 protein (Benayoun B A, Caburet S, Dipietromaria A, Georges A, D'Haene B, et al. (2010) Functional exploration of the adult ovarian granulosa cell tumor-associated somatic FOXL2 mutation p.Cys134Trp (c.402C>G). PloS one 5: e8789). Therefore, it is believed that the pathogenicity of mutant FOXL2 occurs through changes to its interactions with other proteins. Such candidate proteins include the SMAD transcription factors and the effectors of TGF-β and BMP family signalling (Kobel M, Gilks C B, Huntsman D G (2009) Adult-type granulosa cell tumors and FOXL2 mutation. Cancer Res 69: 9160-9162). In addition, many of the transcriptional targets of mutant FOXL2 are known RT-β signalling genes. Therefore, deregulation of this key antiproliferative pathway is one-way mutant FOXL2 contribute to the pathogenesis of adult-type GCTs (Rosario R, Araki H, Print C G, Shelling A N (2012) The transcriptional targets of mutant FOXL2 in granulosa cell tumors. PloS one; https://doi.org/10.1371/journal.pone.0046270).

Activin receptor-like kinases have been implicated as an important therapeutic target in proliferative diseases such as cancer because of their roles in promoting tumor growth, survival, and metastasis. For example, many small molecule ALK-5 inhibitors have been shown to have anti-proliferative activity in a variety of cancer and tumor types. Small molecule SB-431542 was developed as an ALK-5 inhibitor and was found to inhibit other activin receptor-like kinases, ALK-4 and ALK-7. See, e.g., Inman et al., “SB-431542 is a Potent and Specific Inhibitor of Transforming Growth Factor-β Superfamily Type I Activin Receptor-Like Kinase (ALK) Receptors ALK4, ALK5, and ALK7”, Molecular Pharmacology, 2002, 62, 65. Additionally, small molecule ALK-4, ALK-5, and ALK-7 inhibitor A-83-01 was developed, and was found to inhibit SMAD signaling and epithelial-to-mesenchymal transition (EMT), suggesting that such inhibitors are useful for treating a variety of advanced-stage cancers. See, e.g., Tojo et al. “The ALK-5 inhibitor A-83-01 inhibits SMAD signaling and epithelial-to-mesenchymal transition by transforming growth factor-β”, Cancer Sci., 2005, 96, 791. In the same manner, the role of ALK-5 in TGF-β signaling may play a role in the production of cancer-associated fibroblasts and other fibrotic conditions. See for example, Blobe, G. C., et al., “Role of Transforming Growth Factor β in Human Disease”, N Engl J Med (342), pp 1350-1358 (2000); Ballester, B. et al, “Idiopathic Pulmonary Fibrosis and Lung cancer: Mechanisms and Molecular Targets”, Int. J. of Molecular Sciences 20(593), doi:10.3390/ijms20030593 (2019), Liu, L et al., “Smad2 and SMAD3 Have Differential Sensitivity in Relyaing TGFb Signaling and Inversely Regulate Early Linage Specification”, Scientific Reports [6:21602/DOI: 10.1038/srep21602], February 2015-14 pages, Huang, J. J. and Blobe, G. C., “Dichotomous Roles of TGF-β in Human Cancer”, Biochem Soc. Trans 342(2016); 1441-1454 (https://doi.org/10.1042/BST20160065), Akhurst, R. J. and Hata, A., “Targeting the TGF-β Signalling Pathway in Disease”, Nat. Rev. Drug Disc., 11 pp 790-811 (2012), Leslie, K. O., “Idiopathic Pulmonary Fibrosis May Be a Disease of Recurrent, Tractional Injury to the Periphery of the Aging Lung—A Unifying Hypothesis Regarding Etiology and Pathogenesis” Arch Pathol Lab Med (136) [[591-600 (2012), Knuppel, L. et al., “A Novel Antifibrotic Mechanism of Nintedanib and Pirfenidone—Inhibition of Collagen Fibril Assembly”, Am. J. of Resp. Cell and Mole. Bio. 1 (57), pp 77-90 (2017), Laping, N. J. et al., “Inhibition of TGF-b1-Induced Extracellular Matrix”, Mol. Pharmacol. Vol 62, No1, pp 580-64 (2002), Moore, B. B. and Moore, T. A., Viruses in Idiopathic Pulmonary Fibrosis—Etiology and Exacerbation, Ann Am Thorac. Soc., Vol 12 (Suppl 2) pp S186-S192 (2015)—[DOI: 10.1513/AnnalsATS.201502-088AW], Cho, M. E. and Kopp, J. B., “Pirfenidone: an Anti-Fibrotic and Cytoprotective Agent as Therapy for Progressive Kidney Disease”, Expert Opin. Investig. Drugs, 19(2), pp 275-283 (2010) [DOI:10.1517/13543780903501539], and B. Rybinski et al., “The Wound Healing, Chronic Fibrosis, and Cancer Progresion Triad, Physiol Genomics. 46(7); 2014, 223-244 PMID:24520152.

Galunisertib, a small molecule ALK-5 inhibitor, was found to inhibit tumor growth in a breast cancer model. Galunisertib in combination with a PD-L1 inhibitor showed tumor growth inhibition and regression in a colon carcinoma model, signaling synergy between ALK-5 inhibition and PD-1/PD-L1 inhibition. See, e.g., Holmgaard et al., “Targeting the TGFβ pathway with galunisertib, a TGFβRI small molecule inhibitor, promotes anti-tumor immunity leading to durable, complete responses, as monotherapy and in combination with checkpoint blockade”, Journal for ImmunoTherapy of Cancer, 2018, 6, 47. In addition, galunisertib has been under investigation for use in treating various other cancers, including glioblastoma, pancreatic carcinoma, hepatocellular carcinoma (HCC), and myelodysplastic syndromes, sometimes in combination with a PD-1/PD-L1 inhibitor. See, e.g., Herbertz et al., “Clinical development of galunisertib (LY2 IS7299 monohydrate), a small molecule inhibitor of transforming growth factor-beta signaling pathway”, Drug Design, Development, and Therapy, 2015, 9, 4479.

Another small molecule ALK-5 inhibitor, TEW-7197, also known as vactosertib, has also been under investigation for treating cancers such as melanoma, prostate cancer, breast cancer, HCC, and glioblastoma. See, e.g., Herbertz et al., “Clinical development of galunisertib (LY2 IS7299 monohydrate), a small molecule inhibitor of transforming growth factor-beta signaling pathway”, Drug Design, Development, and Therapy, 2015, 9, 4479.

ALK inhibitors, especially ALK-5 inhibitors, are promising therapeutics for a variety of indications that are still being explored. For example, studies have shown that TGFβR1/ALK-5 mutants can induce Foxp3 expression, which has been found to play a key role in the immune resistance of different tumor types, including pancreatic carcinoma. See, e.g., Hinz et al. “Foxp3 Expression in Pancreatic Carcinoma Cells as Novel Mechanism of Immune Evasion in Cancer”, Cancer Res. 2007, 67, 8344. Therefore, cancers that have traditionally been resistant to apoptosis via chemo- and/or radiation-based therapies may respond when combined with ALK-5 inhibition.

Research has also shown that ALK-5 inhibitors are also useful for treating proliferative diseases other than cancer, including systemic sclerosis and other fibrotic conditions, including fibrotic conditions associated with cancer, see for example, those conditions described in Mori et al. “Activin Receptor-Like Kinase 5 Signaling Blocks Profibrotic Transforming Growth Factor β Responses in Skin Fibroblasts”, Arthritis & Rheumatism, 2004, 8, 4008, Akhurst, R. J. and Hata, A., “Targeting the TGF-β Signalling Pathway in Disease”, Nat. Rev. Drug Disc., 11 pp 790-811 (2012), and Cox, T. R and Erler, J. T., “Molecular Pathways Connecting Fibrosis and Solid Tumor Methastasis”, Clin Cancer Res., 2014, 20(14), pp 3637-3643.

Epithelial to mesenchymal transition (ENT) is a term to describe epithelial cells losing their cell-to-cell adhesion and polarity while gaining migratory and invasive properties. These cells display a more undifferentiated mesenchymal phenotype that may then differentiate into a variety of other cell types. The process of EMT is required for normal embryonic development and wound healing but has also been implicated in disease such as cancer and fibrosis. In cancer, EMT is thought to contribute specifically to metastasis and resistance to chemotherapy. Hao, Y., et al, Int. J. dol. Sci. 2019 June; 20(11); 2767. TGF-beta, a pleiotropic cytokine, has been implicated as the main driver of EMT, and inhibiting this pathway may be beneficial in various diseases such as cancer and fibrosis. Katsuno, Y. and Derynck, R. Dev. Cell 2021 Mar. 22; 56(6):726-746.

Increased levels of ALK-5 have also been implicated in cardiac pathologies and cardiovascular disease, including not only cardiac remodeling and fibrosis, e.g., following myocardial infarction, and cardiac hypertrophy, but also dilated, ischemic and hypertrophic cardiomyopathies, valvular disease and arrhythmia, such as atrial fibrillation. Khan, R. and Sheppard, R. “Fibrosis in heart disease: understanding the role of transforming growth factor-β₁ in cardiomyopathy, valvular disease and arrhythmia”, Immunology 2006, 118:10-24; Bujak, M. and Frangogiannis, N. G., “The role of TGF-β in myocardial infarction and cardiac remodeling,” Cardiovascular Research 74 (2007), 184-195; Dobaczewski, M., et al., “Transforming Growth Factor (TGF)-β signaling in cardiac remodeling”, J. Mol. Cell Cardiol., 2011, 51(4):600-606; and Accornero, F., et al., “Genetic Analysis of Connective Tissue Growth Factor as an Effector of Transforming Growth Factor β Signaling and Cardiac Remodeling”, Molecular and Cellular Biology 2015, 35(12): 2154-2164.

Despite the progress made, additional compounds are needed to progress research and medical care of patients with proliferative diseases such as tumors and cancer, and fibrotic diseases, both those associated with proliferative diseases and those that are not associated with proliferative diseases.

### SUMMARY

Provided herein are inhibitors of activin receptor-like kinases (e.g., ALK-5), including compounds of any of the formulae herein and pharmaceutically acceptable salts thereof, pharmaceutical compositions and kits comprising the same, and methods of using any of the aforementioned compounds, salts, compositions and kits (e.g., for the treatment and/or prevention of disease in a subject). Also provided herein are methods of preparing the compounds, pharmaceutically acceptable salts and pharmaceutical compositions described herein.

In some embodiments there is provided compounds of Formula (I):

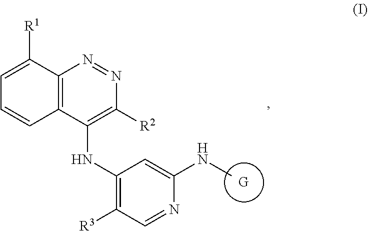

or a pharmaceutically acceptable salt thereof, wherein R¹, R², R³ and Ring G are as defined herein.

The compounds provided herein are activin receptor-like kinase (e.g., ALK-5) inhibitors useful for treating and/or preventing diseases (e.g., that involve regulating or targeting the TGFβ signaling pathway, for example, as it pertains to treatment, amelioration, or prevention of fibrotic, inflammatory and/or proliferative diseases (e.g., cancer, pulmonary fibrosis and cardiac diseases associated with TGFβ1 signaling)). See, for example, the relationship of these diseases and conditions and role of various signaling pathways that may be implicated in treating the same that are described in, for example, Akhurst, R. J. and Hata, A., “Targeting the TGF-β Signalling Pathway in Disease”, Nat. Rev. Drug Disc., 11 pp 790-811 (2012); Cox, T. R and Erler, J. T., “Molecular Pathways Connecting Fibrosis and Solid Tumor Metastasis”, Clin Cancer Res., 2014, 20(14), pp 3637-3643; Radisky, D. C., et al., “Fibrosis and Cancer: Do Myofibroblasts Come Also From Epithelial Cells via EMT?”, J. Cell Biochem., 2101(4), pp 830-839 [DOI: 10.1002/jcb.21186], and the role of viral complications in IPF, for example, as described in Moore, B. B. and Moore, T. A., Viruses in Idiopathic Pulmonary Fibrosis—Etiology and Exacerbation, Ann Am Thorac. Soc., Vol 12 (Suppl 2) pp S186-S192 (2015)—[DOI: 10.1513/AnnalsATS.201502-088AW], and the role of TGF signaling in cardiac remodeling described, for example, in Dobaczewski, M., et al., “Transforming Growth Factor (TGF)-β signaling in cardiac remodeling”, J. Mol. Cell Cardiol., 2011, 51(4):600-606.

In certain embodiments, the compounds provided herein are selective ALK-5 inhibitors, e.g., selective for ALK-5 over other kinases (e.g., over other activin receptor-like kinases, such as ALK-2, and/or JAK2). In certain embodiments, for example, a compound of Formula (I) is selected from the compounds recited in Table 1 (infra), and pharmaceutically acceptable salts thereof.

In various aspects and embodiments disclosed herein, express reference to a compound of Formula (I) is understood alternatively to refer to a compound of any disclosed subgenus thereof, for example, compounds of Formula (II) (infra), Formula (III) (infra), Formula (IV) (infra), a compound of Table 1 (infra), or any of the specific compounds disclosed herein.

In another aspect, provided herein are pharmaceutical compositions comprising a compound of Formula (I), or a pharmaceutically acceptable salt thereof, and one or more pharmaceutically acceptable carriers or excipients. In certain embodiments, a pharmaceutical composition provided herein comprises a therapeutically and/or prophylactically effective amount of a compound of Formula (I), or a pharmaceutically acceptable salt thereof. The pharmaceutical compositions described herein may be useful for treating and/or preventing a disease (e.g., an inflammatory, fibrotic, or proliferative disease, e.g., cancer or a combination of two or more thereof, as described further herein) in a subject. The pharmaceutical compositions provided herein may further comprise one or more additional therapeutic agents (e.g., anti-proliferative agents, e.g., anti-cancer agents).

In another aspect, provided herein are methods of treating and/or preventing a disease in a subject, the methods comprising administering to the subject a therapeutically and/or prophylactically effective amount of a compound of Formula (I) (II), (III) or (IV), or of Table 1, or any of said compounds in the form of a pharmaceutically acceptable salt, or a pharmaceutical composition of any of the foregoing. For example, provided herein are methods for treating a disease, for example, an inflammatory, fibrotic, or proliferative disease (e.g., cancer) in a subject, the methods comprising administering to the subject a therapeutically effective amount of a compound of Formula (I), or a pharmaceutically acceptable salt thereof, or a pharmaceutical composition comprising any of the foregoing, for example, a compound of Formula (I), Formula (II), Formula (III), Formula (IV), or Table 1, or any of the specific compounds disclosed herein, or any of the aforementioned compounds in the form of a pharmaceutically acceptable salt, or a pharmaceutical composition of any of the foregoing.

In certain embodiments, the proliferative disease is cancer. In certain embodiments, the proliferative disease is a solid tumor cancer. In some embodiments, the proliferative disease is a hematological cancer. In some embodiments, the cancer is associated with the activity (e.g., aberrant or increased activity) of an activin receptor-like kinase (e.g., ALK-5) in a subject or cell. In some embodiments, the cancer has associated with it a TGFβ signaling pathway that is critical to the progress of the disease and which can be ameliorated by ALK-5 inhibition. In some embodiments, the cancer has associated with it a FOXL2 mutation, for example, a tumor-associated somatic FOXL2 mutation p.Cys134Trp (c.402C>G). In some embodiments, the FOXL2 mutation affects one or more transcriptional targets which are TGF-β signalling genes.

In certain embodiments, the cancer is lung cancer (e.g., non-small cell lung cancer (NSCLC)), brain cancer (e.g., neuroblastoma, glioblastoma), thyroid cancer (e.g., anaplastic thyroid cancer (ATC)), breast cancer, colorectal cancer (e.g., colon carcinoma), liver cancer (e.g., hepatocellular carcinoma (HCC)), pancreatic cancer (e.g., pancreatic carcinoma), skin cancer (e.g., melanoma), prostate cancer, or a hematological cancer (e.g., anaplastic large cell lymphoma (ALCL), myelodysplastic syndrome (MDS), myelofibrosis (MF)). In certain embodiments, the cancer is myelofibrosis (MF).

In some embodiments, the proliferative disease is cancer, for example, anaplastic astrocytoma, pancreatic cancer, for example, pancreatic ductal adenocarcinoma and associated CAF, metastatic melanoma, colorectal cancer, breast cancer, prostate cancer, renal cancer, hepatocellular cancer, ovarian cancer, HPV-associated cancers (e.g., cervical cancer, oropharyngeal cancer, anal cancer, vulvar/vaginal cancer, penile cancer), multiple myeloma, myelodysplastic syndrome, or myelofibrosis. In some embodiments, the cancer is treated by targeting a tumor stromal cell (e.g., in a tumor microenvironment), such as a cancer-associated fibroblast (CAF), stellate cell or myofibroblast, and/or a tumor-associated immune cell (e.g., in the tumor-immune microenvironment), for example, to thereby modulate the tumor-stroma microenvironment and/or the tumor-immune microenvironment.

In some embodiments, the disease is a fibrotic condition, for example, idiopathic pulmonary fibrosis, cardiac fibrosis or a condition associated with cardiac fibrosis, liver fibrosis, liver cirrhosis, nonalcoholic steatohepatitis, Peyronie's, Dupuytren's contracture, cystic fibrosis, beta thalassemia, actinic keratosis, hypertension, general inflammatory disorders, dry eye, ulcers, corneal fibrosis, wet age-related macular degeneration, psoriasis, wound closure, chronic kidney disease, renal fibrosis, systemic sclerosis, or chronic Chagas' heart disease. In some embodiments, the fibrotic condition is cardiac fibrosis or a condition associated with cardiac fibrosis, for example, valvular disease, arrhythmia (e.g., atrial fibrillation), myocardial remodeling (e.g., after infarction), cardiomyopathy (e.g., dilated, ischaemic or hypertrophic cardiomyopathy), restenosis (e.g., in-stent restenosis, post-angioplasty restenosis). In some embodiments, the fibrotic condition is Dupuytren's contracture. In some embodiments, the fibrotic condition is, for example, acute exacerbation of idiopathic pulmonary fibrosis or familial pulmonary fibrosis, vascular fibrosis, kidney fibrosis (renal fibrosis), skin fibrosis (cutaneous fibrosis or endometrial fibrosis, e.g., keloids, scleroderma, or nephrogenic systemic fibrosis), gastrointestinal fibrosis (e.g., Crohn's disease), bone marrow fibrosis (myelofibrosis), athrofibrosis (e.g., of the knee, the shoulder or another joint), Dupuytren's contracture, mediastinal fibrosis, retroperitoneal fibrosis, systemic sclerosis, or autoimmune hepatitis. In some embodiments, the fibrotic condition is cancer-associated fibrosis; lung fibrosis, commonly known as “scarring of the lungs” (e.g., pulmonary fibrosis, for example, acute exacerbation of idiopathic pulmonary fibrosis or familial pulmonary fibrosis). In some embodiments, the fibrotic conditions is lung fibrosis, for example, pulmonary fibrosis, such as idiopathic pulmonary fibrosis, acute exacerbation of idiopathic pulmonary fibrosis or familial pulmonary fibrosis. In an embodiment, the liver fibrosis is hepatic fibrosis, e.g., keloids, scleroderma, nephrogenic systemic fibrosis, bile duct fibrosis (biliary fibrosis), or liver cirrhosis, for example, primary biliary cholangitis (biliary cirrhosis) or primary sclerosing cholangitis.

Also provided herein are methods of inhibiting or preventing tumor growth in a subject, the methods comprising administering to the subject a therapeutically effective amount of a compound of Formula (I), or a pharmaceutically acceptable salt thereof, or a pharmaceutical composition of the foregoing.

Also provided herein are methods of treating cachexia in a subject (e.g., a subject in need thereof), the methods comprising administering to the subject a therapeutically effective amount of a compound of Formula (I), or a pharmaceutically acceptable salt thereof, or a pharmaceutical composition comprising any of the foregoing, for example, a compound of Formula (I), Formula (II), Formula (III), Formula (IV), or Table 1, or any of the specific compounds disclosed herein, or any of the aforementioned compounds in the form of a pharmaceutically acceptable salt, or a pharmaceutical composition of any of the foregoing.

Also provided herein are methods for promoting immune infiltration in a tumor-immune microenvironment in a subject in need thereof, comprising administering to the subject a therapeutically effective amount of a compound of Formula (I), or a pharmaceutically acceptable salt thereof, or a pharmaceutical composition comprising any of the foregoing, for example, a compound of Formula (I), Formula (II), Formula (III), Formula (IV), or Table 1, or any of the specific compounds disclosed herein, or any of the aforementioned compounds in the form of a pharmaceutically acceptable salt, or a pharmaceutical composition of any of the foregoing.

Also provided herein are methods for inhibiting epithelial-to-mesenchymal transition in a tumor (e.g., in a subject in need thereof), comprising contacting the tumor with (e.g., an effective amount of) a compound of Formula (I), or a pharmaceutically acceptable salt thereof, or a pharmaceutical composition comprising any of the foregoing, for example, a compound of Formula (I), Formula (II), Formula (III), Formula (IV), or Table 1, or any of the specific compounds disclosed herein, or any of the aforementioned compounds in the form of a pharmaceutically acceptable salt, or a pharmaceutical composition of any of the foregoing. In some embodiments, the tumor is in a subject in need thereof and the method comprises administering to the subject a therapeutically effective amount of a compound of Formula (I), or a pharmaceutically acceptable salt thereof, or a pharmaceutical composition thereof.

Also provided herein are methods for modulating (e.g., promoting, upregulating) the antigen presentation pathway in a tumor (e.g., in a subject in need thereof), comprising contacting the tumor with (e.g., an effective amount of) a compound of Formula (I), or a pharmaceutically acceptable salt thereof, or a pharmaceutical composition comprising any of the foregoing, for example, a compound of Formula (I), Formula (II), Formula (III), Formula (IV), or Table 1, or any of the specific compounds disclosed herein, or any of the aforementioned compounds in the form of a pharmaceutically acceptable salt, or a pharmaceutical composition of any of the foregoing. In some embodiments, the tumor is in a subject in need thereof and the method comprises administering to the subject a therapeutically effective amount of a compound of Formula (I), or a pharmaceutically acceptable salt thereof, or a pharmaceutical composition thereof.

Also provided herein are methods of modulating the tumor-immune microenvironment in a subject, the methods comprising administering to the subject a therapeutically effective amount of a compound of Formula (I), or a pharmaceutically acceptable salt thereof, or a pharmaceutical composition comprising any of the foregoing, for example, a compound of Formula (I), Formula (II), Formula (III), Formula (IV), or Table 1, or any of the specific compounds disclosed herein, or any of the aforementioned compounds in the form of a pharmaceutically acceptable salt, or a pharmaceutical composition of any of the foregoing.

Also provided herein are methods increasing tumor vasculature or blood flow to a tumor or both in a subject, the methods comprising administering to the subject a therapeutically effective amount of a compound of Formula (I), or a pharmaceutically acceptable salt thereof, or a pharmaceutical composition comprising any of the foregoing, for example, a compound of Formula (I), Formula (II), Formula (III), Formula (IV), or Table 1, or any of the specific compounds disclosed herein, or any of the aforementioned compounds in the form of a pharmaceutically acceptable salt, or a pharmaceutical composition of any of the foregoing.

Also provided herein are methods of inhibiting metastasis of a cancer in a subject, the methods comprising administering to the subject a therapeutically effective amount of a compound of Formula (I), or a pharmaceutically acceptable salt thereof, or a pharmaceutical composition comprising any of the foregoing, for example, a compound of Formula (I), Formula (II), Formula (III), Formula (IV), or Table 1, or any of the specific compounds disclosed herein, or any of the aforementioned compounds in the form of a pharmaceutically acceptable salt, or a pharmaceutical composition of any of the foregoing.

Also provided herein are methods for inhibiting activin receptor-like kinase (e.g., ALK-5) activity in vivo or in vitro, the methods comprising contacting the activin receptor-like kinase (e.g., ALK-5) with a compound of Formulae (I) (II), (III), (IV), or Table 1, or a pharmaceutically acceptable salt thereof, or a pharmaceutical composition of the foregoing. In certain embodiments, the inhibition occurs in vivo in a subject. In certain embodiments, the inhibition occurs in vitro (e.g., in a cell line or biological sample). In certain embodiments, the inhibition is selective ALK-5 inhibition.

In another aspect, provided herein are compounds of Formulae (I) (II), (III), (IV), or Table 1, or a pharmaceutically acceptable salt thereof, or a pharmaceutical composition of the foregoing, for a use described herein, including, but not limited to, treating and/or preventing a disease (e.g., a proliferative disease, e.g., cancer, a fibrotic disease, e.g., a cardiac fibrosis or hypertrophic condition, or an inflammatory condition) in a subject, for inhibiting tumor growth in a subject, or for inhibiting activin receptor-like kinase (e.g., ALK-5) activity in vitro or in vivo. In yet another aspect, provided herein are uses of compounds of Formulae (I) (II), (III), (IV), or Table 1, pharmaceutically acceptable salts thereof, and pharmaceutical compositions of the foregoing, for the preparation of medicaments for treating and/or preventing a disease (e.g., an inflammatory condition, a fibrotic disease, e.g., a cardiac fibrosis or hypertrophic condition, or proliferative disease, e.g., cancer or two or more thereof in combination) in a subject, for inhibiting tumor growth in a subject, or for inhibiting ALK-5 activity in a subject.

The methods and uses provided herein may further comprise administering one or more additional therapeutic agents (e.g., anti-cancer agents or immunotherapies or other agents described herein) to the subject. In certain embodiments, a PD-1 or PD-L1 inhibitor is administered in combination with a compound, pharmaceutically acceptable salt or pharmaceutical composition described herein. The methods provided herein may further comprise treating the subject with radiation therapy or surgery.

Also provided herein are methods for enhancing the activity of one or more therapeutic agents for treating cancer (e.g., an anti-cancer agent and/or immunotherapy) in a subject (e.g., a subject in need thereof, such as a subject having cancer and/or receiving the one or more therapeutic agents), comprising administering to the subject a therapeutically effective amount of a compound of Formulae (I) (II), (III), (IV), or Table 1), or a pharmaceutically acceptable salt thereof, or a pharmaceutical composition thereof.

In another aspect, provided herein are kits comprising a compound of Formula (I), or a pharmaceutically acceptable salt thereof, or pharmaceutical composition of the foregoing. The kits described herein may include a single dose or multiple doses of the compound, pharmaceutically acceptable salt or pharmaceutical composition. The provided kits may be useful in a method of the disclosure (e.g., a method of treating and/or preventing a disease in a subject). A kit of the disclosure may further include instructions for using the kit (e.g., instructions for using the compound, pharmaceutically acceptable salt or composition included in the kit).

Also provided herein are methods of preparing compounds of the present disclosure, for example, compounds of Formulae (I) (II), (III), (IV), or Table 1, and pharmaceutically acceptable salts thereof.

### BRIEF DESCRIPTION OF THE DRAWINGS

The patent or application file contains at least one drawing executed in color. Copies of this patent or patent application publication with color drawings will be provided by the Office upon request and payment of the necessary fee.

The accompanying Figures, which are incorporated in and constitute a part of this specification, and may illustrate several embodiments of the disclosure and together with the description, may provide non-limiting examples of the disclosure.

FIG. 1A is a graphic illustration of the LanthaScreen™ Eu Kinase Binding Assay.

FIG. 1B shows ALK5 inhibition of selected compounds as a function of compound concentration using RDSR cellular-based assay techniques described in Example 3.

FIG. 2A shows that Compound No. 04 showed a full concentration-dependent inhibition of TGF-β1-mediated αSMA expression in all three IPF donors. Data are displayed as normalized data for αSMA staining (percentage inhibition, PIN) and nuclear count (percent remaining cells). Marginal loss (<25%) of nuclei observed in donor IPF03 at the highest test concentration (10 μM). No modulation of the number of nuclei was observed in donors IPF06 and IPF08, indicative of absence of potential cytotoxicity or anti-proliferative effects.

FIG. 2B shows that Compound No. 01 showed a full concentration-dependent inhibition of TGF-β1-mediated αSMA expression in all three IPF donors. Data are displayed as normalized data for αSMA staining (percentage inhibition, PIN) and nuclear count (percent remaining cells). Loss of nuclei (>25%) observed at the highest test concentration (10 μM) in donor IPF03 indicative of potential cytotoxicity or anti-proliferative effects at this concentration. No modulation of the number of nuclei was observed in donors IPF06 and IPF08, indicative of absence of potential cytotoxicity or anti-proliferative effects.

FIG. 3A shows p-SMAD-2 levels normalized to β-tubulin in tumors harvested from mice treated with the indicated compound in the A549 xenograft assay.

FIG. 3B shows p-STAT3 levels normalized to β-tubulin in tumors harvested from mice treated with the indicated compound in the A549 xenograft assay.

FIG. 4A shows p-SMAD-2 levels normalized to GAPDH in tumors harvested from mice treated with 50 mpk (PO) of the indicated compound in the longitudinal A549 xenograft study described in Example 8.

FIG. 4B shows PK/PD profile of vactosertib in the longitudinal A549 xenograft study described in Example 8.

FIG. 4C shows PK/PD profile of Compound 04 in the longitudinal A549 xenograft study described in Example 8.

FIG. 5 shows inhibition of SMAD signaling triggered by myostatin in the presence of Compound 01, Compound 04 or vactosertib.

FIG. 6A shows relative p-SMAD-2 levels normalized to GAPDH (as a percentage of vehicle) in tumors harvested from mice treated with 50 mpk (PO) of the indicated compound in the longitudinal A549 xenograft study described in Example 10.

FIG. 6B shows PK/PD profile of vactosertib in the longitudinal A549 xenograft study described in Example 10.

FIG. 6C shows PK/PD profile of Compound 04 in the longitudinal A549 xenograft study described in Example 10.

FIG. 6D shows PK/PD profile of Compound 01 in the longitudinal A549 xenograft study described in Example 10.

FIG. 7A shows the overall study design of the study described in Example 11.

FIG. 7B shows the amount of hydroxyproline measured from a portion of lung tissue harvested from mice remaining on day twenty-one of the study described in Example 11.

FIG. 7C shows histological analysis applying the modified Ashcroft scale of five randomly chosen animals from each treatment group in the study described in Example 11.

FIG. 7D shows representative images of lung tissue stained with hematoxylin and eosin (H&E) (top row) or Masson's trichrome (bottom row) stain obtained from the indicated treatment group in the study described in Example 11.

FIG. 7E shows representative images of lung tissue stained with H&E (top row) or Masson's trichrome (bottom row) stain obtained from the indicated treatment group in the study described in Example 11.

FIG. 8A shows fold-change in CDH1 (E-Cadherin) gene expression level in A549 lung fibroblasts upon treatment with the indicated compound in the assay described in Example 12.

FIG. 8B shows fold-change in CDH2 (N-Cadherin) gene expression level in A549 lung fibroblasts upon treatment with the indicated compound in the assay described in Example 12.

FIG. 8C shows fold-change in SNAI1 (Snail) gene expression level in A549 lung fibroblasts upon treatment with the indicated compound in the assay described in Example 12.

FIG. 8D shows fold-change in SNAI2 (Slug) gene expression level in A549 lung fibroblasts upon treatment with the indicated compound in the assay described in Example 12.

FIG. 8E shows fold-change in VIM (vimentin) gene expression level in A549 lung fibroblasts upon treatment with the indicated compound in the assay described in Example 12.

FIG. 8F shows fold-change in SPARC gene expression level in A549 lung fibroblasts upon treatment with the indicated compound in the assay described in Example 12.

FIG. 8G shows fold-change in GALNT6 gene expression level in A549 lung fibroblasts upon treatment with the indicated compound in the assay described in Example 12.

FIG. 8H shows fold-change in CTNNB1 (β-catenin) gene expression level in A549 lung fibroblasts upon treatment with the indicated compound in the assay described in Example 12.

FIG. 8I shows fold-change in TGFB1 gene expression level in A549 lung fibroblasts upon treatment with the indicated compound in the assay described in Example 12.

FIG. 8J shows fold-change in MAML3 gene expression level in A549 lung fibroblasts upon treatment with the indicated compound in the assay described in Example 12.

FIG. 9A shows percent body weight change and subject outcome of subject treated with Compound 01 or Compound 04 in the acute MTD study described in Example 13.

FIG. 9B shows percent body weight change and subject outcome of subjects treated with Compound 04 in the chronic MTD study described in Example 13.

FIG. 9C shows percent body weight change and subject outcome of subjects treated with Compound 01 in the chronic MTD study described in Example 13.

FIG. 9D shows survival outcome of subjects treated with Compound 04 in the chronic MTD study described in Example 13.

FIG. 9E shows survival outcome of subjects treated with Compound 01 in the chronic MTD study described in Example 13.

FIG. 10 shows median fluorescent intensity (MFI) of phospho-SMAD2 as a function of concentration of the indicated compound from the phospho-SMAD2 assay described in Example 14.

FIG. 11 shows the results of the JAK selectivity assay described in Example 15

FIG. 12 shows the results of the HepaRG™ NP 3D model of fibrosis described in Example 16.

### DETAILED DESCRIPTION

A description of example embodiments follows.

Provided herein are compounds (e.g., compounds of Formulae (I), (II), (III), (IV), or Table 1 or any of the compounds specifically exemplified herein, herein also referred to as “the exemplified compounds”), and pharmaceutically acceptable salts thereof, pharmaceutical compositions of the foregoing, and kits comprising one or more of the foregoing. The compounds provided herein are activin receptor-like kinase (e.g., ALK-5) inhibitors and are, therefore, useful for treating and/or preventing diseases (e.g., proliferative diseases, e.g., cancer, fibrotic diseases, inflammatory diseases) in a subject, for inhibiting tumor growth in a subject, and/or for inhibiting the activity of an activin receptor-like kinase (e.g., ALK-5) in vitro or in vivo. In certain embodiments, the compounds provided herein are ALK-5 inhibitors (e.g., selective ALK-5 inhibitors). Also provided herein are methods and synthetic intermediates useful in the preparation of compounds described herein.

### Definitions

The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

Unless specified otherwise, reference herein to a “compound of the present disclosure” and the like refers to a compound of any structural formula depicted herein (e.g., a compound of Formula (I), a subformula of a compound of Formula (I)), includes the compound depicted as well as isomers, such as stereoisomers (including diastereoisomers, enantiomers and racemates), geometrical isomers, conformational isomers (including rotamers and astropisomers), tautomers, isotopically labeled compounds (including deuterium substitutions), and inherently formed moieties (e.g., polymorphs and/or solvates, such as hydrates) thereof. When a moiety is present that is capable of forming a salt, then salts are included as well, in particular, pharmaceutically acceptable salts.

Compounds of the present disclosure may have asymmetric centers, chiral axes, and chiral planes (e.g., as described in: E. L. Eliel and S. H. Wilen, Stereo-chemistry of Carbon Compounds, John Wiley & Sons, New York, 1994, pages 1119-1190), and occur as racemic mixtures, individual isomers (e.g., diastereomers, enantiomers, geometrical isomers, conformational isomers (including rotamers and atropisomers), tautomers) and intermediate mixtures, with all possible isomers and mixtures thereof being included in the present disclosure.

As used herein, the term “isomers” refers to different compounds that have the same molecular formula but differ in arrangement and configuration of the atoms.

“Enantiomers” are a pair of stereoisomers that are non-superimposable mirror images of each other. A 1:1 mixture of a pair of enantiomers is a “racemic” mixture. “Racemate” or “racemic” is used to designate a racemic mixture where appropriate. When designating the stereochemistry for the compounds of the present disclosure, a single stereoisomer with known relative and absolute configuration of the two chiral centers is designated using the conventional RS system (e.g., (1S,2S)); a single stereoisomer with known relative configuration but unknown absolute configuration is designated with stars (e.g., (1R*,2R*)); and a racemate with two letters (e.g., (1RS,2RS) as a racemic mixture of (1R,2R) and (1S,2S); (1RS,2SR) as a racemic mixture of (1R,2S) and (1S,2R)). “Diastereoisomers” are stereoisomers that have at least two asymmetric atoms, but which are not mirror-images of each other. The absolute stereochemistry is specified according to the Cahn-Ingold-Prelog R-S system. When a compound is a pure enantiomer, the stereochemistry at each chiral carbon may be specified by either R or S. Resolved compounds whose absolute configuration is unknown can be designated (+) or (−) depending on the direction (dextro- or levorotatory) which they rotate plane polarized light at the wavelength of the sodium D line. Alternatively, the resolved compounds can be defined by the respective retention times for the corresponding enantiomers/diastereomers via chiral HPLC.

Geometric isomers may occur when a compound contains a double bond or some other feature that gives the molecule a certain amount of structural rigidity. If the compound contains a double bond, the double bond may be E- or Z-configuration. If the compound contains a disubstituted cycloalkyl, the cycloalkyl substituent may have a cis- or trans-configuration.

Conformational isomers (or conformers) are isomers that can differ by rotations about one or more bonds. Rotamers are conformers that differ by rotation about only a single bond.

The term “atropisomer,” as used herein, refers to a structural isomer based on axial or planar chirality resulting from restricted rotation in the molecule.

Optically active (R)- and (S)-isomers may be prepared using chiral synthons or chiral reagents, or resolved using conventional techniques (e.g., separated on chiral SFC or HPLC chromatography columns, such as CHIRALPAK® and CHIRALCEL® columns available from DAICEL Corp. or other equivalent columns, using the appropriate solvent or mixture of solvents to achieve suitable separation).

The compounds of the present disclosure can be isolated in optically active or racemic forms. Optically active forms may be prepared by resolution of racemic forms or by synthesis from optically active starting materials. All processes used to prepare compounds of the present disclosure and intermediates made therein are considered to be part of the present disclosure. When enantiomeric or diastereomeric products are prepared, they may be separated by conventional methods, for example, by chromatography or fractional crystallization.

Depending on the process conditions, the end products of the present disclosure are obtained either in free (neutral) or salt form. Both the free form and the salts of these end products are within the scope of the present disclosure. If so desired, one form of a compound may be converted into another form. A free base or acid may be converted into a salt; a salt may be converted into the free compound or another salt; a mixture of isomeric compounds of the present disclosure may be separated into the individual isomers.

Unless otherwise indicated, any formula given herein is also intended to represent unlabeled forms as well as isotopically labeled forms of the compounds. Isotopically labeled compounds have structures depicted by the formulas given herein except that one or more atoms are replaced by an atom having a selected atomic mass or mass number. Examples of isotopes that can be incorporated into compounds of the present disclosure include isotopes of hydrogen, carbon, nitrogen, oxygen, phosphorous, fluorine, chlorine and iodine, such as ²H ³H, ¹¹C, ¹³C, ¹⁴C, ¹⁵N, ¹⁸F, ³¹P, ³²p, ³⁵S, ³⁶Cl, ¹²³I, ¹²⁴I and ¹²⁵I, respectively. The present disclosure includes various isotopically labeled compounds as defined herein, for example those into which radioactive isotopes, such as ³H and ¹⁴C, or those into which non-radioactive isotopes, such as ²H and ¹³C are present. Such isotopically labelled compounds are useful in metabolic studies (with ¹⁴C), reaction kinetic studies (with, for example ²H or ³H), detection or imaging techniques, such as positron emission tomography (PET) or single-photon emission computed tomography (SPECT) including drug or substrate tissue distribution assays, or in radioactive treatment of patients. In particular, an ¹⁸F or labeled compound may be particularly desirable for PET or SPECT studies.

Further, substitution with heavier isotopes, particularly deuterium (i.e., ²H or D) may afford certain therapeutic advantages resulting from greater metabolic stability, for example, increased in vivo half-life or reduced dosage requirements or an improvement in therapeutic index. It is understood that deuterium in this context is regarded as a substituent of a compound of the present disclosure. The concentration of such a heavier isotope, specifically deuterium, may be defined by the isotopic enrichment factor. The term “isotopic enrichment factor,” as used herein, means the ratio between the isotopic abundance and the natural abundance of a specified isotope. If a substituent in a compound of this present disclosure is denoted deuterium, such compound has an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation).

Isotopically labeled compounds of the present disclosure can generally be prepared by conventional techniques known to those skilled in the art or by processes disclosed in the schemes or in the examples and preparations described below (or analogous processes to those described hereinbelow), by substituting an appropriate or readily available isotopically labeled reagent for a non-isotopically labeled reagent otherwise employed. Such compounds have a variety of potential uses, e.g., as standards and reagents in determining the ability of a potential pharmaceutical compound to bind to target proteins or receptors, or for imaging compounds of this disclosure bound to biological receptors in vivo or in vitro.

When a range of values is listed, it is intended to encompass each value and sub-range within the range. For example, “C₁₋₆ alkyl” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

The term “alkyl” refers to a radical of a straight-chain or branched saturated hydrocarbon group. In some embodiments, an alkyl group has 1 to 6 carbon atoms (“C₁₋₆ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆ alkyl”). Examples of C₁₋₆ alkyl groups include methyl (C₁), ethyl (C₂), propyl (C₃) (e.g., n-propyl, iso-propyl), butyl (C₄) (e.g., n-butyl, tert-butyl, sec-butyl, iso-butyl), pentyl (C) (e.g., n-pentyl, 3-pentanyl, amyl, neopentyl, 3-methyl-2-butanyl, tertiary amyl), and hexyl (C₆) (e.g., n-hexyl and all branched alkyls comprising 6 carbon atoms), and the like. When an alkyl group is defined as being “substituted” herein, in conjunction with any limitations presented at the point of definition herein, and unless otherwise specified, a “substituted alkyl” indicates that one or more positions on the carbon backbone of the alkyl group normally occupied by a proton is replaced with another substituent (e.g., a methyl group which is substituted by one or more halogen includes —F, —Cl, and/or —Br and, for example, when substituted with F, includes —CH₂F, —CHF₂, and —CF₃).

The term “carbocyclyl”, “carbocycle” or “carbocyclic” refers to a non-aromatic cyclic hydrocarbon substituent (meaning the defining ring contains no heteroatoms), where the defining ring has the specified number of ring carbon atoms in a monocyclic, bicyclic, bridged, or spirocyclic configuration. While carbocycles are non-aromatic, they may contain one or more double bonds located within the ring such that they aren't conjugated. In some embodiments one or more of the ring carbon atoms may be oxidized (e.g., a cycloketone). In some embodiments, a carbocycle group (moiety) has 3 to 10 ring carbon atoms (“C₃₋₁₀ carbocycle”). In some embodiments, a carbocycle group has 3 to 8 ring carbon atoms (“C₃₋₈ carbocycle”). In some embodiments, a carbocycle group has 3 to 7 ring carbon atoms (“C₃₋₇ carbocycle”). In some embodiments, a carbocycle group has 3 to 6 ring carbon atoms (“C₃₋₆ carbocycle”). In some embodiments, a carbocycle group has 4 to 6 ring carbon atoms (“C₄₋₆ carbocycle”). Exemplary C₃₋₆ carbocycle groups include, without limitation, cyclopropyl (C₃), cyclopropenyl (C₃), cyclobutyl (C₄), cyclobutenyl (C₄), cyclopentyl (C), cyclopentenyl (C₅), cyclohexyl (C₆), cyclohexenyl (C₆), cyclohexadienyl (C₆), and the like. In some embodiments, the carbocycle group is a cyclopropyl (C₃). As the foregoing examples illustrate, in certain embodiments, the carbocycle group is either monocyclic (“monocyclic carbocycle”) or polycyclic (e.g., containing a fused, bridged or spiro ring system such as a bicyclic system (“bicyclic carbocycle”) or tricyclic system (“tricyclic carbocycle”)) and can be saturated or can contain one or more carbon-carbon double or triple bonds. In some embodiments, the carbocycle group is a bicyclic carbocycle, for example a spiro ring preferably comprising from 6 to 9 carbon atoms. It will be understood that the minimum number of carbon atoms in a bicyclic carbocycle is four, and the minimum number of carbon atoms in a spirocyclic carbocycle is five. Thus, it will be understood that recitation of a monocyclic, bicyclic or spirocyclic C₃-C₁₀ carbocycle refers to a monocyclic C₃-C₁₀ carbocyclyl, bicyclic C₄-C₁₀ carbocyclyl or spirocyclic C₅-C₁₀ carbocyclyl. In some embodiments of a spirocyclic carbocyclyl, the carbocycle is preferably a C₅₋₁₀ spirocyclic carbocyclyl, e.g., C₆₋₉ spirocyclic carbocyclyl.

The term “hydroxy” or “hydroxyl” refers to —OH.

The term “heterocyclyl”, “heterocycle” or “heterocyclic” refers to a non-aromatic substituent defined by a ring having the specified number of atoms selected from carbon atoms and at least 1, up to 3, heteroatoms which are the same, or independently selected from, N, S, and O, selected to be bonded such that they form a stable chemical entity. Thus, “C₅-C₁₀ heterocyclyl” refers to a heterocyclyl group having from 5 to 10 ring atoms selected from carbon atoms and at least 1, up to 3, heteroatoms which are the same, or independently selected from, N, S, and O, selected to be bonded such that they form a stable chemical entity. The heterocycle ring may be saturated or may contain one or more sites of unsaturation so long as the bonding pattern does not provide aromatic delocalization. Heterocycle cores can either be monocyclic (“monocyclic heterocycle”) or polycyclic (e.g., a fused, bridged or spiro ring system such as a bicyclic system (“bicyclic heterocycle”) or tricyclic system (“tricyclic heterocycle”)) so long as at least one cyclic moiety defined by ring members contains a heteroatom, and polycyclic heterocycle substituents can, but need not, include one or more heteroatoms in multiple rings. If a heterocycle is indicated to be substituted, substituent(s) bonded to the “substituted heterocycle” core can be bonded via any of the ring member atoms that provide a stable bonding arrangement. In certain embodiments, the heterocycle group is an unsubstituted 3-10 membered heterocycle. In certain embodiments, the heterocycle group is a substituted 3-10 membered heterocycle. In some embodiments, a heterocyclyl group has from 5 to 10 ring atoms. In some embodiments it is preferred to select heterocycle substituents which are 6-membered ring systems. In some embodiments, it is preferred to select heterocycle substituents which are 10-membered spirocycle substituents. It will be understood that the minimum number of ring atoms in a bicyclic heterocycle is four, and the minimum number of ring atoms in a spirocyclic heterocycle is five. Thus, it will be understood that recitation of a monocyclic, bicyclic or spirocyclic C₃-C₁₀ heterocycle refers to a monocyclic C₃-C₁₀ heterocyclyl, bicyclic C₄-C₁₀ heterocyclyl or spirocyclic C₅-C₁₀ heterocyclyl. In some embodiments of a spirocyclic heterocyclyl, the heterocycle is preferably a C₅₋₁₀ spirocyclic heterocyclyl, e.g., C₆₋₉ spirocyclic heterocyclyl.

The term “aryl” refers to an aromatic moiety of up to 10 carbon atoms defining the aromatic ring system. Such substituents are bonded to a substrate via any ring carbon atom providing a stable structure. As defined or limited at the point of use, these moieties may comprise monocyclic or bicyclic structures (e.g., fused rings). In some embodiments, an aryl group has 6 ring carbon atoms (“C₆ aryl”; e.g., phenyl). In some embodiments, an aryl group has 10 ring carbon atoms (“C₁₀ aryl”; e.g., naphthyl such as 1-naphthyl and 2-naphthyl). In some embodiments, as defined or illustrated at the point of use herein, an aryl moiety includes substituents on the aryl ring, as defined above, which are bonded to form a fused carbocyclic structure with the aryl moiety, the size of the carbocyclic ring in the fused structure being defined at the point of use. If an aryl moiety is defined herein as substituted, it means the specified substituents may replace one or more protons bonded to a carbon atom defining the aryl ring in a manner the provides a stable species. In some embodiments, aryl moieties are 6-membered aryl rings (e.g., optionally substituted phenyl).

The term “heteroaryl” refers to an aromatic moiety of up to 10 carbon atoms defining the aromatic ring system wherein one or more of the atoms defining said aromatic ring system are independently selected from O, N or S. Heteroaryl substituents may be bonded to the substrate via any atom in the heteroaryl ring that affords a stable bond. Heteroaryl substituents may optionally be substituted as defined at the point of use herein. In some embodiments, a heteroaryl group has from 5 to 10 ring atoms (“C₅-C₁₀ heteroaryl”). In some embodiments, a heteroaryl group has from 6 to 10 ring atoms (“C₆-C₁₀ heteroaryl”), for example, 6 ring atoms (“C₆ heteroaryl”; e.g., pyridinyl) or 10 ring atoms (“C₁₀ heteroaryl”).

The term “optionally substituted” used in substituent definitions herein indicates that the defined moiety may be present without any substituents (i.e., unsubstituted) or may be present in a form having one or more bonding positions therein normally occupied by a proton being replaced (i.e., substituted) with one or more of the specified optional substituents. In all embodiments, when optional substituents are present, they are present in an amount and a bonding configuration that provides stable compounds, e.g., a compound which does not spontaneously undergo transformation such as by rearrangement, cyclization, elimination, or other reaction; however, it does contemplate arrangements which provide tautomers or other like bonding arrangements. Unless otherwise indicated, a “substituted” moiety has a substituent at one or more substitutable positions of the moiety, and when more than one position in any given structure is substituted, the substituent is independently selected from the stated allowable substituents. Unless defined differently at the point of use, the term “substituted” is contemplated to include substitution with all permissible substituents of organic compounds, and includes any of the substituents described herein that results in the formation of a stable compound. The present disclosure contemplates any and all such combinations in order to arrive at a stable compound. For purposes of this disclosure, heteroatoms such as nitrogen may have hydrogen substituents and/or any suitable substituent as described herein which satisfy the valencies of the heteroatoms and results in the formation of a stable moiety. In some embodiments, where a trivalent nitrogen can be quaternized or where a quaternary nitrogen can be deprotonated to a trivalent form, a representation of either form contemplates the transformation between the two forms and such representation is not intended to be limited in any manner by the exemplary substituents described herein.

As used herein, unless specified differently at the point of definition, the term “halo” or “halogen” refers to fluorine (fluoro, —F), chlorine (chloro, —Cl), bromine (bromo, —Br), or iodine (iodo, —I) unless the term is more limited at the point of use herein. In some embodiments, halo is fluorine, chlorine or bromine. In some embodiments, halo is fluorine or chlorine.

The term “sulfonamide” refers to —SO₂R′R″, wherein R′ and R″ are the same or different, and are each independently selected from hydrogen, alkyl or carbocyclyl. In some embodiments, R′ and R″ are each independently selected from hydrogen, C₁-C₅ alkyl or C₃-C₅ cycloalkyl. In some embodiments, sulfonamide is —SO₂NH₂.

In certain embodiments, certain features of compound substituents may be protected with a protecting group known to the ordinarily skilled practitioner, for example, those described in detail in Protecting Groups in Organic Synthesis, T. W. Greene and P. G. M. Wuts, 3^{rd} edition, John Wiley & Sons, 1999, incorporated herein by reference. All such transformations are contemplated by representation of the unprotected form of the compound.

As used herein, the term “salt” refers to any and all salt forms that compounds disclosed herein can be prepared as, and encompasses pharmaceutically acceptable salts. Pharmaceutically acceptable salts are preferred. However, other salts may be useful, e.g., in isolation or purification steps which may be employed during preparation, and thus, are contemplated to be within the scope of the present disclosure. In general, salts of a compound described herein will be those that provide a composition suitable for administration to a human or animal subject via any suitable route of administration of a pharmaceutical composition.

The phrase “pharmaceutically acceptable” means that the substance or composition the phrase modifies must be, within the scope of sound medical judgment, suitable for use in contact with the tissues of humans and lower animals without undue toxicity, irritation, allergic response and the like, and are commensurate with a reasonable benefit/risk ratio. If a substance is part of a composition or formulation, the substance must also be compatible chemically and/or toxicologically with the other ingredients in the composition or formulation.

The term “pharmaceutically acceptable salt” refers to those salts which are, within the scope of sound medical judgment, suitable for use in contact with the tissues of humans and lower animals without undue toxicity, irritation, allergic response, and the like, and are commensurate with a reasonable benefit/risk ratio. Pharmaceutically acceptable salts are well known in the art. For example, Berge et al. describe pharmaceutically acceptable salts in detail in J. Pharmaceutical Sciences, 1977, 66, 1-19, incorporated herein by reference, and for example, lists of suitable salts are found in Allen, L. V., Jr., ed., Remington: The Science and Practice of Pharmacy, 22nd Edition, Pharmaceutical Press, London, UK (2012).

Pharmaceutically acceptable salts include those derived from suitable inorganic and organic acids and inorganic and organic bases. Examples of pharmaceutically acceptable, nontoxic acid addition salts are salts of an amino group formed with inorganic acids, such as hydrochloric acid, hydrobromic acid, phosphoric acid, sulfuric acid, and perchloric acid or with organic acids, such as acetic acid, oxalic acid, maleic acid, tartaric acid, citric acid, succinic acid, or malonic acid or by using other methods known in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium, and N⁺(C₁₋₄ alkyl)₄⁻ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate. Compounds described herein are also provided, and can be administered, as a free base. In general, salts of a compound described herein will be those that provide a composition suitable for administration to a human or animal subject via any suitable route of administration of a pharmaceutical composition comprising the salt.

A salt (e.g., pharmaceutically acceptable salt) of a compound described herein can be synthesized from the parent compound that contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred.

It will be understood that when the compound described herein contains more than one basic moiety or more than one acidic moiety, each such moiety can independently be involved in forming an acid addition salt form or base addition salt form, with all possible salt forms being included in this disclosure. Further, when two or more moieties of a compound are in salt form, the anions or cations forming the two or more salt forms can be the same or different. Typically, the anions or cations forming the two or more salt forms are the same. Typical molar ratios of an anion or cation in a salt of a compound of the present disclosure to a compound described herein are 3:1, 2:1, 1:1, 2:1, 3:1, 4:1 and 5:1. In some embodiments, the molar ratio of an anion or cation (e.g., anion) in a salt of a compound described herein to the compound is 1:1.

Compounds described herein are also provided, and can be administered, as a free base.

A “pharmaceutically acceptable carrier” refers to media generally accepted in the art for the delivery of biologically active agents to animals, in particular, mammals, including, generally recognized as safe (GRAS) solvents, dispersion media, coatings, surfactants, antioxidants, preservatives (e.g., antibacterial agents, antifungal agents), isotonic agents, absorption delaying agents, salts, preservatives, drug stabilizers, binders, buffering agents (e.g., maleic acid, tartaric acid, lactic acid, citric acid, acetic acid, sodium bicarbonate, sodium phosphate, and the like), disintegration agents, lubricants, sweetening agents, flavoring agents, dyes, and the like, and combinations thereof, as would be known to those skilled in the art (see, for example, Allen, L. V., Jr. et al., Remington: The Science and Practice of Pharmacy (2 Volumes), 22nd Edition, Pharmaceutical Press (2012).

The terms “composition” and “formulation” are used interchangeably.

A “subject” to which administration is contemplated refers to a human (i.e., male or female of any age group, e.g., pediatric subject (e.g., infant, child, or adolescent) or adult subject (e.g., young adult, middle-aged adult, or senior adult)) or non-human animal. In certain embodiments, the non-human animal is a mammal (e.g., primate (e.g., cynomolgus monkey or rhesus monkey), commercially relevant mammal (e.g., cattle, pig, horse, sheep, goat, cat, or dog)), or bird (e.g., commercially relevant bird, such as chicken, duck, goose, or turkey). In certain embodiments, the non-human animal is a fish, reptile, or amphibian. The non-human animal may be a male or female at any stage of development. The non-human animal may be a transgenic animal or genetically engineered animal. The term “patient” refers to a human subject in need of treatment of a disease.

The term “administer,” “administering,” or “administration” refers to implanting, absorbing, ingesting, injecting, inhaling, or otherwise introducing a compound, or a pharmaceutically acceptable salt thereof, or a composition of the foregoing, in or on a subject.

The terms “treatment,” “treat,” and “treating” refer to administration of a medication or medical care to a subject, such as a human, having a disease or condition of interest, e.g., a cancer, and includes: (i) preventing the disease or condition from occurring in a subject, in particular, when such subject is predisposed to the condition but has not yet been diagnosed as having it; (ii) inhibiting the disease or condition, e.g., arresting its development; (iii) relieving the disease or condition, e.g., causing regression of the disease or condition; and/or (iv) relieving the symptoms resulting from the disease or condition (e.g., pain, weight loss, cough, fatigue, weakness, etc.). Treating thus includes reversing, alleviating, delaying the onset of, and/or inhibiting the progress of a disease (e.g., a disease described herein). In some embodiments, treatment may be administered after one or more signs or symptoms of the disease have developed or have been observed. In other embodiments, treatment may be administered in the absence of signs or symptoms of the disease. For example, treatment may be administered to a susceptible subject prior to the onset of symptoms. Treatment may also be continued after symptoms have resolved, for example, to delay or prevent recurrence.

An “effective amount” of a compound refers to an amount sufficient to elicit the desired biological response. An effective amount of a compound may vary depending on such factors as the desired biological endpoint, the pharmacokinetics of the compound, the condition being treated, the mode of administration, and the age and health of the subject. In certain embodiments, an effective amount is a therapeutically effective amount. In certain embodiments, an effective amount is a prophylactically effective amount. In certain embodiments, an effective amount is the amount of a compound in a single dose. In certain embodiments, an effective amount is the combined amounts of a compound in multiple doses.

A “therapeutically effective amount” of a compound is an amount sufficient to provide a therapeutic benefit in the treatment of a condition or to delay or minimize one or more symptoms associated with the condition. A therapeutically effective amount of a compound means an amount of therapeutic agent, alone or in combination with other therapies, which provides a therapeutic benefit in the treatment of the condition. The term “therapeutically effective amount” can encompass an amount that improves overall therapy, reduces or avoids symptoms, signs, or causes of the condition, and/or enhances the therapeutic efficacy of another therapeutic agent. In certain embodiments, a therapeutically effective amount is an amount sufficient for treating any disease or condition described herein.

A “prophylactically effective amount” of a compound is an amount sufficient to prevent a condition, or one or more symptoms associated with the condition or prevent its recurrence. A prophylactically effective amount of a compound means an amount of a therapeutic agent, alone or in combination with other agents, which provides a prophylactic benefit in the prevention of the condition. The term “prophylactically effective amount” can encompass an amount that improves overall prophylaxis or enhances the prophylactic efficacy of another prophylactic agent.

As used herein, “inhibition”, “inhibiting”, “inhibit” and “inhibitor”, and the like, refer to the ability of a compound to reduce, slow, halt, or prevent the activity of a biological process (e.g., the activity of an activin receptor-like kinase (e.g., ALK-5) in a subject or cell) or change thereby the progress of a disease by, for example, altering a signaling pathway, for example, altering TGF-β1 signaling.

In certain embodiments, a compound described herein is a “selective inhibitor” that “selectively inhibits” one protein kinase over other kinases. In certain embodiments, the compounds provided herein are selective ALK-5 inhibitors, i.e., selective for ALK-5 over other kinases (e.g., over other activin receptor-like kinases, such as ALK-2; a Janus kinase (JAK), such as JAK1, JAK2 and/or JAK3). The selectivity of a compound described herein in inhibiting the activity of ALK-5 over a different kinase (e.g., a different activin receptor-like kinase) may be measured by the quotient of the EC₅₀ or IC₅₀ value of the compound in inhibiting the activity of the different kinase over the EC₅₀ or IC₅₀ value of the compound in inhibiting the activity of ALK-5. The selectivity of a compound described herein for ALK-5 over a different kinase (e.g., a different activin receptor-like kinase) may also be measured by the quotient of the K_{d} value of an adduct of the compound and the different kinase over the K_{d} value of an adduct of the compound and ALK-5, for example, IC₅₀ inhibition for ALK-5 which is at least 2-fold, at least 3-fold, at least 5-fold, at least 10-fold, at least 30-fold, at least 50-fold, at least 100-fold or greater than 100-fold of the IC₅₀ observed for ALK-2 under comparable testing conditions.

### Compounds

In some embodiments, provided are compounds of Formula (I):

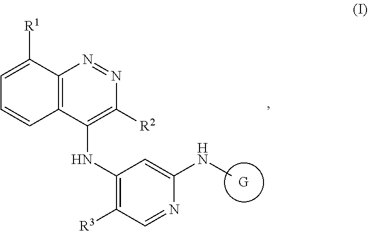

or a pharmaceutically acceptable salt thereof, wherein:
R¹ is a C₁-C₅ alkyl, C₃-C₅ carbocycle, or halogen;
R² is —H, a halogen, a C₁-C₃ alkyl optionally substituted with one or more —F, or a cyclopropyl optionally substituted with one or more —F;
R³ is —H, a halogen, a C₁-C₃ alkyl optionally substituted with one or more —F, or a cyclopropyl optionally substituted with one or more —F; and
Ring G is

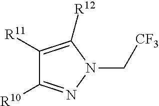

wherein one of R¹⁰, R¹¹ and R¹² is a bond to the —N(H)— to which Ring G is attached in Formula (I), one of R¹⁰, R¹¹ and R¹² is —H, and one of R¹⁰, R¹¹ and R¹² is a C₁-C₄ alkyl; or
Ring G is a C₆-C₁₀ aryl optionally substituted with:
   (i) one or more halogens;
   (ii) a sulfonamide;
   (iii) a monocyclic, bicyclic or spirocyclic C₃-C₁₀ carbocycle which is optionally substituted with one or more C₁-C₆ alkyl or C₃-C₆ carbocycle which are optionally substituted with hydroxy or one or more halogen, wherein said carbocycle is attached to Ring G by a single bond or a methylene or ethylene linker at a position on Ring G which is meta- or para- to the —N(H)— attached to Ring G; or
   (iv) a monocyclic, bicyclic, bridged or spirocyclic C₃-C₁₀ heterocycle which may contain up to 3 heteroatoms independently selected from N and O and which is optionally and independently substituted with one or more C₁-C₆ alkyl or C₃-C₆ carbocycle which are optionally substituted with hydroxy or one or more halogen, wherein said heterocycle is attached to Ring G by a single bond or a methylene or ethylene linker at a position on Ring G which is meta- or para- to the —N(H)— attached to Ring G.

In some embodiments, Ring G is

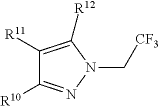

wherein one of R¹⁰, R¹¹ and R¹² is a bond to the —N(H)— to which Ring G is attached in Formula (I), one of R¹⁰, R¹¹ and R¹² is —H, and one of R¹⁰, R¹¹ and R¹² is a C₁-C₄ alkyl. In further embodiments, R¹⁰ is a C₁-C₄ alkyl, R¹¹ is a bond to the —N(H)— to which Ring G is attached in Formula (I), and R¹² is —H.

In some embodiments, provided are compounds of Formula (I):

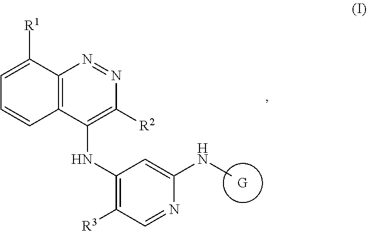

or a pharmaceutically acceptable salt thereof, wherein:
R¹ is a C₁-C₅ alkyl, C₃-C₅ carbocycle, or halogen;
R² is —H, a halogen, a C₁-C₃ alkyl optionally substituted with one or more —F, or a cyclopropyl optionally substituted with one or more —F;
R³ is —H, a halogen, a C₁-C₃ alkyl optionally substituted with one or more —F, or a cyclopropyl optionally substituted with one or more —F; and
Ring G is

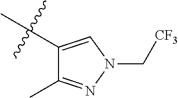 [SMILES: *c1cn(CC(F)(F)F)nc1C]

wherein

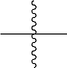

indicates the point of attachment of Ring G to the —N(H)—; or
Ring G is a C₆-C₁₀ aryl optionally substituted with:
   (v) one or more halogens;
   (vi) a sulfonamide;
   (vii) a monocyclic, bicyclic or spirocyclic C₃-C₁₀ carbocycle which is optionally substituted with one or more C₁-C₆ alkyl or C₃-C₆ carbocycle which are optionally substituted with hydroxy or one or more halogen, wherein said carbocycle is attached to Ring G by a single bond or a methylene or ethylene linker at a position on Ring G which is meta- or para- to the —N(H)— attached to Ring G; or
   (viii) a monocyclic, bicyclic, bridged or spirocyclic C₃-C₁₀ heterocycle which may contain up to 3 heteroatoms independently selected from N and O and which is optionally and independently substituted with one or more C₁-C₆ alkyl or C₃-C₆ carbocycle which are optionally substituted with hydroxy or one or more halogen, wherein said heterocycle is attached to Ring G by a single bond or a methylene or ethylene linker at a position on Ring G which is meta- or para- to the —N(H)— attached to Ring G.

In some embodiments, R¹ is a C₁-C₅ alkyl or C₃-C₅ carbocycle.

In some embodiments, R¹ is methyl or cyclopropyl.

In some embodiments, R¹ is a halogen.

In some embodiments, R¹ is methyl or a halogen.

In some embodiments, R¹ is methyl or chloro.

In some embodiments, R² is —H, a halogen, —CH₃, —CF₃ or cyclopropyl.

In some embodiments, R² is —H.

In some embodiments, R³ is —H, a halogen, —CH₃, —CF₃ or cyclopropyl.

In some embodiments, R³ is —H or a halogen.

In some embodiments, R³ is —H or fluoro.

In some embodiments, R³ is —H.

In some embodiments, Ring G is

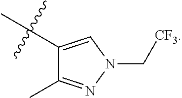 [SMILES: *c1cn(CC(F)(F)F)nc1C]

In some embodiments, Ring G is a phenyl optionally substituted with:
(i) one or more halogens;
(ii) a sulfonamide;
(iii) a monocyclic, bicyclic or spirocyclic C₃-C₁₀ carbocycle which is optionally substituted with one or more C₁-C₆ alkyl or C₃-C₆ carbocycle which are optionally substituted with hydroxy or one or more halogen, wherein said carbocycle is attached to Ring G by a single bond or a methylene or ethylene linker at a position on Ring G which is meta- or para- to the —N(H)— attached to Ring G; or
(iv) a monocyclic, bicyclic, bridged or spirocyclic C₃-C₁₀ heterocycle which may contain up to 3 heteroatoms independently selected from N and O and which is optionally and independently substituted with one or more C₁-C₆ alkyl or C₃-C₆ carbocycle which are optionally substituted with hydroxy or one or more halogen, wherein said heterocycle is attached to Ring G by a single bond or a methylene or ethylene linker at a position on Ring G which is meta- or para- to the —N(H)— attached to Ring G.

In some embodiments, Ring G is a C₆-C₁₀ aryl (in some embodiments, phenyl) substituted with:
(i) one or more halogens;
(ii) a sulfonamide;
(iii) a monocyclic, bicyclic or spirocyclic C₃-C₁₀ carbocycle which is optionally substituted with one or more C₁-C₆ alkyl or C₃-C₆ carbocycle which are optionally substituted with hydroxy or one or more halogen, wherein said carbocycle is attached to Ring G by a single bond or a methylene or ethylene linker at a position on Ring G which is meta- or para- to the —N(H)— attached to Ring G; or
(iv) a monocyclic, bicyclic, bridged or spirocyclic C₃-C₁₀ heterocycle which may contain up to 3 heteroatoms independently selected from N and O and which is optionally and independently substituted with one or more C₁-C₆ alkyl or C₃-C₆ carbocycle which are optionally substituted with hydroxy or one or more halogen, wherein said heterocycle is attached to Ring G by a single bond or a methylene or ethylene linker at a position on Ring G which is meta- or para- to the —N(H)— attached to Ring G.

In some embodiments, Ring G is substituted with one or more halogens.

In some embodiments, Ring G is substituted with a sulfonamide.

In some embodiments, Ring G is substituted with a monocyclic, bicyclic or spirocyclic C₃-C₁₀ carbocycle which is optionally substituted with one or more C₁-C₆ alkyl or C₃-C₆ carbocycle which are optionally substituted with hydroxy or one or more halogen, wherein said carbocycle is attached to Ring G by a single bond or a methylene or ethylene linker at a position on Ring G which is meta- or para- to the —N(H)— attached to Ring G.

In some embodiments, Ring G is substituted with a monocyclic C₃-C₇ carbocycle which is optionally substituted with one or more C₁-C₆ alkyl or C₃-C₆ carbocycle which are optionally substituted with hydroxy or one or more halogen.

In some embodiments, Ring G is substituted with a cyclohexyl which is optionally substituted with one or more C₁-C₆ alkyl or C₃-C₆ carbocycle which are optionally substituted with hydroxy or one or more halogen.

In some embodiments, the carbocycle that is attached to Ring G is unsubstituted.

In some embodiments, the carbocycle that is attached to Ring G is attached to Ring G by a single bond.

In some embodiments, Ring G is substituted with a monocyclic, bicyclic, bridged or spirocyclic C₃-C₁₀ heterocycle which may contain up to 3 heteroatoms independently selected from N and O and which is optionally and independently substituted with one or more C₁-C₆ alkyl or C₃-C₆ carbocycle which are optionally substituted with hydroxy or one or more halogen, wherein said heterocycle is attached to Ring G by a single bond or a methylene or ethylene linker at a position on Ring G which is meta- or para- to the —N(H)— attached to Ring G.

In some embodiments, Ring G is substituted with a monocyclic C₅-C₆ heterocycle which may contain up to 3 heteroatoms independently selected from N and O and which is optionally and independently substituted with one or more C₁-C₆ alkyl or C₃-C₆ carbocycle which are optionally substituted with hydroxy or one or more halogen.

In some embodiments, Ring G is substituted with a monocyclic C₆ heterocycle which may contain up to 2 heteroatoms independently selected from N and O and which is optionally and independently substituted with one or more C₁-C₆ alkyl or C₃-C₆ carbocycle which are optionally substituted with hydroxy or one or more halogen.

In some embodiments, Ring G is substituted with a piperazinyl, morpholinyl, piperidinyl or oxanyl, which is optionally and independently substituted with one or more C₁-C₆ alkyl or C₃-C₆ carbocycle which are optionally substituted with hydroxy or one or more halogen.

In some embodiments, the heterocycle that is attached to Ring G is unsubstituted or monosubstituted.

In some embodiments, the heterocycle that is attached to Ring G is unsubstituted.

In some embodiments, the heterocycle that is attached to Ring G is attached to Ring G by a single bond.

In some embodiments, the carbocycle or heterocycle that is attached to Ring G is optionally and independently substituted with methyl, CF₃CH₂— or HOCH₂CH₂—.

In some embodiments, the carbocycle or heterocycle attached to Ring G is attached to Ring G at a position on Ring G which is meta- to the —N(H)— attached to Ring G.

In some embodiments, the carbocycle or heterocycle attached to Ring G is attached to Ring G at a position on Ring G which is para- to the —N(H)— attached to Ring G.

In some embodiments, provided are compounds of Formula (II):

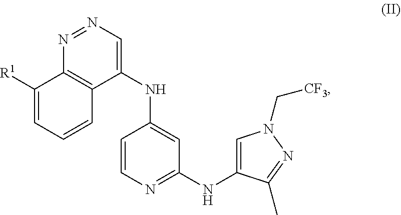

or a pharmaceutically acceptable salt thereof. Values and alternative values for variable R¹ are as described with respect to compounds of Formula (I).

In some embodiments, provided are compounds of Formula (III):

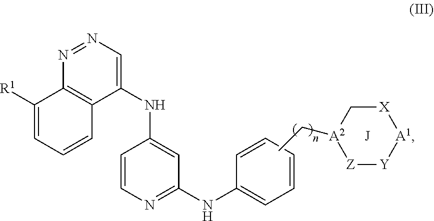

or a pharmaceutically acceptable salt thereof, wherein:
Ring J is attached to the phenylene at a position which is meta- or para- to the —N(H)— attached to the phenylene;
A¹ is —N(R⁴)—, —O— or >C(H)(R⁴);
R⁴ is —H, or a C₁-C₆ alkyl or C₃-C₆ carbocycle, each of which is optionally substituted with hydroxy or one or more halogen;
A² is >N— or >C(H)—;
Z is >CH₂; and X and Y are independently >CH₂ or >C(CH₃)₂, or X and Y are both >CH— and are bonded together through a methylene or ethylene bridge; or
Y is >CH₂ or >C(CH₃)₂, and X and Z are both >CH— and are bonded together through a methylene or ethylene bridge; and
n is 0, 1 or 2. Values and alternative values for variable R¹ are as described with respect to compounds of Formula (I).

In some embodiments, A¹ is >C(H)(R⁴).

In some embodiments, A¹ is —N(R⁴)— or —O—.

In some embodiments, A¹ is —N(R⁴)—.

In some embodiments, A¹ is —O—.

In some embodiments, R⁴ is —H, or a C₁-C₆ alkyl, which is optionally substituted with hydroxy or one or more halogen.

In some embodiments, R⁴ is —H, methyl, CF₃CH₂— or HOCH₂CH₂—.

In some embodiments, R⁴ is —H or methyl.

In some embodiments, A² is >C(H)—.

In some embodiments, wherein A² is >N—.

In some embodiments, Ring J is:

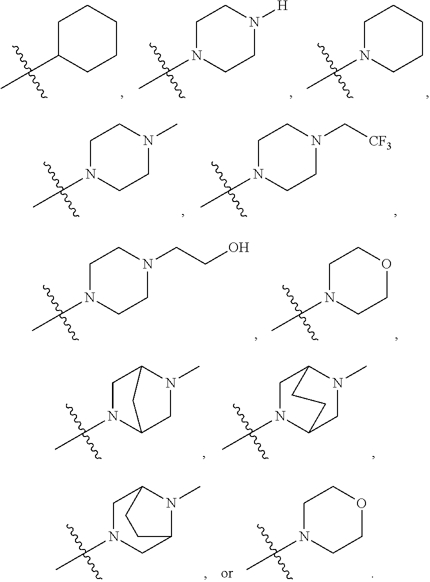

In some embodiments, Ring J is:

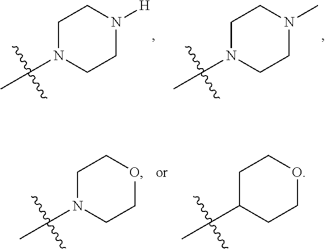

In some embodiments, Ring J is attached to the phenylene at a position which is meta- to the —N(H)— attached to the phenylene.

In some embodiments, Ring J is attached to the phenylene at a position which is para- to the —N(H)— attached to the phenylene.

In some embodiments, n is 0 or 1.

In some embodiments, n is 0.

In some embodiments, provided are compounds of Formula (IV):

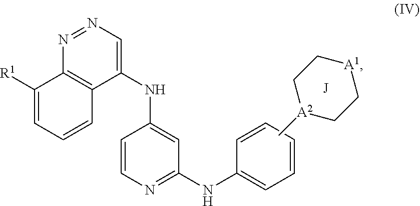

or a pharmaceutically acceptable salt thereof, wherein values and alternative values for the variables (e.g., R¹, Ring J, A¹, A²) are as described with respect to compounds of Formula (I) and/or Formula (III).

In some embodiments, provided is a compound, or a pharmaceutically acceptable salt thereof, having one of the following structures:

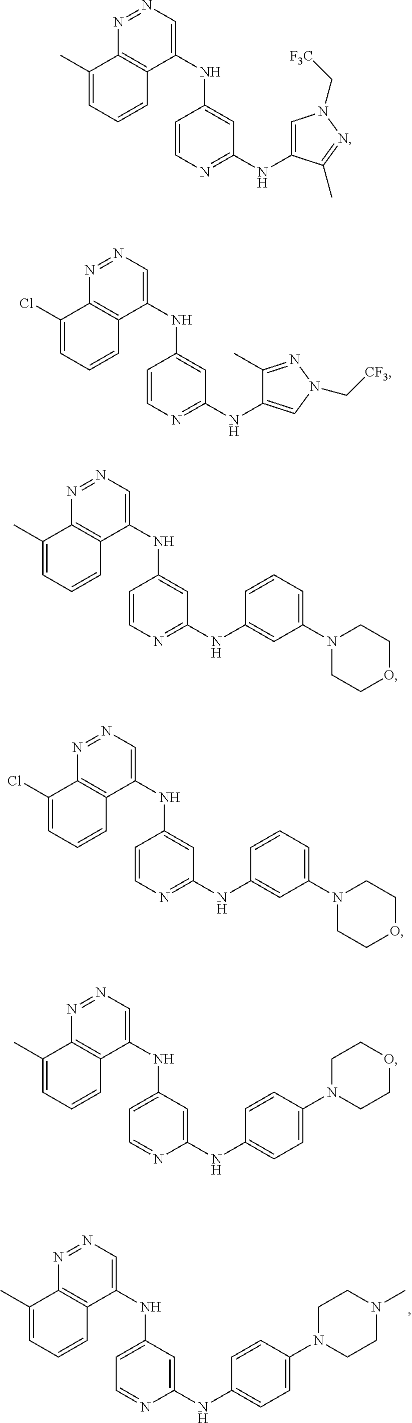

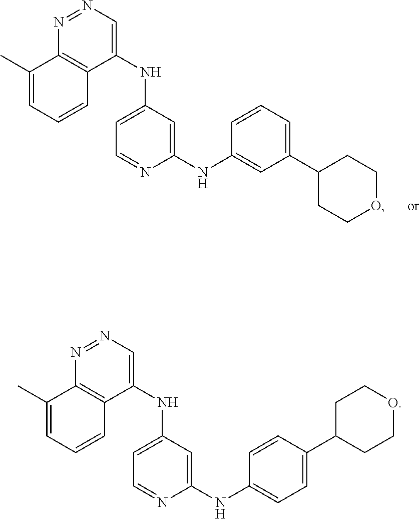 [SMILES: Cc1cccc2c(Nc3ccnc(Nc4cccc(C5CCOCC5)c4)c3)cnnc12 ; Cc1cccc2c(Nc3ccnc(Nc4ccc(C5CCOCC5)cc4)c3)cnnc12]

The recitation of a listing of chemical groups in any definition of a variable herein includes definitions of that variable as any single group or combination of listed groups. The recitation of an embodiment for a variable herein includes that embodiment as any single embodiment or in combination with any other embodiments or portions thereof. The recitation of an embodiment herein includes that embodiment as any single embodiment or in combination with any other embodiments or portions thereof.

### Pharmaceutical Compositions, Kits, and Administration

Provided herein are pharmaceutical compositions comprising a compound of Formula (I), or a pharmaceutically acceptable salt thereof, and a pharmaceutically acceptable carrier or excipient. In certain embodiments, a pharmaceutical composition provided herein comprises a therapeutically and/or prophylactically effective amount of a compound of Formula (I), or a pharmaceutically acceptable salt thereof. In certain embodiments, the pharmaceutical composition comprises a therapeutically effective amount. The pharmaceutical compositions provided herein may further comprise one or more additional therapeutic agents (e.g., anti-proliferative agents, e.g., anti-cancer agents), including any of the additional therapeutic agents described herein in connection with combination therapies.

Pharmaceutical compositions described herein can be prepared by any method known in the art of pharmacology. In general, such preparatory methods include bringing a compound described herein (e.g., the “active ingredient”) into association with a carrier or excipient, and/or one or more other accessory ingredients, and then, if necessary and/or desirable, shaping, and/or packaging the product into a desired single- or multi-dose unit. In some embodiments, pharmaceutical compositions are adapted for oral administration.

Pharmaceutical compositions can be prepared, packaged, and/or sold in bulk, as a single unit dose, and/or as a plurality of single unit doses. A “unit dose” is a discrete amount of the pharmaceutical composition comprising a predetermined amount of the active ingredient. The amount of the active ingredient is generally equal to the dosage of the active ingredient which would be administered to a subject and/or a convenient fraction of such a dosage, such as one-half or one-third of such a dosage.

Relative amounts of the active ingredient (e.g., the compound of Formula (I) or pharmaceutically acceptable salt thereof), the pharmaceutically acceptable excipient, and/or any additional ingredients in a pharmaceutical composition described herein will vary, depending, for example, on the identity, size, and/or condition of the subject treated and upon the route by which the composition is to be administered. The composition may comprise between 0.1% and 100% (w/w) active ingredient.

Pharmaceutically acceptable excipients used in the manufacture of provided pharmaceutical compositions include inert diluents, dispersing and/or granulating agents, surface active agents and/or emulsifiers, disintegrating agents, binding agents, preservatives, buffering agents, lubricating agents, and/or oils. Excipients such as cocoa butter and suppository waxes, coloring agents, coating agents, sweetening, flavoring, and perfuming agents may also be present in the composition.

Examples of diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and mixtures thereof.

Examples of granulating and/or dispersing agents include potato starch, corn starch, tapioca starch, sodium starch glycolate, clays, alginic acid, guar gum, citrus pulp, agar, bentonite, cellulose, and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, and mixtures thereof.

Examples of surface active agents and/or emulsifiers include natural emulsifiers (e.g., acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g., bentonite (aluminum silicate) and Veegum (magnesium aluminum silicate)), long chain amino acid derivatives, high molecular weight alcohols (e.g., stearyl alcohol, cetyl alcohol, oleyl alcohol, triacetin monostearate, ethylene glycol distearate, glyceryl monostearate, and propylene glycol monostearate, polyvinyl alcohol), carbomers (e.g., carboxy polymethylene, polyacrylic acid, acrylic acid polymer, and carboxyvinyl polymer), carrageenan, cellulosic derivatives (e.g., carboxymethylcellulose sodium, powdered cellulose, hydroxymethyl cellulose, hydroxypropyl cellulose, hydroxypropyl methylcellulose, methylcellulose), sorbitan fatty acid esters (e.g., polyoxyethylene sorbitan monolaurate (Tween® 20), polyoxyethylene sorbitan (Tween® 60), polyoxyethylene sorbitan monooleate (Tween® 80), sorbitan monopalmitate (Span® 40), sorbitan monostearate (Span® 60), sorbitan tristearate (Span® 65), glyceryl monooleate, sorbitan monooleate (Span® 80), polyoxyethylene esters (e.g., polyoxyethylene monostearate (Myrj® 45), polyoxyethylene hydrogenated castor oil, polyethoxylated castor oil, polyoxymethylene stearate, and Solutol®), sucrose fatty acid esters, polyethylene glycol fatty acid esters (e.g., Cremophor®), polyoxyethylene ethers, (e.g., polyoxyethylene lauryl ether (Brij® 30)), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic® F-68, poloxamer P-188, cetrimonium bromide, cetylpyridinium chloride, benzalkonium chloride, docusate sodium, and/or mixtures thereof.

Examples of binding agents include starch (e.g., cornstarch and starch paste), gelatin, sugars (e.g., sucrose, glucose, dextrose, dextrin, molasses, lactose, lactitol, mannitol, etc.), natural and synthetic gums (e.g., acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum®), and larch arabogalactan), alginates, polyethylene oxide, polyethylene glycol, inorganic calcium salts, silicic acid, polymethacrylates, waxes, water, alcohol, and/or mixtures thereof.

Examples of preservatives include antioxidants, chelating agents, antimicrobial preservatives, antifungal preservatives, antiprotozoan preservatives, alcohol preservatives, acidic preservatives, and other preservatives. In certain embodiments, the preservative is an antioxidant. In other embodiments, the preservative is a chelating agent.

Examples of antioxidants include alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Examples of chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Examples of antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Examples of alcohol preservatives include ethanol, polyethylene glycol, phenol, phenolic compounds, bisphenol, chlorobutanol, hydroxybenzoate, and phenylethyl alcohol.

Examples of acidic preservatives include vitamin A, vitamin C, vitamin E, beta-carotene, citric acid, acetic acid, dehydroacetic acid, ascorbic acid, sorbic acid, and phytic acid.

Other preservatives include tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, Glydant® Plus, Phenonip®, methylparaben, Germall® 115, Germaben® II, Neolone®, Kathon®, and Euxyl®.

Examples of buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, and mixtures thereof.

Examples of lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, and mixtures thereof.

Examples of natural oils include almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary synthetic oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and mixtures thereof.

Liquid dosage forms for oral and parenteral administration include pharmaceutically acceptable emulsions, microemulsions, solutions, suspensions, syrups and elixirs. In addition to the active ingredients, the liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (e.g., cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can include adjuvants such as wetting agents, emulsifying and suspending agents, sweetening, flavoring, and perfuming agents. In certain embodiments for parenteral administration, the active ingredient is mixed with solubilizing agents such as Cremophor®, alcohols, oils, modified oils, glycols, polysorbates, cyclodextrins, polymers, and mixtures thereof.

Injectable preparations, for example, sterile injectable aqueous or oleaginous suspensions can be formulated according to the known art using suitable dispersing or wetting agents and suspending agents. The sterile injectable preparation can be a sterile injectable solution, suspension, or emulsion in a nontoxic parenterally acceptable diluent or solvent, for example, as a solution in 1,3-butanediol. Among the acceptable vehicles and solvents that can be employed are water, Ringer's solution, U.S.P., and isotonic sodium chloride solution. In addition, sterile, fixed oils are conventionally employed as a solvent or suspending medium. For this purpose, any bland fixed oil can be employed including synthetic mono- or di-glycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This can be accomplished by the use of a liquid suspension of crystalline or amorphous material with poor water solubility. The rate of absorption of the drug then depends upon its rate of dissolution, which, in turn, may depend upon crystal size and crystalline form. Alternatively, delayed absorption of a parenterally administered drug form may be accomplished by dissolving or suspending the drug in an oil vehicle.

Solid dosage forms for oral administration include capsules, tablets, pills, powders, and granules. In such solid dosage forms, the active ingredient is mixed with at least one inert, pharmaceutically acceptable excipient or carrier such as sodium citrate or dicalcium phosphate and/or (a) fillers or extenders such as starches, lactose, sucrose, glucose, mannitol, and silicic acid, (b) binders such as, for example, carboxymethylcellulose, alginates, gelatin, polyvinylpyrrolidinone, sucrose, and acacia, (c) humectants such as glycerol, (d) disintegrating agents such as agar, calcium carbonate, potato or tapioca starch, alginic acid, certain silicates, and sodium carbonate, (e) solution retarding agents such as paraffin, (f) absorption accelerators such as quaternary ammonium compounds, (g) wetting agents, such as, for example, cetyl alcohol and glycerol monostearate, (h) absorbents such as kaolin and bentonite clay, and (i) lubricants such as talc, calcium stearate, magnesium stearate, solid polyethylene glycols, sodium lauryl sulfate, and mixtures thereof. In the case of capsules, tablets, and pills, the dosage form may include a buffering agent.

Solid compositions of a similar type can be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the art of pharmacology. They may optionally comprise opacifying agents and can be of a composition that they release the active ingredient(s) only, or preferentially, in a certain part of the intestinal tract, optionally, in a delayed manner. Examples of encapsulating compositions which can be used include polymeric substances and waxes. Solid compositions of a similar type can be employed as fillers in soft and hard-filled gelatin capsules using such excipients as lactose or milk sugar as well as high molecular weight polethylene glycols and the like.

The active ingredient can be in a micro-encapsulated form with one or more excipients as noted above. The solid dosage forms of tablets, dragees, capsules, pills, and granules can be prepared with coatings and shells such as enteric coatings, release controlling coatings, and other coatings well known in the pharmaceutical formulating art. In such solid dosage forms the active ingredient can be admixed with at least one inert diluent such as sucrose, lactose, or starch. Such dosage forms may comprise, as is normal practice, additional substances other than inert diluents, e.g., tableting lubricants and other tableting aids such a magnesium stearate and microcrystalline cellulose. In the case of capsules, tablets and pills, the dosage forms may comprise buffering agents. They may optionally comprise opacifying agents and can be of a composition that they release the active ingredient(s) only, or preferentially, in a certain part of the intestinal tract, optionally, in a delayed manner. Examples of encapsulating agents which can be used include polymeric substances and waxes.

Dosage forms for topical and/or transdermal administration of a compound described herein may include ointments, pastes, creams, lotions, gels, powders, solutions, sprays, inhalants, and/or patches. Generally, the active ingredient is admixed under sterile conditions with a pharmaceutically acceptable carrier or excipient and/or any needed preservatives and/or buffers as can be required. Additionally, the present disclosure contemplates the use of transdermal patches, which often have the added advantage of providing controlled delivery of an active ingredient to the body. Such dosage forms can be prepared, for example, by dissolving and/or dispensing the active ingredient in the proper medium. Alternatively or additionally, the rate can be controlled by either providing a rate controlling membrane and/or by dispersing the active ingredient in a polymer matrix and/or gel.

Suitable devices for use in delivering intradermal pharmaceutical compositions described herein include short needle devices. Intradermal compositions can be administered by devices which limit the effective penetration length of a needle into the skin. Alternatively or additionally, conventional syringes can be used in the classical mantoux method of intradermal administration. Jet injection devices which deliver liquid formulations to the dermis via a liquid jet injector and/or via a needle which pierces the stratum corneum and produces a jet which reaches the dermis are suitable. Ballistic powder/particle delivery devices which use compressed gas to accelerate the compound in powder form through the outer layers of the skin to the dermis are suitable.

Formulations suitable for topical administration include, but are not limited to, liquid and/or semi-liquid preparations such as liniments, lotions, oil-in-water and/or water-in-oil emulsions such as creams, ointments, and/or pastes, and/or solutions and/or suspensions. Topically administrable formulations may, for example, comprise from about 1% to about 10% (w/w) active ingredient, although the concentration of the active ingredient can be as high as the solubility limit of the active ingredient in the solvent. Formulations for topical administration may further comprise one or more of the additional ingredients described herein.

Compositions for rectal or vaginal administration are typically suppositories which can be prepared by mixing the conjugates described herein with suitable non-irritating excipients or carriers such as cocoa butter, polyethylene glycol, or a suppository wax which are solid at ambient temperature but liquid at body temperature and therefore melt in the rectum or vaginal cavity and release the active ingredient.

A pharmaceutical composition described herein can be prepared, packaged, and/or sold in a formulation suitable for pulmonary administration via the buccal cavity. Such a formulation may comprise dry particles which comprise the active ingredient and which have a diameter in the range from about 0.5 to about 7 nanometers, or from about 1 to about 6 nanometers. Such compositions are conveniently in the form of dry powders for administration using a device comprising a dry powder reservoir to which a stream of propellant can be directed to disperse the powder and/or using a self-propelling solvent/powder dispensing container such as a device comprising the active ingredient dissolved and/or suspended in a low-boiling propellant in a sealed container. Such powders comprise particles wherein at least 98% of the particles by weight have a diameter greater than 0.5 nanometers and at least 95% of the particles by number have a diameter less than 7 nanometers. Alternatively, at least 95% of the particles by weight have a diameter greater than 1 nanometer and at least 90% of the particles by number have a diameter less than 6 nanometers. Dry powder compositions may include a solid fine powder diluent such as sugar and are conveniently provided in a unit dose form.

Low boiling propellants generally include liquid propellants having a boiling point of below 65° F. at atmospheric pressure. Generally, the propellant may constitute 50 to 99.9% (w/w) of the composition, and the active ingredient may constitute 0.1 to 20% (w/w) of the composition. The propellant may further comprise additional ingredients such as a liquid non-ionic and/or solid anionic surfactant and/or a solid diluent (which may have a particle size of the same order as particles comprising the active ingredient).

Pharmaceutical compositions described herein formulated for pulmonary delivery may provide the active ingredient in the form of droplets of a solution and/or suspension. Such formulations can be prepared, packaged, and/or sold as aqueous and/or dilute alcoholic solutions and/or suspensions, optionally sterile, comprising the active ingredient, and may conveniently be administered using any nebulization and/or atomization device. Such formulations may further comprise one or more additional ingredients including, but not limited to, a flavoring agent such as saccharin sodium, a volatile oil, a buffering agent, a surface active agent, and/or a preservative such as methylhydroxybenzoate. The droplets provided by this route of administration may have an average diameter in the range from about 0.1 to about 200 nanometers.

Formulations described herein as being useful for pulmonary delivery are useful for intranasal delivery of a pharmaceutical composition described herein. Another formulation suitable for intranasal administration is a coarse powder comprising the active ingredient and having an average particle from about 0.2 to 500 micrometers. Such a formulation is administered by rapid inhalation through the nasal passage from a container of the powder held close to the nares.

Formulations for nasal administration may, for example, comprise from about as little as 0.1% (w/w) to as much as 100% (w/w) of the active ingredient, and may comprise one or more of the additional ingredients described herein.

A pharmaceutical composition described herein can be prepared, packaged, and/or sold in a formulation for buccal administration. Such formulations may, for example, be in the form of tablets and/or lozenges made using conventional methods, and may contain, for example, 0.1 to 20% (w/w) active ingredient, the balance comprising an orally dissolvable and/or degradable composition and, optionally, one or more of the additional ingredients described herein. Alternately, formulations for buccal administration may comprise a powder and/or an aerosolized and/or atomized solution and/or suspension comprising the active ingredient. Such powdered, aerosolized, and/or aerosolized formulations, when dispersed, may have an average particle and/or droplet size in the range from about 0.1 to about 200 nanometers, and may further comprise one or more of the additional ingredients described herein.

A pharmaceutical composition described herein can be prepared, packaged, and/or sold in a formulation for ophthalmic administration. Such formulations may, for example, be in the form of eye drops including, for example, a 0.1-1.0% (w/w) solution and/or suspension of the active ingredient in an aqueous or oily liquid carrier or excipient. Such drops may further comprise buffering agents, salts, and/or one or more other of the additional ingredients described herein. Other ophthalmically-administrable formulations which are useful include those which comprise the active ingredient in microcrystalline form and/or in a liposomal preparation. Ear drops and/or eye drops are also contemplated as being within the scope of this disclosure.

Although the descriptions of pharmaceutical compositions provided herein are principally directed to pharmaceutical compositions which are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with ordinary experimentation.

The compounds, salts and compositions provided herein can be administered by any route, including enteral (e.g., oral), parenteral, intravenous, intramuscular, intra-arterial, intramedullary, intrathecal, subcutaneous, intraventricular, transdermal, interdermal, rectal, intravaginal, intraperitoneal, topical (as by powders, ointments, creams, and/or drops), ophthalmic, mucosal, nasal, bucal, sublingual; by intratracheal instillation, bronchial instillation, and/or inhalation; and/or as an oral spray, nasal spray, and/or aerosol. Specifically contemplated routes are oral administration, intravenous administration (e.g., systemic intravenous injection), regional administration via blood and/or lymph supply, and/or direct administration to an affected site. In general, the most appropriate route of administration will depend upon a variety of factors, such as the nature of the agent (e.g., its stability in the environment of the gastrointestinal tract), and/or the condition of the subject (e.g., whether the subject is able to tolerate oral administration). In some embodiments, a pharmaceutical composition is formulated for oral administration.

Compounds, salts and compositions provided herein are typically formulated in dosage unit form for ease of administration and uniformity of dosage. It will be understood, however, that the total daily usage of the compositions described herein will be decided by a physician within the scope of sound medical judgment. The specific therapeutically effective dose level for any particular subject or organism will depend upon a variety of factors including the disease being treated and the severity of the disorder; the activity of the specific active ingredient employed; the specific composition employed; the age, body weight, general health, sex, and diet of the subject; the time of administration, route of administration, and rate of excretion of the specific active ingredient employed; the duration of the treatment; drugs used in combination or coincidental with the specific active ingredient employed; and like factors well known in the medical arts.

The exact amount of a compound, salt or composition required to achieve an effective amount will vary from subject to subject, depending, for example, on species, age, and general condition of a subject, severity of the side effects or disorder, identity of the particular compound, mode of administration, and the like. An effective amount may be included in a single dose (e.g., single oral dose) or multiple doses (e.g., multiple oral doses). In certain embodiments, when multiple doses are administered to a subject or applied to a tissue or cell, any two doses of the multiple doses include different or substantially the same amounts of a compound described herein. In certain embodiments, when multiple doses are administered to a subject or applied to a tissue or cell, the frequency of administering the multiple doses to the subject or applying the multiple doses to the tissue or cell is three doses a day, two doses a day, one dose a day, one dose every other day, one dose every third day, one dose every week, one dose every two weeks, one dose every three weeks, or one dose every four weeks. In certain embodiments, the frequency of administering the multiple doses to the subject or applying the multiple doses to the tissue or cell is one dose per day. In certain embodiments, the frequency of administering the multiple doses to the subject or applying the multiple doses to the tissue or cell is two doses per day. In certain embodiments, the frequency of administering the multiple doses to the subject or applying the multiple doses to the tissue or cell is three doses per day. In certain embodiments, when multiple doses are administered to a subject or applied to a tissue or cell, the duration between the first dose and last dose of the multiple doses is one day, two days, four days, one week, two weeks, three weeks, one month, two months, three months, four months, six months, nine months, one year, two years, three years, four years, five years, seven years, ten years, fifteen years, twenty years, or the lifetime of the subject, tissue, or cell. In certain embodiments, the duration between the first dose and last dose of the multiple doses is three months, six months, or one year. In certain embodiments, the duration between the first dose and last dose of the multiple doses is the lifetime of the subject, tissue, or cell. In certain embodiments, a dose (e.g., a single dose, or any dose of multiple doses) described herein includes independently between 0.1 μg and 1 μg, between 0.001 mg and 0.01 mg, between 0.01 mg and 0.1 mg, between 0.1 mg and 1 mg, between 1 mg and 3 mg, between 3 mg and 10 mg, between 10 mg and 30 mg, between 30 mg and 100 mg, between 100 mg and 300 mg, between 300 mg and 1,000 mg, or between 1 g and 10 g, inclusive, of a compound described herein. In certain embodiments, a dose described herein includes independently between 1 mg and 3 mg, inclusive, of a compound described herein. In certain embodiments, a dose described herein includes independently between 3 mg and 10 mg, inclusive, of a compound described herein. In certain embodiments, a dose described herein includes independently between 10 mg and 30 mg, inclusive, of a compound described herein. In certain embodiments, a dose described herein includes independently between 30 mg and 100 mg, inclusive, of a compound described herein. In some embodiments the dosage is expected to be in the range of 1 mg/Kg subject mass and 150 mg/Kg subject mass, for example, at least about 1 mg/Kg, at least about 10 mg/Kg, at least about 20 mg/Kg, at least about 30 mg/Kg, at least about 40 mg/Kg, at least about 50 mg/Kg, at least about 60 mg/Kg, at least about 70 mg/Kg, at least about 80 mg/Kg, at least about 90 mg/Kg, at least about 100 mg/Kg, at least about 110 mg/Kg, at least about 120 mg/Kg, at least about 130 mg/Kg, at least about 140 mg/Kg, or about 150 mg/Kg.

Dose ranges as described herein provide guidance for the administration of provided pharmaceutical compositions to an adult. The amount to be administered to, for example, a child or an adolescent can be determined by a medical practitioner or person skilled in the art and can be lower or the same as that administered to an adult.

Also encompassed by the disclosure are kits (e.g., pharmaceutical packs). The kits provided may comprise a compound, salt or pharmaceutical composition described herein and a container (e.g., a vial, ampule, bottle, syringe, and/or dispenser package, or other suitable container). In some embodiments, provided kits may optionally further include a second container comprising a pharmaceutical excipient for dilution or suspension of a pharmaceutical composition or compound or salt described herein. In some embodiments, the pharmaceutical composition or compound or salt described herein provided in the first container and the second container are combined to form one unit dosage form.

Thus, in one aspect, provided are kits including a first container comprising a compound, salt or pharmaceutical composition described herein. In certain embodiments, the kits are useful for treating a disease (e.g., a proliferative disease such as cancer) in a subject in need thereof. In certain embodiments, the kits are useful for preventing a disease in a subject in need thereof. In certain embodiments, the kits are useful for reducing the risk of developing a disease in a subject in need thereof.

In certain embodiments, a kit described herein further includes instructions for using the kit. A kit described herein may also include information as required by a regulatory agency such as the U.S. Food and Drug Administration (FDA). In certain embodiments, the information included in the kits is prescribing information. A kit described herein may include one or more additional therapeutic agents described herein as a separate composition or in a combination comprising a compound or pharmaceutical composition described herein.

In the combinations and/or kits described herein, the compound of the present disclosure and the other therapeutic agent may be manufactured and/or formulated by the same or different manufacturers. Moreover, the compound of the present disclosure and the other therapeutic agent may be brought together into a combination therapy: (i) prior to release of the combination product to physicians (e.g., in the case of a kit comprising the compound of the present disclosure and the other therapeutic agent); (ii) by the physician (or under the guidance of a physician) shortly before administration; (iii) in the patient themselves, e.g., during sequential administration of the compound of the present disclosure and the other therapeutic agent.

A pharmaceutical composition (or formulation) for application may be packaged in a variety of ways depending upon the method used for administering the drug. Generally, an article for distribution includes a container having deposited therein the pharmaceutical formulation in an appropriate form. Suitable containers are well-known to those skilled in the art and include materials such as bottles (plastic and glass), sachets, ampoules, plastic bags, metal cylinders, and the like. The container may also include a tamper-proof assemblage to prevent indiscreet access to the contents of the package. In addition, the container has deposited thereon a label that describes the contents of the container. The label may also include appropriate warnings.

In some embodiments, the concentration of one or more therapeutic agents provided in a pharmaceutical composition is less than 100%, 90%, 80%, 70%, 60%, 50%, 40%, 30%, 20%, 19%, 18%, 17%, 16%, 15%, 14%, 13%, 12%, 11%, 10%, 9%, 8%, 7%, 6%, 5%, 4%, 3%, 2%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002%, or 0.00010% w/w, w/v or v/v.

In some embodiments, the concentration of one or more therapeutic agents provided in a pharmaceutical composition is greater than 90%, 80%, 70%, 60%, 50%, 40%, 30%, 20%, 19.75%, 19.50%, 19.25% 19%, 18.75%, 18.50%, 18.25% 18%, 17.75%, 17.50%, 17.25% 17%, 16.75%, 16.50%, 16.25% 16%, 15.75%, 15.50%, 15.25% 15%, 14.75%, 14.50%, 14.25% 14%, 13.75%, 13.50%, 13.25% 13%, 12.75%, 12.50%, 12.25% 12%, 11.75%, 11.50%, 11.25% 11%, 10.75%, 10.50%, 10.25% 10%, 9.75%, 9.50%, 9.25% 9%, 8.75%, 8.50%, 8.25% 8%, 7.75%, 7.50%, 7.25% 7%, 6.75%, 6.50%, 6.25% 6%, 5.75%, 5.50%, 5.25% 5%, 4.75%, 4.50%, 4.25%, 4%, 3.75%, 3.50%, 3.25%, 3%, 2.75%, 2.50%, 2.25%, 2%1, 1.75%, 1.50%, 125%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002%, or 0.0001% w/w, w/v, or v/v.

In some embodiments, the concentration of one or more therapeutic agents provided in a pharmaceutical composition is in the range from about 0.0001% to about 50%, about 0.001% to about 40%, about 0.01% to about 30%, about 0.02% to about 29%, about 0.03% to about 28%, about 0.04% to about 27%, about 0.05% to about 26%, about 0.06% to about 25%, about 0.07% to about 24%, about 0.08% to about 23%, about 0.09% to about 22%, about 0.1% to about 21%, about 0.2% to about 20%, about 0.3% to about 19%, about 0.4% to about 18%, about 0.5% to about 17%, about 0.6% to about 16%, about 0.7% to about 15%, about 0.8% to about 14%, about 0.9% to about 12%, about 1% to about 10% w/w, w/v or v/v.

In some embodiments, the concentration of one or more therapeutic agents provided in a pharmaceutical composition is in the range from about 0.001% to about 10%, about 0.01% to about 5%, about 0.02% to about 4.5%, about 0.03% to about 4%, about 0.04% to about 3.5%, about 0.05% to about 3%, about 0.06% to about 2.5%, about 0.07% to about 2%, about 0.08% to about 1.5%, about 0.09% to about 1%, about 0.1% to about 0.9% w/w, w/v or v/v.

### Methods of Treatment and Uses

As shown herein, compounds provided herein are activin receptor-like kinase (e.g., ALK-5) inhibitors. In some embodiments, the compounds provided herein are useful for treating and/or preventing diseases (e.g., fibrotic diseases, for example, IPF or cardiac fibrosis or a cardiac disease associated with TGFβ signaling, and proliferative diseases, e.g., a cancer) in a subject, for example, inhibiting tumor growth in a subject, or inhibiting the activity of an activin receptor-like kinase (e.g., ALK-5) in vitro or in vivo. In some embodiments, the compounds provided herein are useful in moderating, preventing, or providing treatment for conditions and/or diseases the progress of which is driven by, or utilizes the TGFβ-signaling pathway for disease progression, as described herein.

Also as shown herein, compounds provided herein can inhibit epithelial to mesenchymal transition (EMT). In some embodiments, the compounds provided herein are useful for inhibiting EMT in vitro or in vivo. In some embodiments, the compounds provided herein are useful in moderating, preventing, or providing treatment for conditions and/or diseases the progress of which is driven by EMT, as described herein. Examples of such conditions and/or diseases include cancer (e.g., metastatic cancer, chemotherapy-resistant cancer) and fibrosis (e.g., cancer-associated fibrosis, idiopathic pulmonary fibrosis).

Provided herein are methods of treating and/or preventing (e.g., treating) a disease or condition (e.g., a fibrotic disease or condition, which is present by itself or comorbid with an infectious, inflammatory or proliferative disease or condition (either benign or malignant); an inflammatory disease or condition; or a proliferative disease or condition, e.g., cancer) in a subject (e.g., a subject in need thereof), the methods comprising administering to the subject a therapeutically and/or prophylactically effective amount of a compound of Formula (I), or a pharmaceutically acceptable salt thereof, or a pharmaceutical composition thereof. Also provided herein are compounds of Formula (I), and pharmaceutically acceptable salts thereof, and pharmaceutical compositions of the foregoing, for use in treating and/or preventing a disease or condition described herein. Also provided herein are uses of compounds of Formula (I), and pharmaceutically acceptable salts thereof, and pharmaceutical compositions of the foregoing, for the manufacture of a medicament for treating and/or preventing a disease or condition described herein. In certain embodiments, the disease is a disease associated with activin receptor-like kinase (e.g., ALK-5) activity in a subject or cell. In certain embodiments, the activity is aberrant (e.g., increased) activity.

In certain embodiments, the disease or condition is a proliferative disease. Provided herein are methods for treating a proliferative disease (e.g., cancer) in a subject (e.g., a subject in need thereof), the methods comprising administering to the subject a therapeutically effective amount of a compound of Formula (I), or a pharmaceutically acceptable salt thereof, or a pharmaceutical composition thereof. Also provided herein are compounds of Formula (I), and pharmaceutically acceptable salts thereof, and pharmaceutical compositions of the foregoing, for use in treating a proliferative disease (e.g., cancer). Also provided herein are uses of compounds of Formula (I), and pharmaceutically acceptable salts thereof, and pharmaceutical compositions of the foregoing, for the manufacture of a medicament for treating a proliferative disease (e.g., cancer). In certain embodiments, the proliferative disease is associated with activin receptor-like kinase (e.g., ALK-5) activity in a subject or cell. In certain embodiments, the activity is aberrant or increased activity.

A “proliferative disease” refers to a disease that occurs due to abnormal growth or extension by the multiplication of cells (Walker, Cambridge Dictionary ofBiology; Cambridge University Press: Cambridge, UK, 1990). A proliferative disease may be associated with: 1) the pathological proliferation of normally quiescent cells; 2) the pathological migration of cells from their normal location (e.g., metastasis of neoplastic cells); 3) the pathological expression of proteolytic enzymes such as the matrix metalloproteinases (e.g., collagenases, gelatinases, and elastases); and/or 4) pathological angiogenesis as in proliferative retinopathy and tumor metastasis. Exemplary proliferative diseases include cancers (i.e., “malignant neoplasms”), benign neoplasms, angiogenesis, inflammatory diseases, and autoimmune diseases.

The terms “neoplasm” and “tumor” are used herein interchangeably and refer to an abnormal mass of tissue wherein the growth of the mass surpasses and is not coordinated with the growth of a normal tissue. A neoplasm or tumor may be “benign” or “malignant,” depending on the following characteristics: degree of cellular differentiation (including morphology and functionality), rate of growth, local invasion, and metastasis.

A “benign neoplasm” is generally well differentiated, has characteristically slower growth than a malignant neoplasm, and remains localized to the site of origin. In addition, a benign neoplasm does not have the capacity to infiltrate, invade, or metastasize to distant sites. Exemplary benign neoplasms include, but are not limited to, lipoma, chondroma, adenomas, acrochordon, senile angiomas, seborrheic keratoses, lentigos, and sebaceous hyperplasias. In some cases, certain “benign” tumors may later give rise to malignant neoplasms, which may result from additional genetic changes in a subpopulation of the tumor's neoplastic cells, and these tumors are referred to as “pre-malignant neoplasms.” An exemplary pre-malignant neoplasm is a teratoma.

In contrast, a “malignant neoplasm” is generally poorly differentiated (anaplasia) and has characteristically rapid growth accompanied by progressive infiltration, invasion, and destruction of the surrounding tissue. Furthermore, a malignant neoplasm generally has the capacity to metastasize to distant sites. The term “metastasis,” “metastatic,” or “metastasize” refers to the spread or migration of cancerous cells from a primary or original tumor to another organ or tissue and is typically identifiable by the presence of a “secondary tumor” or “secondary cell mass” of the tissue type of the primary or original tumor and not of that of the organ or tissue in which the secondary (metastatic) tumor is located.

In certain embodiments, the disease or condition to be treated is cancer. Provided herein are methods for treating cancer in a subject (e.g., a subject in need thereof), the methods comprising administering to the subject a therapeutically effective amount of one or more of the exemplified compounds, or one or more of these in the form of a pharmaceutically acceptable salt, or a pharmaceutical composition of the foregoing. In some embodiments the exemplified compounds are those of compounds of Formula (I) (II), (III), (IV), or Table 1. Also provided herein are compounds of Formulae (I) (II), (III), (IV), or Table 1, and pharmaceutically acceptable salts thereof, and pharmaceutical compositions of the foregoing for use in treating cancer. Also provided herein are uses of compounds of Formulae (I) (II), (III), (IV), or Table 1, and pharmaceutically acceptable salts thereof, and pharmaceutical compositions of the foregoing, for the manufacture of a medicament for treating cancer. In certain embodiments, cancer is associated with the activity of an activin receptor-like kinase (e.g., ALK-5) in a subject or cell. In certain embodiments, the cancer is associated with the activity of ALK-5 in a subject or cell. In certain embodiments, the activity is aberrant (e.g., increased) activity.

The term “cancer” refers to a class of diseases characterized by the development of abnormal cells that proliferate uncontrollably and have the ability to infiltrate and destroy normal body tissues. In certain embodiments, the cancer is a solid tumor. In certain embodiments, the cancer is a hematological cancer.

In certain embodiments, cancer is associated with the activity of an activin receptor-like kinase (e.g., ALK-5) in a subject or cell. In certain embodiments, the cancer is associated with the activity of ALK-5 in a subject or cell. In certain embodiments, the activity is increased (e.g., aberrant) activin receptor-like kinase (e.g., ALK-5) activity.

In certain embodiments, the cancer expresses or has mutant forkhead box L2 (FOXL2) and/or FOXL2 (e.g., FOXL2^{C134W}). FOXL2^{C134W} is characteristic of approximately 97% of AGCT, a rare ovarian cancer subtype (>5%). An example of a cancer that expresses or has mutant FOXL2 is ovarian cancer (e.g., AGCT). Other sex cord stromal tumors, such as JGCT, thecoma, SLCT, male AGCT, and gynandroblastoma, are other examples of cancers that express or have mutant FOXL2 and/or FOXL2.

In some embodiments, provided herein is a method for treating a cancer (e.g., ovarian cancer, such as adult granulosa cell tumor), comprising determining whether a subject carries a FOXL2 mutation (e.g., FOXL2^{C134W}); and treating the subject with a therapeutically effective amount of a compound of the present disclosure, for example, a compound of Formula (I) (II), (III), or (IV), or Table 1 or Table 4, for example, one or more of Ex-10, Ex-11, Ex-12, Ex-13, Ex-33, Ex-34, Ex-57, or Ex-58, or a pharmaceutically acceptable salt of the foregoing, or a composition thereof, if the subject is identified as having the FOXL2 mutation.

In some embodiments, the cancer has FOXL2 driven tumor growth.

In some embodiments, the cancer is associated with an elevated level of pSmad2 and/or αVβ6 and/or alpha smooth muscle actin (α-SMA). In some embodiments, the cancer is associated with an elevated level of phosphorylated SMAD 2 (pSMAD2) or alpha smooth muscle actin (α-SMA).

In addition to FOXL2 mutants (e.g., FOXL2^{C134W}), pSMAD2, αVβ6, and α-SMA, other biomarkers that may be predictive (e.g., and used as a patient selection criterion) and/or indicative (e.g., and used during and/or after treatment to assess some aspect of the treatment) of efficacy of a treatment disclosed herein include CD31 (e.g., an elevated level of CD31), CD45 (e.g., an elevated level of CD45), and/or HLA (e.g., a low level of HLA).

In some embodiments, the cancer (e.g., solid tumor cancer) exhibits an excluded or desert phenotype. In some embodiments, the cancer (e.g., solid tumor cancer) exhibits an excluded phenotype. In some embodiments, the cancer (e.g., solid tumor cancer) exhibits a desert phenotype.

A wide variety of cancers, including solid tumors, leukemias, lymphomas, and myelomas are amenable to the methods disclosed herein. In some embodiments, the cancer is a solid tumor cancer. In some embodiments, the cancer comprises a solid tumor (e.g., a colorectal, breast, prostate, lung, pancreatic, renal or ovarian tumor). Accordingly, in some embodiments, the cancer is a solid tumor cancer. In some embodiments, the cancer is selected from one or more of a cancer of the pulmonary system, a brain cancer, a cancer of the gastrointestinal tract, a skin cancer, a genitourinary cancer, head and neck cancer, a sarcoma, a carcinoma, and a neuroendocrine cancer. In various embodiments, the solid tumor cancer is breast cancer, bladder cancer, endometrial cancer, esophageal cancer, liver cancer, pancreatic cancer, lung cancer, cervical cancer, colon cancer, colorectal cancer, gastric cancer, kidney cancer, ovarian cancer, prostate cancer, testicular cancer, uterine cancer, a viral-induced cancer, melanoma or sarcoma. In some embodiments, the cancer is bladder cancer. In some embodiments, the cancer is lung cancer (e.g., non-small cell lung cancer). In other embodiments, the cancer is liver cancer. In some embodiments, the cancer is a sarcoma, bladder cancer or renal cancer. In some embodiments, the cancer is prostate cancer (e.g., castration-resistant prostate cancer, castration-sensitive prostate cancer). In other embodiments, the cancer is bladder cancer, pancreatic cancer, colorectal cancer, glioblastoma, kidney cancer, non-small cell lung carcinoma, prostate cancer, sarcoma, skin cancer, thyroid cancer, testicular cancer or vulvar cancer. In some embodiments, the cancer is endometrial cancer, pancreatic cancer, testicular cancer, renal cancer, melanoma, colorectal cancer, thyroid cancer, bladder cancer, pancreatic cancer, vulvar cancer, sarcoma, prostate cancer, lung cancer or anal cancer. In some embodiments, the cancer is a sarcoma. In some embodiments, the cancer is a renal cell carcinoma.

In some embodiments, the cancer is a non-solid tumor cancer. In some embodiments, the cancer is a hematologic cancer. Hematologic cancers that can be treated according to the methods described herein include leukemias (e.g., acute leukemias, chronic leukemias), lymphomas (e.g., B-cell lymphoma, T-cell lymphoma) and multiple myeloma. In some embodiments, the cancer is a leukemia. In some embodiments, the cancer is an acute leukemia. In some embodiments, the cancer is acute myeloid leukemia or acute lymphocytic leukemia. In some embodiments, the cancer is a chronic leukemia. In some embodiments, the cancer is chronic myeloid leukemia or chronic lymphocytic leukemia. In some embodiments, the cancer is a lymphoma. In some embodiments, the cancer is multiple myeloma. In some embodiments, the hematologic cancer is selected from multiple myeloma, myelodysplastic syndrome (MDS), acute myeloid leukemia (AML), acute lymphoblastic leukemia (ALL), acute lymphocytic leukemia, lymphocytic lymphoma, mycosis fungoides, chronic lymphogenous leukemia, chronic lymphocytic leukemia (CLL), mantle cell lymphoma, diffuse large B-cell lymphoma, follicular lymphoma, Hodgkin's lymphoma, non-Hodgkin's lymphoma or myelofibrosis.

In some embodiments, the cancer is a pre-metastatic cancer. In some embodiments, the cancer is a metastatic cancer.

Examples of cancer treatable according to the methods described herein include, but are not limited to, adenocarcinoma of the breast, prostate, and colon; all forms of bronchogenic carcinoma of the lung; myeloid; melanoma; hepatoma; neuroblastoma; papilloma; apudoma; choristoma; branchioma; malignant carcinoid syndrome; carcinoid heart disease; and carcinoma (e.g., Walker, basal cell, basosquamous, Brown-Pearce, ductal, Ehrlich tumor, Krebs 2, merkel cell, mucinous, lung cancer (e.g., large cell lung cancer, such as squamous cell carcinoma, non-small cell lung), oat cell, papillary, scirrhous, bronchiolar, bronchogenic, squamous cell, and transitional cell). Additional examples of cancer treatable according to the methods described herein include, but are not limited to, histiocytic disorders; leukemia; histiocytosis malignant; Hodgkin's disease; hypereosinophilia, immunoproliferative small; non-Hodgkin's lymphoma; plasmacytoma; reticuloendotheliosis; melanoma; chondroblastoma; chondroma; chondrosarcoma; dermatofibrosarcoma protuberans, fibrotic cancer (myelofibrosis, pancreatic cancer (e.g., pancreatic ductal adenocarcinoma), kidney cancer, liver cancer, lung cancer (e.g., large cell lung cancer, such as squamous cell carcinoma), breast cancer (e.g., inflammatory breast cancer), ovarian cancer (e.g., high grade serious ovarian carcinoma), endometrial cancer, uterine cancer, uterine sarcoma (e.g., uterine leiomyosarcoma), renal cell cancer, sarcoma (e.g., soft tissue sarcoma), malignant fibrous histiocytoma, fibrosarcoma (e.g., dermatofibrosarcoma protuberans) and hepatocellular carcinoma); fibroma; fibrosarcoma; giant cell tumors; histiocytoma; lipoma; liposarcoma; mesothelioma; myxoma; myxosarcoma; osteoma; osteosarcoma; pediatric malignancy, chordoma; craniopharyngioma; dysgerminoma; hamartoma; mesenchymoma; mesonephroma; myosarcoma; ameloblastoma; cementoma; odontoma; teratoma; thymoma; trophoblastic tumor. Further, the following types of cancers are also contemplated as amenable to treatment: adenoma; cholangioma; cholesteatoma; cyclindroma; cystadenocarcinoma; cystadenoma; granulosa cell tumor; gynandroblastoma; hepatocellular cancer, hepatoma; hidradenoma; islet cell tumor; Leydig cell tumor; papilloma; sertoli cell tumor; theca cell tumor; leiomyoma; leiomyosarcoma; myoblastoma; myomma; myosarcoma; rhabdomyoma; rhabdomyosarcoma; ependymoma; ganglioneuroma; glioma; medulloblastoma; meningioma; neurilemmoma; neuroblastoma; neuroepithelioma; neurofibroma; neuroma; paraganglioma; paraganglioma nonchromaffin. Yet more examples of cancer treatable according to the methods described herein include, but are not limited to, angiokeratoma; angiolymphoid hyperplasia with eosinophilia; angioma sclerosing; angiomatosis; glomangioma; hemangioendothelioma; hemangioma; hemangiopericytoma; hemangiosarcoma; lymphangioma; lymphangiomyoma; lymphangiosarcoma; pinealoma; carcinosarcoma; chondrosarcoma; cystosarcoma phyllodes; fibrosarcoma; hemangiosarcoma; leiomyosarcoma; leukosarcoma; liposarcoma; lymphangiosarcoma; myosarcoma; myxosarcoma; ovarian carcinoma; rhabdomyosarcoma; sarcoma; neoplasms; nerofibromatosis; and cervical dysplasia.

Further examples of cancers treatable according to the methods described herein include, but are not limited to, Acute Lymphoblastic Leukemia (ALL); Acute Myeloid Leukemia (AML); Adrenocortical Carcinoma; Adrenocortical Carcinoma, Childhood; AIDS-Related Cancer (e.g., Kaposi Sarcoma, AIDS-Related Lymphoma, Primary CNS Lymphoma); Cancer of the anal region; Anal Cancer; Appendix Cancer; Astrocytomas, Childhood; Atypical Teratoid/Rhabdoid Tumor, Childhood, Central Nervous System (CNS); Neoplasms of the CNS (e.g., primary CNS lymphoma, spinal axis tumors, medulloblastoma, brain stem gliomas or pituitary adenomas), Barrett's esophagus (e.g., pre-malignant syndrome), and mycoses fungoides, Basal Cell Carcinoma of the Skin; Bile Duct Cancer; Bladder Cancer; Bladder Cancer, Childhood; Bone Cancer (including Ewing Sarcoma, Osteosarcoma and Malignant Fibrous Histiocytoma); Brain Tumors/Cancer; Breast Cancer; Burkitt Lymphoma; Carcinoid Tumor (Gastrointestinal); Carcinoid Tumor, Childhood; Cardiac (Heart) Tumors, Childhood; Embryonal Tumors, Childhood; Germ Cell Tumor, Childhood; Primary CNS Lymphoma; Cervical Cancer; Childhood Cervical Cancer; Cholangiocarcinoma; Chordoma, Childhood; Chronic Lymphocytic Leukemia (CLL); Chronic Myelogenous Leukemia (CML); Chronic Myeloproliferative Neoplasms; Colorectal Cancer; Childhood Colorectal Cancer; Craniopharyngioma, Childhood; Cutaneous T-Cell Lymphoma (e.g., Mycosis Fungoides and Sezary Syndrome); Ductal Carcinoma In Situ (DCIS); Embryonal Tumors, Central Nervous System, Childhood; Cancer of the Endocrine system (e.g., cancer of the thyroid, pancreas, parathyroid or adrenal glands), Endometrial Cancer (Uterine Cancer); Ependymoma, Childhood; Esophageal Cancer; Childhood Esophageal Cancer; Esthesioneuroblastoma; Ewing Sarcoma; Extracranial Germ Cell Tumor, Childhood; Extragonadal Germ Cell Tumor; Eye Cancer; Childhood Intraocular Melanoma; Intraocular Melanoma; Retinoblastoma; Fallopian Tube Cancer; Fibrous Histiocytoma of Bone, Malignant, and Osteosarcoma; Gallbladder Cancer; Gastric (Stomach) Cancer; Childhood Gastric (Stomach) Cancer; Gastrointestinal Carcinoid Tumor; Gastrointestinal Stromal Tumors (GIST); Childhood Gastrointestinal Stromal Tumors; Germ Cell Tumors; Childhood Central Nervous System Germ Cell Tumors (e.g., Childhood Extracranial Germ Cell Tumors, Extragonadal Germ Cell Tumors, Ovarian Germ Cell Tumors, Testicular Cancer); Gestational Trophoblastic Disease; Gynecologic Tumors ((e.g., uterine sarcomas, carcinoma of the fallopian tubes, carcinoma of the endometrium, carcinoma of the cervix, carcinoma of the vagina or carcinoma of the vulva), Hairy Cell Leukemia; Head and Neck Cancer; Heart Tumors, Childhood; Hepatocellular (Liver) Cancer; Histiocytosis, Langerhans Cell; Hodgkin Lymphoma; Hypopharyngeal Cancer; Cutaneous or Intraocular Melanoma; Childhood Intraocular Melanoma; Islet Cell Tumors, Pancreatic Neuroendocrine Tumors; Kaposi Sarcoma; Kidney (Renal Cell) Cancer; Langerhans Cell Histiocytosis; Laryngeal Cancer; Leukemia; Lip and Oral Cavity Cancer; Liver Cancer; Lung Cancer (Non-Small Cell and Small Cell); Childhood Lung Cancer; Lymphoma; Male Breast Cancer; Malignant Fibrous Histiocytoma of Bone and Osteosarcoma; Melanoma; Childhood Melanoma; Melanoma, Intraocular (Eye); Childhood Intraocular Melanoma; Merkel Cell Carcinoma; Mesothelioma, Malignant; Childhood Mesothelioma; Metastatic Cancer; Metastatic Squamous Neck Cancer with Occult Primary; Midline Tract Carcinoma With NUT Gene Changes; Mouth Cancer; Multiple Endocrine Neoplasia Syndromes; Multiple Myeloma/Plasma Cell Neoplasms; Mycosis Fungoides; Myelodysplastic Syndromes, Myelodysplastic/Myeloproliferative Neoplasms; Myelogenous Leukemia, Chronic (CML); Myeloid Leukemia, Acute (AML); Myeloproliferative Neoplasms, Chronic; Nasal Cavity and Paranasal Sinus Cancer; Nasopharyngeal Cancer; Neuroblastoma; Non-Hodgkin Lymphoma; Non-Small Cell Lung Cancer; Oral Cancer, Lip and Oral Cavity Cancer and Oropharyngeal Cancer; Osteosarcoma and Malignant Fibrous Histiocytoma of Bone; Ovarian Cancer; Childhood Ovarian Cancer; Pancreatic Cancer; Childhood Pancreatic Cancer; Pancreatic Neuroendocrine Tumors; Papillomatosis (Childhood Laryngeal); Paraganglioma; Childhood Paraganglioma; Paranasal Sinus and Nasal Cavity Cancer; Parathyroid Cancer; Penile Cancer; Pharyngeal Cancer; Pheochromocytoma; Childhood Pheochromocytoma; Pituitary Tumor; Plasma Cell Neoplasm/Multiple Myeloma; Pleuropulmonary Blastoma; Pregnancy and Breast Cancer; Primary Central Nervous System (CNS) Lymphoma; Primary Peritoneal Cancer; Prostate Cancer; Rectal Cancer; Recurrent Cancer; Renal Cell (Kidney) Cancer; Retinoblastoma; Rhabdomyosarcoma, Childhood; Salivary Gland Cancer; Sarcoma (e.g., Childhood Rhabdomyosarcoma, Childhood Vascular Tumors, Ewing Sarcoma, Kaposi Sarcoma, Osteosarcoma (Bone Cancer), Soft Tissue Sarcoma, Uterine Sarcoma); Sezary Syndrome; Skin Cancer; Childhood Skin Cancer; Small Cell Lung Cancer; Small Intestine Cancer; Soft Tissue Sarcoma; Squamous Cell Carcinoma of the Skin; Squamous Neck Cancer with Occult Primary, Metastatic; Stomach (Gastric) Cancer; Childhood Stomach (Gastric) Cancer; T-Cell Lymphoma, Cutaneous (e.g., Mycosis Fungoides and Sezary Syndrome); Testicular Cancer; Childhood Testicular Cancer; Throat Cancer (e.g., Nasopharyngeal Cancer, Oropharyngeal Cancer, Hypopharyngeal Cancer); Thymoma and Thymic Carcinoma; Thyroid Cancer; Transitional Cell Cancer of the Renal Pelvis and Ureter; Ureter and Renal Pelvis (e.g., renal cell carcinoma, carcinoma of the renal pelvis), benign prostatic hypertrophy, parathyroid cancer, Transitional Cell Cancer; Urethral Cancer; Uterine Cancer, Endometrial; Uterine Sarcoma; Vaginal Cancer; Childhood Vaginal Cancer; Vascular Tumors; Vulvar Cancer; and Wilms Tumor and Other Childhood Kidney Tumors.

Metastases of the aforementioned cancers can also be treated in accordance with the methods described herein.

In certain embodiments, the cancer is a hematologic cancer (e.g., leukemia (e.g., acute lymphocytic leukemia (ALL) (e.g., B-cell ALL, T-cell ALL), acute myelocytic leukemia (AML) (e.g., B-cell AML, T-cell AML), chronic myelocytic leukemia (CML) (e.g., B-cell CML, T-cell CML), chronic lymphocytic leukemia (CLL) (e.g., B-cell CLL, T-cell CLL)); lymphoma (e.g., Hodgkin lymphoma (HL) (e.g., B-cell HL, T-cell HL)), non-Hodgkin lymphoma (NHL) (e.g., B-cell NHL such as diffuse large cell lymphoma (DLCL) (e.g., diffuse large B-cell lymphoma)), follicular lymphoma, chronic lymphocytic leukemia/small lymphocytic lymphoma (CLL/SLL), mantle cell lymphoma (MCL), marginal zone B-cell lymphomas (e.g., mucosa-associated lymphoid tissue (MALT) lymphomads, nodal marginal zone B-cell lymphoma, splenic marginal zone B-cell lymphoma), primary mediastinal B-cell lymphoma, Burkitt lymphoma, lymphoplasmacytic lymphoma (i.e., Waldenstrom's macroglobulinemia), hairy cell leukemia (HCL), immunoblastic large cell lymphoma, precursor B-lymphoblastic lymphoma and primary central nervous system (CNS) lymphoma, T-cell NHL such as precursor T-lymphoblastic lymphoma/leukemia, peripheral T-cell lymphoma (PTCL) (e.g., cutaneous T-cell lymphoma (CTCL) (e.g., mycosis fungoides, Sezary syndrome)), angioimmunoblastic T-cell lymphoma, extranodal natural killer T-cell lymphoma, enteropathy type T-cell lymphoma, subcutaneous panniculitis-like T-cell lymphoma, anaplastic large cell lymphoma); heavy chain disease (e.g., alpha chain disease, gamma chain disease, mu chain disease); a myeloproliferative disorder (MPD) (e.g., polycythemia vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)); multiple myeloma (MM); plasma cell neoplasia; familiar hypereosinophilia; inflammatory myofibroblastic tumors; immunocytic amyloidosis). In certain embodiments, the cancer is leukemia. In certain embodiments, the cancer is acute lymphoblastic leukemia (ALL). In certain embodiments, the cancer is early T-cell precursor (ETP)-acute lymphoblastic leukemia (ALL).

In certain embodiments, the cancer is anaplastic astrocytoma, pancreatic cancer, skin cancer, melanoma, metastatic melanoma, colorectal cancer, breast cancer, prostate cancer, renal cancer, hepatocellular cancer, ovarian cancer, HPV-associated cancers (e.g. cervical cancer, oropharyngeal cancer, anal cancer, vulvar/vaginal cancer, and penile cancer), multiple myeloma, myelodysplastic syndrome, myelofibrosis

In certain embodiments, the cancer is liver cancer (e.g., hepatocellular cancer (HCC) (e.g., hepatocellular carcinoma, hepatoblastoma, hepatocellular adenoma), malignant hepatoma, hemangiomas, biliary cancer (e.g., cholangiocarcinoma)). In some embodiments where the cancer is liver cancer it is hepatocellular carcinoma (HCC). In some embodiments, the cancer is lung cancer (e.g., non-small cell lung cancer (NSCLC)). In some embodiments, the cancer is brain cancer (e.g., neuroblastoma, glioblastoma). In some embodiments wherein the cancer is a brain cancer, it is an anaplastic astrocytoma. In some embodiments, the cancer is thyroid cancer (e.g., anaplastic thyroid cancer (ATC)). In some embodiments, the cancer is breast cancer. In some embodiments the cancer is renal cancer. In some embodiments, the cancer is ovarian cancer. In some embodiments, the cancer is an HPV-associated cancer, for example, HPV-associated cervical cancer, HPV-associated oropharyngeal cancer, HPV-associated anal cancer, HPV-associated vulvar/vaginal cancer, and HPV-associated penile cancer. In some embodiments the cancer is colorectal cancer (e.g., colon carcinoma). In some embodiments the cancer is pancreatic cancer (e.g., pancreatic carcinoma). In some embodiments wherein the cancer is a pancreatic cancer, it is pancreatic ductal adenocarcinoma and associated fibrosis CAF. In some embodiments the cancer is skin cancer. In some embodiments wherein the cancer is a skin cancer, it is metastatic melanoma. In some embodiments the cancer is prostate cancer.

In some embodiments the proliferative disease is a hematological cancer (e.g., anaplastic large cell lymphoma (ALCL), myelodysplastic syndrome, multiple myeloma, and myelofibrosis).

In certain embodiments, the cancer is musculoskeletal cancer (e.g., bone cancer (e.g., osteosarcoma, osteoid osteoma, malignant fibrous histiocytoma, Ewing's sarcoma, chordoma, malignant giant cell tumor chordoma, chondrosarcoma osteochondroma, benign chondroma, chondroblastoma chondromyxofibroma, myelodysplastic syndrome (MDS)), muscle cancer (e.g., rhabdomyosarcoma, rhabdomyoma), connective tissue cancer, synovioma).

In certain embodiments, the cancer is a nervous system cancer (e.g., brain cancer (e.g., astrocytoma, medulloblastoma, glioma (e.g., astrocytoma, oligodendroglioma), glioblastomas, glioblastoma multiform, medulloblastoma, ependymoma, germinoma (i.e., pinealoma), oligodendroglioma, schwannoma, retinoblastoma, congenital tumors, craniopharyngioma), spinal cord cancer, neurofibroma (e.g., neurofibromatosis (NF) type 1 or type 2, schwannomatosis), neuroblastoma, primitive neuroectodermal tumors (PNT), meningeal cancer (e.g., meningioma, meningiosarcoma, gliomatosis), skull cancer, acoustic neuroma, ependymoma, hemangioblastoma, ocular cancer (e.g., intraocular melanoma, retinoblastoma)). In certain embodiments, the disease to be treated is a brain tumor. In certain embodiments, the disease is pleomorphic xenoanthrocytoma (PXA). In certain embodiments, the disease is pediatric pleomorphic xenoanthrocytoma (PXA).

In certain embodiments, the cancer is selected from endocrine/exocrine cancers (e.g., thyroid cancer (e.g., papillary thyroid carcinoma, follicular thyroid carcinoma; medullary thyroid carcinoma, multiple endocrine neoplasia type 2A, multiple endocrine neoplasia type 2B, familial medullary thyroid cancer, pheochromocytoma, paraganglioma), pancreatic cancer (e.g., pancreatic andenocarcinoma, intraductal papillary mucinous neoplasm (IPMN), Islet cell tumors, ductal adenocarcinoma, insulinoma, glucagonoma, vipoma), adrenal gland cancer, neuroendocrine cancer (e.g., gastroenteropancreatic neuroendoctrine tumor (GEP-NET), carcinoid tumor), sebaceous gland carcinoma, sweat gland carcinoma). In certain embodiments, the cancer is sweat gland cancer (e.g., sweat gland carcinoma).

In certain embodiments, the cancer is head and neck cancer (e.g., squamous cell carcinoma of the head and neck (SCCHN), adenoid cystic carcinoma).

In certain embodiments, the cancer is oral cancer (e.g., buccal cavity cancer, lip cancer, tongue cancer, mouth cancer, pharynx cancer, hypopharynx cancer (e.g., hypopharyngeal carcinoma), throat cancer (e.g., laryngeal cancer, pharyngeal cancer, nasopharyngeal cancer, oropharyngeal cancer), salivary gland cancer).

In certain embodiments, the cancer is esophageal cancer (e.g., esophageal squamous cell carcinoma, esophageal adenocarcinoma, Barrett's adenocarcinoma, esophageal leiomyosarcoma).

In certain embodiments, the cancer is gastrointestinal cancer (e.g., anal cancer, colorectal cancer (e.g., colon cancer, rectal cancer, colorectal adenocarcinoma), gall bladder cancer, gastric cancer (e.g., stomach cancer (e.g., stomach adenocarcinoma)), gastrointestinal stromal tumor (GIST), small bowel cancer (e.g., appendix cancer, small bowel carcinoma, e.g., small bowel adenocarcinoma), small intestine cancer, large bowel cancer, large intestine cancer).

In certain embodiments, the cancer is cardiovascular cancer (e.g., primary cardiac tumors, angiosarcoma (e.g., lymphangiosarcoma, lymphangioendotheliosarcoma, hemangiosarcoma), endotheliosarcoma (e.g., Kaposi's sarcoma, multiple idiopathic hemorrhagic sarcoma), cardiac myxoma, cardiac rhabdomyoma).

In certain embodiments, the cancer is lung cancer (e.g., bronchus cancer (e.g., bronchogenic carcinoma, bronchial adenoma), alveolar carcinoma, mesothelioma, small cell lung cancer (SCLC), non-small cell lung cancer (NSCLC), lung adenocarcinoma, chondromatous hamartoma, papillary adenocarcinoma).

In certain embodiments, the cancer is a genitourinary cancer (e.g., bladder cancer (e.g., urothelial carcinoma), urethral cancer, kidney cancer (e.g., nephroblastoma a.k.a. Wilms' tumor, renal cell carcinoma), testicular cancer (e.g., seminoma, testicular embryonal carcinoma), germ cell cancer, prostate cancer (e.g., prostate adenocarcinoma), penile cancer (e.g., Paget's disease of the penis and scrotum)).

In certain embodiments, the cancer is a gynecological cancer (e.g., breast cancer (e.g., adenocarcinoma of the breast, papillary carcinoma of the breast, mammary cancer, medullary carcinoma of the breast, triple negative breast cancer, HER-2 positive breast cancer, HER2-negative breast cancer), endometrial cancer (e.g., uterine cancer (e.g., uterine sarcoma, choriocarcinoma), endometrial carcinoma), cervical cancer (e.g., cervical adenocarcinoma), ovarian cancer (e.g., cystadenocarcinoma, ovarian embryonal carcinoma, ovarian adenocarcinoma), germ cell cancer, vulvar cancer (e.g., Paget's disease of the vulva) vaginal cancer, fallopian tube cancer).

In certain embodiments, the cancer is skin cancer (e.g., squamous cell carcinoma (SCC), keratoacanthoma (KA), melanoma, basal cell carcinoma (BCC), dermatofribroma).

In certain embodiments, the cancer is a soft tissue cancer (e.g., intraepithelial neoplasms, epithelial carcinomas, epithelial sarcomas, adenocarcinomas, adenomas, fibrosarcomas, fibromas, liposarcomas, lipomas, myxomas, teratomas).

Myeloproliferative neoplasms are also treatable according to the methods described herein. Non-limiting examples of myeloproliferative neoplasms include myelofibrosis, polycythemia vera and essential thrombocythemia.

In certain embodiments, the cancer is a rare cancer. The term “rare cancer” refers to cancers that occur in a relatively small number of patients.

In certain embodiments, the cancer is lung cancer (e.g., non-small cell lung cancer (NSCLC)), brain cancer (e.g., neuroblastoma, glioblastoma), thyroid cancer (e.g., anaplastic thyroid cancer (ATC)), breast cancer, colorectal cancer (e.g., colon carcinoma), liver cancer (e.g., hepatocellular carcinoma (HCC)), pancreatic cancer (e.g., pancreatic carcinoma), skin cancer (e.g., melanoma), prostate cancer, or a hematological cancer (e.g., anaplastic large cell lymphoma (ALCL), myelodysplastic syndrome).

In some embodiments it is preferred to treat cancers which are driven by TGF-b signaling with one or more ALK-5 inhibitors described herein, for example, compounds of Formulae (I) (II), (III), (IV), or Table 1, or pharmaceutically acceptable salts thereof.

In some embodiments, a proliferative disease, such as cancer, is treated by targeting a tumor stromal cell (e.g., in a tumor microenvironment), such as a cancer-associated fibroblast (CAF), stellate cell or myofibroblast, and/or an immune cell, such as a tumor-associated immune cell (e.g., in the tumor-immune microenvironment), for example, to thereby modulate the tumor-stroma microenvironment and/or the tumor-immune microenvironment.

Cachexia is linked to chronic illness and manifests in involuntary weight loss (e.g., greater than 5% of pre-illness weight) resulting from the atrophy of skeletal muscle and adipose tissues. This condition is distinct from other conditions, like anorexia, where fat stores are depleted but muscle mass remains largely intact. Cachexia affects over half of cancer patients resulting in poor quality of life (fatigue and weakness) and can sometimes even compromise treatment strategies in some individuals. Myostatin, a transforming growth factor-beta (TGF-beta) super-family member, has been well characterized as a negative regulator of muscle growth and development. Without wishing to be bound by any particular theory, it is believed that blocking this pathway would potentially benefit cancer patients, specifically patients with late stage disease and metastasis where cachexia is prominent. Thus, in some embodiments, the disease or condition is cachexia (e.g. cancer cachexia).

In some embodiments, the disease or condition is a fibrotic disease or condition (e.g., fibrotic condition). In some embodiments, the fibrotic condition is associated with a proliferative disease. In some embodiments, the fibrotic condition is present without a comorbidity. In some embodiments, the fibrotic condition is idiopathic pulmonary fibrosis, cardiac fibrosis, a condition associated with cardiac fibrosis (e.g., valvular disease, arrhythmia (e.g., atrial fibrillation), myocardial remodeling (e.g., after infarction), cardiomyopathy (e.g., dilated, ischemic or hypertrophic cardiomyopathy), restenosis (e.g., in-stent restenosis, post-angioplasty restenosis)), liver fibrosis, liver cirrhosis, nonalcoholic steatohepatitis, Peyronie's, Dupuytren's contracture, cystic fibrosis, beta thalassemia, actinic keratosis, hypertension, general inflammatory disorders, dry eye, ulcers, corneal fibrosis, wet age-related macular degeneration, psoriasis, wound closure, chronic kidney disease, renal fibrosis, systemic sclerosis, or chronic Chagas' heart disease. In some embodiments, the fibrotic condition is idiopathic pulmonary fibrosis, liver fibrosis, liver cirrhosis, nonalcoholic steatohepatitis, Peyronie's, cystic fibrosis, beta thalassemia, actinic keratosis, hypertension, general inflammatory disorders, dry eye, ulcers, corneal fibrosis, wet age-related macular degeneration, psoriasis, wound closure, chronic kidney disease, renal fibrosis, systemic sclerosis, or chronic Chagas' heart disease. In some embodiments, the condition is idiopathic pulmonary fibrosis. In some embodiments, the fibrotic condition is cardiac fibrosis or a condition associated with cardiac fibrosis (e.g., valvular disease, arrhythmia (e.g., atrial fibrillation), myocardial remodeling (e.g., after infarction), cardiomyopathy (e.g., dilated, ischemic or hypertrophic cardiomyopathy), restenosis (e.g., in-stent restenosis, post-angioplasty restenosis)). In some embodiments, the fibrotic condition is Dupuytren's contracture. In some embodiments, the fibrotic condition is desmoid tumors (fibromatosis).

As used herein, the terms “fibrosis”, “fibrotic disease,” “fibrotic condition,” “fibrotic lesion” and “fibrotic disease and/or condition” (collectively herein, fibrosis) refer to disease or condition in a subject involving the formation of excess fibrous connective tissue in an organ or tissue. The occurrence of fibrosis may be concomitant with another disease state or condition, for example, inflammation, cancer, viral or bacterial infection or the like.

Fibrosis may be associated with another disease, disorder or condition (e.g., inflammation, an inflammatory disease, disorder or condition, such as psoriasis, a proliferative disease, such as cancer, a viral or bacterial infection or the like) or may occur independently. For example, fibrosis may precede (e.g., be causative of) or follow (e.g., be caused by) another disease, disorder or condition. Fibrosis may also or alternatively be present, whether associated or not, with another disease, disorder or condition (e.g., inflammation, an inflammatory disease, disorder or condition, such as psoriasis, a proliferative disease, such as cancer, a viral or bacterial infection or the like), or may be present without a concomitant disease, disorder or condition (e.g., associated disease, disorder or condition). In some embodiments, the fibrosis is present without an associated disease, disorder or condition. In some embodiments, the fibrosis is present with an associated disease, disorder or condition.

Although the occurrence of fibrosis associated with another disease, disorder or condition is not uncommon, for example, the presence of cancer-associated fibrosis, the etiology of fibrosis is not well understood and fibrosis occurs also independently from and/or in the absence of other diseases, disorders or conditions. However, it is believed that similar mechanisms and signaling pathways are present in both fibrosis and many associated diseases, disorders or conditions affecting organs or tissues in which fibrosis is also present, for example, the presence of IPF with lung cancer. For example, it is believed that fibrosis along with many diseases with which it is often present, progress via the TGFβ protein and the signaling cascade implicated by overexpression of it, see for example, Ballester, B; et al., Idiopathic Pulmonary Fibrosis and lung Cancer: Mechanisms and Molecular targets, Int. J. Mol. Sci. 2019, 20, 593; doi:10.3390/ijms20030593.

Fibrosis can be comorbid with, caused by and/or exacerbated by an associated disease, disorder or condition (e.g., an infection, such as an infection described herein, such as a viral or bacterial infection; an inflammatory disease, disorder or condition, such as an inflammatory disease, disorder or condition described herein, such as psoriasis; or a proliferative disease, such as a proliferative disease described herein, such as cancer, in particular, fibrotic cancer). Thus, in some embodiments, a disease, disorder or condition associated with fibrosis is a comorbid, causative and/or exacerbating disease, disorder or condition. In some embodiments, the fibrosis is comorbid with the associated disease, disorder or condition. For example, fibrosis can be comorbid with an infection, for example, a viral or bacterial infection; an inflammatory disease, disorder or condition, such as an inflammatory disease, disorder or condition described herein, such as psoriasis; or a proliferative disease, such as a proliferative disease described herein, such as cancer, in particular, fibrotic cancer. In some embodiments, the fibrosis is caused by the associated disease, disorder or condition (e.g., the fibrosis is caused by an infection, for example, a viral or bacterial infection; an inflammatory disease, disorder or condition, such as an inflammatory disease, disorder or condition described herein, such as psoriasis; or a proliferative disease, such as a proliferative disease described herein, such as cancer). In some embodiments, the fibrosis is comorbid with and/or caused by the associated disease, disorder or condition (e.g., an infection, for example, a viral or bacterial infection; an inflammatory disease, disorder or condition, such as an inflammatory disease, disorder or condition described herein, such as psoriasis; or a proliferative disease, such as a proliferative disease described herein, such as cancer, in particular, fibrotic cancer). In some embodiments, the fibrosis is exacerbated by the associated disease, disorder or condition. For example, fibrosis can be exacerbated by an infection, for example, a viral or bacterial infection; an inflammatory disease, disorder or condition, such as an inflammatory disease, disorder or condition described herein, such as psoriasis; or a proliferative disease, such as a proliferative disease described herein, such as cancer, in particular, fibrotic cancer.

The formation of excess fibrous connective tissue leading to a fibrosis is believed to occur in an organ or tissue in a reparative or reactive process. This can be a reactive, benign, or pathological state. Physiologically, fibrosis acts to deposit connective tissue, which can interfere with, or totally inhibit the normal architecture and function of the underlying organ or tissue. For example, pulmonary fibrosis is a respiratory disease in which scars are formed in the lung tissues, leading to serious breathing problems. Scar formation typically involves the accumulation of excess fibrous connective tissue, and often leads to thickening of the walls and causes reduced oxygen supply in the blood. Reduced oxygen supply in the blood, in turn, can lead to heart failure, and even death. The replacement of normal lung with scar tissue causes irreversible decrease in oxygen diffusion capacity. Some types of pulmonary fibrosis are believed to be perpetuated by aberrant wound healing, rather than chronic inflammation. Once the scarring has developed, it is often permanent. Idiopathic pulmonary fibrosis (IPF) is a type of pulmonary fibrosis which is a fatal lung disease with an unknown etiology, but can be present with inflammation, cancer, and/or viral infection.

In general, a fibrosis progresses in three stages (illustrated for pulmonary fibrosis, but common across many fibrotic conditions): the injury stage (“Stage 1”), the epithelial-fibroblastic interaction stage (“Stage 2”), and the aberrant repair and fibrosis stage (“Stage 3”). In Stage 1, generally, the epithelium is damaged, and one or more of the following events can occur: epithelial damage, endothelial damage, for example, in pulmonary fibrosis, destruction of an alveolar capillary basilar membrane, vascular leak, platelet activation, and fibrin clot activation. In Stage 2, generally, fibroblasts begin to interact with the damaged epithelium, and one or more of the following events can occur: release of profibrotic cytokines, (myo)fibroblast recruitment, proliferation, and differentiation, provisional matrix formation, angiogenesis, and defective re-epithelialisation. In Stage 3, generally, the epithelial damage is aberrantly repaired resulting in fibrosis, and one or more of the following events can occur: exaggerated extracellular matrix (ECM) accumulation, lack of matrix degradation, for example, in pulmonary fibrosis, progressive lung remodeling and honeycomb changes (in pulmonary fibrosis, the lung tissue comes to resemble a honeycomb).

Although the occurrence of fibrosis concomitant with other disease conditions is not uncommon, for example, the presence of a cancer concomitant with fibrosis, viral infection concomitant with fibrosis or chronic inflammation concomitant with fibrosis, the etiology of fibrosis disease is not well understood and occurs also in the absence of other disease states. However, it is believed that similar mechanisms and signaling pathways are present in both fibrosis conditions and many of the concomitant diseases (including cancers, infections and general inflammation) effecting organs or tissues in which fibrotic disease is also present, for example, the presence of IPF with lung cancer. Accordingly, it is believed that fibrosis along with many diseases with which it is often present progress via the TGFβ protein and the signaling cascade implicated by overexpression of it, see for example, Ballester, B; et al., Idiopathic Pulmonary Fibrosis and lung Cancer: Mechanisms and Molecular targets, Int. J. Mol. Sci. 2019, 20, 593; doi:10.3390/ijms20030593.

Accordingly, in some embodiments, the compounds described herein can be used to treat (e.g., provide therapy for, reverse the course of), ameliorate (e.g., reduce symptoms associated with), prevent (e.g., prophylactically treat) or manage (e.g., slow or halt progression) a fibrotic disease (collectively herein, “treat a fibrotic disease”), such as one or more of the fibrotic diseases described herein. In some embodiments, the fibrosis to be treated is present without any concomitant disease. In some embodiments, the fibrosis to be treated is present with an infection, for example, a viral or bacterial infection. In some embodiments, the fibrosis to be treated is present with an inflammatory condition. In some embodiments, the inflammatory condition present is each and several of those described herein. In some embodiments, treatment comprises identifying a patient who has fibrosis, with or without a concomitant comorbid, causative, or exacerbating condition, or who is at risk of developing a fibrosis, with or without a concomitant comorbid, causative, or exacerbating condition, and administering thereto a therapeutically effective amount of one or more of the compounds described herein, for example one or more compounds of Formulae (I) (II), (III), (IV), or Table 1, or a pharmaceutically acceptable salt of any of the foregoing.

In some embodiments, the fibrosis to be treated is present with a cancer. In some embodiments, the fibrosis is comorbid with the cancerous condition. In some embodiments, the cancer is a cause of the fibrotic condition. In some embodiments, the fibrotic condition is exacerbated by the cancer. In some embodiments, the cancer present is each and several of those described in detail herein, whether as a comorbid, causative or exacerbating condition.

In some embodiments, the fibrosis to be treated is present with a viral infection. In some embodiments the viral infection is comorbid with the fibrotic condition. In some embodiments, the viral infection is a cause of the fibrotic condition. In some embodiments, the fibrotic condition is exacerbated by the viral infection. In some embodiments, the viral infection present is each and several of the viral infections mentioned herein.

In some embodiments, treatment of a fibrotic disease, which can be alone or present with another condition (which can be comorbid, exacerbating or causative of the fibrosis) selected from each and several of a viral infection, a cancer, or an inflammatory condition, for example, each and several of those described herein, is carried out by administering one or more of the compounds described herein, for example, one or more compounds of Formulae (I) (II), (III), (IV), or Table 1, or a pharmaceutically acceptable salt of any of the foregoing. In some embodiments, treatment of a fibrotic disease (with or without a concomitant condition), for example, one or more of those described herein, is carried out by administering two or more compounds as described herein, for example, two or more compounds of Formulae (I) (II), (III), (IV), or of Table 1, or a pharmaceutically acceptable salt of any of the foregoing. In some embodiments, treatment of a fibrotic disease (with or without a concomitant condition), for example, one or more of those described herein, is carried out by administering a combination of therapeutic agents comprising one or more compounds described herein (for example, one or more of the exemplified compounds, or a pharmaceutically acceptable salt thereof), in combination with one or more additional therapeutic agents (e.g., at least one exemplified compound, or a pharmaceutically acceptable salt thereof, at least one additional therapeutic agent). In some embodiments, combination treatment is provided by administering one compound of Formulae (I) (II), (III), (IV), or Table 1, or a pharmaceutically acceptable salt of any thereof, and one or more additional therapeutic agents. In some embodiments, the combination of therapeutic agents comprises one exemplified compound, or a pharmaceutically acceptable salt thereof, and more than one additional therapeutic agent.

In some embodiments, administration of the exemplified compound, or a pharmaceutically acceptable salt thereof, alone or in a combination with one or more additional therapeutic agents occurs during a single stage of the disease (e.g., Stage 1, Stage 2, Stage 3). In some embodiments, fibrosis treatment comprises administration of a combination therapy divided across multiple stages of the disease. As a non-limiting example, an exemplified compound, or a pharmaceutically acceptable salt thereof (for example, one or more of the exemplified compounds), can be administered during Stage 1, Stage 2, or Stage 3 of the disease, while one or more additional therapeutic agents can be administered during a different stage of the disease. For example, in some embodiments, treatment of a fibrotic disease (as described herein) is accomplished by administering an exemplified compound, for example, one or more of the compounds of Formulae (I) (II), (III), (IV), or Table 1, or a pharmaceutically acceptable salt of any of the foregoing. In some embodiments, where a combination is used to treat a proliferative disease, the combination is one or more of the compounds of Formulae (I) (II), (III), (IV), or Table 1, or a pharmaceutically acceptable salt of any thereof, and an immunooncology agent. In some embodiments, the exemplified compound and the additional therapeutic agent(s) of the combination therapy are administered during all stages of the fibrosis. In some embodiments, the exemplified compound, or a pharmaceutically acceptable salt thereof, is provided during some stages and not others. In some embodiments, wherein a combination therapy is employed, the exemplified compound, or pharmaceutically acceptable salt thereof, is administered during all stages of the disease and the additional therapeutic agents with which it is combined are administered during some stages of the disease and not others.

In some embodiments, an exemplified compound, or a pharmaceutically acceptable salt thereof, is administered to a subject in need thereof in an amount effective to treat a fibrotic disease, for example, an amount effective to slow down or stop the progression of a disease or condition (e.g., idiopathic pulmonary fibrosis, acute exacerbation of IPF, cardiac disease, liver fibrosis, liver cirrhosis, nonalcoholic steatohepatitis, Peyronie's, Dupuytren's contracture, cystic fibrosis, beta thalassemia, actinic keratosis, hypertension, general inflammatory disorders, dry eye, ulcers, corneal fibrosis, wet age-related macular degeneration, psoriasis, wound closure, chronic kidney disease, renal fibrosis, systemic sclerosis, and chronic Chagas' heart disease), increase the survival time of a subject suffering with a disease or condition (e.g., by at least 10%, 20%, 30%, 40%, 50%, 60%, 70%, 80%, 90%, or 100%, when compared with a subject not administered the exemplified compound, or a pharmaceutically acceptable salt thereof), increase the survival rate in a subject population (e.g., survival after being admitted to the intensive care unit increase by at least 10%, 20%, 30%, 40%, 50%, 60%, 70%, 80%, 90%, or 100% when compared with a subject population that was not administered the exemplified compound, or a pharmaceutically acceptable salt thereof), reduce the risk of a subject developing a fibrotic condition (e.g., pulmonary fibrosis or IPF) when compared with a subject that was not administered the exemplified compound, or a pharmaceutically acceptable salt thereof, preserve organ function (e.g., lung function or liver function) when compared with a subject that was not administered the exemplified compound, or a pharmaceutically acceptable salt thereof, and/or prevent or reduce the risk of acute exacerbation of a condition when compared with a subject that was not administered the exemplified compound, or a pharmaceutically acceptable salt thereof.

In some embodiments, provided are methods of inhibiting fibrosis in a tissue, comprising contacting the tissue (e.g., in vitro, ex vivo, in vivo) with a compound of the present disclosure (e.g., an effective amount of a compound of the present disclosure). In various embodiments, an effective amount is an amount effective to inhibit the formation or deposition of tissue fibrosis, and/or reduce the size, cellularity, composition, cellular or collagen content of a fibrotic lesion. In some embodiments of the methods described herein, the method involves contacting the tissue with a compound of Formulae (I) (II), (III), (IV), or Table 1, or a pharmaceutically acceptable salt of any thereof, in an amount sufficient to decrease or inhibit fibrosis. In some embodiments of the methods described herein, the methods can include inhibiting the formation or deposition of tissue fibrosis, and/or reducing the size, cellularity, composition, cellular or collagen content of a fibrotic lesion. In some embodiments, the fibrotic lesion is in a subject (e.g., human subject). In some embodiments, the method of inhibiting is applied to a subject which has present a concomitant condition, for example, cancer, inflammation, or viral infection, which is comorbid with, causative of, or exacerbating said fibrosis.

In some embodiments, provided are methods of treating fibrosis in a tissue comprising administering a compound described herein, for example, one or more of the compounds of Formulae (I) (II), (III), (IV), or Table 1, or a pharmaceutically acceptable salt of any of the foregoing. In some embodiments of the methods described herein, the method involves contacting the tissue with the compound described herein, or a pharmaceutically acceptable salt thereof, in an amount sufficient to reverse the progression or eliminate fibrosis. In some embodiments of the methods described herein, the methods can include reversing or eliminating the formation or deposition of tissue fibrosis, and/or reducing the size, cellularity, composition, cellular or collagen content of a fibrotic lesion. In some embodiments, the fibrotic lesion is in a subject (e.g., human subject). In some embodiments, the method of treating is applied to a subject which has present a concomitant condition, for example, cancer, inflammation, or viral infection, which is comorbid with, causative, or exacerbating said fibrosis.

In some embodiments, treatment, amelioration, or prevention (e.g., prophylactic treatment) of a fibrotic condition (e.g., pulmonary fibrosis) which is present with (comorbid, caused by, and/or exacerbated by) a cancer, is provided by administering one or more compounds as described herein, for example, one or more compounds of Formulae (I) (II), (III), (IV), or Table 1, or a pharmaceutically acceptable salt of any of the foregoing.

In some embodiments, treatment, amelioration, or prevention of a fibrotic condition, for example, acute exacerbation of idiopathic pulmonary fibrosis, which is present with a cancerous condition is carried out by administering one or more compounds, for example compounds of Formulae (I) (II), (III), (IV), or Table 1, or a pharmaceutically acceptable salt of any of the foregoing.

In some embodiments, treatment of a fibrotic disease which is present with a cancer, for example, one or more of those described herein, is carried out by administering a combination of therapeutic agents comprising one or more compounds described herein, for example, compounds of Formulae (I) (II), (III), (IV), or Table 1, or a pharmaceutically acceptable salt of any of the foregoing, in combination with one or more additional therapeutic agents. In some embodiments, combination treatment of fibrosis present with a cancer is provided by administering two or more ALK-5 inhibitors, for example, two or more compounds of Formulae (I) (II), (III), (IV), or of Table 1, or a pharmaceutically acceptable salt of any thereof, and one or more additional therapeutic agents.

In some embodiments, treatment, amelioration, or prevention of fibrosis which is comorbid with a viral infection, is carried out by administering one or more ALK-5 inhibitor compounds, for example, compounds of Formulae (I) (II), (III), (IV), or Table 1, or a pharmaceutically acceptable salt of any of the foregoing. In some embodiments, treatment of a fibrotic disease present with a viral infection, for example, one or more of those described herein, is carried out by administering two or more ALK-5 inhibitor compounds, for example, two or more compounds of Formulae (I), (II), (III), (IV), or of Table 1, or a pharmaceutically acceptable salt of any thereof.

In some embodiments, treatment of a fibrotic disease present with a viral infection, for example, one or more of those described herein, is carried out by administering a combination of therapeutic agents comprising one or more compounds described herein, for example, compounds of Formulae (I), (II), (III), (IV), or Table 1, or a pharmaceutically acceptable salt of any of the foregoing, in combination with one or more additional therapeutic agents.

In some embodiments, treatment, amelioration, or prevention of a fibrotic condition present with a viral infection, for example, acute exacerbation of idiopathic pulmonary fibrosis, is carried out by administering one or more compounds, for example, compounds of Formulae (I), (II), (III), (IV), or Table 1, or a pharmaceutically acceptable salt of any of the foregoing.

In some embodiments, treatment, amelioration, or prevention of a fibrotic condition (e.g., pulmonary fibrosis) which is comorbid with, caused by, and/or exacerbated by, an inflammatory condition, is provided by administering one or more compounds described herein, for example, compounds of Formulae (I), (II), (III), (IV), or Table 1, or a pharmaceutically acceptable salt of any of the foregoing. In some embodiments, treatment, amelioration, or prevention of a fibrotic condition (e.g., pulmonary fibrosis) which is present with an inflammatory condition, for example, each and several of those described herein, is carried out by administering two or more compounds described herein, for example, compounds of Formulae (I), (II), (III), (IV), or Table 1, or a pharmaceutically acceptable salt of any of the foregoing.

In some embodiments, treatment, amelioration, or prevention of a fibrotic condition (e.g., pulmonary fibrosis) which is comorbid with, caused by, and/or exacerbated by an inflammatory condition, for example, each and several of those described herein, is carried out by administering a combination of therapeutic agents comprising one or more compounds described herein, for example, one or more compounds of Formulae (I) (II), (III), (IV), or Table 1, or a pharmaceutically acceptable salt of any of the foregoing, in combination with one or more additional therapeutic agents. In some embodiments, combination treatment is provided by administering two or more ALK-5 inhibitor compounds, for example, two or more compounds of Formulae (I) (II), (III), (IV), or Table 1, or a pharmaceutically acceptable salt of any of the foregoing, and one or more additional therapeutic agents. In some embodiments, treatment, amelioration, or prevention of a fibrotic condition which is present with an inflammatory condition, for example, acute exacerbation of idiopathic pulmonary fibrosis, is carried out by administering one or more compounds, for example, compounds of Formulae (I) (II), (III), (IV), or of Table 1, or a pharmaceutically acceptable salt of any of the foregoing.

In some embodiments, a fibrotic condition (e.g., pulmonary fibrosis) is present with one or more additional conditions (a concomitant condition), e.g., an inflammatory condition, a cancer, and/or a viral infection. A concomitant condition may be a cause of, or an exacerbation of, the fibrotic condition, or may be a comorbidity with the fibrotic condition. In some embodiments, the concomitant condition is a viral infection. In some embodiments, the concomitant condition is cancer. In some embodiments, the concomitant condition is an inflammatory condition. In some embodiments, where treatment, amelioration, or prevention of a fibrotic condition (e.g., pulmonary fibrosis) which is present with, caused by, and/or exacerbated by, a cancer, viral infection, or an inflammatory condition is provided, the fibrotic condition is pulmonary fibrosis. In some embodiments, the fibrotic condition is idiopathic pulmonary fibrosis. In some embodiments, the fibrotic condition is an acute exacerbation of idiopathic pulmonary fibrosis.

In some embodiments, a fibrotic condition for which treatment is administered (e.g., pulmonary fibrosis) is present without a concomitant disease state. In some embodiments, treatment of a fibrotic condition present without a concomitant disease state is provided by administering a compound described herein, or a pharmaceutically acceptable salt thereof, for example, a compound of Formulae (I) (II), (III), (IV), or Table 1, or a pharmaceutically acceptable salt of any of the foregoing. In some embodiments, treatment of a fibrotic condition present without a concomitant disease state is provided by administering a therapeutically effective amount of a compound described herein, or a pharmaceutically acceptable salt thereof, for example, a compound of Formulae (I) (II), (III), (IV), or Table 1, or a pharmaceutically acceptable salt of any of the foregoing. In some embodiments, treatment, amelioration, or prevention of a fibrotic condition (e.g., pulmonary fibrosis) which is not present with a concomitant cancer, viral infection, or an inflammatory condition is provided. In some embodiments, the fibrotic condition is pulmonary fibrosis. In some embodiments, the fibrotic condition is idiopathic pulmonary fibrosis. In some embodiments, the fibrotic condition is an acute exacerbation of idiopathic pulmonary fibrosis.

In some embodiments, a fibrotic condition which is treated in accordance with the methods described herein by administration of a compound described herein (alone or as part of a combination therapy), for example, individually or in combinations of two or more of the compounds of Formulae (I) (II), (III), (IV), or Table 1, or a pharmaceutically acceptable salt of any of the foregoing, is, for example but not limited to, a lung fibrosis, commonly known as “scarring of the lungs” (e.g. pulmonary fibrosis, for example, an idiopathic pulmonary fibrosis, an acute exacerbation of an idiopathic pulmonary fibrosis, or a familial pulmonary fibrosis), a liver fibrosis (hepatic fibrosis, e.g., keloids, scleroderma, or nephrogenic systemic fibrosis, a bile duct fibrosis (biliary fibrosis), liver cirrhosis, for example, primary biliary cholangitis (biliary cirrhosis), primary sclerosing cholangitis), fibrosis in the heart tissue (a cardiac fibrosis or restenosis (e.g., in-stent restenosis, post-angioplasty restenosis)), a vascular fibrosis, a kidney fibrosis (renal fibrosis), a skin fibrosis (a cutaneous fibrosis or endometrial fibrosis, e.g., keloids, scleroderma, or nephrogenic systemic fibrosis), a gastrointestinal fibrosis (e.g., Crohn's disease), a bone marrow fibrosis (also called myleofibrosis), an athrofibrosis (e.g., of the knee, of the shoulder, or of another joint), Dupuytren's contracture, a mediastinal fibrosis, Peyronie's disease, a retroperitoneal fibrosis, a systemic sclerosis, autoimmune hepatitis, or two or more thereof.

In some embodiments, the fibrotic condition to be treated is pulmonary fibrosis. In some embodiments, the fibrotic condition to be treated is liver fibrosis. In some embodiments, the fibrotic condition to be treated is liver cirrhosis. In some embodiments, the fibrotic condition to be treated is nonalcoholic steatohepatitis. In some embodiments, the fibrotic condition to be treated is Peyronie's disease. In some embodiments, the fibrotic condition to be treated is cystic fibrosis. In some embodiments, the fibrotic condition to be treated is beta-thalassemia. In some embodiments, the fibrotic condition to be treated is actinic keratosis. In some embodiments, the fibrotic condition to be treated is hypertension. In some embodiments, the fibrotic condition to be treated is a chronic kidney disease, for example renal fibrosis. In some embodiments, the fibrotic condition to be treated is chronic Chagas' heart disease.

In some embodiments, the fibrotic condition to be treated is dry eye, ulcers, corneal fibrosis, wet age-related macular degeneration, chronic wound (failure to heal) or systemic sclerosis. In some embodiments, the fibrotic condition to be treated is psoriasis. In some embodiments, the fibrotic condition is idiopathic pulmonary fibrosis, liver fibrosis, liver cirrhosis, nonalcoholic steatohepatitis, Peyronie's, Dupuytren's contracture, cystic fibrosis, beta thalassemia, actinic keratosis, hypertension, general inflammatory disorders, dry eye, ulcers, corneal fibrosis, wet age-related macular degeneration, psoriasis, wound closure, chronic kidney disease, renal fibrosis, systemic sclerosis, or chronic Chagas' heart disease. In some embodiments, the fibrotic condition is cardiac fibrosis or a condition associated with cardiac fibrosis, for example, valvular disease, arrhythmia (e.g., atrial fibrillation), myocardial remodeling (e.g., after infarction), cardiomyopathy (e.g., dilated, ischaemic or hypertrophic cardiomyopathy), restenosis (e.g. in-stent restenosis, post-angioplasty restenosis). In some embodiments, the fibrotic condition is Dupuytren's contracture.

In some embodiments, a fibrotic condition (e.g., pulmonary fibrosis) may be present with, may be caused by, and/or may be exacerbated by, a viral infection (concomitant with a viral infection). In some embodiments, the viral infection present may be an Orthomyxoviridae viral infection (e.g., an influenza A viral infection or an influenza B viral infection), a Pneumoviridae viral infection (e.g., a metapneumovirus viral infection (e.g., human metapneumovirus (HMPV) infection) or an orthopneumovirus infection (e.g., a respiratory syncytial virus (RSV) (e.g., a human respiratory syncytial virus (HRSV) infection (e.g., a human respiratory syncytial virus A2 (HRSV-A2) infection or a human respiratory syncytial virus B1 (HRSV-B1) infection)))), a Orthohepadnavirus viral infection (e.g., a Hepatitis B virus infection), Hepacivirus viral infection (e.g., a Hepatitis C virus infection), a Paramyxoviridae viral infection (e.g., a Respirovirus infection (e.g., a human parainfluenza virus type 1 (HPIV-1) infection or a human parainfluenza type 3 (HPIV-3) infection) or a Rubulavirus viral infection (e.g., a human parainfluenza virus type 2 (HPIV-2) infection or a human parainfluenza type 4 (HPIV-4) infection)), an Adenoviridae viral infection (e.g., a Mastadenovirus infection (e.g., a human adenovirus B (HAdV-B) infection or a human adenovirus C (HAdV-C) infection)), an Enterovirus viral infection (e.g., a Rhinovirus A infection, a Rhinovirus B infection, or a Rhinovirus C infection).

In some embodiments, treatment is provided for each and several of the fibrosis conditions described herein where each and several of the aforementioned viral infections is present as a comorbid condition, the treatment comprising administering one or more compounds described herein, for example, a compound of Formulae (I) (II), (III), (IV), or Table 1, or a pharmaceutically acceptable salt of any of the foregoing. In some embodiments, treatment of a fibrotic disease, for example, each and several of those described herein, is carried out by administering two or more compounds as described herein, for example two or more compounds of Formulae (I) (II), (III), (IV), or Table 1, or a pharmaceutically acceptable salt of any of the foregoing. In some embodiments, treatment of a fibrotic disease comorbid with a viral infection, for example, each and several of those described herein, is carried out by administering a combination of therapeutic agents comprising one or more compounds described herein (for example, one or more compounds of Formulae (I) (II), (III), (IV), or Table 1, or a pharmaceutically acceptable salt of any of the foregoing), in combination with one or more additional therapeutic agents. In some embodiments, combination treatment is provided by administering one compound of Formulae (I) (II), (III), (IV), or Table 1, or a pharmaceutically acceptable salt thereof, and one or more additional therapeutic agents.

In some embodiments, treatment is provided for each and several of the fibrosis conditions described herein where each and several of these viral infections is present as an exacerbating condition, the treatment comprising administering one or more compounds as described herein, for example, one compound of Formulae (I) (II), (III), (IV), or Table 1, or a pharmaceutically acceptable salt thereof. In some embodiments, treatment of a fibrotic disease present with an exacerbating viral infection, for example, each and several of those described herein, is carried out by administering two or more compounds as described herein, for example, two or more compounds of Formulae (I) (II), (III), (IV), or Table 1, or a pharmaceutically acceptable salt of any of the foregoing. In some embodiments, treatment of a fibrotic disease present with an exacerbating viral infection, for example, one or more of those described herein, is carried out by administering a combination of therapeutic agents comprising one or more compounds described herein (for example, one or more compounds of Formulae (I) (II), (III), (IV), or Table 1, or a pharmaceutically acceptable salt of any of the foregoing), in combination with one or more additional therapeutic agents. In some embodiments, combination treatment is provided by administering one compound of Formulae (I) (II), (III), (IV), or Table 1, or a pharmaceutically acceptable salt thereof, and one or more additional therapeutic agents.

In some embodiments, treatment is provided for fibrosis present with each and several of these viral infections as a cause of the fibrosis, the treatment comprising administering one or more ALK-5 inhibitor compounds as described herein, for example, one ALK-5 inhibitor compound of Formulae (I) (II), (III), (IV), or Table 1, or a pharmaceutically acceptable salt thereof. In some embodiments, treatment of a fibrotic disease present with a causative viral infection, for example, each and several of those described herein, is carried out by administering two or more compounds as described herein, for example, two or more compounds of Formulae (I) (II), (III), (IV), or Table 1, or a pharmaceutically acceptable salt of any of the foregoing. In some embodiments, treatment of a fibrotic disease present with a causative viral infection, for example, one or more of those described herein, is carried out by administering a combination of therapeutic agents comprising one or more compounds described herein (for example, one or more compounds of Formulae (I) (II), (III), (IV), or Table 1, or a pharmaceutically acceptable salt of any of the foregoing), in combination with one or more additional therapeutic agents. In some embodiments, combination treatment is provided by administering one ALK-5 inhibitor compound of Formulae (I) (II), (III), (IV), or Table 1, or a pharmaceutically acceptable salt thereof, and one or more additional therapeutic agents.

In some embodiments, a fibrotic condition (e.g., pulmonary fibrosis) may be present with, may be caused by, and/or may be exacerbated by, an inflammatory condition. As used herein, the terms “inflammatory disease”, “inflammatory condition”, and “inflammatory disease and/or condition” refer to disease or condition in a subject involving the response of one or more body tissues to stimuli recognized as harmful by the body. In some embodiments, an inflammatory condition is an autoimmune condition. Exemplary inflammatory conditions include non-alcoholic fatty liver disease (NAFLD), alcoholic steatohepatitis (ASH), non-alcoholic steatohepatitis (NASH), primary biliary cholangitis (PBC), primary sclerosing cholangitis, and autoimmune hepatitis. NAFLD is a condition in which fat is deposited in the liver due to causes other than excessive alcohol use, and NASH is an advanced form of NAFLD, wherein the liver is both enflamed and damaged. Aberrant damage repair in NASH can lead to cirrhosis. ASH is a condition in which the liver is enflamed and damaged associated with alcohol use, and it can include liver fibrosis and/or cirrhosis. PBC is an autoimmune disease of the liver, and aberrant repair of liver damage can lead to scarring, fibrosis, and/or cirrhosis. Primary sclerosing cholangitis can be characterized by inflammation and scarring of the bile ducts, which can lead to fibrosis and/or cirrhosis. Autoimmune hepatitis can cause inflammation of the liver, aberrant repair of which can lead to fibrosis and/or cirrhosis.

In some embodiments, treatment is provided for fibrosis present with each and several of these inflammatory conditions present as a comorbid condition of fibrosis, the treatment comprising administering one or more compounds as described herein, for example one compound of Formulae (I) (II), (III), (IV), or Table 1, or a pharmaceutically acceptable salt thereof. In some embodiments, treatment of a fibrotic disease comorbid with an inflammatory condition, for example, each and several of those described herein, is carried out by administering two or more compounds as described herein, for example two or more compounds of Formulae (I) (II), (III), (IV), or Table 1, or a pharmaceutically acceptable salt of any of the foregoing. In some embodiments, treatment of a fibrotic disease comorbid with an inflammatory condition, for example, one or more of those described herein, is carried out by administering a combination of therapeutic agents comprising one or more compounds described herein (for example, one or more compounds of Formulae (I) (II), (III), (IV), or Table 1, or a pharmaceutically acceptable salt of any of the foregoing), in combination with one or more additional therapeutic agents. In some embodiments, combination treatment is provided by administering one compound of Formulae (I) (II), (III), (IV), or Table 1, or a pharmaceutically acceptable salt thereof, and one or more additional therapeutic agents.

In some embodiments, treatment is provided for each of these inflammatory conditions present as an exacerbating condition of fibrosis, the treatment comprising administering one or more compounds as described herein, for example, one compound of Formulae (I) (II), (III), (IV), or Table 1, or a pharmaceutically acceptable salt thereof. In some embodiments, treatment of a fibrotic disease present with an exacerbating inflammatory condition, for example, each and several of those described herein, is carried out by administering two or more compounds of Formulae (I) (II), (III), (IV), or Table 1, or a pharmaceutically acceptable salt of any of the foregoing. In some embodiments, treatment of a fibrotic disease present with an exacerbating inflammatory condition, for example, one or more of those described herein, is carried out by administering a combination of therapeutic agents comprising one or more compounds described herein (for example, one or more compounds of Formulae (I) (II), (III), (IV), or Table 1, or a pharmaceutically acceptable salt of any of the foregoing), in combination with one or more additional therapeutic agents. In some embodiments, combination treatment is provided by administering one compound of Formulae (I) (II), (III), (IV), or Table 1, or a pharmaceutically acceptable salt thereof, and one or more additional therapeutic agents.

In some embodiments, treatment is provided for each of these inflammatory conditions present as a cause of the fibrosis, the treatment comprising administering one or more compounds as described herein, for example, one compound of Formulae (I) (II), (III), (IV), or Table 1, or a pharmaceutically acceptable salt thereof. In some embodiments, treatment of a fibrotic disease present with a causative inflammatory condition, for example, each and several of those described herein, is carried out by administering two or more compounds of Formulae (I) (II), (III), (IV), or Table 1, or a pharmaceutically acceptable salt of any of the foregoing. In some embodiments, treatment of a fibrotic disease present with a causative inflammatory condition, for example, one or more of those described herein, is carried out by administering a combination of therapeutic agents comprising one or more compounds described herein (for example, one or more compounds of Formulae (I) (II), (III), (IV), or Table 1, or a pharmaceutically acceptable salt of any of the foregoing), in combination with one or more additional therapeutic agents. In some embodiments, combination treatment is provided by administering one compound of Formulae (I) (II), (III), (IV), or Table 1, or a pharmaceutically acceptable salt thereof, and one or more additional therapeutic agents.

Provided herein is a method of treating a fibrotic, inflammatory or proliferative disease or condition which is susceptible to inhibition of the TGFβ signaling pathway, the method comprising administering to a subject suffering from said fibrotic, inflammatory or proliferative disease or condition an amount of a compound, or a pharmaceutically acceptable salt form thereof, or a pharmaceutical composition of the foregoing, as described herein, effective to inhibit TGFβ signaling.

Also provided herein is a method of inhibiting TGFβ signaling in a subject suffering from a disease or condition which is promoted by TGFβ signaling, in particular TGF-β1 signaling, comprising administering an amount of at least one compound, or a pharmaceutically acceptable salt thereof, or a pharmaceutical composition of the foregoing, as described herein, effective to sufficiently suppress said TGFβ signaling to alter the course of the disease or condition.

Also provided herein is a method of inhibiting epithelial to mesenchymal transition (EMT) in a subject suffering from a disease or condition which is promoted by EMT, comprising administering an amount of at least one compound, or a pharmaceutically acceptable salt thereof, or a pharmaceutical composition of the foregoing, as described herein, effective to sufficiently inhibit EMT to alter the course of the disease or condition (e.g., a therapeutically effective amount).

Additionally, provided herein are methods of inhibiting tumor growth in a subject (e.g., a subject in need thereof), the methods comprising administering to the subject a therapeutically effective amount of a compound of Formula (I), or a pharmaceutically acceptable salt thereof, or a pharmaceutical composition of the foregoing. Also provided herein are compounds of Formula (I), and pharmaceutically acceptable salts thereof, and pharmaceutical compositions of the foregoing, for use in inhibiting tumor growth. Also provided herein are uses of compounds of Formula (I), and pharmaceutically acceptable salts thereof, and pharmaceutical compositions of the foregoing, for the manufacture of a medicament for inhibiting tumor growth.

Also provided herein are methods for inhibiting activin receptor-like kinase (e.g., ALK-5) activity in vivo or in vitro, the methods comprising contacting the activin receptor-like kinase (e.g., ALK-5) with one or more of the exemplified compounds, for example, one or more compounds of Formulae (I) (II), (III), (IV), or Table 1, or a pharmaceutically acceptable salt thereof, or a pharmaceutical composition of the foregoing. Also provided herein are compounds of compounds of Formulae (I) (II), (III), (IV), or Table 1, and pharmaceutically acceptable salts thereof, and pharmaceutical compositions of the foregoing, for use in inhibiting activin receptor-like kinase (e.g., ALK-5) activity in vivo or in vitro. Also provided herein are uses of compounds of Formulae (I) (II), (III), (IV), or Table 1, and pharmaceutically acceptable salts thereof, and pharmaceutical compositions of the foregoing, for the manufacture of a medicament for inhibiting activin receptor-like kinase (e.g., ALK-5) activity in vivo or in vitro. In certain embodiments, the inhibition occurs in vivo in a subject. In certain embodiments, the inhibition occurs in vitro (e.g., in a cell line or biological sample). In certain embodiments, the methods and uses are for inhibiting ALK-5. In certain embodiments, the inhibition is selective for ALK-5, i.e., selective for ALK-5 over other kinases (e.g., selective for ALK-5 over other activin receptor-like kinases, such as ALK-2; JAK2). In certain embodiments, the inhibition is selective for ALK-5 over ALK-2.

Also provided herein are methods for targeting a tumor stromal cell or immune cell (e.g., tumor-associated immune cell), and/or (e.g., and thereby) modulating (e.g., normalizing) tumor microenvironment (e.g., tumor-stroma microenvironment and/or tumor-immune microenvironment) in vivo or in vitro, the methods comprising contacting a tumor stromal cell or an immune cell (e.g., a tumor-associated immune cell) with one or more of the exemplified compounds, for example, one or more compounds of Formulae (I) (II), (III), (IV), or Table 1, or a pharmaceutically acceptable salt thereof, or a pharmaceutical composition of the foregoing. Also provided herein are compounds of compounds of Formulae (I) (II), (III), (IV), or Table 1, and pharmaceutically acceptable salts thereof, and pharmaceutical compositions of the foregoing, for use in targeting a tumor stromal cell or immune cell (e.g., tumor-associated immune cell), and/or (e.g., and thereby) modulating (e.g., normalizing) tumor microenvironment (e.g., tumor-stroma microenvironment and/or tumor-immune microenvironment) in vivo or in vitro. Also provided herein are uses of compounds of Formulae (I) (II), (III), (IV), or Table 1, and pharmaceutically acceptable salts thereof, and pharmaceutical compositions of the foregoing, for the manufacture of a medicament for targeting a tumor stromal cell or immune cell (e.g., tumor-associated immune cell), and/or (e.g., and thereby) modulating (e.g. normalizing) tumor microenvironment (e.g., tumor-stroma microenvironment and/or tumor-immune microenvironment) in vivo or in vitro. In certain embodiments, the inhibition occurs in vivo in a subject. In certain embodiments, the inhibition occurs in vitro (e.g., in a cell line or biological sample). In certain embodiments, the tumor stromal cell is a cancer-associated fibroblast (CAF), a stellate cell or a myofibroblast.

The tumor microenvironment often favors tumor growth and survival by favoring cancer biology over healthy cellular function. In particular, “excluded” or “desert” phenotypes create optimal microenvironments for cancer cells to avoid immune surveillance, for the microenvironment to have high acidity and hypoxia, and for there to be high interstitial pressure. This tumor microenvironment prevents the beneficial effects of, for example, immunooncology agents, while poor perfusion and interstitial pressure hinder drug delivery.

“Desert phenotype,” as used herein to describe a cancer, refers to an immune phenotype of a tumor characterized by absence or substantial absence of T cells within the tumor and at its margin(s). This phenotype may be caused by factors including, but not limited to, insufficient priming, defects in antigen presentation, and/or lack of antigen.

“Excluded phenotype” as used herein to describe a cancer, refers to an immune phenotype of a tumor characterized by T cells located only or substantially only at the margin(s) of the tumor. In an “excluded phenotype,” T cells are absent or substantially absent from the tumor bed. This phenotype may be caused by factors including, but not limited to, stromal barriers, aberrant vasculature, lack of chemokines, oncogenic pathways, and/or hypoxia.

The tumor microenvironment can be beneficially modulated by promoting an infiltrated phenotype. “Infiltrated phenotype” and “immune-inflamed phenotype,” as used herein to refer to a cancer, refer to an immune phenotype of a tumor characterized by T cells located throughout or substantially throughout the tumor bed. Promotion of this desirable phenotype may be affected, for example, by inhibiting TGFβ, increasing vascularization (e.g., angiogenesis), decreasing tumor induration, increasing antigen presentation, de-activating cancer-associated fibroblasts, increasing T cell infiltration into a tumor bed, or any combination thereof.

Without wishing to be bound by any particular theory, it is believed that compounds of the present disclosure can modulate the tumor microenvironment (e.g., tumor-stroma microenvironment and/or tumor-immune microenvironment) as, for example, by promoting an infiltrated phenotype. Accordingly, in some embodiments, provided herein is a method for modulating (e.g., normalizing) a tumor microenvironment (e.g., tumor-stroma microenvironment and/or tumor-immune microenvironment) in vitro or in vivo (e.g., in a subject, such as a subject having a tumor), the method comprising contacting the tumor and/or the tumor microenvironment with an effective amount of a compound of the disclosure, for example, one or more compounds of Formulae (I), (II), (III), (IV), or Table 1, or a pharmaceutically acceptable salt thereof, or a pharmaceutical composition thereof. In some embodiments wherein modulating occurs in vivo in a subject in need thereof, the method comprises administering to the subject a therapeutically effective amount of the compound of the disclosure or the pharmaceutical composition thereof.

Without wishing to be bound by any particular theory, it is believed that the exemplified compounds can normalize the tumor microenvironment and thereby improve blood vessel perfusion and drug delivery. Enhanced drug delivery is expected, in turn, to enhance the efficacy of a drug, such as an immunomodulator (e.g., immunooncology agent), including any immunomodulators described herein. Accordingly, also provided herein are methods for modulating (e.g., normalizing) tumor microenvironment (e.g., tumor-stroma microenvironment and/or tumor-immune microenvironment) in vivo or in vitro, the methods comprising contacting a tumor with one or more of the exemplified compounds, for example, one or more compounds of Formulae (I) (II), (III), (IV), or Table 1, or a pharmaceutically acceptable salt thereof, or a pharmaceutical composition of the foregoing. Also provided herein are compounds of compounds of Formulae (I) (II), (III), (IV), or Table 1, and pharmaceutically acceptable salts thereof, and pharmaceutical compositions of the foregoing, for use in modulating (e.g., normalizing) tumor microenvironment (e.g., tumor-stroma microenvironment and/or tumor-immune microenvironment) in vivo or in vitro. Also provided herein are uses of compounds of Formulae (I) (II), (III), (IV), or Table 1, and pharmaceutically acceptable salts thereof, and pharmaceutical compositions of the foregoing, for the manufacture of a medicament for modulating (e.g., normalizing) tumor microenvironment (e.g., tumor-stroma microenvironment and/or tumor-immune microenvironment) in vivo or in vitro. In certain embodiments, the inhibition occurs in vivo in a subject. In certain embodiments, the inhibition occurs in vitro (e.g., in a cell line or biological sample).

Also provided is a method for promoting immune infiltration (e.g., immune cell, such as T-cell, infiltration) into a tumor in vitro or in vivo (e.g., in a subject, such as a subject having a tumor), the method comprising contacting the tumor with an effective amount of a compound of the disclosure, for example, one or more compounds of Formulae (I), (II), (III), (IV), or Table 1, or a pharmaceutically acceptable salt thereof, or a pharmaceutical composition thereof. In some embodiments wherein the method occurs in vivo in a subject in need thereof, the method comprises administering to the subject a therapeutically effective amount of the compound of the disclosure or the pharmaceutical composition thereof.

Also provided is a method for promoting tumor vascularization (e.g., angiogenesis) in vitro or in vivo (e.g., in a subject, such as a subject having a tumor), the method comprising contacting the tumor with an effective amount of a compound of the disclosure, for example, one or more compounds of Formulae (I) (II), (III), (IV), or Table 1, or a pharmaceutically acceptable salt thereof, or a pharmaceutical composition thereof. In some embodiments wherein the method occurs in vivo in a subject in need thereof, the method comprises administering to the subject a therapeutically effective amount of the compound of the disclosure or the pharmaceutical composition thereof.

In some embodiments, provided herein is a method for inhibiting metastasis of a cancer, the method comprising administering to the subject (e.g., a therapeutically effective amount of) a compound of the present disclosure, or a pharmaceutical composition thereof.

In one embodiment, provided herein is a method of treating cachexia in a subject in need thereof, comprising administering to the subject an effective amount (e.g., therapeutically effective amount) of a compound of any one of Formula (I) (II), (III), (IV), or Table 1, or a pharmaceutically acceptable salt thereof, or a pharmaceutical composition comprising one or more compounds of Formula (I) (II), (III), (IV), or Table 1, or a pharmaceutically acceptable salt thereof.

In one embodiment, provided herein is a method of promoting immune infiltration in a tumor-immune microenvironment in a subject in need thereof, comprising administering to the subject an effective amount (e.g., therapeutically effective amount) of a compound of any one of Formula (I) (II), (III), (IV), or Table 1, or a pharmaceutically acceptable salt thereof, or a pharmaceutical composition comprising one or more compounds of Formula (I) (II), (III), (IV), or Table 1, or a pharmaceutically acceptable salt thereof.

In one embodiment, provided herein is a method of inhibiting epithelial-to-mesenchymal transition in a tumor in a subject in need thereof, comprising administering to the subject an effective amount (e.g., therapeutically effective amount) of a compound of any one of Formula (I) (II), (III), (IV), or Table 1, or a pharmaceutically acceptable salt thereof, or a pharmaceutical composition comprising one or more compounds of Formula (I) (II), (III), (IV), or Table 1, or a pharmaceutically acceptable salt thereof.

In one embodiment, provided herein is a method for modulating the tumor-immune microenvironment in a subject in need thereof, comprising administering to the subject an effective amount (e.g., therapeutically effective amount) of a compound of any one of Formula (I) (II), (III), (IV), or Table 1, or a pharmaceutically acceptable salt thereof, or a pharmaceutical composition comprising one or more compounds of Formula (I) (II), (III), (IV), or Table 1, or a pharmaceutically acceptable salt thereof.

In one embodiment, provided herein is a method for increasing tumor vasculature or blood flow to a tumor or both in a subject in need thereof, comprising administering to the subject an effective amount (e.g., therapeutically effective amount) of a compound of any one of Formula (I) (II), (III), (IV), or Table 1, or a pharmaceutically acceptable salt thereof, or a pharmaceutical composition comprising one or more compounds of Formula (I) (II), (III), (IV), or Table 1, or a pharmaceutically acceptable salt thereof.

In one embodiment, provided herein is a method for inhibiting metastasis of a cancer in a subject in need thereof, comprising administering to the subject an effective amount (e.g., therapeutically effective amount) of a compound of any one of Formula (I) (II), (III), (IV), or Table 1, or a pharmaceutically acceptable salt thereof, or a pharmaceutical composition comprising one or more compounds of Formula (I) (II), (III), (IV), or Table 1, or a pharmaceutically acceptable salt thereof.

Exemplified compounds, and pharmaceutically acceptable salts thereof, and pharmaceutical compositions of the foregoing, can be administered via a variety of routes of administration, including, for example, oral, dietary, topical, transdermal, rectal, parenteral (e.g., intra-arterial, intravenous, intramuscular, subcutaneous injection, intradermal injection), intravenous infusion and inhalation (e.g., intrabronchial, intranasal or oral inhalation, intranasal drops) routes of administration, depending on the compound and the particular disease to be treated. Administration can be local or systemic as indicated. The preferred mode of administration can vary depending on the particular compound chosen. In some embodiments, the compound of the present disclosure is administered orally. In some embodiments, the compound of the present disclosure is administered intravenously.

### Combination Therapies

Besides administration as monotherapy, the exemplified compounds, and pharmaceutically acceptable salts thereof, and pharmaceutical compositions of the foregoing, can be administered in combination with other therapeutic agents and/or treatment modalities. Accordingly, in some embodiments, the methods further comprise administering to the subject one or more additional therapies (e.g., therapeutic agents). Suitable additional therapies (e.g., therapeutic agents) for use in the methods, compositions and combinations disclosed herein include those discussed herein.

The term “combination therapy” refers to the administration of two or more therapeutic agents to treat a disease, disorder or condition described herein. Such administration encompasses co-administration of the therapeutic agents in a substantially simultaneous manner, such as in a single capsule having a fixed ratio of active ingredients. Alternatively, such administration encompasses co-administration in multiple, or in separate containers (e.g., capsules, powders, and liquids) for each active ingredient. Such administration also encompasses use of each type of therapeutic agent in a sequential manner, either at approximately the same time or at different times. An exemplified compound, or a pharmaceutically acceptable salt thereof, or a composition of the foregoing, and an additional therapeutic agent(s) can be administered via the same administration route or via different administration routes. Powders and/or liquids may be reconstituted or diluted to a desired dose prior to administration. Typically, the treatment regimen will provide beneficial effects of the drug combination in treating the diseases, conditions or disorders described herein.

In some embodiments, the compound of the disclosure and the additional therapy(ies) are co-administered, e.g., in a simultaneous or substantially simultaneous manner. In some embodiments, the compound of the disclosure and the additional therapy(ies) are administered sequentially, either at approximately the same time or at different times. For example, the compound of the disclosure can be administered before the additional therapy(ies). Or, the compound of the disclosure can be administered after the additional therapy(ies).

In some embodiments, a therapy for use in combination with a compound of the present disclosure provides an agent known to modulate other pathways, or other components of the same pathway, or even overlapping sets of target enzymes when used in combination with a compound as described herein. The compounds of Formulae (I) (II), (III), (IV), or Table 1 or a pharmaceutically acceptable salt thereof or a composition of the foregoing, can be administered in combination with one or more additional therapies (e.g., therapeutic agents), for example, that improve the activity, potency and/or efficacy in treating a disease in a subject in need thereof, in preventing a disease in a subject in need thereof, in reducing the risk to develop a disease in a subject in need thereof, and/or in inhibiting the activity of a protein kinase in a subject or cell; improve bioavailability; improve safety; reduce drug resistance; reduce and/or modify metabolism; inhibit excretion; and/or modify distribution in a subject or cell of the compounds of Formulae (I) (II), (III), (IV), or Table 1 or a pharmaceutically acceptable salt thereof or a composition of the foregoing. It will also be appreciated that the additional therapy(ies) employed may achieve a desired effect for the same disorder, and/or it may achieve different effects. In one aspect, such therapy includes but is not limited to the combination of a compound as described herein with chemotherapeutic agents, therapeutic antibodies, and radiation treatment, to provide a synergistic or additive therapeutic effect.

Compositions for use in combination therapies will either be formulated together as a pharmaceutical combination, or provided for separate administration (e.g., associated in a kit). Accordingly, a further embodiment is a pharmaceutical combination comprising an exemplified compound, or a pharmaceutically acceptable salt thereof, or a composition of the foregoing (e.g., a therapeutically effective amount of an exemplified compound, or a pharmaceutically acceptable salt thereof, or a composition of the foregoing), and one or more other therapeutic agents (e.g., a therapeutically effective amount of one or more other therapeutic agents). A pharmaceutical combination can further comprise one or more pharmaceutically acceptable carriers, such as one or more of the pharmaceutically acceptable carriers described herein.

When administered in combination with another therapy, an exemplified compound, or a pharmaceutically acceptable salt thereof, or a composition of the foregoing, can be administered before, after or concurrently with the other therapy (e.g., an additional therapeutic agent(s)). When two or more therapeutic agents are co-administered simultaneously (e.g., concurrently), the exemplified compound, or a pharmaceutically acceptable salt thereof, and other therapeutic agent(s) can be in separate formulations or the same formulation. Alternatively, the exemplified compound, or a pharmaceutically acceptable salt thereof, or a composition of the foregoing, and other therapy can be administered sequentially (e.g., as separate compositions) within an appropriate time frame as determined by a skilled clinician (e.g., a time sufficient to allow an overlap of the pharmaceutical effects of the exemplified compound, or a pharmaceutically acceptable salt thereof, or a composition of the foregoing, and the other therapy).

Additional therapeutic agents include therapeutically active agents. Therapeutic agents also include prophylactically active agents. Therapeutic agents include small organic molecules such as drug compounds (e.g., compounds approved for human or veterinary use by the U.S. Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids, DNAs, RNAs, nucleotides, nucleosides, oligonucleotide, antisense oligonucleotides, lipids, hormones, vitamins, and cells. Each additional therapeutic agent may be administered at a dose and/or on a time schedule determined for that therapeutic agent. The additional therapeutic agents may also be administered together with each other and/or with the compound or composition described herein in a single dose or administered separately in different doses. The particular combination to employ in a regimen will take into account, for example, compatibility of the compound described herein with the additional therapeutic agent(s) and/or the desired therapeutic and/or prophylactic effect to be achieved. In general, it is expected that the additional therapeutic agent(s) in combination be utilized at levels that do not exceed the levels at which they are utilized individually. In some embodiments, the levels utilized in combination will be lower than those utilized individually.

In certain embodiments, the additional therapeutic agent is selected from the group consisting of anti-metabolites, DNA-fragmenting agents, DNA-crosslinking agents, intercalating agents, protein synthesis inhibitors, topoisomerase I poisons, (e.g., camptothecin or topotecan), topoisomerase II poisons, microtubule-directed agents, kinase inhibitors, hormones, and hormone antagonists.

Examples of therapies for use in combination with a compound of the present disclosure (e.g., in combination therapy, in a pharmaceutical combination) include standard of care therapies and/or regimens (e.g., standard of care agents), such as first-line standard of care therapies (e.g., chemotherapies) or last-line standard of care therapies (e.g., chemotherapies). Standard of care therapies are therapies that a clinician should use for a certain type of patient, illness and/or clinical circumstance.

In some embodiments, a compound of the present disclosure is administered in combination with a standard of care therapy for ovarian cancer. For example, non-limiting examples of standard of care therapies for ovarian cancer include a platinum analogue (e.g., cisplatin, paclitaxel, carboplatin) or a combination including a platinum analogue (e.g., docetaxel and carboplatin; paclitaxel and carboplatin; carboplatin and liposomal doxorubicin (dox); paclitaxel, carboplatin and bevacizumab (bev); carboplatin and gemcitabine (gem)/(bev); carboplatin, liposomal dox and bev; carboplatin, paclitaxel and bev; cisplatin and gemcitabine; oxaliplatin); altretamine; capecitabine; ifosfamide; irinotecan; melphalan; paclitaxel (e.g., albumin-bound paclitaxel); pemetrexed; or vinorelbine. Non-limiting examples of standard of care therapies for ovarian cancer also include a targeted therapy, such as an antibody therapy (e.g., bevacizumab); a PARP inhibitor (e.g., olaparib, rucaparib, niraparib, veliparib, talazoparib); a tyrosine kinase inhibitor (TKI) (e.g, pazopanib); an immunotherapy; an immune checkpoint inhibitor (e.g., PD-1 or PD-L1 inhibitor); pembrolizumab; or a hormone therapy (e.g., tamoxifen, anastrozole, exemestane, letrozole, an LHRH agonist, such as leuprolide acetate, megestrol acetate). Non-limiting examples of standard of care therapies for ovarian cancer further include a hormone therapy (e.g., anastrozole, exemestane, letrozole, leuprolide acetate, megestrol acetate, tamoxifen). Non-limiting examples of standard of care therapies for ovarian cancer additionally include cyclophosphamide; etoposide; sorafenib; or vinorelbine.

In some embodiments, a compound of the present disclosure is administered in combination with a standard of care therapy for pancreatic cancer. Non-limiting examples of standard of care therapies for pancreatic cancer include FOLFIRINOX (a chemotherapy regimen made up of folinic acid, bolus fluorouracil, irinotecan and oxaliplatin); modified FOLFIRINOX regimen (a chemotherapy regimen made up of folinic acid, continuous infusion fluorouracil, irinotecan and oxaliplatin); gemcitabine and nab-paclitaxel; gemcitabine and capecitabine; olaparib; gemcitabine and erlotinib; gemcitabine, docetaxel and capecitabine; larotrectinib; pembrolizumab; gemcitabine; and the triple combination of nab-paclitaxel, gemcitabine and cisplatin.

In some embodiments, a compound of the present disclosure is administered in combination with a standard of care therapy for prostate cancer, including castration resistant prostate cancer. Non-limiting examples of standard of care therapies for prostate cancer include PARP inhibitors (e.g., olaparib, rucaparib, niraparib, veliparib, talazoparib), LHRH agonists (e.g., goserelin acetate, histrelin acetate, leuprolide acetate, and triptorelin pamoate); LHRH antagonists (e.g., degarelix); anti-androgen receptors (e.g., bicalutamide, flutamide, nilutamide, enzalutamide, apalutamide, darolutamide); corticosteroids (e.g., prednisone, methylprednisolone, hydrocortisone, dexamethasone); estrogens (e.g., diethylstilbestrol); androgen synthesis inhibitors (e.g., ketoconazole, abiraterone acetate); and androgen deprivation therapies.

In some embodiments, a compound of the present disclosure is administered in combination with a standard of care therapy for multiple myeloma. Non-limiting examples of standard of care therapies for multiple myeloma include proteasome inhibitors such as bortezomib, carfilzomib and marizomib.

Often, organizations such as National Comprehensive Cancer Network (NCCN) publish guidelines and/or treatment algorithms setting forth best practices for treatment of certain patients, illnesses and/or clinical circumstances. See nccn.org. These guidelines often establish, set forth and/or summarize standard of care therapies.

Radiation therapy can be administered in combination with a compound as described herein in some embodiments. Exemplary radiation therapies include external-beam therapy, internal radiation therapy, implant radiation, stereotactic radiosurgery, systemic radiation therapy, radiotherapy and permanent or temporary interstitial brachytherapy. The term “brachytherapy,” as used herein, refers to radiation therapy delivered by a spatially confined radioactive material inserted into the body at or near a tumor or other proliferative tissue disease site. The term is intended without limitation to include exposure to radioactive isotopes (e.g., At211, I131, I125, Y90, Re186, Re188, Sm153, Bi212, P32, and radioactive isotopes of Lu). Suitable radiation sources for use as a cell conditioner of the present disclosure include both solids and liquids. By way of non-limiting example, the radiation source can be a radionuclide, such as I125, I131, Yb169, Ir192 as a solid source, I125 as a solid source, or other radionuclides that emit photons, beta particles, gamma radiation, or other therapeutic rays. The radioactive material can also be a fluid made from any solution of radionuclide(s), e.g., a solution of I125 or I131, or a radioactive fluid can be produced using a slurry of a suitable fluid containing small particles of solid radionuclides, such as Au198, Y90. Moreover, the radionuclide(s) can be embodied in a gel or radioactive micro spheres.

Without being limited by any theory, a compound of the present disclosure can render abnormal cells more sensitive to treatment with radiation for purposes of killing and/or inhibiting the growth of such cells. Accordingly, some embodiments include a method for sensitizing abnormal cells in a mammal to treatment with radiation which comprises administering to the mammal an amount of a compound as described herein, which amount is effective insensitizing abnormal cells to treatment with radiation. The amount of a compound of the present disclosure in this method can be determined according to the means for ascertaining effective amounts of such compounds and salts described herein. In some embodiments, standard of care therapy includes radiation therapy.

DNA damaging agents can also be used in combination with a compound of the present disclosure. Non-limiting examples of DNA damaging agents include radiation, topoisomerase inhibitors, PARP inhibitors, DNA crosslinking agents and standard of care agents that induce DNA damage, such as DNA crosslinking agents. Particular non-limiting examples of DNA damaging agents include abraxane, gemcitabine, paclitaxel and temozolomide.

Agents that induce endoplasmic reticulum (ER) stress can also be used in combination with a compound of the present disclosure. Non-limiting examples of agents that induce ER stress include agents that increase levels of reactive oxygen species (ROS) (e.g., napabucasin), chaperone inhibitors, HSP90 inhibitors, HSP70 inhibitors, PDI inhibitors and proteasome inhibitors. Further non-limiting examples of agents that induce ER stress include GSK2606414, GSK2656157, STF-083010, tyrosine kinase inhibitor (e.g., sorafenib), phospho-eif2α phosphatase (e.g., Sal003), diindolylmethane derivatives, proteasome inhibitors (e.g., bortezomib), levistolide A, andrographolide, tolfenamic acid, cantharidin, carnosic acid, casticin, cryptotanshinone, curcumin, flavokawain B, fucoidan, 2-3,4-dihydroxyphenylethanol, 7-dimethoxyflavone, SMIP004 (N-(4-butyl-2-methyl-phenylacetamide), licochalcone A, neferine, paeonol, pardaxin, parthenolide, piperine, polyphenon E, polyphyllin D, resveratrol, dehydrocostuslactone, γ-tocotrienol, Ω-hydroxyundec-9-enoic acid, ampelopsin, ardisianone, genistein, guttiferone H, guggulsterone, marchantin M, sarsasapogenin, saxifragifolin, prodigiosin, quercetin, honokiol, brefeldin A, A-tocopheryl succinate, verrucarin A, vitamin E succinate, ultrafine and zerumbone. See, for example, Walczak, A., et al. Oxidative Medicine and Cellular Longevity Volume 2019, Article ID 5729710, the entire content of which is incorporated herein by reference.

Non-limiting examples of chemotherapeutic agents for use in combination with a compound of the present disclosure (e.g., in combination therapy, in a pharmaceutical combination) include capecitabine (Xeloda®), N4-pentoxycarbonyl-5-deoxy-5-fluorocytidine, carboplatin (Paraplatin®), cisplatin (Platinol®), cladribine (Leustatin®), cyclophosphamide (Cytoxan® or Neosar®), cytarabine, cytosine arabinoside (Cytosar-U®), cytarabine liposome injection (DepoCyt®), dacarbazine (DTIC-Dome®), doxorubicin hydrochloride (Adriamycin®, Rubex®), fludarabine phosphate (Fludara®), 5-fluorouracil (Adrucil®, Efudex®), gemcitabine (difluorodeoxycitidine), irinotecan (Camptosar®), L-asparaginase (ELSPAR®), 6-mercaptopurine (Purinethol®), methotrexate (Folex®), pentostatin, 6-thioguanine, thiotepa, and topotecan hydrochloride for injection (Hycamptin®). A further example is bortezomib. Yet further examples include gemcitabine, nab-paclitaxel (Abraxane®), erlotinib, fluorouracil and FOLFIRINOX (a chemotherapy regimen made up of folinic acid, fluorouracil, irinotecan and oxaliplatin), or any combination of two or more of the foregoing, e.g., to treat pancreatic cancer (e.g., advanced pancreatic cancer, pancreatic ductal adenocarcinoma).

Anti-cancer agents of particular interest for use in combination with the compounds of the present disclosure include:

Topoisomerase inhibitors, including Type I topoisomerase inhibitors, such as irinotecan, topotecan, and camptothecin, and Type 2 topoisomerase inhibitors, such as etoposide, doxorubicin, and epirubicin.

Poly(ADP-ribose) polymerase (PARP) inhibitors, such as olaparib, rucaparib, niraparib, talazoparib, veliparib, pamiparib and iniparib.

DNA crosslinking agents, such as cisplatin, carboplatin and oxaliplatin.

Agents that increase levels of reactive oxygen species (ROS), such as napabucasin.

PARP inhibitors such as olaparib, rucaparib, niraparib, veliparib and talazoparib.

Purine antimetabolites and/or inhibitors of de novo purine synthesis: pemetrexed (Alimta®), gemcitabine (Gemzar®), 5-fluorouracil (Adrucil®, Carac® and Efudex®), methotrexate (Trexall®), capecitabine (Xeloda®), floxuridine (FUDR®), decitabine (Dacogen®), azacitidine (Vidaza® and Azadine®), 6-mercaptopurine (Purinethol®), cladribine (Leustatin®, Litak® and Movectro®), fludarabine (Fludara®), pentostatin (Nipent®), nelarabine (Arranon®), clofarabine (Clolar® and Evoltra®), and cytarabine (Cytosar®).

Anti-angiogenesis agents include, for example, MMP-2 (matrix-metalloproteinase 2) inhibitors, rapamycin, temsirolimus (CCI-779), everolimus (RAD001), sorafenib, sunitinib, and bevacizumab. Examples of useful COX-II inhibitors include CELEBREX™ (alecoxib), valdecoxib, and rofecoxib. Examples of useful matrix metalloproteinase inhibitors are described in WO 96/33172 (published Oct. 24, 1996), WO 96/27583 (published Mar. 7, 1996), European Patent Application No. 97304971.1 (filed Jul. 8, 1997), European Patent Application No. 99308617.2 (filed Oct. 29, 1999), WO 98/07697 (published Feb. 26, 1998), WO 98/03516 (published Jan. 29, 1998), WO 98/34918 (published Aug. 13, 1998), WO 98/34915 (published Aug. 13, 1998), WO 98/33768 (published Aug. 6, 1998), WO 98/30566 (published Jul. 16, 1998), European Patent Publication 606,046 (published Jul. 13, 1994), European Patent Publication 931, 788 (published Jul. 28, 1999), WO 90/05719 (published May 31, 1990), WO 99/52910 (published Oct. 21, 1999), WO 99/52889 (published Oct. 21, 1999), WO 99/29667 (published Jun. 17, 1999), PCT International Application No. PCT/IB98/01113 (filed Jul. 21, 1998), European Patent Application No. 99302232.1 (filed Mar. 25, 1999), Great Britain Patent Application No. 9912961.1 (filed Jun. 3, 1999), U.S. Provisional Application No. 60/148,464 (filed Aug. 12, 1999), U.S. Pat. No. 5,863,949 (issued Jan. 26, 1999), U.S. Pat. No. 5,861,510 (issued Jan. 19, 1999), and European Patent Publication 780,386 (published Jun. 25, 1997), all of which are incorporated herein in their entireties by reference. Embodiments of MMP-2 and MMP-9 inhibitors include those that have little or no activity inhibiting MMP-1. Other embodiments include those that selectively inhibit MMP-2 and/or AMP-9 relative to the other matrix-metalloproteinases (i.e., MAP-1, MMP-3, MMP-4, MMP-5, MMP-6, MMP-7, MMP-8, MMP-10, MMP-11, MMP-12, and MMP-13). Some specific examples of MMP inhibitors useful in some embodiments are AG-3340, RO 323555, and RS 13-0830.

Autophagy inhibitors include, but are not limited to chloroquine, 3-methyladenine, hydroxychloroquine (Plaquenil™), bafilomycin A1, 5-amino-4-imidazole carboxamide riboside (AICAR), okadaic acid, autophagy-suppressive algal toxins which inhibit protein phosphatases of type 2A or type 1, analogues of cAMP, and drugs which elevate cAMP levels such as adenosine, LY204002, N6-mercaptopurine riboside, and vinblastine. In addition, antisense or siRNA that inhibits expression of proteins including but not limited to ATG5 (which are implicated in autophagy), may also be used.

In other embodiments, agents useful in methods for combination therapy with a compound as described herein include, but are not limited to: erlotinib, afatinib, Iressa (gefitinib), GDC0941, MLN1117, BYL719 (alpelisib), BKM120 (buparlisib), CYT387, GLPG0634, baricitinib, lestaurtinib, momelotinib, pacritinib, ruxolitinib, TG101348, crizotinib, tivantinib, AMG337, cabozantinib, foretinib, onartuzumab, NVP-AEW541, dasatinib, ponatinib, saracatinib, bosutinib, trametinib, selumetinib, cobimetinib, PD0325901, RO5126766, axitinib, bevacizumab, bostutinib, cetuximab, fostamatinib, imatinib, lapatinib, lenvatinib, ibrutinib, nilotinib, panitumumab, pazopanib, pegaptanib, ranibizumab, ruxolitinib, sorafenib, sunitinib, SU6656, trastuzumab, tofacitinib, vandetanib, vemurafenib, irinotecan, taxol, docetaxel, rapamycin or MLN0128.

B-cell receptor signaling antagonists (e.g., a Bruton's tyrosine kinase (BTK) inhibitors): ibrutinib.

Bromodomain inhibitors. A bromodomain inhibitor inhibits at least one bromodomain protein, such as Brd2, Brd3, Brd4 and/or BrdT, for example Brd4. In some of these embodiments, the bromodomain inhibitor is JQ-1 (Nature 2010 Dec. 23; 468(7327):1067-73), BI2536 (ACS Chem. Biol. 2014 May 16; 9(5):1160-71; Boehringer Ingelheim), TG101209 (ACS Chem. Biol. 2014 May 16; 9(5):1160-71), OTX015 (Mol. Cancer Ther. November 201312; C244; Oncoethix), IBET762 (J Med Chem. 2013 Oct. 10; 56(19):7498-500; GlaxoSmithKline), IBET151 (Bioorg. Med. Chem. Lett. 2012 Apr. 15; 22(8):2968-72; GlaxoSmithKline), PFI-1 (J. Med. Chem. 2012 Nov. 26; 55(22):9831-7; Cancer Res. 2013 Jun. 1; 73(11):3336-46; Structural Genomics Consortium) of CPI-0610 (Constellation Pharmaceuticals). In some embodiments, the bromodomain inhibitor is TG101209, BI2536, OTX015, C244, IBET762, IBET151, or PFI-1.

Histone deacetylase (HDAC) inhibitors. A HDAC inhibitor inhibits at least one HDAC protein. HDAC proteins may be grouped into classes based on homology to yeast HDAC proteins with Class I made up of HDAC1, HDAC2, HDAC3 and HDAC 8; Class IIa made up of HDAC4, HDAC5, HDAC7 and HDAC 9; Class IIb made up of HDAC6 and HDAC10; and Class IV made up of HDAC11. In some of these embodiments, the HDAC inhibitor is trichostatin A, vorinostat (Proc. Natl. Acad. Sci. U.S.A. 1998 Mar. 17; 95(6):3003-7), givinostat, abexinostat (Mol. Cancer Ther. 2006 May; 5(5):1309-17), belinostat (Mol. Cancer Ther. 2003 August; 2(8):721-8), panobinostat (Clin. Cancer Res. 2006 Aug. 1; 12(15):4628-35), resminostat (Clin. Cancer Res. 2013 Oct. 1; 19(19):5494-504), quisinostat (Clin. Cancer Res. 2013 Aug. 1; 19(15):4262-72), depsipeptide (Blood. 2001 Nov. 1; 98(9):2865-8), entinostat (Proc. Natl. Acad. Sci. U.S.A. 1999 Apr. 13; 96(8):4592-7), mocetinostat (Bioorg. Med. Chem. Lett. 2008 Feb. 1; 18(3):106771) or valproic acid (EMBO J. 2001 Dec. 17; 20(24):6969-78). For example, in some embodiments the HDAC inhibitor is panobinostat, vorinostat, MS275, belinostat, or LBH589. In some embodiments, the HDAC inhibitor is panobinostat or SAHA.

Epidermal growth factor receptor (EGFR) inhibitors: erlotinib hydrochloride (Tarceva®), osimertinib, lapatinib, neratinib, vandetanib and gefitinib (Iressa®). A combination of a compound as described herein and an EGFR inhibitor and/or EGFR antibody may be useful, for example, in the treatment of cancers that are related to EGFR dysregulation, such as non-small-cell lung cancer (NSCLC), pancreatic cancer, breast cancer, and colon cancer. EGFR may be dysregulated, for example, due to activating mutations in exons 18, 19, 20, or 21. In particular embodiments, the EGFR inhibitor is erlotinib or osimertinib. In particular embodiments, the combination of a compound as described herein and an EGFR inhibitor is used to treat EGFR-mutated NSCLC. In particular embodiments, the combination of a compound as described herein and an EGFR inhibitor is used to treat an EGFR inhibitor-resistant cancer, and the compound as described herein sensitized the cancer to the EGFR inhibitor.

EGFR antibodies: cetuximab (Erbitux®), necitumumab, panitumumab.

MTAP inhibitors: (3R,4S)-1-((4-amino-5H-pyrrolo[3,2-d]pyrimidin-7-yl)methyl)-4-((methylthio)methyl)pyrrolidin-3-ol (MT-DADMe-Immucillin-A, CAS 653592-04-2).

Methylthioadenosine: ((2R,3R,4S,5S)-2-(6-amino-9H-purin-9-yl)-5-((methylthio)methyl)tetrahydrofuran-3,4-diol, CAS 2457-80-9).

MET inhibitors: capmatinib (INC280, CAS 1029712-80-8).

Platelet-derived growth factor (PDGF) receptor inhibitors: imatinib (Gleevec®); linifanib (N-[4-(3-amino-1H-indazol-4-yl)phenyl]-N′-(2-fluoro-5-methylphenyl)urea, also known as ABT 869, available from Genentech); sunitinib malate (Sutent®); quizartinib (AC220, CAS 950769-58-1); pazopanib (Votrient®); axitinib (Inlyta®); sorafenib (Nexavar®); vargatef (BIBF1120, CAS 928326-83-4); telatinib (BAY57-9352, CAS 332012-40-5); vatalanib dihydrochloride (PTK787, CAS 212141-51-0); and motesanib diphosphate (AMG706, CAS 857876-30-3, N-(2,3-dihydro-3,3-dimethyl-1H-indol-6-yl)-2-[(4-pyridinylmethyl)amino]-3-pyridinecarboxamide, described in PCT Publication No. WO 02/066470).

Phosphoinositide 3-kinase (PI3K) inhibitors: 4-[2-(1H-Indazol-4-yl)-6-[[4-(methylsulfonyl)piperazin-1-yl]methyl]thieno[3,2-d]pyrimidin-4-yl]morpholine (also known as GDC 0941 and described in PCT Publication Nos. WO 09/036082 and WO 09/055730); 4-(trifluoromethyl)-5-(2,6-dimorpholinopyrimidin-4-yl)pyridin-2-amine (also known as BKM120 or NVP-BKM120, and described in PCT Publication No. WO 2007/084786); alpelisib (BYL719): (5Z)-5-[[4-(4-Pyridinyl)-6-quinolinyl]methylene]-2,4-thiazolidinedione (GSK1059615, CAS 958852-01-2); 5-[8-methyl-9-(1-methylethyl)-2-(4-morpholinyl)-9H-purin-6-yl]-2-pyrimidinamine (VS-5584, CAS 1246560-33-7) and everolimus (AFINITOR®).

Cyclin-dependent kinase (CDK) inhibitors: ribociclib (LEE011, CAS 1211441-98-3); aloisine A; alvocidib (also known as flavopiridol or HMR-1275, 2-(2-chlorophenyl)-5,7-dihydroxy-8-[(3S,4R)-3-hydroxy-1-methyl-4-piperidinyl]-4-chromenone, and described in U.S. Pat. No. 5,621,002); crizotinib (PF-02341066, CAS 877399-52-5); 2-(2-chlorophenyl)-5,7-dihydroxy-8-[(2R,3S)-2-(hydroxymethyl)-1-methyl-3-pyrrolidinyl]-4H-1-benzopyran-4-one, hydrochloride (P276-00, CAS 920113-03-7); 1-methyl-5-[[2-[5-(trifluoromethyl)-1H-imidazol-2-yl]-4-pyridinyl]oxy]-N-[4-(trifluoromethyl)phenyl]-1H-benzimidazol-2-amine (RAF265, CAS 927880-90-8); indisulam (E7070); roscovitine (CYC202); 6-acetyl-8-cyclopentyl-5-methyl-2-(5-piperazin-1-yl-pyridin-2-ylamino)-8H-pyrido[2,3-d]pyrimidin-7-one, hydrochloride (PD0332991); dinaciclib (SCH727965); N-[5-[[(5-tert-butyloxazol-2-yl)methyl]thio]thiazol-2-yl]piperidine-4-carboxamide (BMS 387032, CAS 345627-80-7); 4-[[9-chloro-7-(2,6-difluorophenyl)-5H-pyrimido[5,4-d][2]benzazepin-2-yl]amino]-benzoic acid (MLN8054, CAS 869363-13-3); 5-[3-(4,6-difluoro-1H-benzimidazol-2-yl)-1H-indazol-5-yl]-N-ethyl-4-methyl-3-pyridinemethanamine (AG-024322, CAS 837364-57-5); 4-(2,6-dichlorobenzoylamino)-1H-pyrazole-3-carboxylic acid N-(piperidin-4-yl)amide (AT7519, CAS 844442-38-2); 4-[2-methyl-1-(1-methylethyl)-1H-imidazol-5-yl]-N-[4-(methylsulfonyl)phenyl]-2-pyrimidinamine (AZD5438, CAS 602306-29-6); palbociclib (PD-0332991); and (2R,3R)-3-[[2-[[3-[[S(R)]-S-cyclopropylsulfonimidoyl]-phenyl]amino]-5-(trifluoromethyl)-4-pyrimidinyl]oxy]-2-butanol (BAY 10000394).

p53-MDM2 inhibitors: (S)-1-(4-chloro-phenyl)-7-isopropoxy-6-methoxy-2-(4-{methyl-[4-(4-methyl-3-oxo-piperazin-1-yl)-trans-cyclohexylmethyl]-amino}-phenyl)-1,4-dihydro-2H-isoquinolin-3-one, (S)-5-(5-chloro-1-methyl-2-oxo-1,2-dihydro-pyridin-3-yl)-6-(4-chloro-phenyl)-2-(2,4-dimethoxy-pyrimidin-5-yl)-1-isopropyl-5, 6-dihydro-1H-pyrrolo[3,4-d]imidazol-4-one, [(4S,5R)-2-(4-tert-butyl-2-ethoxyphenyl)-4,5-bis(4-chlorophenyl)-4,5-dimethylimidazol-1-yl]-[4-(3-methylsulfonylpropyl)piperazin-1-yl]methanone (RG7112), 4-[[(2R,3S,4R,5S)-3-(3-chloro-2-fluorophenyl)-4-(4-chloro-2-fluorophenyl)-4-cyano-5-(2,2-dimethylpropyl)pyrrolidine-2-carbonyl]amino]-3-methoxybenzoic acid (RG7388), SAR299155, 2-((3R,5R,6S)-5-(3-chlorophenyl)-6-(4-chlorophenyl)-1-((S)-1-(isopropylsulfonyl)-3-methylbutan-2-yl)-3-methyl-2-oxopiperidin-3-yl)acetic acid (AMG232), {(3R,5R,6S)-5-(3-chlorophenyl)-6-(4-chlorophenyl)-1-[(2S,3S)-2-hydroxy-3-pentanyl]-3-methyl-2-oxo-3-piperidinyl}acetic acid (AM-8553), (+)-4-[4,5-bis(4-chlorophenyl)-2-(2-isopropoxy-4-methoxy-phenyl)-4,5-dihydro-imidazole-1-carbonyl]-piperazin-2-one (Nutlin-3), 2-methyl-7-[phenyl(phenylamino)methyl]-8-quinolinol (NSC 66811), 1-N-[2-(1H-indol-3-yl)ethyl]-4-N-pyridin-4-ylbenzene-1,4-diamine (JNJ-26854165), 4-[4,5-bis(3,4-chlorophenyl)-2-(2-isopropoxy-4-methoxy-phenyl)-4,5-dihydro-imidazole-1-carboxyl]-piperazin-2-one (Caylin-1), 4-[4,5-bis(4-trifluoromethyl-phenyl)-2-(2-isopropoxy-4-methoxy-phenyl)-4,5-dihydro-imidazole-1-carboxyl]-piperazin-2-one (Caylin-2), 5-[[3-dimethylamino)propyl]amino]-3,10-dimethylpyrimido[4,5-b]quinoline-2,4(3H,10H)-dione dihydrochloride (HLI373) and trans-4-iodo-4′-boranyl-chalcone (SC204072).

Mitogen-activated protein kinase (MEK) inhibitors: XL-518 (also known as GDC-0973, CAS No. 1029872-29-4, available from ACC Corp.); selumetinib (5-[(4-bromo-2-chlorophenyl)amino]-4-fluoro-N-(2-hydroxyethoxy)-1-methyl-1H-benzimidazole-6-carboxamide, also known as AZD6244 or ARRY 142886, described in PCT Publication No. WO 2003/077914); 2-[(2-chloro-4-iodophenyl)amino]-N-(cyclopropylmethoxy)-3,4-difluoro-benzamide (also known as CI-1040 or PD184352 and described in PCT Publication No. WO 2000/035436); N-[(2R)-2,3-dihydroxypropoxy]-3,4-difluoro-2-[(2-fluoro-4-iodophenyl)amino]-benzamide (also known as PD0325901 and described in PCT Publication No. WO 2002/006213); 2,3-bis[amino[(2-aminophenyl)thio]methylene]-butanedinitrile (also known as U0126 and described in U.S. Pat. No. 2,779,780); N-[3,4-difluoro-2-[(2-fluoro-4-iodophenyl)amino]-6-methoxyphenyl]-1-[(2R)-2,3-dihydroxypropyl]-cyclopropanesulfonamide (also known as RDEA119 or BAY869766 and described in PCT Publication No. WO 2007/014011); (3S,4R,5Z,8S,9S,11E)-14-(ethylamino)-8,9,16-trihydroxy-3,4-dimethyl-3,4,9; 19-tetrahydro-1H-2-benzoxacyclotetradecine-1,7(8H)-dione] (also known as E6201 and described in PCT Publication No. WO 2003/076424); 2′-amino-3′-methoxyflavone (also known as PD98059 available from Biaffin GmbH & Co., KG, Germany); (R)-3-(2,3-dihydroxypropyl)-6-fluoro-5-(2-fluoro-4-iodophenylamino)-8-methylpyrido[2,3-d]pyrimidine-4,7(3H,8H)-dione (TAK-733, CAS 1035555-63-5); pimasertib (AS-703026, CAS 1204531-26-9); trametinib dimethyl sulfoxide (GSK-1120212, CAS 1204531-25-80); 2-(2-fluoro-4-iodophenylamino)-N-(2-hydroxyethoxy)-1,5-dimethyl-6-oxo-1,6-dihydropyridine-3-carboxamide (AZD 8330); 3,4-difluoro-2-[(2-fluoro-4-iodophenyl)amino]-N-(2-hydroxyethoxy)-5-[(3-oxo-[1,2]oxazinan-2-yl)methyl]benzamide (CH 4987655 or Ro 4987655); and 5-[(4-bromo-2-fluorophenyl)amino]-4-fluoro-N-(2-hydroxyethoxy)-1-methyl-1H-benzimidazole-6-carboxamide (MEK162).

B-RAF inhibitors: regorafenib (BAY73-4506, CAS 755037-03-7); tuvizanib (AV951, CAS 475108-18-0); vemurafenib (ZELBORAF®, PLX-4032, CAS 918504-65-1); encorafenib (also known as LGX818); 1-methyl-5-[[2-[5-(trifluoromethyl)-1H-imidazol-2-yl]-4-pyridinyl]oxy]-N-[4-(trifluoromethyl)phenyl-1H-benzimidazol-2-amine (RAF265, CAS 927880-90-8); 5-[1-(2-hydroxyethyl)-3-(pyridin-4-yl)-1H-pyrazol-4-yl]-2,3-dihydroinden-1-one oxime (GDC-0879, CAS 905281-76-7); 5-[2-[4-[2-(dimethylamino)ethoxy]phenyl]-5-(4-pyridinyl)-1H-imidazol-4-yl]-2,3-dihydro-1H-inden-1-one oxime (GSK2118436 or SB590885); (+/−)-methyl (5-(2-(5-chloro-2-methylphenyl)-1-hydroxy-3-oxo-2,3-dihydro-1H-isoindol-1-yl)-1H-benzimidazol-2-yl)carbamate (also known as XL-281 and BMS908662), dabrafenib (TAFINLAR®), and N-(3-(5-chloro-1H-pyrrolo[2,3-b]pyridine-3-carbonyl)-2,4-difluorophenyl)propane-1-sulfonamide (also known as PLX4720).

ALK inhibitors: crizotinib (XALKORI®).

PIM kinase inhibitors:

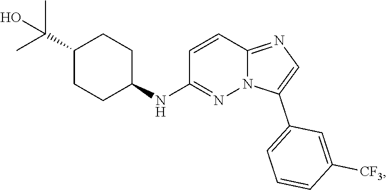 [SMILES: CC(C)(O)[C@H]1CC[C@H](Nc2ccc3ncc(-c4cccc(C(F)(F)F)c4)n3n2)CC1]

or a pharmaceutically acceptable salt thereof.

Proteasome inhibitors: bortezomib (VELCADE®), N-5-benzyloxycarbonyl-Ile-Glu(O-tert-butyl)-Ala-leucinal (PSI), carfilzomib and ixazomib, marizomib (NPI-0052), delanzomib (CEP-18770), and O-methyl-N-[(2-methyl-5-thiazolyl)carbonyl]-L-seryl-O-methyl-N-[(1S)-2-[(2R)-2-methyl-2-oxiranyl]-2-oxo-1-(phenylmethyl)ethyl]-L-serinamide (oprozomib, ONX-0912, PR-047) (e.g., bortezomib). An RNAi screen identified TNK1 as a potential modulator of proteasome inhibitor sensitivity in myeloma. Zhu et al., Blood (2011) 117 (14): 3847-3857. In some embodiments, a compound described herein (e.g., a compound of Formula I, or a subformula thereof, or a pharmaceutically acceptable salt of the foregoing) is administered in combination with a proteasome inhibitor described herein, such as bortezomib, e.g., for the treatment of multiple myeloma.

Further non-limiting examples of therapeutic agents that can be used in combinations with a compound as described herein are chemotherapeutic agents, cytotoxic agents, and non-peptide small molecules such as Gleevec® (Imatinib Mesylate), Velcade® (bortezomib), Casodex (bicalutamide), Iressa® (gefitinib), and Adriamycin as well as a host of chemotherapeutic agents. Non-limiting examples of chemotherapeutic agents include alkylating agents such as thiotepa and cyclosphosphamide (CYTOXAN®); alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphaoramide and trimethylolomelamine; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, carminomycin, carzinophilin, Casodex®, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elfomithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK®; razoxane; sizofiran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (“Ara-C”); cyclophosphamide; thiotepa; taxanes, e.g., paclitaxel (TAXOL™, Bristol-Myers Squibb Oncology, Princeton, N.J.), docetaxel (TAXOTERE™, Rhone-Poulenc Rorer, Antony, France) and cabazitaxel (JEVTANA, Sanofi Genzyme); retinoic acid; esperamicins; capecitabine; and pharmaceutically acceptable salts, acids or derivatives of any of the above.

Many chemotherapeutics are presently known in the art and can be used in combination with a compound as described herein. In some embodiments, the chemotherapeutic is selected from the group consisting of mitotic inhibitors, alkylating agents, anti-metabolites, intercalating antibiotics, growth factor inhibitors, cell cycle inhibitors, enzymes, topoisomerase inhibitors, biological response modifiers, anti-hormones, angiogenesis inhibitors, and anti-androgens.

More non-limiting examples of chemotherapeutic agents for use in combination with a compound as described herein (e.g., in combination therapy, in a pharmaceutical combination) include capecitabine (Xeloda®), N4-pentoxycarbonyl-5-deoxy-5-fluorocytidine, carboplatin (Paraplatin®), cisplatin (Platinol®), cladribine (Leustatin®), cyclophosphamide (Cytoxan® or Neosar®), cytarabine, cytosine arabinoside (Cytosar-U®), cytarabine liposome injection (DepoCyt®), dacarbazine (DTIC-Dome®), doxorubicin hydrochloride (Adriamycin®, Rubex®), fludarabine phosphate (Fludara®), 5-fluorouracil (Adrucil®, Efudex®), gemcitabine (difluorodeoxycitidine), irinotecan (Camptosar®), L-asparaginase (ELSPAR®), 6-mercaptopurine (Purinethol®), methotrexate (Folex®), pentostatin, 6-thioguanine, thiotepa, and topotecan hydrochloride for injection (Hycamptin®).

Further non-limiting examples of commonly prescribed anti-cancer drugs include Herceptin®, Avastin®, Erbitux®, Rituxan®, Taxol®, Arimidex®, Taxotere®, ABVD, AVICINE, Abagovomab, Acridine carboxamide, Adecatumumab, 17-N-Allylamino-17-demethoxygeldanamycin, Alpharadin, Alvocidib, 3-Aminopyridine-2-carboxaldehyde thiosemicarbazone, Amonafide, Anthracenedione, Anti-CD22 immunotoxins, Antineoplastic, Antitumorigenic herbs, Apaziquone, Atiprimod, Azathioprine, Belotecan, Bendamustine, BIBW 2992, Biricodar, Brostallicin, Bryostatin, Buthionine sulfoximine, CBV (chemotherapy), Calyculin, cell-cycle nonspecific antineoplastic agents, Dichloroacetic acid, Discodermolide, Elsamitrucin, Enocitabine, Epothilone, Eribulin, Everolimus, Exatecan, Exisulind, Ferruginol, Forodesine, Fosfestrol, ICE chemotherapy regimen, IT-101, Imexon, Imiquimod, Indolocarbazole, Irofulven, Laniquidar, Larotaxel, Lenalidomide, Lucanthone, Lurtotecan, Mafosfamide, Mitozolomide, Nafoxidine, Nedaplatin, Olaparib, Ortataxel, PAC-1, Pawpaw, Pixantrone, Proteasome inhibitor, Rebeccamycin, Resiquimod, Rubitecan, SN-38, Salinosporamide A, Sapacitabine, Stanford V, Swainsonine, Talaporfin, Tariquidar, Tegafur-uracil, Temodar, Tesetaxel, Triplatin tetranitrate, Tris(2-chloroethyl)amine, Troxacitabine, Uramustine, Vadimezan, Vinflunine, ZD6126 or Zosuquidar.

Also included as suitable chemotherapeutic cell conditioners are anti-hormonal agents that act to regulate or inhibit hormone action on tumors such as anti-estrogens including for example tamoxifen, (Nolvadex™), raloxifene, aromatase inhibiting 4(5)-imidazoles, 4hydroxytamoxifen, trioxifene, keoxifene, LY 117018, onapristone, and toremifene (Fareston); and anti-androgens such as flutamide, nilutamide, bicalutamide, leuprolide, and goserelin; chlorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; camptothecin-11 (CPT-11); topoisomeRASe inhibitor RFS 2000; difluoromethylornithine (DMFO).

Non-limiting examples of therapeutic agents that can be used in combinations with a compound as described herein are mTOR inhibitors. Exemplary mTOR inhibitors include, e.g., temsirolimus; ridaforolimus (formally known as deferolimus, (1R,2R,4S)-4-[(2R)-2 [(1R,9S,12S,15R,16E,18R,19R,21R, 23S,24E,26E,28Z,30S,32S,35R)-1,18-dihydroxy-19,30-dimethoxy-15,17,21,23,29,35-hexamethyl-2,3,10,14,20-pentaoxo-11,36-dioxa-4-azatricyclo[30.3.1.0^{4,9}] hexatriaconta-16,24,26,28-tetraen-12-yl]propyl]-2-methoxycyclohexyl dimethylphosphinate, also known as AP23573 and MK8669, and described in PCT Publication No. WO 03/064383); everolimus (Afinitor® or RAD001); rapamycin (AY22989, Sirolimus®); simapimod (CAS 164301-51-3); emsirolimus, (5-{2,4-Bis[(3S)-3-methylmorpholin-4-yl]pyrido[2,3-d]pyrimidin-7-yl}-2-methoxyphenyl)methanol (AZD8055); 2-Amino-8-[trans-4-(2-hydroxyethoxy)cyclohexyl]-6-(6-methoxy-3-pyridinyl)-4-methyl-pyrido[2,3-d]pyrimidin-7(8H)-one (PF04691502, CAS 1013101-36-4); and N²-[1,4-dioxo-4-[[4-(4-oxo-8-phenyl-4H-1-benzopyran-2-yl)morpholinium-4-yl]methoxy]butyl]-L-arginylglycyl-L-α-aspartylL-serine-inner salt (SEQ ID NO: 1482) (SF1126, CAS 936487-67-1), and XL765.

In certain other embodiments, a method for treating cancer is provided, the method comprising administering an effective amount of a compound as described herein and a CDK inhibitor to a subject in need thereof.

In embodiments, the CDK inhibitor is a CDK2, CDK4, CDK6, CDK7, CDK8, CDK9, CDK10, and/or CDK11 inhibitor. In some embodiments, the CDK inhibitor is a CDK7, CDK9 inhibitor, or both. In some embodiments, the CDK inhibitor is dinaciclib (ACS Med. Chem. Lett. 2010 May 17; 1(5):204-8; Mol. Cancer Ther. 2010 August; 9(8):2344-53; Merck, Sharp and Dohme), AT7519 (J. Med. Chem. 2008 Aug. 28; 51(16):4986-99; Astex Pharmaceutical) or palbociclib (J. Med. Chem. 2005 Apr. 7; 48(7):2388-406; Pfizer). In certain embodiments, the CDK inhibitor is a CDK9 inhibitor, such as alvocidib. The alvocidib may be administered as the free bases, as a pharmaceutically acceptable salt or as a prodrug. In certain embodiments, the CDK9 inhibitor is alvocidib. In other embodiments, the CDK9 inhibitor is a pharmaceutically acceptable salt of alvocidib. In other embodiments, the CDK9 inhibitor is a prodrug of alvocidib. Prodrugs of alvocidib include those disclosed in WO 2016/187316, the full disclosure of which is hereby incorporated by reference in its entirety.

In one embodiment, a compound as described herein is administered to a subject in need thereof in combination with an ATR inhibitor, such as AZD6738 or VX-970. The administration may be before, concurrently or after administration of the ATR inhibitor. In one specific embodiment, a compound as described herein is administered to a subject in need thereof in combination with an ATR inhibitor, such as AZD6738 or VX-970 for treatment of non-small cell lung cancer. In a related specific embodiment, a pharmaceutically acceptable salt of a compound as described herein is administered to a subject in need thereof in combination with an ATR inhibitor, such as AZD6738 or VX-970 for treatment of non-small cell lung cancer. In some of the foregoing embodiments, the salt is a tartrate salt. In some of the foregoing embodiments, the ATR inhibitor is AZD6738. In some of the foregoing embodiments, the ATR inhibitor is VX-970. In some embodiments, the salt is a tartrate salt and the ATR inhibitor is AZD6738. In some embodiments, the salt is a tartrate salt and the ATR inhibitor is VX-970. In some of the foregoing embodiments, the ATR inhibitor is a combination of AZD6738 and VX-970.

In some of the foregoing embodiments, the non-small cell lung cancer comprises TCGA lung adenocarcinoma, one or more LUAD tumors, TCGA lung squamous cell carcinoma, one or more LUSC tumors, one or more MDACC PROSPECT tumors, one or more MDACC BATTLE1 tumors, one or more BATTLE2 tumors, or combinations thereof. In some embodiments, the non-small cell lung cancer comprises TCGA LUAD tumors, for example, tumors enriched in ALK translocations. In some embodiments, the non-small cell lung cancer comprises TCGA LUAD tumors, for example, tumors comprising one or more EGFR mutations.

In one embodiment, a compound as described herein is administered to a subject in need thereof thereby sensitizing the subject to administration of an ATR inhibitor, such as AZD6738 or VX-970. In a related embodiment, a pharmaceutically acceptable salt of a compound as described herein is administered to a subject in need thereof thereby sensitizing the subject to administration of an ATR inhibitor, such as AZD6738 or VX-970. In one specific embodiment, a compound as described herein is administered to a subject in need thereof thereby sensitizing the subject to administration of an ATR inhibitor, such as AZD6738 or VX-970 for treatment of non-small cell lung cancer. In a related specific embodiment, a pharmaceutically acceptable salt of a compound as described herein is administered to a subject in need thereof thereby sensitizing the subject to administration of an ATR inhibitor, such as AZD6738 or VX-970 for treatment of non-small cell lung cancer. In some of the foregoing embodiments, the salt is a tartrate salt. In some of the foregoing embodiments, the ATR inhibitor is AZD6738. In some of the foregoing embodiments, the ATR inhibitor is VX-970. In some embodiments, the salt is a tartrate salt and the ATR inhibitor is AZD6738. In some embodiments, the salt is a tartrate salt and the ATR inhibitor is VX-970. In some of the foregoing embodiments, the ATR inhibitor is a combination of AZD6738 and VX-970.

Some patients may experience allergic reactions to compounds as described herein and/or other therapeutic agent(s) (e.g., anti-cancer agent(s)) during or after administration. Therefore, anti-allergic agents can be administered in combination with compounds as described herein and/or other therapeutic agent(s) (e.g., anti-cancer agent(s)) to minimize the risk of an allergic reaction. Suitable anti-allergic agents include corticosteroids (Knutson, S., et al., PLoS One, DOI:10.1371/journal.pone.0111840 (2014)), such as dexamethasone (e.g., DECADRON®), beclomethasone (e.g., BECLOVENT®), hydrocortisone (also known as cortisone, hydrocortisone sodium succinate, hydrocortisone sodium phosphate, sold under the tradenames ALA-CORT®, hydrocortisone phosphate, SOLU-CORTEF®, HYDROCORT ACETATE® and LANACORT®), prednisolone (sold under the tradenames DELTA-CORTEL®, ORAPRED®, PEDIAPRED® and PRELONE®), prednisone (sold under the tradenames DELTASONE®, LIQUID RED®, METICORTEN® and ORASONE®), methylprednisolone (also known as 6-methylprednisolone, methylprednisolone acetate, methylprednisolone sodium succinate, sold under the tradenames DURALONE®, MEDRALONE®, MEDROL®, M-PREDNISOL® and SOLU-MEDROL®); antihistamines, such as diphenhydramine (e.g., BENADRYL®), hydroxyzine, and cyproheptadine; and bronchodilators, such as the beta-adrenergic receptor agonists, albuterol (e.g., PROVENTIL®), and terbutaline (BRETHINE®).

Some patients may experience nausea during and after administration of the compounds described herein and/or other therapeutic agent(s) (e.g., anti-cancer agent(s)). Therefore, anti-emetics can be used in combination with compounds as described herein and/or other therapeutic agent(s) (e.g., anti-cancer agent(s)) to prevent nausea (upper stomach) and vomiting. Suitable anti-emetics include aprepitant (EMEND®), ondansetron (ZOFRAN®), granisetron HCl (KYTRIL®), lorazepam (ATIVAN®, dexamethasone (DECADRON®), prochlorperazine (COMPAZINE®), casopitant (REZONIC® and ZUNRISA®), and combinations thereof.

Medication to alleviate the pain experienced during the treatment period is often prescribed to make the patient more comfortable. Common over-the-counter analgesics, such TYLENOL®, can also be used in combination with compounds described herein and/or other therapeutic agent(s) (e.g., anti-cancer agent(s)). Opioid analgesic drugs such as hydrocodone/paracetamol or hydrocodone/acetaminophen (e.g., VICODIN®), morphine (e.g., ASTRAMORPH® or AVINZA®), oxycodone (e.g., OXYCONTIN® or PERCOCET®), oxymorphone hydrochloride (OPANA®), and fentanyl (e.g., DURAGESIC®) can be useful for moderate or severe pain, and can be used in combination with compounds described herein and/or other therapeutic agent(s) (e.g., anti-cancer agent(s)).

Immunomodulators (e.g., immunooncology agents) of particular interest for use in combination with compounds described herein include: afutuzumab (available from ROCHE®); pegfilgrastim (NEULASTA®); lenalidomide (CC-5013, REVLIMID®); thalidomide (THALOMID®); actimid (CC4047); and IRX-2 (mixture of human cytokines including interleukin 1, interleukin 2, and interferon 7, CAS 951209-71-5, available from IRX Therapeutics).

Chimeric antigen receptor T-cell (CAR-T) therapies of particular interest for use in combination with compounds described herein include: tisagenlecleucel (Novartis), axicabtagene ciloleucel (Kite), and tocilizumab (atlizumab; Roche).

Immune checkpoint inhibitors of interest for use in combination with compounds described herein include: PD-1 inhibitors, such as pembrolizumab (also known as Lambrolizumab, MK-3475, MK03475, SCH-900475, or KEYTRUDA®) and other anti-PD-1 antibodies (as disclosed in Hamid, O. et al. (2013) New England Journal of Medicine 369 (2): 134-44, U.S. Pat. No. 8,354,509, and WO 2009/114335, incorporated by reference in their entirety), nivolumab (also known as MDX-1106, MDX-1106-04, ONO-4538, BMS-936558, or OPDIVO®) and other anti-PD-1 antibodies (as disclosed in U.S. Pat. No. 8,008,449 and WO 2006/121168, incorporated by reference in their entirety), cemiplimab (LIBTAYO®), spartalizumab (PDR001), pidilizumab (CureTech), MEDI0680 (Medimmune), cemiplimab (REGN2810), dostarlimab (TSR-042), PF-06801591 (Pfizer), sinitilimab, toripalimab, tislelizumab (BGB-A317), camrelizumab (INCSHR1210, SHR-1210), AMP-224 (Amplimmune), CBT-501 (CBT Pharmaceuticals), CBT-502 (CBT Pharmaceuticals), JS001 (Junshi Biosciences), IBI308 (Innovent Biologics), INCSHR1210 (Incyte), also known as SHR-1210 (Hengrui Medicine), BGBA317 (Beigene), BGB-108 (Beigene), BAT-I306 (Bio-Thera Solutions), GLS-010 (Gloria Pharmaceuticals; WuXi Biologics), AK103, AK104, AK105 (Akesio Biopharma; Hangzhou Hansi Biologics; Hanzhong Biologics), LZM009 (Livzon), HLX-10 (Henlius Biotech), MEDI0680 (Medimmune), PDF001 (Novartis), PF-06801591 (Pfizer), Pidilizumab (CureTech) also known as CT-O11 and other anti-PD-1 antibodies (as disclosed in Rosenblatt, J. et al. (2011) J Immunotherapy 34(5): 409-18, U.S. Pat. Nos. 7,695,715, 7,332,582, and 8,686,119, incorporated by reference in their entirety), REGN2810 (Regeneron), TSR-042 (Tesaro) also known as ANBO11, or CS1003 (CStone Pharmaceuticals). MEDI0680 (Medimmune), is also known as AMP-514. MEDI0680 and other anti-PD-1 antibodies are disclosed in U.S. Pat. No. 9,205,148 and WO 2012/145493, incorporated by reference in their entirety. Further known anti-PD-1 antibody molecules include those described, e.g., in WO 2015/112800, WO 2016/092419, WO 2015/085847, WO 2014/179664, WO 2014/194302, WO 2014/209804, WO 2015/200119, U.S. Pat. Nos. 8,735,553, 7,488,802, 8,927,697, 8,993,731, and 9,102,727, incorporated by reference in their entirety. In one embodiment, the PD-1 inhibitor is an anti-PD-1 antibody molecule as described in US 2015/0210769, published on Jul. 30, 2015, entitled “Antibody Molecules to PD-1 and Uses Thereof,” incorporated by reference in its entirety. In one embodiment, the anti-PD-1 antibody molecule comprises the CDRs, variable regions, heavy chains and/or light chains of BAP049-Clone-E or BAP049-Clone-B disclosed in US 2015/0210769. The antibody molecules described herein can be made by vectors, host cells, and methods described in US 2015/0210769, incorporated by reference in its entirety. In one embodiment, the PD-1 inhibitor is a peptide that inhibits the PD-1 signaling pathway, e.g., as described in U.S. Pat. No. 8,907,053, incorporated by reference in its entirety. In one embodiment, the PD-1 inhibitor is an immunoadhesin (e.g., an immunoadhesin comprising an extracellular or PD-1 binding portion of PD-L1 or PD-L2 fused to a constant region (e.g., an Fc region of an immunoglobulin sequence). In one embodiment, the PD-1 inhibitor is AMP-224 (B7-DCIg (Amplimmune), e.g., disclosed in WO 2010/027827 and WO 2011/066342, incorporated by reference in their entirety).

Immune checkpoint inhibitors of interest for use in combination with compounds described herein also include: PD-L1 inhibitors, such as atezolizumab (also known as MPDL3280A, RG7446, R05541267, YW243.55.570, or TECENTRIQ®) and other anti-PD-L1 antibodies as disclosed in U.S. Pat. No. 8,217,149, incorporated by reference in its entirety, avelumab (BAVENCIO® also known as MSB0010718C) and other anti-PD-L1 antibodies as disclosed in WO 2013/079174, incorporated by reference in its entirety, durvalumab (IMFINZI® or MEDI4736) and other anti-PD-L1 antibodies as disclosed in U.S. Pat. No. 8,779,108, incorporated by reference in its entirety), FAZ053 (Novartis), and BMS-936559 (Bristol-Myers Squibb). In certain embodiments, the PD-L1 inhibitor is KN035 (Alphamab; 3DMed; Ascletis Pharma), Envafolimab (TRACON Pharmaceuticals), BMS 936559 (Bristol-Myers Squibb), CS1001 (CStone Pharmaceuticals, Ligand Pharmaceuticals), CX-072 (CytomX Therapeutics), FAZ053 (Novartis), SHR-1316 (Hengrui Medicine), TQB2450 (Chiatai Tianqing), STI-A1014 (Zhaoke Pharm; Lee's Pharm, Lonza, Sorrento Therapeutics, NantWorks), LYN00102 (Lynkcell), A167 (Harbour BioMed, Kelun Group), BGB-A333 (Beigene), MSB2311 (Mabspace Biosciences), or HLX-20 (Henlius Biotech). In one embodiment, the anti-PD-L1 antibody molecule is BMS-936559 (Bristol-Myers Squibb), also known as MDX-1105 or 12A4. BMS-936559 and other anti-PD-L1 antibodies are disclosed in U.S. Pat. No. 7,943,743 and WO 2015/081158, incorporated by reference in their entirety. In certain embodiments, the PD-L1 inhibitor is Cosibelimab (Fortress Biotech), LY3300054 or Iodapolimab (Eli Lilly), GS-4224 (Gilead Sciences), STI-A1015 (Yuhan, Sorrento Therapeutics), BCD-135 (BIOCAD), Cosibelimab (Dana-Farber Cancer Institute, TG Therapeutics), APL-502 (Apollomics), AK106 (Akeso Biopharma), MSB2311 (Transcenta Holding), TG-1501 (TG Therapeutics), FAZ053 (Novartis). In certain embodiments, the PD-L1 inhibitor is MT-6035 (Molecular Templates), Icaritin and ZKAB001 (Lonza, Lee's Pharmaceutical Holdings, Sorrento Therapeutics, Shenogen Pharma Group), TRIDENT Antibody (MacroGenics, Zai Lab), YBL-007 (Anh-Gook Pharmaceutical, Y-Biologics), HTI-1316 (Hengrui Therapeutics), PD-L1 Oncology Project (Weizmann Institute of Sciences), JS003 (Shanghai Junshi Biosciences), ND021 (Numab Therapeutics, CStone Pharmaceuticals), Toca 521 (Tocagen), STTO1 (STCube). In certain embodiments, the PD-L1 inhibitor is DB004 (DotBio), MT-5050 (Molecular Templates), KD036 (Kadmon). In one embodiment, the PD-L1 inhibitor is an anti-PD-L1 antibody molecule. In one embodiment, the PD-L1 inhibitor is an anti-PD-L1 antibody molecule as disclosed in US 2016/0108123, published on Apr. 21, 2016, entitled “Antibody Molecules to PD-L1 and Uses Thereof,” incorporated by reference in its entirety. In one embodiment, the anti-PD-L1 antibody molecule comprises the CDRs, variable regions, heavy chains and/or light chains of BAP058-Clone O or BAP058-Clone N disclosed in US 2016/0108123.

Further known anti-PD-L1 antibodies include those described, e.g., in WO 2015/181342, WO 2014/100079, WO 2016/000619, WO 2014/022758, WO 2014/055897, WO 2015/061668, WO 2013/079174, WO 2012/145493, WO 2015/112805, WO 2015/109124, WO 2015/195163, U.S. Pat. Nos. 8,168,179, 8,552,154, 8,460,927, and 9,175,082, incorporated by reference in their entirety.

In some embodiments, the immune checkpoint inhibitor is a cytotoxic T-lymphocyte-associated modulator. In some embodiments, the immune checkpoint inhibitor targets CTLA-4, such as ipilimumab (YERVOY®), tremelimumab, ALPN-202 (Alpine Immune Sciences), RP2 (Replimune), BMS-986249 (Bristol-Myers Squibb), BMS-986218 (Bristol-Myers Squibb), zalifrelimab (Agenus, Ludwig Institute for Cancer Research, UroGen Pharma, Recepta Biopharma), BCD-217 (BIOCAD), Onc-392 (Pfizer, OncoImmune), IBI310 (Innovent Biologics), KN046 (Alphamab), MK-1308 (Merck & Co), REGN4659 (Regeneron Pharmaceuticals), XmAb207l7 (Xencor), XmAb22841 (Xencor), Anti-CTLA-4 NF (Bristol-Myers Squibb), MEDI5752 (AstraZeneca), AGEN1181 (Agenus), MGD019 (MacroGenics), ATOR-1015 (Alligator Bioscience), BCD-145 (BIOCAD), PSB205 (Sound Biologics), CS1002 (CStone Pharmaceuticals), ADU-1604 (Aduro Biotech), PF-06753512 (Pfizer), BioInvent-Transgene Research Program (Transgene), AGEN2041 (Agenus, Recepta Biopharam), ATOR-1144 (Alligator Bioscience), CTLA-4 Research Project (Sorrento Therapeutics), PD-L1/CTLA-4 Research Project (Sorrento Therapeutics), HLX13 (Shanghai Henlius Biotech), ISA203 (ISA Pharmaceuticals), PRS-300 Series A (Pieris Pharmaceuticals), BA3071 (BioAtla), CTLA4 Cancer Research Program (Biosortia Pharmaceuticals), RP3 (Replimune), CGO161 (Cold Genesys), APL-509 (Apollomics, JSR), AGEN2041 (Ludwig Institute for Cancer Research), APC 101 (Advanced Proteome), CTLA-4 Inhibitor (Advanced Proteome), BA3071 (BeiGene), BPI-002 (BeyondSpring Pharmaceuticals), CTLA-4 Antibody (Tikcro Technologies), Immuno-Oncology Research Program II (OliPass), PBP1701 (Prestige BioPharma), DB002 (DotBio), DB003 (DotBio), OR-2299 (OncoResponse), NK044 (Alphamab). In certain embodiments, the CTLA-4 inhibitor is ipilimumab. In other embodiments, the CTLA4 inhibitor is tremelimumab.

Immune checkpoint inhibitors of interest for use in combination with compounds described herein also include: LAG-3 inhibitors. In some embodiments, the LAG-3 inhibitor is chosen from LAG525 (Novartis), BMS-986016 (Bristol-Myers Squibb), or TSR-033 (Tesaro). In one embodiment, the LAG-3 inhibitor is an anti-LAG-3 antibody molecule. In one embodiment, the LAG-3 inhibitor is an anti-LAG-3 antibody molecule as disclosed in US 2015/0259420, published on Sep. 17, 2015, entitled “Antibody Molecules to LAG-3 and Uses Thereof,” incorporated by reference in its entirety. In one embodiment, the anti-LAG-3 antibody molecule comprises the CDRs, variable regions, heavy chains and/or light chains of BAP050-Clone I or BAP050-Clone J disclosed in US 2015/0259420. In one embodiment, the anti-LAG-3 antibody molecule is BMS-986016 (Bristol-Myers Squibb), also known as BMS986016. BMS-986016 and other anti-LAG-3 antibodies are disclosed in WO 2015/116539 and U.S. Pat. No. 9,505,839, incorporated by reference in their entirety. In one embodiment, the anti-LAG-3 antibody molecule is TSR-033 (Tesaro). In one embodiment, the anti-LAG-3 antibody molecule is IMP731 or GSK2831781 (GSK and Prima BioMed). IMP731 and other anti-LAG-3 antibodies are disclosed in WO 2008/132601 and U.S. Pat. No. 9,244,059, incorporated by reference in their entirety. In one embodiment, the anti-LAG-3 antibody molecule is IMP761 (Prima BioMed). Further known anti-LAG-3 antibodies include those described, e.g., in WO 2008/132601, WO 2010/019570, WO 2014/140180, WO 2015/116539, WO 2015/200119, WO 2016/028672, U.S. Pat. Nos. 9,244,059, 9,505,839, incorporated by reference in their entirety. In one embodiment, the anti-LAG-3 inhibitor is a soluble LAG-3 protein, e.g., IMP321 (Prima BioMed), e.g., as disclosed in WO 2009/044273, incorporated by reference in its entirety.

Immune checkpoint inhibitors of interest for use in combination with compounds described herein also include: Tim-3 inhibitors. In some embodiments, the TIM-3 inhibitor is MGB453 (Novartis) or TSR-022 (Tesaro). In one embodiment, the TIM-3 inhibitor is an anti-TIM-3 antibody molecule. In one embodiment, the TIM-3 inhibitor is an anti-TIM-3 antibody molecule as disclosed in US 2015/0218274, published on Aug. 6, 2015, entitled “Antibody Molecules to TIM-3 and Uses Thereof,” incorporated by reference in its entirety. In one embodiment, the anti-TIM-3 antibody molecule comprises the CDRs, variable regions, heavy chains and/or light chains of ABTIM3-hum11 or ABTIM3-hum03 disclosed in US 2015/0218274. In one embodiment, the anti-TIM-3 antibody molecule is TSR-022 (AnaptysBio/Tesaro). In one embodiment, the anti-TIM-3 antibody molecule comprises one or more of the CDR sequences (or collectively all of the CDR sequences), the heavy chain or light chain variable region sequence, or the heavy chain or light chain sequence of APE5137 or APE5121. APE5137, APE5121, and other anti-TIM-3 antibodies are disclosed in WO 2016/161270, incorporated by reference in its entirety. In one embodiment, the anti-TIM-3 antibody molecule is the antibody clone F38-2E2. Further known anti-TIM-3 antibodies include those described, e.g., in WO 2016/111947, WO 2016/071448, WO 2016/144803, U.S. Pat. Nos. 8,552,156, 8,841,418, and 9,163,087, incorporated by reference in their entirety.

In an effort to protect normal cells from treatment toxicity and to limit organ toxicities, cytoprotective agents (such as neuroprotectants, free-radical scavengers, cardioprotectors, anthracycline extravasation neutralizers, nutrients and the like) may be used as an adjunct therapy in combination with compounds of the present disclosure. Suitable cytoprotective agents include amifostine (ETHYOL®), glutamine, dimesna (TAVOCEPT®), mesna (MESNEX®), dexrazoxane (ZINECARD® or TOTECT®), xaliproden (XAPRILA®), and leucovorin (also known as calcium leucovorin, citrovorum factor and folinic acid).

A compound described herein may also be used to advantage in combination with known therapeutic processes, for example, the administration of hormones or especially radiation. A compound described herein may in particular be used as a radiosensitizer, especially for the treatment of tumors which exhibit poor sensitivity to radiotherapy.

In some embodiments, a compound as described herein and BTK inhibitor are co-administered. In other embodiments, a compound as described herein is administered after the BTK inhibitor. In still different embodiments, a compound as described herein is administered before the BTK inhibitor.

In various embodiments, the BTK inhibitor is ibrutinib. In some particular embodiments, the cancer is chronic lymphocytic leukemia (CLL), small lymphocytic lymphoma (SLL), or both. In some embodiments, the subject has received a prior treatment regimen for CLL, SLL, or both. In some embodiments, the subject was refractory after the prior treatment regimen, the subject has relapsed CLL, SLL, or both after a response to the prior treatment regimen, or the subject has detectable minimal residual disease (MRD).

In another embodiment, a compound as described herein, is administered to a subject in need thereof in combination with a Bcl-2 inhibitor, such as venetoclax. The administration may be before, concurrently or after administration of the Bcl-2 inhibitor. In certain embodiments the subject is insensitive to treatment with a Bcl-2 inhibitor, is ineligible for treatment with a Bcl-2 inhibitor or has relapsed after treatment with a Bcl-2 inhibitor. In one specific embodiment, a compound as described herein is administered to a subject in need thereof in combination with a Bcl-2 inhibitor, such as venetoclax for treatment of leukemia (e.g., CLL, SLL, or both).

In another embodiment, a compound as described herein is administered to a subject in need thereof in combination with an immunomodulator (e.g., a CAR-T therapy, an immune checkpoint inhibitor, such as a PD-1, PD-L1 or CTLA4 inhibitor). In some embodiments, the immunomodulator is a CAR-T therapy, including any of the CAR-T therapies described herein. In some embodiments, the immunomodulator is an immune checkpoint inhibitor, for example, a PD-1, PD-L1 or CTLA4 inhibitor, including any of the immune checkpoint inhibitors described herein. Without wishing to be bound by any particular theory, it is believed that the exemplified compounds can improve blood vessel perfusion to a tumor and thereby enhance drug delivery to the tumor. Enhanced drug delivery is expected, in turn, to enhance the efficacy of a drug, such as an immunomodulator (e.g., immunooncology agent), including any immunomodulators described herein, for example, by making the tumor more susceptible to circulating drug.

In still another embodiment, a compound as described herein, is administered to a subject in need thereof in combination with an immune checkpoint inhibitor (e.g., a PD-1 inhibitor (such as Pembrolizumab or Nivolumab), a PD-L1 inhibitor (such as Atezolizumab, Avelumab, or Durvalumab), a CTLA-4 inhibitor, a LAG-3 inhibitor, or a Tim-3 inhibitor). Accordingly, methods of the present disclosure include methods for treating cancer comprising administering an effective amount of a compound as described herein and an immune checkpoint inhibitor to a subject in need thereof. The administration of a compound as described herein may be before, concurrently or after administration of the immune checkpoint inhibitor (e.g., a PD-1 inhibitor (such as Pembrolizumab or Nivolumab), a PD-L1 inhibitor (such as Atezolizumab, Avelumab, or Durvalumab), a CTLA-4 inhibitor, a LAG-3 inhibitor, or a Tim-3 inhibitor).

In some embodiments, a compound as described herein and an immune checkpoint inhibitor are co-administered. In other embodiments, a compound as described herein is administered after the immune checkpoint inhibitor. In still different embodiments, a compound as described herein is administered before the immune checkpoint inhibitor.

In embodiments, a compound as described herein, is administered to a subject in need thereof in combination with a bromodomain inhibitor, a histone deacetylase (HDAC) inhibitor, or both.

In some embodiments, methods of the present disclosure further comprise administering radiation therapy to the subject.

Embodiments further relate to a method of administering a compound as described herein to a subject in need thereof in combination with a BTK inhibitor (e.g., Ibrutinib) or a CDK9 inhibitor (e.g., Alvocidib) provided herein, in combination with radiation therapy for inhibiting abnormal cell growth or treating the hyperproliferative disorder in the mammal. Techniques for administering radiation therapy are known in the art, and these techniques can be used in the combination therapy described herein. The administration of a pharmaceutically acceptable salt of a compound as described herein in this combination therapy can be determined as described herein.

The compound as described herein can also be used in combination with an amount of one or more substances selected from anti-angiogenesis agents, signal transduction inhibitors, antiproliferative agents, glycolysis inhibitors, or autophagy inhibitors.

In certain embodiments, a compound as described herein is administered in combination with Erlotinib. In some embodiments, such a combination is used to treat pancreatic cancer. In other embodiments, such a combination is used to treat lung cancer. In further embodiments, the lung cancer is non-small cell lung cancer.

In certain embodiments, a compound as described herein is administered in combination with osmertinib. In some embodiments, such a combination is used to treat lung cancer. In further embodiments, the lung cancer has an EGFR mutation.

The structure of the active compounds identified by code numbers, generic or trade names may be taken from the actual edition of the standard compendium “The Merck Index” or from databases, e.g., Patents International (e.g., IMS World Publications).

### NUMBERED EMBODIMENTS

1. A compound of Formula (I):

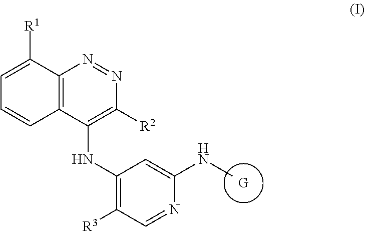

or a pharmaceutically acceptable salt thereof, wherein:
   R¹ is a C₁-C₅ alkyl or C₃-C₅ carbocycle, or a halogen;
   R² is —H, a halogen, a C₁-C₃ alkyl optionally substituted with one or more —F, or a cyclopropyl optionally substituted with one or more —F;
   R³ is —H, a halogen, a C₁-C₃ alkyl optionally substituted with one or more —F, or a cyclopropyl optionally substituted with one or more —F; and
   Ring G is

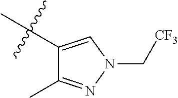 [SMILES: *c1cn(CC(F)(F)F)nc1C]

wherein

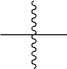

indicates the point of attachment of Ring G to the —N(H)—; or
Ring G is a C₆-C₁₀ aryl optionally substituted with:
   (i) one or more halogens;
   (ii) a sulfonamide;
   (iii) a monocyclic, bicyclic or spirocyclic C₃-C₁₀ carbocycle which is optionally substituted with one or more C₁-C₆ alkyl or C₃-C₆ carbocycle which are optionally substituted with hydroxy or one or more halogen, wherein said carbocycle is attached to Ring G by a single bond or a methylene or ethylene linker at a position on Ring G which is meta- or para- to the —N(H)— attached to Ring G; or
   (iv) a monocyclic, bicyclic, bridged or spirocyclic C₃-C₁₀ heterocycle which may contain up to 3 heteroatoms independently selected from N and O and which is optionally and independently substituted with one or more C₁-C₆ alkyl or C₃-C₆ carbocycle which are optionally substituted with hydroxy or one or more halogen, wherein said heterocycle is attached to Ring G by a single bond or a methylene or ethylene linker at a position on Ring G which is meta- or para- to the —N(H)— attached to Ring G.
2. The compound of numbered embodiment 1, wherein R¹ is a C₁-C₅ alkyl or C₃-C₅ carbocycle.
3. The compound of numbered embodiment 2, wherein R¹ is methyl or cyclopropyl.
4. The compound of numbered embodiment 1, wherein R¹ is a halogen.
5. The compound of numbered embodiment 1, wherein R¹ is methyl or a halogen.
6. The compound of numbered embodiment 5, wherein R¹ is methyl or chloro.
7. The compound of any one of numbered embodiments 1-6, wherein R² is —H, a halogen, —CH₃, —CF₃ or cyclopropyl.
8. The compound of numbered embodiment 7, wherein R² is —H.
9. The compound of any one of numbered embodiments 1-8, wherein R³ is —H, a halogen, —CH₃, —CF₃ or cyclopropyl.
10. The compound of numbered embodiment 9, wherein R³ is —H or a halogen.
11. The compound of numbered embodiment 9, wherein R³ is —H or fluoro.
12. The compound of numbered embodiment 9, wherein R³ is —H.
13. The compound of any one of numbered embodiments 1-12, wherein Ring G is

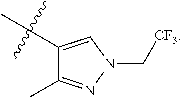 [SMILES: *c1cn(CC(F)(F)F)nc1C]

14. The compound of any one of numbered embodiments 1-12, wherein Ring G is a phenyl optionally substituted with:
   (i) one or more halogens;
   (ii) a sulfonamide;
   (iii) a monocyclic, bicyclic or spirocyclic C₃-C₁₀ carbocycle which is optionally substituted with one or more C₁-C₆ alkyl or C₃-C₆ carbocycle which are optionally substituted with hydroxy or one or more halogen, wherein said carbocycle is attached to Ring G by a single bond or a methylene or ethylene linker at a position on Ring G which is meta- or para- to the —N(H)— attached to Ring G; or
   (iv) a monocyclic, bicyclic, bridged or spirocyclic C₃-C₁₀ heterocycle which may contain up to 3 heteroatoms independently selected from N and O and which is optionally and independently substituted with one or more C₁-C₆ alkyl or C₃-C₆ carbocycle which are optionally substituted with hydroxy or one or more halogen, wherein said heterocycle is attached to Ring G by a single bond or a methylene or ethylene linker at a position on Ring G which is meta- or para- to the —N(H)— attached to Ring G.
15. The compound of numbered embodiment 14, wherein Ring G is a phenyl substituted with:
   (i) one or more halogens;
   (ii) a sulfonamide;
   (iii) a monocyclic, bicyclic or spirocyclic C₃-C₁₀ carbocycle which is optionally substituted with one or more C₁-C₆ alkyl or C₃-C₆ carbocycle which are optionally substituted with hydroxy or one or more halogen, wherein said carbocycle is attached to Ring G by a single bond or a methylene or ethylene linker at a position on Ring G which is meta- or para- to the —N(H)— attached to Ring G; or
   (iv) a monocyclic, bicyclic, bridged or spirocyclic C₃-C₁₀ heterocycle which may contain up to 3 heteroatoms independently selected from N and O and which is optionally and independently substituted with one or more C₁-C₆ alkyl or C₃-C₆ carbocycle which are optionally substituted with hydroxy or one or more halogen, wherein said heterocycle is attached to Ring G by a single bond or a methylene or ethylene linker at a position on Ring G which is meta- or para- to the —N(H)— attached to Ring G.
16. The compound of numbered embodiment 14 or 15, wherein Ring G is substituted with one or more halogens.
17. The compound of numbered embodiment 14 or 15, wherein Ring G is substituted with a sulfonamide.
18. The compound of numbered embodiment 14 or 15, wherein Ring G is substituted with a monocyclic, bicyclic or spirocyclic C₃-C₁₀ carbocycle which is optionally substituted with one or more C₁-C₆ alkyl or C₃-C₆ carbocycle which are optionally substituted with hydroxy or one or more halogen, wherein said carbocycle is attached to Ring G by a single bond or a methylene or ethylene linker at a position on Ring G which is meta- or para- to the —N(H)— attached to Ring G.
19. The compound of numbered embodiment 18, wherein Ring G is substituted with a monocyclic C₃-C₇ carbocycle which is optionally substituted with one or more C₁-C₆ alkyl or C₃-C₆ carbocycle which are optionally substituted with hydroxy or one or more halogen.
20. The compound of numbered embodiment 18 or 19, wherein Ring G is substituted with a cyclohexyl which is optionally substituted with one or more C₁-C₆ alkyl or C₃-C₆ carbocycle which are optionally substituted with hydroxy or one or more halogen.
21. The compound of any one of numbered embodiments 18-20, wherein the carbocycle that is attached to Ring G is unsubstituted.
22. The compound of any one of numbered embodiments 18-21, wherein the carbocycle that is attached to Ring G is attached to Ring G by a single bond.
23. The compound of numbered embodiment 14 or 15, wherein Ring G is substituted with a monocyclic, bicyclic, bridged or spirocyclic C₃-C₁₀ heterocycle which may contain up to 3 heteroatoms independently selected from N and O and which is optionally and independently substituted with one or more C₁-C₆ alkyl or C₃-C₆ carbocycle which are optionally substituted with hydroxy or one or more halogen, wherein said heterocycle is attached to Ring G by a single bond or a methylene or ethylene linker at a position on Ring G which is meta- or para- to the —N(H)— attached to Ring G.
24. The compound of numbered embodiment 23, wherein Ring G is substituted with a monocyclic C₅-C₆ heterocycle which may contain up to 3 heteroatoms independently selected from N and O and which is optionally and independently substituted with one or more C₁-C₆ alkyl or C₃-C₆ carbocycle which are optionally substituted with hydroxy or one or more halogen.
25. The compound of numbered embodiment 23 or 24, wherein Ring G is substituted with a monocyclic C₆ heterocycle which may contain up to 2 heteroatoms independently selected from N and O and which is optionally and independently substituted with one or more C₁-C₆ alkyl or C₃-C₆ carbocycle which are optionally substituted with hydroxy or one or more halogen.
26. The compound of any one of numbered embodiments 23-25, wherein Ring G is substituted with a piperazinyl, morpholinyl, piperidinyl or oxanyl, which is optionally and independently substituted with one or more C₁-C₆ alkyl or C₃-C₆ carbocycle which are optionally substituted with hydroxy or one or more halogen.
27. The compound of any one of numbered embodiments 23-26, wherein the heterocycle that is attached to Ring G is unsubstituted or monosubstituted.
28. The compound of numbered embodiment 27, wherein the heterocycle that is attached to Ring G is unsubstituted.
29. The compound of any one of numbered embodiments 23-28, wherein the heterocycle that is attached to Ring G is attached to Ring G by a single bond.
30. The compound of any one of numbered embodiments 18-20, 22-27 and 29, wherein the carbocycle or heterocycle that is attached to Ring G is optionally and independently substituted with methyl, CF₃CH₂— or HOCH₂CH₂—.
31. The compound of any one of numbered embodiments 1-12 and 14-30, wherein the carbocycle or heterocycle attached to Ring G is attached to Ring G at a position on Ring G which is meta- to the —N(H)— attached to Ring G.
32. The compound of any one of numbered embodiments 1-12 and 14-30, wherein the carbocycle or heterocycle attached to Ring G is attached to Ring G at a position on Ring G which is para- to the —N(H)— attached to Ring G.
33. The compound of any one of numbered embodiments 1-6, having the following structure:

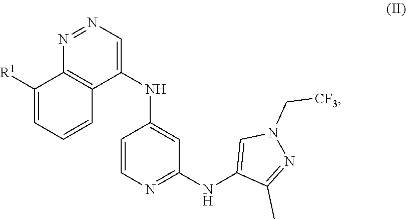

or a pharmaceutically acceptable salt thereof.
34. The compound of any one of numbered embodiments 1-6, having the following structure:

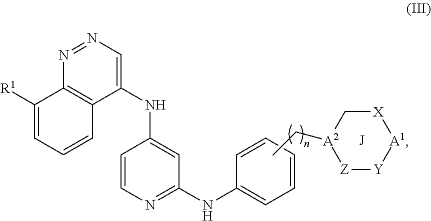

or a pharmaceutically acceptable salt thereof, wherein:
Ring J is attached to the phenylene at a position which is meta- or para- to the —N(H)— attached to the phenylene;
A¹ is —N(R⁴)—, —O— or >C(H)(R⁴);
R⁴ is —H, or a C₁-C₆ alkyl or C₃-C₆ carbocycle, each of which is optionally substituted with hydroxy or one or more halogen;
A² is >N— or >C(H)—;
Z is >CH₂; and X and Y are independently >CH₂ or >C(CH₃)₂, or X and Y are both >CH— and are bonded together through a methylene or ethylene bridge; or
Y is >CH₂ or >C(CH₃)₂, and X and Z are both >CH— and are bonded together through a methylene or ethylene bridge; and
n is 0, 1 or 2.
35. The compound of numbered embodiment 34, wherein A¹ is >C(H)(R⁴).
36. The compound of numbered embodiment 34, wherein A¹ is —N(R⁴)— or —O—.
37. The compound of numbered embodiment 36, wherein A¹ is —N(R⁴)—.
38. The compound of numbered embodiment 36, wherein A¹ is —O—.
39. The compound of any one of numbered embodiments 34-38, wherein R⁴ is —H, or a C₁-C₆ alkyl, which is optionally substituted with hydroxy or one or more halogen.
40. The compound of numbered embodiment 39, wherein R⁴ is —H, methyl, hydroxyethyl or trifluoroethyl.
41. The compound of numbered embodiment 40, wherein R⁴ is —H or methyl.
42. The compound of any one of numbered embodiments 34-41, wherein A² is >C(H)—.
43. The compound of any one of numbered embodiments 34-41, wherein A² is >N—.
44. The compound of numbered embodiment 34, wherein Ring J is:

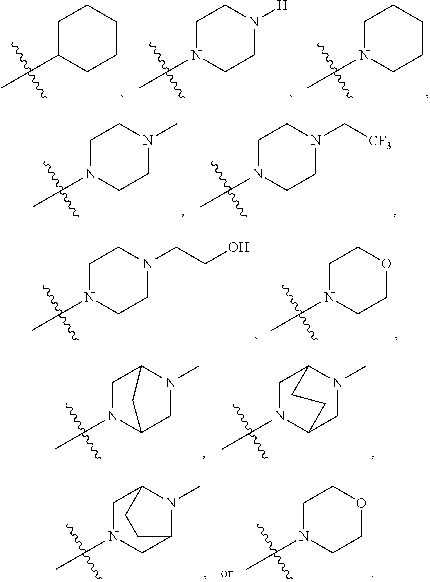

45. The compound of any one of numbered embodiments 34-44, wherein Ring J is attached to the phenylene at a position which is meta- to the —N(H)— attached to the phenylene.
46. The compound of any one of numbered embodiments 34-44, wherein Ring J is attached to the phenylene at a position which is para- to the —N(H)— attached to the phenylene.
47. The compound of any one of numbered embodiments 34-46, wherein n is 0 or 1.
48. The compound of numbered embodiment 47, wherein n is 0.
49. The compound of any one of numbered embodiments 1-6, 34-43, 45 and 46, having the following structure:

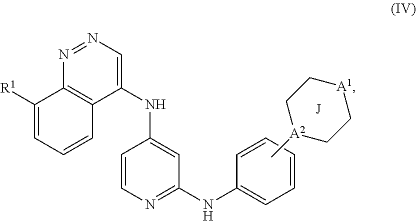

or a pharmaceutically acceptable salt thereof
50. The compound of numbered embodiment 49, wherein Ring J is:

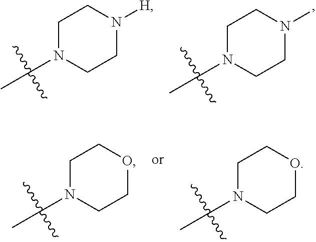

51. A compound, or a pharmaceutically acceptable salt thereof, having the structure:

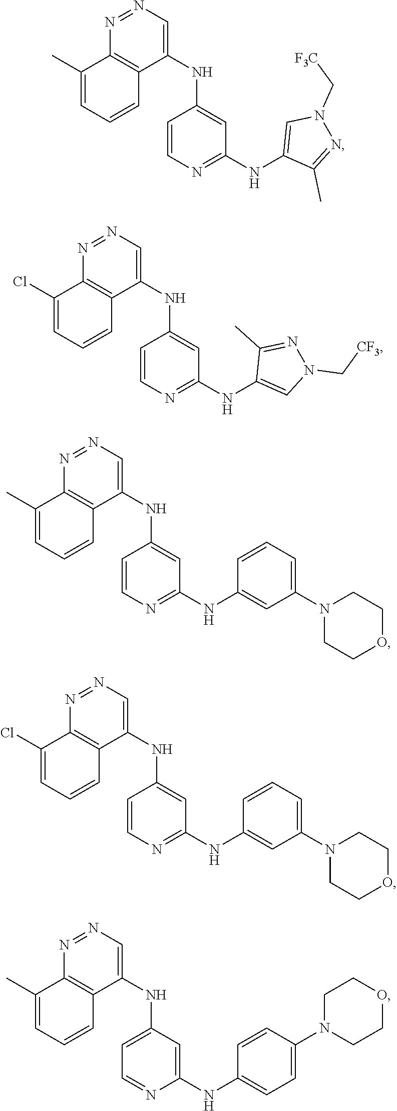 [SMILES: Cc1nn(CC(F)(F)F)cc1Nc1cc(Nc2cnnc3c(C)cccc23)ccn1 ; Cc1nn(CC(F)(F)F)cc1Nc1cc(Nc2cnnc3c(Cl)cccc23)ccn1 ; Cc1cccc2c(Nc3ccnc(Nc4cccc(N5CCOCC5)c4)c3)cnnc12 ; Clc1cccc2c(Nc3ccnc(Nc4cccc(N5CCOCC5)c4)c3)cnnc12 ; Cc1cccc2c(Nc3ccnc(Nc4ccc(N5CCOCC5)cc4)c3)cnnc12]

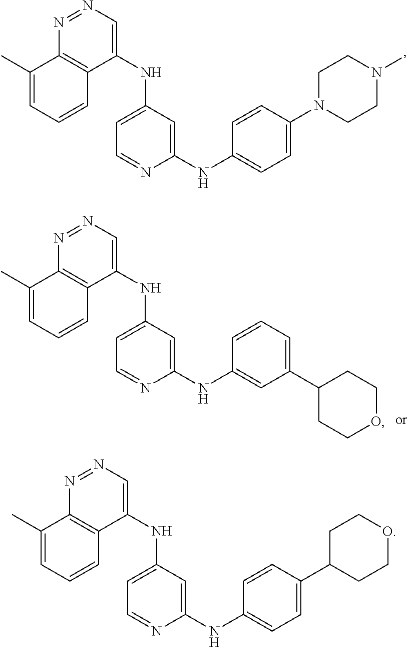

52. The compound of numbered embodiment 51, wherein the compound is of the following formula:

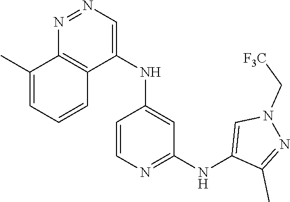 [SMILES: Cc1nn(CC(F)(F)F)cc1Nc1cc(Nc2cnnc3c(C)cccc23)ccn1]

or a pharmaceutically acceptable salt thereof.
53. The compound of numbered embodiment 51, wherein the compound is of the following formula:

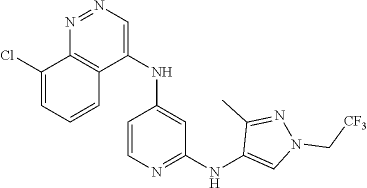 [SMILES: Cc1nn(CC(F)(F)F)cc1Nc1cc(Nc2cnnc3c(Cl)cccc23)ccn1]

or a pharmaceutically acceptable salt thereof.
54. The compound of numbered embodiment 51, wherein the compound is of the following formula:

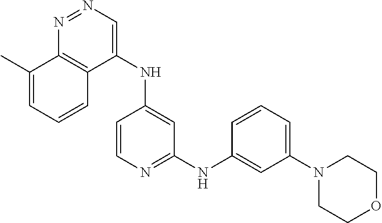 [SMILES: Cc1cccc2c(Nc3ccnc(Nc4cccc(N5CCOCC5)c4)c3)cnnc12]

or a pharmaceutically acceptable salt thereof.
55. The compound of numbered embodiment 51, wherein the compound is of the following formula:

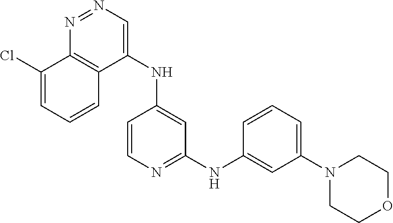 [SMILES: Clc1cccc2c(Nc3ccnc(Nc4cccc(N5CCOCC5)c4)c3)cnnc12]

or a pharmaceutically acceptable salt thereof.
56. The compound of numbered embodiment 51, wherein the compound is of the following formula:

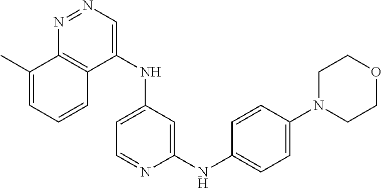 [SMILES: Cc1cccc2c(Nc3ccnc(Nc4ccc(N5CCOCC5)cc4)c3)cnnc12]

or a pharmaceutically acceptable salt thereof.
57. The compound of numbered embodiment 51, wherein the compound is of the following formula:

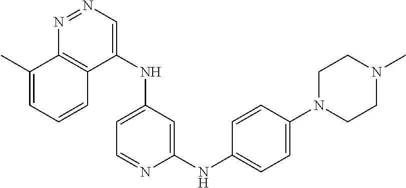

or a pharmaceutically acceptable salt thereof.
58. The compound of numbered embodiment 51, wherein the compound is of the following formula:

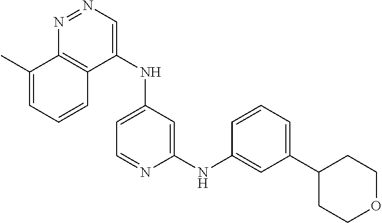 [SMILES: Cc1cccc2c(Nc3ccnc(Nc4cccc(C5CCOCC5)c4)c3)cnnc12]

or a pharmaceutically acceptable salt thereof.
59. The compound of numbered embodiment 51, wherein the compound is of the following formula:

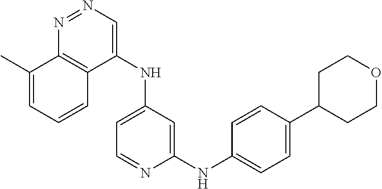 [SMILES: Cc1cccc2c(Nc3ccnc(Nc4ccc(C5CCOCC5)cc4)c3)cnnc12]

or a pharmaceutically acceptable salt thereof.
60. A pharmaceutical composition comprising a compound of any one of numbered embodiments 1-59, or a pharmaceutically acceptable salt thereof, and at least one pharmaceutically acceptable excipient.
61. A pharmaceutical combination comprising a compound of any one of numbered embodiments 1-59, or a pharmaceutically acceptable salt thereof, or a pharmaceutical composition of numbered embodiment 60, and one or more additional therapeutic agents.
62. A method of treating a proliferative disease in a subject, the method comprising administering to the subject a compound of any one of numbered embodiments 1-59, or a pharmaceutically acceptable salt thereof, or a pharmaceutical composition of numbered embodiment 60.
63. The method of numbered embodiment 62, wherein the proliferative disease is cancer.
64. The method of numbered embodiment 63, wherein the cancer is lung cancer, brain cancer, thyroid cancer, anaplastic astrocytoma, liver cancer, pancreatic cancer, skin cancer, melanoma, metastatic melanoma, colorectal cancer, breast cancer, prostate cancer, renal cancer, hepatocellular cancer, ovarian cancer, an HPV-associated cancer, multiple myeloma, myelodysplastic syndrome, a hematological cancer, or myelofibrosis.
65. The method of numbered embodiment 64, wherein the cancer is non-small cell lung cancer (NSCLC).
66. The method of numbered embodiment 64, wherein the cancer is neuroblastoma or glioblastoma.
67. The method of numbered embodiment 64, wherein the cancer is anaplastic thyroid cancer (ATC).
68. The method of numbered embodiment 64, wherein the cancer is colon carcinoma.
69. The method of numbered embodiment 64, wherein the cancer is hepatocellular carcinoma (HCC).
70. The method of numbered embodiment 64, wherein the cancer is pancreatic carcinoma.
71. The method of numbered embodiment 64, wherein the cancer is anaplastic large cell lymphoma (ALCL) or myelodysplastic syndrome.
72. The method of numbered embodiment 64, wherein the cancer is anaplastic astrocytoma.
73. The method of numbered embodiment 64, wherein the cancer is pancreatic ductal adenocarcinoma.
74. The method of numbered embodiment 64, wherein the cancer is an associated CAF cancer, metastatic melanoma, colorectal cancer, breast cancer, prostate cancer, renal cancer, hepatocellular cancer, ovarian cancer, an HPV-associated cancer, multiple myeloma, myelodysplastic syndrome, or myelofibrosis.
75. The method of numbered embodiment 64, wherein the HPV-associated cancer is selected from: cervical cancer, oropharyngeal cancer, anal cancer, vulvar/vaginal cancer, or penile cancer.
76. The method of any one of numbered embodiments 63-75, wherein the cancer is driven by TGF-β signaling.
77. The method of numbered embodiment 62, wherein the proliferative disease is a fibrotic condition.
78. The method of numbered embodiment 77, wherein the fibrotic condition is idiopathic pulmonary fibrosis, cardiac fibrosis, a condition associated with cardiac fibrosis, valvular disease, arrhythmia, atrial fibrillation, myocardial remodeling, cardiomyopathy, dilated cardiomyopathy, ischemic cardiomyopathy, hypertrophic cardiomyopathy, restenosis, liver fibrosis, liver cirrhosis, nonalcoholic steatohepatitis, Peyronie's, Dupuytren's contracture, cystic fibrosis, beta thalassemia, actinic keratosis, hypertension, a general inflammatory disorder, dry eye, ulcer, corneal fibrosis, wet age-related macular degeneration, psoriasis, wound closure, chronic kidney disease, renal fibrosis, systemic sclerosis, or chronic Chagas' heart disease.
79. A method of inhibiting tumor growth in a subject, the method comprising administering to the subject a compound of any one of numbered embodiments 1-59, or a pharmaceutically acceptable salt thereof, or a pharmaceutical composition of numbered embodiment 60.
80. The method of any one of numbered embodiments 62-79, further comprising administering one or more additional therapeutic agents to the subject.
81. The method of numbered embodiment 80, wherein at least one of the additional therapeutic agents is an anti-cancer agent.
82. The method of numbered embodiment 80 or 81, wherein at least one of the additional therapeutic agents is a PD-1 or PD-L1 inhibitor.
83. The method of any one of numbered embodiments 62-82, further comprising treating the subject with radiation therapy or surgery.
84. A method of inhibiting ALK-5 activity in vivo or in vitro, the method comprising contacting ALK-5 with a compound of any one of numbered embodiments 1-59, or a pharmaceutically acceptable salt thereof, or a pharmaceutical composition of numbered embodiment 60.
85. The method of numbered embodiment 84, wherein the inhibiting occurs in vivo in a subject.
86. The method of numbered embodiment 84, wherein the inhibiting occurs in vitro.
87. The method of any one of numbered embodiments 62-85, wherein the subject is a human.
88. The method of any one of numbered embodiments 84-87, wherein ALK-5 inhibition is at least 2-fold greater than ALK-2 inhibition under the same conditions.
89. A method of treating a fibrotic, inflammatory or proliferative disease or condition which is susceptible to inhibition of the TGFβ signaling pathway, the method comprising administering to a subject suffering from the fibrotic, inflammatory or proliferative disease or condition a compound of any one of numbered embodiments 1-59, or a pharmaceutically acceptable salt thereof, or a pharmaceutical composition of numbered embodiment 60, in an amount effective to inhibit TGFβ signaling.
90. The method of numbered embodiment 89, wherein the disease or condition is a fibrotic disease or condition.
91. The method of numbered embodiment 90, wherein said fibrotic disease or condition is selected from idiopathic pulmonary fibrosis, cardiac fibrosis, a condition associated with cardiac fibrosis, liver fibrosis, liver cirrhosis, nonalcoholic steatohepatitis, Peyronie's, Dupuytren's contracture, cystic fibrosis, beta thalassemia, actinic keratosis, hypertension, general inflammatory disorders, dry eye, ulcers, corneal fibrosis, wet age-related macular degeneration, psoriasis, wound closure, chronic kidney disease, renal fibrosis, systemic sclerosis, or chronic Chagas' heart disease.
92. The method of numbered embodiment 91, wherein said fibrotic disease or condition is idiopathic pulmonary fibrosis.
93. The method of numbered embodiment 89, wherein the disease or condition is an inflammatory disease or condition.
94. The method of numbered embodiment 89, wherein the disease or condition is a proliferative disease or condition.
95. The method of numbered embodiment 94, wherein the proliferative disease or condition is selected from anaplastic astrocytoma, pancreatic cancer, metastatic melanoma, colorectal cancer, breast cancer, prostate cancer, renal cancer, hepatocellular cancer, ovarian cancer, an HPV-associated cancer, cervical cancer, oropharyngeal cancer, anal cancer, vulvar/vaginal cancer, penile cancer, multiple myeloma, myelodysplastic syndrome, or myelofibrosis.
96. A method of suppressing TGFβ signaling in a subject suffering from a disease or condition which is promoted by TGFβ-signaling, comprising administering at least one compound of any one of numbered embodiments 1-59, or a pharmaceutically acceptable salt thereof, or a pharmaceutical composition of numbered embodiment 60 to the subject in an amount effective to sufficiently suppress TGFβ signaling to alter the course of the disease or condition.
97. A method of treating cachexia in a subject, comprising administering to the subject a compound of any one of numbered embodiments 1-59, or a pharmaceutically acceptable salt thereof, or a pharmaceutical composition of numbered embodiment 60.
98. A method of inhibiting epithelial to mesenchymal transition (EMT) in a subject suffering from a disease or condition which is promoted by EMT, comprising administering at least one compound of any one of numbered embodiments 1-59, or a pharmaceutically acceptable salt thereof, or a pharmaceutical composition of numbered embodiment 60 to the subject in an amount effective to sufficiently inhibit EMT to alter the course of the disease or condition.

### EXAMPLES

### Synthesis Examples

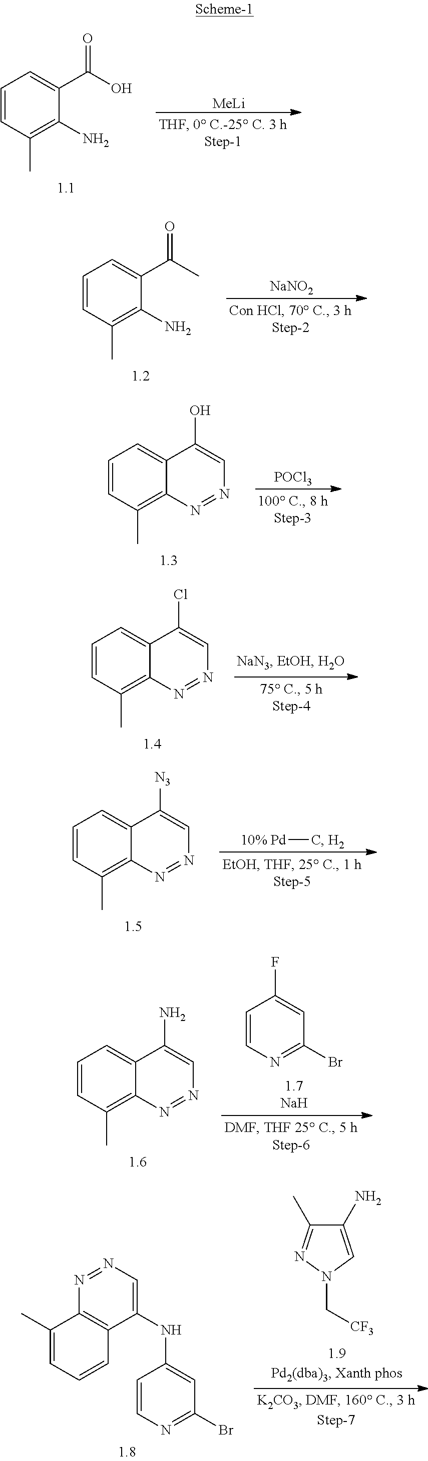

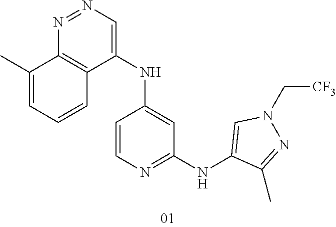 [SMILES: Cc1nn(CC(F)(F)F)cc1Nc1cc(Nc2cnnc3c(C)cccc23)ccn1]

### Synthesis of 1-(2-amino-3-methylphenyl)ethan-1-one (1.2)

To a suspension of 2-amino-3-methylbenzoic acid (1) (150 g, 993.3 mmol) in tetrahydrofuran (2.5 L) was added MeLi (1.6 M in diethyl ether) (2.48 L, 3973.5 mmol) at 0° C. and the resulting mixture was stirred at 25° C. temperature for 3 h. The reaction mixture was quenched with saturated ammonium chloride solution (2000 mL) and extracted with EtOAc (2×10 L). The combined organic layers were washed with water (1.0 L), brine (1.0 L), dried over anhydrous sodium sulfate and concentrated under vacuum to afford crude compound which was triturated with n-pentane (2×500 mL) to afford the title compound (1.2), which was characterized by 1H NMR (CDCl₃, 400 MHz): δ 7.65 (d, J=8.4 Hz, 1H), 7.21 (d, J=6.8 Hz, 1H), 6.59 (t, J=8.0 Hz, 1H), 6.41 (bs, 2H), 2.59 (s, 3H), 2.16 (s, 3H); and LCMS (M+H): 150.1.

### Synthesis of 8-methylcinnolin-4-ol (1.3)

To a stirred solution of 1-(2-amino-3-methylphenyl) ethan-1-one (1.2) (126 g, 845.6 mmol), in concentrated HCl (1.26 L) was added drop wise a solution of NaNO₂ (70 g 1014.7 mmol) in water (95 mL) at −5° C. and was stirred for 3 h at 70° C. The reaction mixture was cooled to room temperature, filtered and the residue was washed with diethyl ether (1.5 L). The filtrate was neutralized with saturated sodium bicarbonate up to pH 7 and solid precipitated was filtered and dried under vacuum to afford the title compound (3), which was characterized by 1H NMR (CDCl₃, 500 MHz): δ 10.06 (bs, 1H), 8.14 (d, J=8.0 Hz, 1H), 7.87 (s, 1H), 7.54 (d, J=7.0 Hz, 1H), 7.32-7.29 (m, 1H), 2.56 (s, 3H); and LCMS (M+H): 161.1.

### Synthesis of 4-chloro-8-methylcinnoline (1.4)

POCl₃ (380 mL) was added to the compound (1.3) (38 g, 187.0 mmol) at room temperature and allowed to stir at 100° C. for 8 h. The reaction mixture was cooled to room temperature and the excess POCl₃ was distilled off, residue was poured in to ice water (750 mL) and neutralized with saturated sodium bicarbonate up to pH 7, the precipitated solid was filtered off and dried under vacuum to afford the title compound (4), which was characterized by 1H NMR (CDCl₃, 400 MHz): δ 9.35 (s, 1H), 8.05 (d, J=7.6 Hz, 1H), 7.77-7.71 (m, 2H), 3.05 (s, 3H); and LCMS (M+H): 179.1.

### Synthesis of 4-azido-8-methylcinnoline (1.5)

To a stirred solution of compound (1.4) (30 g, 168.5 mmol) in ethanol (400 mL), water (100 mL), was added NaN₃ (54.77 g, 842.69 mmol) and stirred at 75° C. for 5 h. The reaction mixture was cooled to room temperature and concentrated under vacuum. The residue was diluted with water (500 mL), the precipitated solid was filtered off and dried under vacuum to afford the title compound (5), which was characterized by 1H NMR (CDCl₃, 400 MHz): δ 9.23 (s, 1H), 7.89 (d, J=8.4 Hz, 1H), 7.69-7.61 (m, 2H), 3.02 (s, 3H); and LCMS (M+H): 186.1.

### Synthesis of 8-methylcinnolin-4-amine (1.6)

To a stirred solution of 4-azido-8-methylcinnoline (1.5) (25 g, 135.13 mmol) in ethanol, THE (750 mL, 500 mL) was added 10% Pd/C (50% moisture) (5.0 g) and the reaction was allowed to stir under hydrogen gas for 1 h. The reaction mixture was filtered through a pad of celite, the residue was washed with methanol (2×1.0 L). The filtrate was concentrated under reduced pressure and co-distilled with toluene (2×500 mL) and triturated with ether (2×500 mL) to afford the title compound (6), which was characterized by ¹H NMR (DMSO-d₆, 400 MHz): δ 8.63 (s, 1H), 8.01 (d, J=8.4 Hz, 1H), 7.56 (d, J=6.8 Hz, 1H), 7.45 (t, J=8.0 Hz, 1H), 7.08 (bs, 2H), 2.76 (s, 3H); and LCMS (M+H): 160.1.

### Synthesis of N-(2-bromopyridin-4-yl)-8-methylcinnolin-4-amine (1.8)

A solution of 8-methylcinnolin-4-amine (1.6) (10 g, 62.8 mmol), 2-bromo-4-fluoropyridine (1.7) (13.2 g 75.00 mmol) in DMF (125 ml) & THE (125 mL) was added to a suspension of NaH (6.28 g, 157.00 mmol) in THE (125 mL). The resulting reaction mixture was stirred for 3 h at 25° C. The reaction mixture was quenched with saturated ammonium chloride solution and concentrated under vacuum, the residue was diluted with water (500 mL), the precipitated solid was filtered, washed with diethyl ether (2×200 mL) and dried under vacuum to afford the title compound (8), which was characterized by ¹H NMR (DMSO-d₆, 400 MHz): δ 10.40 (s, 1H), 9.11 (s, 1H), 8.19 (d, J=7.6 Hz, 1H), 8.09 (d, J=11.2 Hz, 1H), 7.73-7.63 (m, 2H), 7.38 (s, 1H), 7.27 (d, J=5.2 Hz, 1H), 2.83 (s, 3H); and LCMS (M+H): 315.04.

### Synthesis of N2-(3-methyl-1-(2,2,2-trifluoroethyl)-1H-pyrazol-4-yl)-N4-(8-methylcinnolin-4-yl)pyridine-2,4-diamine (Compound 01)

A mixture of N-(2-bromopyridin-4-yl)-8-methylcinnolin-4-amine (1.8) (35 g, 111.11 mmol), 3-methyl-1-(2,2,2-trifluoroethyl)-1H-pyrazol-4-amine (1.9) (19.88 g 111.11 mmol) and potassium carbonate (46.0 g, 333.33 mmol) in DMF (250 mL) was degassed for 20 min and added Pd₂(dba)₃ (10.176 g, 11.11 mmol), Xantphos (6.42 g, 11.11 mmol) and resulting reaction mixture was stirred for 3 h at 160° C. The reaction mixture was cooled to room temperature and diluted with water (750 mL), the precipitated solid was filtered and dried under vacuum to afford crude compound which was purified by flash column chromatography (100-200 silica mesh) using 1-5% methanol/dichloromethane as a eluent, followed by trituration with a DCM (100 mL) to afford the title compound (10), which was characterized by ¹H NMR (DMSO-d₆, 400 MHz): δ 9.35 (s, 1H), 9.31 (s, 1H), 8.21-8.19 (m, 2H), 8.14 (s, 1H), 8.02 (d, J=5.6 Hz, 1H), 7.74-7.67 (m, 2H), 6.80 (s, 1H), 6.66 (d, J=5.2 Hz, 1H), 4.99 (q, J=9.2 Hz, 2H), 2.88 (s, 3H), 2.17 (s, 3H); LCMS (M+H): 414.1; and HPLC: 98.06%.

### N4-(8-methylcinnolin-4-yl)-N2-(3-morpholinophenyl)pyridine-2,4-diamine (Compound 03)

Using a procedure analogous to that described for the synthesis of Compound 01 of Scheme 1, N-(2-bromopyridin-4-yl)-8-methylcinnolin-4-amine (1.8) (0.55 g, 1.74 mmol) was reacted with 3-morpholinoaniline (0.310 g, 1.74 mmol) in DMSO and purified by flash column chromatography (100-200 silica mesh) using 60-70% ethyl acetate/hexanes as eluent to give the title compound (03), which was characterized by 1H NMR (DMSO-d₆, 400 MHz): δ 9.37 (s, 1H), 9.36 (s, 1H), 8.85 (s, 1H), 8.20 (d, J=8.0 Hz, 1H), 8.06 (d, J=5.6 Hz, 1H), 7.74-7.68 (m, 2H), 7.23 (s, 1H), 7.15-7.07 (m, 2H), 6.82 (s, 1H), 6.73 (d, J=5.2 Hz, 1H), 6.50 (d, J=7.2 Hz, 1H), 3.74 (t, J=4.0 Hz, 4H), 3.07 (t, J=4.4 Hz, 4H), 2.89 (s, 3H); LCMS (M+H): 413.22; and HPLC: 98.49%.

### Synthesis of N4-(8-methylcinnolin-4-yl)-N2-(4-(tetrahydro-2H-pyran-4-yl)phenyl)pyridine-2,4-diamine (Compound 08)

Using a procedure analogous to that described for the synthesis of Compound 01 of Scheme 1, N-(2-bromopyridin-4-yl)-8-methylcinnolin-4-amine (1.8) (0.5 g, 1.58 mmol) was reacted with 4-(tetrahydro-2H-pyran-4-yl)aniline (0.28 g, 1.58 mmol) in DMSO and purified by flash column chromatography (100-200 silica mesh) using 60-70% ethyl acetate/hexanes as eluent to give the title compound (08), which was characterized by ¹H NMR (DMSO-d₆, 400 MHz): δ 9.36 (s, 2H), 8.89 (s, 1H), 8.20 (d, J=7.6 Hz, 1H), 8.04 (d, J=5.6 Hz, 1H), 7.74-7.68 (m, 2H), 7.55 (d, J=8.4 Hz, 2H), 7.13 (d, J=8.4 Hz, 2H), 6.80 (s, 1H), 6.74 (t, J=4.0 Hz, 1H), 3.95-3.92 (m, 2H), 3.45-3.39 (m, 2H), 2.89 (s, 3H), 2.71-2.66 (m, 1H), 1.67-1.62 (m, 4H); LCMS (M+H): 412.2; and HPLC: 98.84%.

### Synthesis of N4-(8-methylcinnolin-4-yl)-N2-(3-(tetrahydro-2H-pyran-4-yl)phenyl)pyridine-2,4-diamine (Compound 07)

Using a procedure analogous to that described for the synthesis of Compound 01 of Scheme 1, N-(2-bromopyridin-4-yl)-8-methylcinnolin-4-amine (1.8) (0.5 g, 1.58 mmol) was reacted with 3-(tetrahydro-2H-pyran-4-yl)aniline (0.28 g, 1.58 mmol) in DMSO and purified by flash column chromatography (100-200 silica mesh) using 60-70% ethyl acetate/hexanes as eluent to give the title compound (07), which was characterized by 1H NMR (DMSO-d₆, 400 MHz): δ 9.37 (s, 2H), 8.84 (s, 1H), 8.20 (d, J=7.6 Hz, 1H), 8.06 (d, J=5.6 Hz, 1H), 7.75-7.68 (m, 2H), 7.54 (d, J=8.4 Hz, 1H), 7.47 (s, 1H), 7.18 (t, J=8.0 Hz, 1H), 6.82 (d, J=1.6 Hz, 1H), 6.79-6.73 (m, 2H), 3.96-3.93 (m, 2H), 3.47-3.41 (m, 2H), 2.89 (s, 3H), 2.71-2.66 (m, 1H), 1.71-1.60 (m, 4H); LCMS (M+H): 412.2; and HPLC: 98.84%.

### Synthesis of N4-(8-methylcinnolin-4-yl)-N2-(4-(4-methylpiperazin-1-yl)phenyl)pyridine-2,4-diamine (Compound 06)

Using a procedure analogous to that described for the synthesis of Compound 01 of Scheme 1, N-(2-bromopyridin-4-yl)-8-methylcinnolin-4-amine (1.8) (0.2 g, 0.63 mmol) was treated with 4-(4-methylpiperazin-1-yl)aniline (0.12 g, 0.63 mmol) in DMF and purified by preparative HPLC to afford the title compound (06), which was characterized by 1H NMR (DMSO-d₆, 400 MHz): δ 9.33 (s, 1H), 9.31 (s, 1H), 8.66 (s, 1H), 8.19 (d, J=8.0 Hz, 1H), 8.00 (d, J=5.6 Hz, 1H), 7.74-7.67 (m, 2H), 7.44 (d, J=8.0 Hz, 2H), 6.87 (d, J=9.2 Hz, 2H), 6.71-6.67 (m, 2H)), 3.04 (t, J=4.4 Hz, 4H), 2.88 (s, 3H), 2.44 (t, J=4.8 Hz, 4H), 2.21 (s, 3H); LCMS (M+H): 426.2; and HPLC: 95.52%.

### Synthesis of N4-(8-methylcinnolin-4-yl)-N2-(4-morpholinophenyl)pyridine-2,4-diamine (Compound 05)

Using a procedure analogous to that described for the synthesis of Compound 01 of Scheme 1, N-(2-bromopyridin-4-yl)-8-methylcinnolin-4-amine (1.8) (10.0 g, 31.74 mmol) was treated with 4-morpholinoaniline (5.65 g, 31.74 mmol) in DMSO, and purified by flash column chromatography (100-200 silica mesh) using 1-5% methanol/DCM as eluent to give the title compound (05), which was characterized by 1H NMR DMSO-d₆, 400 MHz): δ 9.33 (s, 2H), 8.69 (s, 1H), 8.20 (d, J=8.0 Hz, 1H), 8.01 (d, J=5.6 Hz, 1H), 7.74-7.67 (m, 2H), 7.47 (d, J=8.8 Hz, 2H), 6.88 (d, J=8.8 Hz, 2H), 6.71-6.72-6.68 (m, 2H)), 3.73 (t, J=4.4 Hz, 4H), 3.01 (t, J=4.4 Hz, 4H), 2.88 (s, 3H); LCMS (M+H): 413.2; and HPLC: 96.01%.

### Synthetic Schemes for Intermediates

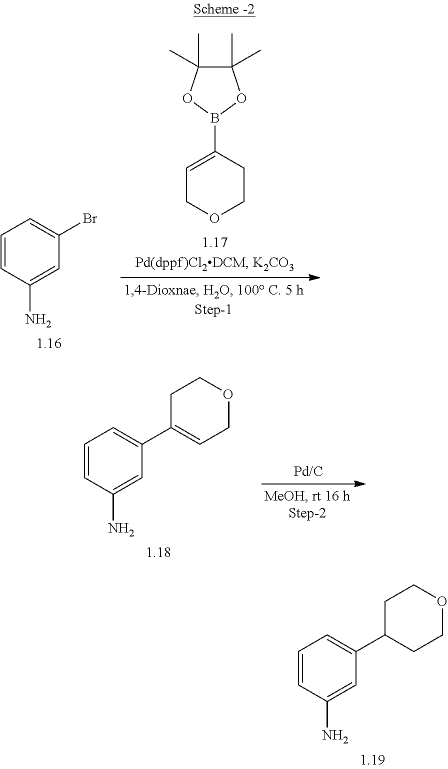

### Synthesis of 3-(3,6-dihydro-2H-pyran-4-yl)aniline (1.18)

A mixture of 3-bromoaniline (1.16) (5.0 g, 29.0 mmol), 2-(3,6-dihydro-2H-pyran-4-yl)-4,4,5,5-tetramethyl-1,3,2-dioxaborolane (1.17) (9.15 g, 43.6 mmol) and K₂CO₃ (12.0 g 87.0 mmol) in 1,4-dioxane (150 mL) and water (15 mL) was degassed using argon for 20 minutes. Pd(dppf)Cl₂·DCM (2.36 g, 2.90 mmol) was added, and the resulting reaction mixture was stirred at 100° C. for 5 h. The reaction mixture was cooled to room temperature and then extracted with ethyl acetate (2×200 mL). Combined organic layers were washed with water (100 mL) and brine (100 mL), dried over anhydrous sodium sulfate and concentrated under vacuum to afford crude compound which was purified by flash column chromatography on silica gel (100-200 mesh) using 30% ethyl acetate and hexanes as eluent to afford title compound 1.18, which was characterized by 1H NMR (CDCl₃. 400 MHz): δ 7.12 (t, J=8.0 Hz, 1H), 6.81-6.79 (m, 1H), 6.70 (t, J=2.0 Hz, 1H), 6.61-6.58 (m, 1H), 6.08-6.06 (m, 1H), 4.31-4.29 (m, 2H), 3.91 (t, J=5.2 Hz, 2H) 3.66 (bs, 2H), 2.50-2.46 (m, 2H); and LCMS (M+H): 176.04.

### Synthesis of 3-(tetrahydro-2H-pyran-4-yl) aniline (1.19

To a stirred solution of 3-(3,6-dihydro-2H-pyran-4-yl)aniline (1.18) (3.5 g, 20.0 mmol) in methanol (200 mL) was added 10% Pd/C (50% moisture) (1.0 g). The resulting reaction mixture was stirred under hydrogen par apparatus at 20 Psi for 16 h. The reaction mixture was filtered through a pad of celite and washed with methanol (2×100 mL). The filtrate was concentrated under reduced pressure, co-distilled with toluene (2×25 mL) and washed with ether (2×25 mL) to afford the title compound (1.19), which was characterized by 1H NMR (CDCl₃, 400 MHz): δ 7.12-7.08 (m, 1H), 6.64-6.62 (m, 1H), 6.55-6.53 (m, 2H), 4.07-4.04 (m, 2H), 6.34 (bs, 2H), 3.54-3.47 (m, 2H), 2.68-2.62 (m, 1H), 1.84-1.72 (m, 4H); and LCMS (M+H): 178.22.

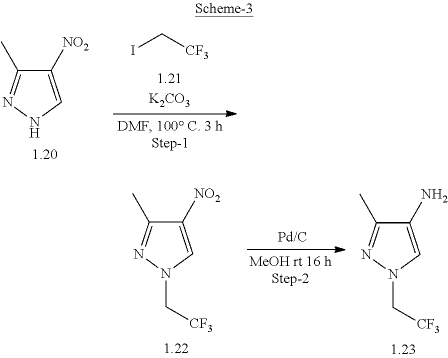

### Synthesis 3-methyl-4-nitro-1-(2,2,2-trifluoroethyl)-1H-pyrazole (1.22)

A mixture of 3-methyl-4-nitro-1H-pyrazole (1.20) (30.0 g, 236.22 mmol), 1, 1, 1-trifluoro-2-iodoethane (1.21) (148 g 708.66 mmole) and K₂CO₃ (97.79 g 708.66 mmol) in DMF (100 mL), was stirred at 100° C. for 3 h. The reaction mixture was cooled to room temperature extracted with ethyl acetate (2×200 mL). Combined organic layers were washed with water (100 mL), brine (100 mL), dried over anhydrous sodium sulfate and concentrated under vacuum to afford crude compound which was purified by flash column chromatography on silica gel (100-200 mesh) using 10% ethyl acetate and hexanes as eluent to afford title compound 1.22, which was characterized by 1H NMR (CDCl₃, 300 MHz): δ 8.25 (s, 1H), 4.70-4.62 (m, 2H), 2.56 (s, 3H); and LCMS (M+H): 210.01.

### Synthesis of 3-methyl-1-(2,2,2-trifluoroethyl)-1H-pyrazol-4-amine (1.23)

To a stirred solution of 3-methyl-4-nitro-1-(2, 2, 2-trifluoroethyl)-1H-pyrazole (1.22) (25 g, 119.04 mmol) in ethanol (750 mL) was added 10% Pd/C (50% moisture) (5.0 g) and stirred under hydrogen par apparatuses at 40 Psi for 6 h. The reaction mixture was filtered through a pad of celite and washed with methanol (2×1000 mL), the filtrate was concentrated under reduced pressure to afford crude compound which was purified by flash column chromatography on silica gel (100-200 mesh) using 50-100% ethyl acetate and hexanes as eluent to afford title compound 1.23, which was characterized by 1H NMR (CDCl₃, 400 MHz): δ 7.02 (s, 1H), 4.53-4.46 (m, 2H), 2.80 (bs, 2H), 2.18 (s, 3H); and LCMS (M+H): 180.01.

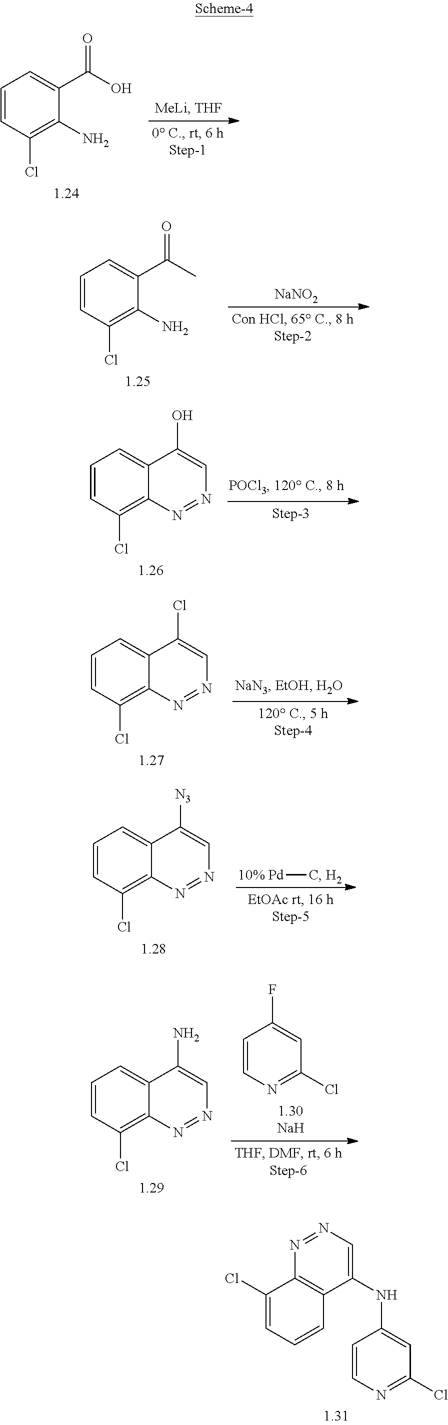

### 1-(2-amino-3-chlorophenyl)ethan-1-one (1.25)

To a suspension of 2-amino-3-chlorobenzoic acid (1.24) (20.0 g, 116.95 mmol) in tetrahydrofuran (300 mL) was added MeLi (1.6 M in diethyl ether, 293 mL, 467.83 mmol) at 0° C. and the resulting reaction mixture was stirred at 25° C. temperature for 2 h. The reaction mixture was quenched with saturated ammonium chloride solution (50 mL) and extracted with ethyl acetate (2×200 mL). The combined organic layers were washed with water (100 mL), brine (100 mL), dried over sodium sulfate and concentrated under vacuum to afford the crude product which was purified by flash column chromatography on silica gel (100-200 mesh) using 30% ethyl acetate and hexanes as eluent to afford title compound (1.25), which was characterized by 1H NMR (DMSO-d₆, 400 MHz): δ 7.66 (dd, J=1.6 Hz, 1.2 Hz, 1H), 7.41 (dd, J=1.6 Hz, 1.2 Hz, 1H), 7.82 (bs, 2H), 6.59 (t, J=8 Hz, 1H), 2.59 (s, 3H); and LCMS (M+H): 170.06.

### Synthesis of 8-chlorocinnolin-4-ol (1.26)

To a stirred solution of 1-(2-amino-3-chlorophenyl)ethan-1-one (1.25) (15.0 g, 88.75 mmol) in concentrated HCl (100 mL) was added a solution of NaNO₂ (7.40 g 106.50 mmol) in water (25 mL) drop wise at −5° C. and the resulting reaction mixture was stirred for 3 h at 70° C. The reaction mixture was cooled to room temperature and filtered, the residue was washed with diethyl ether (1.5 L) and the filtrate was neutralized with saturated sodium bicarbonate up to pH 7, the precipitated solid was filtered and dried under vacuum to afford the title compound (1.26), which was characterized by ¹H NMR CDCl₃, 300 MHz): δ 10.40 (bs, 1H), 8.18 (d, J=6.0 Hz, 1H), 7.88 (s, 1H), 7.77-7.74 (m, 1H), 7.34 (t, J=8.1 Hz, 1H); and LCMS (M−H): 181.7.

### Synthesis of 4, 8-dichlorocinnoline (1.27)

POCl₃ (50 mL) was added to the compound 8-chlorocinnolin-4-ol (1.26) (4.5 g, 25.0 mmol) at room temperature and allowed to stir at 100° C. for 8 h. The reaction mixture was cooled to room temperature and excess of POCl₃ was distilled off. The residue was poured in to ice water (50 mL) and basified with saturated sodium bicarbonate solution up to pH 7, the precipitated solid was filtered and dried under vacuum to afford the title compound (1.27), which was characterized by ¹H NMR CDCl₃, 400 MHz): δ 9.46 (s, 1H), 8.17-8.13 (m, 1H), 8.02-8.00 (m, 1H), 7.81-7.34 (m, 1H); and LCMS (M+H): 198.97.

### Synthesis of 4-azido-8-chlorocinnoline (1.28)

To a stirred solution 4,8-dichlorocinnoline (1.27) (4.3 g, 21.82 mmol) in ethanol (50 mL), water (5 mL), was added NaN₃ (7.10 g, 109.13 mmol) and stirred for 6 h at 75° C. The reaction mixture was cooled to room temperature and concentrated under vacuum. The residue was diluted with water (50 mL) and the precipitated solid was filtered and dried under vacuum to afford the title compound (1.28), which was characterized by 1H NMR (CDCl₃, 400 MHz): δ 9.31 (s, 1H), 7.99-7.95 (m, 2H) 7.68-7.63 (m, 1H); and LCMS (M+H): 205.95.

### Synthesis of 8-chlorocinnolin-4-amine (1.29)

To a stirred solution of 4-azido-8-chlorocinnoline (1.28) (4.0 g, 19.51 mmol) in ethyl acetate (100 mL) was added 10% Pd/C (50% moisture) (0.5 g) and stirred under hydrogen par apparatuses at 20 Psi for 16 h. The reaction mixture was filtered through a pad of celite and washed with methanol (2×100 mL), the filtrate was concentrated under reduced pressure and co-distilled with toluene (2×25 mL) and washed with ether (2×25 mL) to afford the title compound (1.29), which was characterized by 1H NMR (CDCl₃, 300 MHz): δ 8.71 (s, 1H), 8.18 (dd J=7.8 Hz, 1.2 Hz, 1H) 7.91 (dd J=6.6 Hz, 6.0 Hz, 1H) 7.56-7.51 (m, 1H), 7.45 (bs, 2H); and LCMS (M+H): 180.11.

### Synthesis of 8-chloro-N-(2-chloropyridin-4-yl) cinnolin-4-amine (1.31)

A solution of 8-chlorocinnolin-4-amine (1.29) (10 g, 55.86 mmol), 2-chloro-4-fluoropyridine (1.30) (8.88 g 67.03 mmol) in DMF (200 ml), THE (50 mL) was added a solution of NaH (5.6 g, 139.65 mmol) in THE (50 mL), the resulting reaction mixture was stirred for 6 h at 25° C. The reaction mixture was quenched with cold water and concentrated under vacuum, the residue was diluted with water (500 mL), the precipitated solid was filtered, washed with diethyl ether (2×200 mL) and dried under vacuum to afford the title compound (31), which was characterized by 1H NMR (DMSO-d₆, 400 MHz): δ 10.40 (s, 1H), 9.21 (s, 1H), 8.27-8.24 (m, 2H), 8.06 (d, J=6.6 Hz, 1H), 7.73 (t, J=7.5 Hz, 1H), 7.31.-7.29 (m, 2H); and LCMS (M+H): 290.94.

### Synthesis of N4-(8-chlorocinnolin-4-yl)-N2-(3-morpholinophenyl)pyridine-2,4-diamine (Compound 04)

Using a procedure analogous to that described for the synthesis of Compound 01 of Scheme 1, 8-chloro-N-(2-chloropyridin-4-yl)cinnolin-4-amine (60.0 g, 206.89 mmol) was treated with 3-morpholinoaniline (36.82 g, 206.89 mmol) in DMSO purified by flash column chromatography (100-200 silica mesh) using 1-5% methanol/DCM as eluent to give the title compound (04), which was characterized by 1H NMR (DMSO-d₆, 400 MHz): δ 9.58 (s, 1H), 9.43 (s, 1H), 8.89 (s, 1H), 8.38 (d, J=7.6 Hz, 1H), 8.11-8.07 (m, 2H), 7.79-7.76 (m, 1H), 7.24 (s, 1H), 7.16-7.12 (m, 1H), 7.10-7.08 (m, 1H), 6.86 (s, 1H), 6.76 (s, 1H), 6.51 (d, J=5.8 Hz, 1H), 3.75 (t, J=4.4 Hz, 4H), 3.08 (t, J=4.8 Hz, 4H); LCMS (M+H): 433.00; and HPLC: 98.71%.

### Synthesis N4-(8-chlorocinnolin-4-yl)-N2-(3-methyl-1-(2,2,2-trifluoroethyl)-1H-pyrazol-4-yl)pyridine-2,4-diamine (Compound 02)

Using a procedure analogous to that described for the synthesis of Compound 01 of Scheme-1, 8-chloro-N-(2-chloropyridin-4-yl)cinnolin-4-amine (2.5 g, 8.59 mmol) was reacted with 4-((4-methylpiperazin-1-yl)methyl)aniline 3-methyl-1-(2,2,2-trifluoroethyl)-1H-pyrazol-4-amine (1.53 g, 8.59 mmol) and purified by preparative HPLC to give the title compound (02), which was characterized by 1H NMR DMSO-d₆, 500 MHz): δ 9.53 (s, 1H), 9.41 (s, 1H), 8.38 (s, 1H), 8.27 (s, 1H), 8.14 (s, 1H), 8.07 (t, J=5.6 Hz, 2H), 7.77 (d, J=8.0 Hz, 1H), 6.83 (s, 1H), 6.70-6.69 (m, 1H), 5.00 (q, J=9.2 Hz, 2H), 2.17 (s, 3H); LCMS (M−H): 434.02; and HPLC: 95.03%.

TABLE 1
Example Compounds

| Compound | | |
|---|---|---|
| No. | Compound Structure | IUPAC Name |
| 01 | 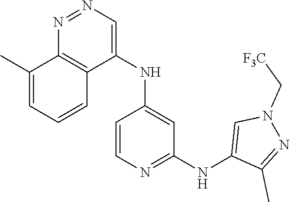 [SMILES: Cc1nn(CC(F)(F)F)cc1Nc1cc(Nc2cnnc3c(C)cccc23)ccn1] | N2-[3-methyl-1-(2,2,2-trifluoroethyl)- 1H-pyrazol-4-yl]-N4-(8- methylcinnolin-4-yl)pyridine-2,4- diamine |
| 02 | 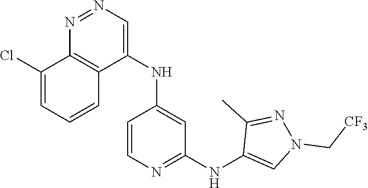 [SMILES: Cc1nn(CC(F)(F)F)cc1Nc1cc(Nc2cnnc3c(Cl)cccc23)ccn1] | N2-[3-methyl-1-(2,2,2-trifluoroethyl)- 1H-pyrazol-4-yl]-N4-(8- chlorocinnolin-4-yl)pyridine-2,4- diamine |
| 03 | 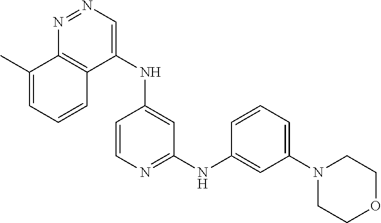 [SMILES: Cc1cccc2c(Nc3ccnc(Nc4cccc(N5CCOCC5)c4)c3)cnnc12] | N4-(8-methylcinnolin-4-yl)-N2-[3- (morpholin-4-yl)phenyl]pyridine-2,4- diamine |
| 04 | 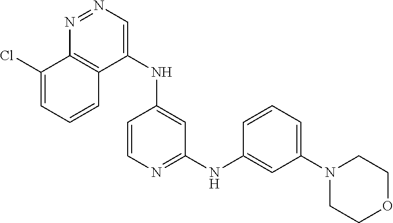 [SMILES: Clc1cccc2c(Nc3ccnc(Nc4cccc(N5CCOCC5)c4)c3)cnnc12] | N4-(8-chlorocinnolin-4-yl)-N2-[3- (morpholin-4-yl)phenyl]pyridine-2,4- diamine |
| 05 | 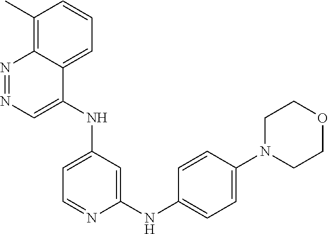 [SMILES: Cc1cccc2c(Nc3ccnc(Nc4ccc(N5CCOCC5)cc4)c3)cnnc12] | N4-(8-methylcinnolin-4-yl)-N2-[4- (morpholin-4-yl)phenyl]pyridine-2,4- diamine |
| 06 | 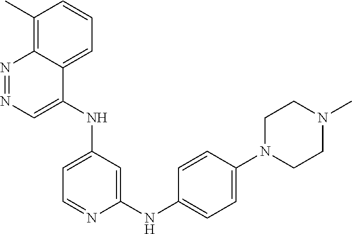 [SMILES: Cc1cccc2c(Nc3ccnc(Nc4ccc(N5CCN(C)CC5)cc4)c3)cnnc12] | N4-(8-methylcinnolin-4-yl)-N2-[4-(4- methylpiperazin-1-yl)phenyl]pyridine- 2,4-diamine |
| 07 | 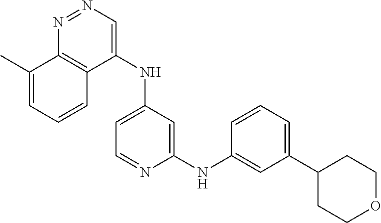 [SMILES: Cc1cccc2c(Nc3ccnc(Nc4cccc(C5CCOCC5)c4)c3)cnnc12] | N4-(8-methylcinnolin-4-yl)-N2-[3- (oxan-4-yl)phenyl]pyridine-2,4- diamine |
| 08 | 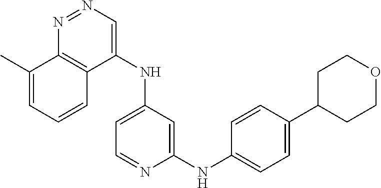 [SMILES: Cc1cccc2c(Nc3ccnc(Nc4ccc(C5CCOCC5)cc4)c3)cnnc12] | N4-(8-methylcinnolin-4-yl)-N2-[4- (oxan-4-yl)phenyl]pyridine-2,4- diamine |

### Assay Examples

### Example 1. ALK5 and ALK2 LanthaScreen™ Kinase Assay

Selected compounds of the present disclosure were assayed for inhibitory activity against ALK5 and ALK2. Activity determinations and selectivity were conducted by Thermo Fisher Scientific SelectScreen™ Biochemical Kinase Profiling Service using their LanthaScreen™ Eu Kinase Binding Assay Screening.

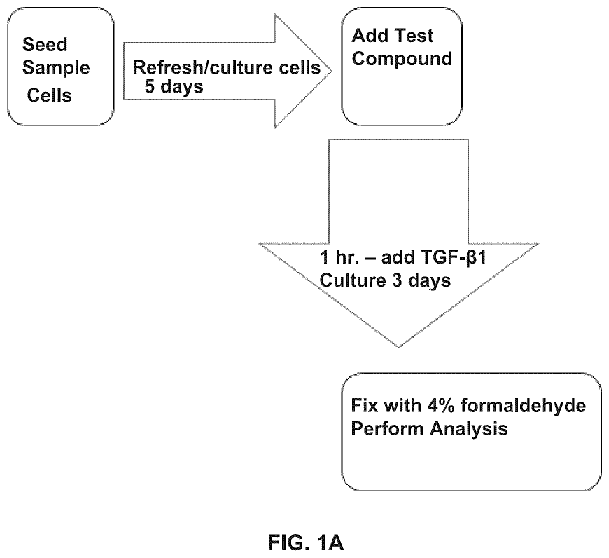

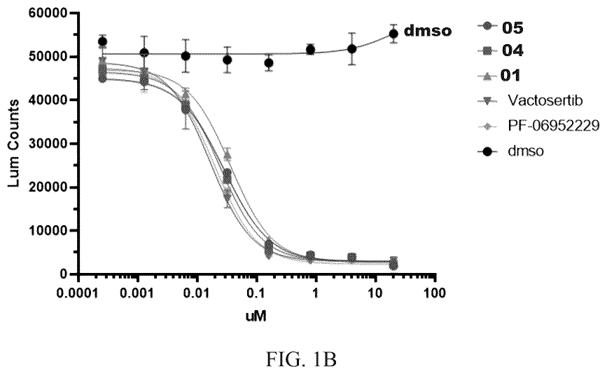

The principle of the LanthaScreen™ Eu Kinase Binding Assay is shown in FIG. 1A. Binding of an Alexa Fluor™ conjugate or “tracer” to a kinase is detected by addition of a Eu-labeled anti-tag antibody. Binding of the tracer and antibody to a kinase results in a high degree of FRET, whereas displacement of the tracer with a kinase inhibitor results in a loss of FRET. This assay is carried out by mixing the compound tested with the reagents and reading; no development step is required.

Life Technologies' Kinase Tracers are based on ATP-competitive kinase inhibitors, making them suitable for detection of any compounds that bind to the ATP site. Inhibitors that bind the ATP site include both Type I kinase inhibitors, which bind solely to the ATP site, and Type II inhibitors (e.g., Gleevec®/Imatinib, Sorafenib, BIRB-796), which bind to both the ATP site and a second site often referred to as the allosteric site.

The test compounds were screened in 1% DMSO (final) in the well. For 10-point titrations, 3-fold serial dilutions were conducted from the starting concentration (see Table 2 below).

TABLE 2
Kinase assay protocol details

| | Kinase | | Antibody | | Tracer | Tracer | | IC |
|---|---|---|---|---|---|---|---|---|
| | Conc | | Conc | | Conc | Kd | Known | 50 |
| Kinase | nM | Antibody | nM | Tracer* | nM | nM | inhibitor | nM |
| TGF-β1 | 5 | EU-anti- | 2 | Tracer | 10 | 30 | Dasatinib | 36.8 |
| (ALK-5) | | GST | | 178 | | | | |
| ACVR1 | 5 | EU-anti- | 2 | Tracer | 100 | 76 | Staurosporine | 48.1 |
| (ALK-2) | | GST | | 236 | | | | |
| ACVR1 | 5 | EU-anti- | 2 | Tracer | 100 | 44 | Staurosporine | 33.8 |
| (ALK-2) | | GST | | 236 | | | | |
| R206H | | | | | | | | |

Buffer used in all determinations was:
50 mM HEPES ph 7.5; 0.01% GRIJ-35; 10 mM MgCl₂; 1 mM EGTA
*Tracers are sourced from ThermoFisher

All Kinase/Antibody Mixtures were diluted to a 2× working concentration in the specified kinase buffer. The 4× AlexaFluor labeled Tracer was prepared in Kinase Buffer.

### Assay Protocol

Bar-coded, low volume, white, 384-well plate (Greiner Cat. #784207)
1. 160 nL—100× Test Compound in 100% DMSO
2. 3.84 μL—Kinase Buffer
3. 8.0 μL—2× Kinase/Antibody Mixture
4. 4.0 μL—4× Tracer
5. 30-second plate shake
6. 60-minute incubation at room temperature
7. Read on fluorescence plate reader and analysis of the data

The following controls were made for each individual kinase and were located on the same plate as the kinase:

0% Displacement Control: The maximum Emission Ratio was established by the 0% Displacement Control wells, which did not contain known inhibitor in the reaction and therefore exhibited no displacement of the tracer.

100% Displacement Control: The minimum Emission Ratio was established by the 100% Displacement Control wells, which contained the highest concentration of the known inhibitor used in that assay.

Known Inhibitor: A known inhibitor control standard curve, 10-point titration, was run for each individual kinase on the same plate as the kinase to ensure the inhibitor was displaced within an expected IC50 range previously determined.

The following equations were used for each set of data points:

| Value | Equation |
|---|---|
| Emission Ratio (ER) | $\frac{AF 647 Emission ( 665 nm )}{Europium Emission ( 615 nm )}$ |
| % Displacement | $\{ \frac{\begin{matrix}E {R}_{0 \% Disp Control} - \\ E {R}_{Sample}\end{matrix}}{\begin{matrix}E {R}_{0 \% DispControl} - \\ E {R}_{1 \; 00 \% Disp Control}\end{matrix}} \} \times 100$ |
| Difference | \|% Displacement_{Point 1} − % Displacement_{Point 2}\| |
| Between Data | |
| Points (single | |
| point only) | |
| Test Compound | For each emission wavelength, fluorescence interference |
| Interference | was flagged for a compound well that was more |
| | than 20% outside the range of the controls |
| Z (using ER values) | $1 - \{ \frac{\begin{matrix}3 * St d e {v}_{0 \% Disp Control} + \\ 3 * St d e {v}_{1 \; 00 \% Disp Ctrl}\end{matrix}}{\begin{matrix}{Mean}_{0 \% Disp Control} - \\ {Mean}_{1 \; 00 \% Disp Control}\end{matrix}} \}$ |

Data generated were plotted using the graphing software XLfit from IDBS. The dose response curve was curve fit to model number 205. If the bottom of the curve did not fit between −20% and 20% inhibition, it was set to 0% inhibition. If the top of the curve did not fit between 70% and 130% inhibition, it was set to 100% inhibition.

### Example 2. HotSpot™ JAK2 Assay Protocol

Reagent. Base Reaction buffer: 20 mM Hepes (pH 7.5), 10 mM MgCl₂, 1 mM EGTA, 0.01% Brij35, 0.02 mg/ml BSA, 0.1 mM Na₃VO₄, 2 mM DTT, 1% DMSO, with the addition of polyamino acid sodium salt (poly(Glu, Tyr) sodium salt Glu:Tyr (4:1), 5000-2000, sourced from Sigma Aldrich, Cat No. P7244) at a final concentration of 0.2 mg/mL.

Reaction Procedure:
1. Prepared substrate in freshly prepared Reaction Buffer.
2. Delivered kinase into the substrate solution and gently mixed.
3. Delivered compounds in 100% DMSO into the kinase reaction mixture by
Acoustic technology (Echo550; nanoliter range), incubated for 20 minutes at room temperature.
4. Delivered ³³P-ATP into the reaction mixture to initiate the reaction.
5. Incubated for 2 hours at room temperature.
6. Detected kinase activity by P81 filter-binding method.

This method was conducted in accordance with published protocol (see Anastassiadis T, et al., “Comprehensive assay of kinase catalytic activity reveals features of kinase inhibitor selectivity”, Nat Biotechnol. 2011 Oct. 30; 29(11):1039-45. doi: 10.1038/nbt.2017. PMID: 22037377; PMCID: PMC3230241), and further details of the method are summarized therein.

The JAK2 enzyme used in this assay was at a concentration of 0.25 nM. The JAK2 enzyme used is a recombinant human protein, catalytic domain (amino acids 808-1132), GST-tagged, expressed in insect cells. The material was obtained from Invitrogen, Cat. No. PV4210. Further details are available from ThermoFisher, catalog product number PV4210.

JAK2 kinase activity determination was performed at Reaction Biology Corporation (Malvern, PA) using the “HotSpot” assay platform. Briefly, the specific kinase/substrate pairs were prepared in reaction buffer. Compounds were delivered into the reaction, followed approximately 20 minutes later by addition of a mixture of ATP (Sigma, St. Louis MO) and ³³P ATP (Perkin Elmer, Waltham MA) to a final concentration of 10 μM. Reactions were carried out at room temperature for 120 minutes, followed by spotting of the reactions onto P81 ion exchange filter paper (Whatman Inc., Piscataway, NJ). Unbound phosphate was removed by extensive washing of filters in 0.75% phosphoric acid. After subtraction of background derived from control reactions containing inactive enzyme, kinase activity data was expressed as the percent remaining kinase activity in test samples compared to vehicle (dimethyl sulfoxide) reactions. IC₅₀ values and curve fits were obtained using Prism (GraphPad Software).

Statistical Methods. Raw data was measured in duplicate as percentage of compound activity for the tested kinase-inhibitor pair. The coefficient of variation (CV) and the difference (D) from duplicate observations were computed for the tested kinase-inhibitor pair.

### Example 3. RDSR Assay

TGF-beta (also referred to as TGF-β1) is a multifunctional, highly conserved cytokine with many key functions in development, cell growth and apoptosis, and plays a key role in the tissue repair response and functions as a potent immune modulator. TGF-β signaling is triggered when the activated TGF-β homodimer binds to the TGF-β receptor 2, which in turn leads to the recruitment and phosphorylation of TGF-β receptor 1 (ALK5). Activated TGF-β receptor 1 phosphorylates the signal transduction molecules SMAD2 and SMAD3. These bind to common mediator SMAD4 and translocate to the nucleus where they bind to short conserved DNA sequences called the SMAD binding element and induce the transcription of various target genes.

A stable cellular reporter, the RD SMAD reporter (RDSR), was generated to test the ability of certain compounds disclosed herein to inhibit the canonical TGF-β1-induced SMAD signaling pathway in a cellular context. The RD SMAD reporter (RDSR) cell line was generated by stably integrating the SMAD cellular reporter plasmid (Promega, pGL4.48[luc2P/SBE/Hygro]) into the human rhabdomyosarcoma cell line RD (ATCC, CCL-136). Once SMAD signaling was triggered with, for example, the addition of TGF-beta1, receptor-activated SMADs bind the SMAD binding elements (SBEs) leading to the expression of intracellular luciferase.

The rhabdomyosarcoma line RD (ATCC, CCL-136) was transfected with a SMAD reporter vector (Promega, E3671) and a polyclonal stable cell line was selected using hygromycin B. The transfected vector contained three copies of a Smad-binding element (SBE) that drives transcription of the luciferase reporter gene luc2P (Photinus pyralis). luc2P is a synthetically-derived luciferase sequence with humanized codon optimization that is designed for high expression and reduced anomalous transcription. The luc2P gene contains hPEST, a protein destabilization sequence, which allows luc2P protein levels to respond more quickly than those of luc2 to induction of transcription. The intracellular luciferase is quantified by the addition of equal volume (100 μl) of ONE-GLO substrate (Promega, E6120) and read within ten minutes on the Envision plate reader. The stable RDSR cell line was tested by evaluating the response to human TGF-beta1 (R&D Systems, 7754-BH-005) as well as myostatin (R&D Systems, 788-G8-010/CF) in a concentration-dependent manner after twenty-four hours of stimulation. IL-1 was used as a negative control and showed no response (data not shown). For compound evaluation, the tool compounds (vactosertib and PF-06952229 (a TGFβR1 inhibitor available from MedChem Express HY-136244) were included as positive controls) or test compounds were incubated with cells for one hour at 37° C. then stimulated with 200 pg/ml rhTGF-beta1 for twenty-four hours. The activity of the reporter was determined with the addition of ONE-GLO (Promega) substrate, and luminescence counts were collected on the Envision plate reader (Perkin Elmer). The IC50 or EC50 was calculated using nonlinear regression curve fitting with graph pad prism software.

The results of the aforementioned biochemical assays are reported in Table 3A.

TABLE 3A

| | | Kinase | | |
|---|---|---|---|---|
| | Kinase | Selectivity | | |
| | Inhibition: | ALK2 IC50/ | | |
| | ALK5 IC50 | ALK5 IC50 | RD SMAD | |
| | (Lantha) +++ = | (Lantha) +++ = | Reporter | Kinase |
| | IC50 ≤ 2 | selectivity > | (RDSR): | Selectivity: |
| | nM ++ = 2 | 500X ++ = | EC50 ++ = | JAK2 |
| | nM < IC50 ≤ | 500X ≥ | 50 nM ≤ | IC50/ |
| | 5 nM + = | selectivity ≥ | EC50 ≤ | ALK-5 |
| | IC50 | 100X + = | 100 nM +++ = | IC50 +++ = |
| Cmpd. | of < | selectivity < | EC50 < | selectivity > |
| No. | 5 nM | 100X | 50 nM | 1000X |
| 01 | +++ | +++ | +++ | +++ |
| 02 | + | ++ | ++ | +++ |
| 03 | ++ | + | +++ | +++ |
| 04 | ++ | + | +++ | +++ |
| 05 | ++ | + | +++ | +++ |
| 06 | ++ | + | +++ | +++ |
| 07 | ++ | + | ++ | +++ |
| 08 | ++ | + | +++ | +++ |

### Example 4. Comparative RDSR Assay

A comparative RDSR analysis was performed using IC50 values for selected compounds and standards obtained using the RDSR assay protocol described in Example 3.

The tool compounds vactosertib (an ALK5 inhibitor sourced from MedChem Express, HY-19928) and PF-06952229 (a TGFβR1 inhibitor) were included in the study as comparator compounds. Selected example compounds or comparator compounds were incubated with cells for one hour at 37° C. then stimulated with 200 pg/ml rhTGF-beta1 for twenty-four hours. The activity of the reporter was determined by adding ONE-GLO (Promega) substrate, and collecting luminescence counts on the Envision plate reader (Perkin Elmer).

Each of the example compounds and comparator compounds were tested at drug concentrations of from 20 micromolar to 1 nanomolar, and the IC50 value for each was calculated using Graph Pad Prism software. DMSO was used as a negative control and all wells contained normalized DMSO concentrations. All drugs were incubated with RDSR cells for one hour at 37° C. before stimulation with 200 pg/ml rhTGF-beta1 for twenty-four hours. The results of the comparative RDSR assay indicate that all compounds had IC50 ALK-5 activity of less than 50 nM, as shown in FIG. 1B.

These data demonstrate the certain example compounds inhibit ALK5 activity with potency similar to that exhibited by the comparator compounds vactosertib and PF-06952229.

### Example 5. ALK2 Off-Target Assay

Bone morphogenetic proteins (BMPs) are a subfamily that belongs to the TGF-β superfamily of ligands. The BMP signaling pathway controls a number of cell processes during development and in adult tissues. At a cellular level, BMP homodimer binds to a hetero-tetrameric receptor complex, composed of two type I receptors (ALK1, ALK 2, or ALK 3) and two type II receptors (ACTRII, ACTRIIB, or BMPRII). Upon complex formation, the constitutively active type II receptors phosphorylate type I receptors. Activated type I receptors phosphorylate and activate receptor-regulated SMAD proteins (SMAD1/5/8), which in turn bind to the common mediator SMAD4, and translocate to the nucleus where they bind to the consensus DNA sequence (SMAD binding element, SBE) in the promoter regions to induce target gene transcription.

Hepcidin is a small peptide synthesized predominantly by the liver. It is the master regulator of iron metabolism. BMP signaling positively regulates hepcidin transcription in liver cells.

To demonstrate that selected compounds do not inhibit ALK2, a close relative to ALK5, in a cellular context, the influence of selected compounds on BMP-6/ALK2/Hepcidin pathway activity was measured using hepcidin mRNA expression as a readout in human hepatocytes. In this study, LDN214117 (an ALK2 inhibitor available from Selleck Chemicals S7627) was included as a positive control, and vactosertib (an ALK5 inhibitor available from MedChem Express HY-19928) was included as a negative control.

HepG2 human hepatocytes (ATCC HB-8065) were seeded in EMEM (ATCC30-2003)+10% FBS in 96-well plate and allowed to grow overnight at 5% CO₂ at 37° C. The next day, cells were pre-treated with selected compounds (Compound 01, 04 or 05) or one of the control compounds at various concentrations, or with vehicle (DMSO) for one hour. Cells were then stimulated with recombinant human BMP-6 (R&D Systems 507-BP-020) at a final concentration of 40 ng/ml, in the presence of test compound or DMSO for three hours. A no-BMP-6 stimulation condition was included for baseline hepcidin level measurement.

Hepcidin mRNA expression was measured by real-time RT-PCR with Cells-to-CT 1-Step TaqMan Kit (ThermoFisher A25603) following manufacturer's instructions. GAPDH levels were measured, and served as a normalization control. Hepcidin primer: TaqMan HAMP-FAM/MGB 20×(ThermoFisher 4331182); GAPDH primer: TaqMan GAPDH-VIC/MGB 20×(ThermoFisher 4326317E). Delta-delta Ct method was used to obtain relative Hepcidin expression.

The selected compounds and control compounds LDN214117 and vactosertib were tested at drug concentrations of 20 micromolar to 1.22 nanomolar at 4-fold serial dilution. All wells contained the same DMSO concentrations. IC50 values were calculated using nonlinear regression model with GraphPad Prism software. The results of the ALK-2 off-target assay are reported in Table 3B, where “++” indicates 0.1 μM<IC₅₀≤1 μM and “+++” indicates IC₅₀≥1 μM.

TABLE 3B

| Compound No. | IC50 |
|---|---|
| 05 | ++ |
| 04 | ++ |
| 01 | +++ |
| LDN214117 | ++ |
| Vactosertib | +++ |

### Example 6. Fibroblast to Myofibroblast Transformation (FMT) Assay

Idiopathic pulmonary fibrosis (IPF) is a respiratory disease characterized by abnormal fibroblast activation and progressive fibrotic remodelling of the lungs. Though the exact pathophysiological mechanisms of IPF remain unknown, TGF-β1 is thought to act as a main driver of the disease by mediating fibroblast-to-myofibroblast transformation (FMT). TGF-β1-induced myofibroblasts are thought to play a major role in fibrosis due to excessive deposition of extracellular matrix.

To test the ability of selected compounds to inhibit the TGF-β1-dependent transition of fibroblasts to myofibroblasts in a relevant disease model of IPF, an FMT assay was performed using lung fibroblasts from IPF patients. In this assay, the transition of fibroblasts to myofibroblasts is determined by the expression of the biomarker alpha smooth muscle actin (α-SMA).

Primary human bronchial fibroblasts derived from IPF patients were seeded on day zero and the media was refreshed on day two. On day five, selected compounds or controls were added at an eight-point concentration response curve starting at 10 μM (semi-log dilutions). Each drug concentration condition was evaluated in biological duplicates. Cells were stimulated with 1.25 ng/ml of TGF-β1 one hour after drug addition. Seventy-two hours following TGF-beta addition, the cells were fixed with formaldehyde. High content imaging evaluating cell number using the nuclear stain DAPI as well as the expression of a-SMA was performed. The following controls were run alongside the selected compounds: vactosertib and the approved IPF drug nintedanib (at an eight-point, semi-log curve with 10 μM starting concentration). As a negative control 0.1% DMSO was also used, matching the DMSO concentration in treated wells. The following calculations were used to determine cell number as well as percent inhibition (PIN) of alpha SMA expression:

### Analysis of αSMA

Segmentation and quantification of αSMA immunoreactivity by an HCA algorithm, with density×area (D×A) output
Data normalization of raw αSMA (D×A) to percentage inhibition (PIN) values, on a plate-to-plate basis

$$PIN = 100 - ( \frac{{μ}_{p} - {X}_{i}}{{μ}_{p} - {μ}_{n}} ) \times 100$$

μₚ is the average αSMA value of the positive control (TGF-β1+1 μM SB525334)
μₙ is the average αSMA value of the vehicle control (TGF-β1+0.1% DMSO)
Xᵢ is the compound αSMA value
IC₅₀ values (if calculable) for all compounds
NB: IC₅₀ values are based on point of inflexion

### Analysis of % Remaining Cells

DAPI fluorescence applied for HCA-based quantification of the number of imaged cells, on a plate-to-plate basis

$$\% remaining cells = ( \frac{{X}_{i}}{{μ}_{n}} ) \times 100$$

μₙ is the average numbers of nuclei of the vehicle control (TGF-β1+0.1% DMSO)
Xᵢ is the compound number of nuclei

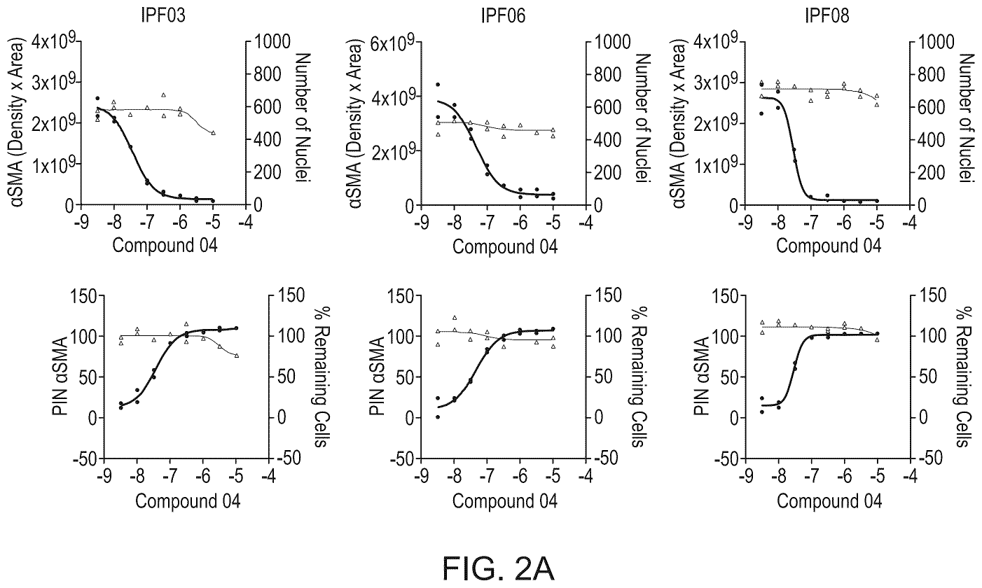

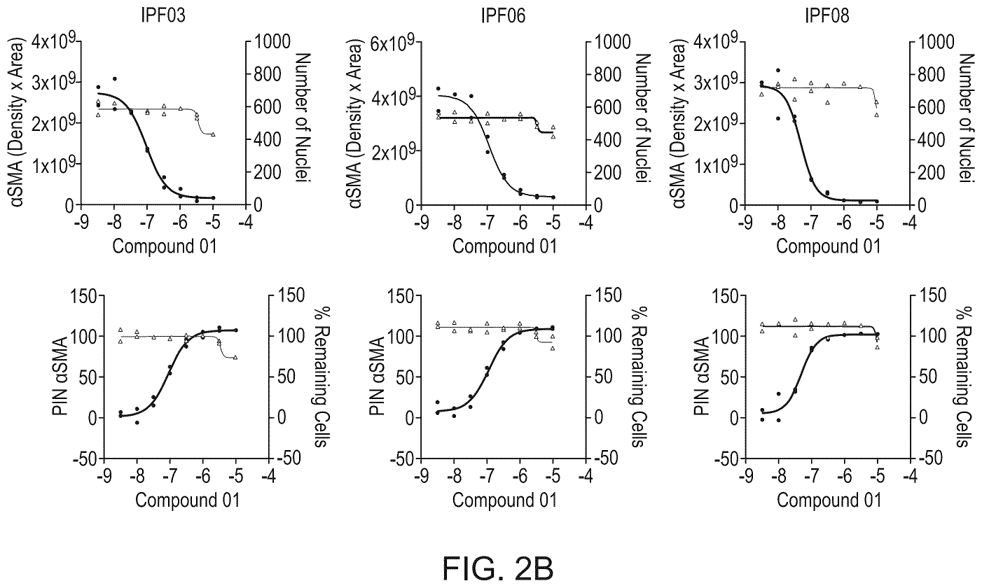

All compounds tested showed a high efficiency by inducing a full inhibition (maximum (max) PIN greater than 75) of TGF-beta1 mediated a-SMA expression, in all donors. Tables 4A-4D summarize the results from all individual donors as well as the controls. FIGS. 2A and 2B show individual experiments using three different IPF donors' cells, and the inhibitory capacity of Compound No. 04 (FIG. 2A) and Compound No. 01 (FIG. 2B) at inhibiting the upregulation of alpha SMA after TGF-beta treatment.

TABLE 4A
Summary for IPF03

| | | Max | Potentially | | |
|---|---|---|---|---|---|
| Compound | pIC50 | PIN | toxic | Spearman's | Assay |
| No. | αSMA | (%) | concentration¹ | correlation | window |
| 05 | 7.2 | 106 | 5.0 | 0.9 | 9.8 |
| 04 | 7.4 | 107 | — | | |
| 01 | 7.0 | 106 | 5.0 | | |
| Vactosertib | 7.3 | 105 | 5.0 | | |
| Nintedanib | 6.2 | 90 | 5.0 | | |

¹Potential cytotoxicity defined as >25% loss of nuclei compared to the average nuclei count of the vehicle control; lowest concentration at which >25% nuclear loss is observed is indicated

TABLE 4B
Summary for IPF06

| | | Max | Potentially | | |
|---|---|---|---|---|---|
| Compound | pIC50 | PIN | toxic | Spearman's | Assay |
| No. | αSMA | (%) | conc* | correlation | window |
| 05 | 7.3 | 106 | — | 0.9 | 7.9 |
| 04 | 7.3 | 106 | — | | |
| 01 | 7.0 | 107 | — | | |
| Vactosertib | 7.4 | 104 | — | | |
| Nintedanib | 5.8** | 104 | — | | |

*Potential cytotoxicity defined as >25% loss of nuclei compared to the average nuclei count of the vehicle control, lowest concentration at which >25% nuclear loss is observed is indicated
**Incomplete sigmoidal curve did not allow reliable potency determination

TABLE 4C
Summary for IPF08

| | | Max | Potentially | | |
|---|---|---|---|---|---|
| Compound | pIC50 | PIN | toxic | Spearman's | Assay |
| No. | αSMA | (%) | conc* | correlation | window |
| 05 | 7.4 | 101 | — | 0.9 | 25.8 |
| 04 | 7.5 | 100 | — | | |
| 01 | 7.3 | 101 | — | | |
| Vactosertib | 7.6 | 102 | — | | |
| Nintedanib | 6.4 | 94 | — | | |

*Potential cytotoxicity defined as >25% loss of nuclei compared to the average nuclei count of the vehicle control, lowest concentration at which >25% nuclear loss is observed is indicated

TABLE 4D

| Compound | | | | Average |
|---|---|---|---|---|
| No. | IPF03 | IPF06 | IPF08 | IC50 (nM) |
| 05 | 60 | 50 | 40 | 50.0 |
| 04 | 40 | 50 | 30 | 40.0 |
| 01 | 100 | 100 | 50 | 83.3 |
| Vactosertib | 50 | 40 | 25 | 38.3 |
| Nintedanib | 600 | 1500 | 400 | 833.3 |

### Example 7. A549 Xenograft Assay

To test compounds for inhibition of in vivo on-target activity (ALK5/TGF-bR1 inhibition) as well as potential Janus Kinase (JAK) signaling off-target activity, the A549 murine xenograft model was utilized. An ALK5 inhibitor (e.g., vactosertib) is expected to reduce the amount of the TGF-beta signaling molecule phospho-SMAD-2 (pSMAD2) in the A549 xenograft cells. The TGF-beta-mediated phosphorylation of SMAD2 in A549 cells takes place at amino acid residue four hundred and sixty-five and four hundred and sixty-seven (both are serine residues). A JAK inhibitor (e.g., ruxolitinib) is expected to reduce the phosphorylation of signal transducer and activator of transcription (STAT) proteins such as STAT3 at amino acid residue 705 (tyrosine).

At eight weeks of age, female athymic nude mice (purchased from Charles River) were injected with approximately 3.5 million A549 cells (ATCC, CCL-185). Specifically, cells were harvested and resuspended in plain RPMI media (no phenol red added) and Matrigel (Corning 356237) at a one to one ratio, and two hundred-microliters were injected into the right hind flank of each mouse. Tumors were measured every three days by caliper and as tumors reached an average of seventy-eighty millimeters cubed, mice were randomized in groups of three. All compounds were resuspended in 1-methyl-2-pyrrolidinone (Sigma 494496) (10%) plus 20% Solutol (Sigma 42966) in water (90%). Vactosertib and ruxolitinib were included as controls as ALK5 signaling inhibitor and JAK signaling inhibitor, respectively. Both controls were given at seventy-five milligrams per kilogram to three mice. The drug suspensions were sonicated for fifteen minutes to generate a fine particle suspension before being given to the mice. Mice were dosed (per oral gavage) with drug at one hundred, seventy-five, fifty, or ten milligrams per kilogram, with three mice per group. A vehicle control group with three mice was used to establish the baseline level of phospho-SMAD-2 in the tumor xenograft.

Four hours after drug administration, tumors were harvested, snap frozen and stored at negative eighty degrees Celsius until further processing. The phospho-SMAD-2 levels were determined using the Bio-Plex Pro™ Phospho-Smad2 (Ser465/Ser467) Set (BioRad 171V50019M). The phospho STAT3 levels were determined using the bead/antibody set from the MILLIPLEXMAP STAT Cell Signaling Magnetic Bead 5-Plex Kit (Millipore 48-610MAG). Both phospho SMAD-2 levels and phospho STAT3 levels were normalized to ß-Tubulin (MILLIPLEX MAP ß-Tubulin Total Magnetic Bead MAPmate™, Millipore 46-713MAG) or GAPDH (MILLIPLEX MAP GAPDH Total Magnetic Bead MAPmate™, Millipore 46-667MAG) levels from each sample. All analytes were analyzed in a multiplex fashion with the Bio-Plex Pro Cell Signaling Reagent Kit (BioRad 171304006M). Briefly, frozen tumor (fifteen to thirty milligrams) was lysed in 100 μl lysis buffer, processed in bead mill tube, and centrifuged. The resulting lysate was used at 1:50 dilution for the assay according to the manufacturer's instructions. Bead suspension was analyzed using the Luminex system (MAGPIX).

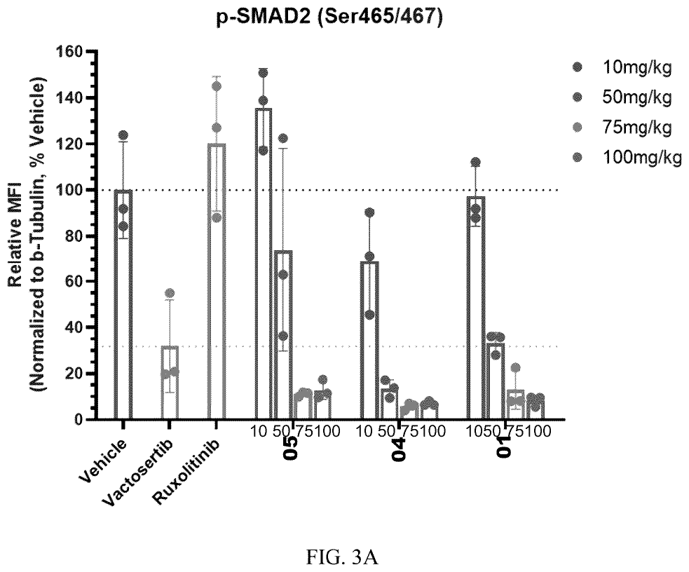

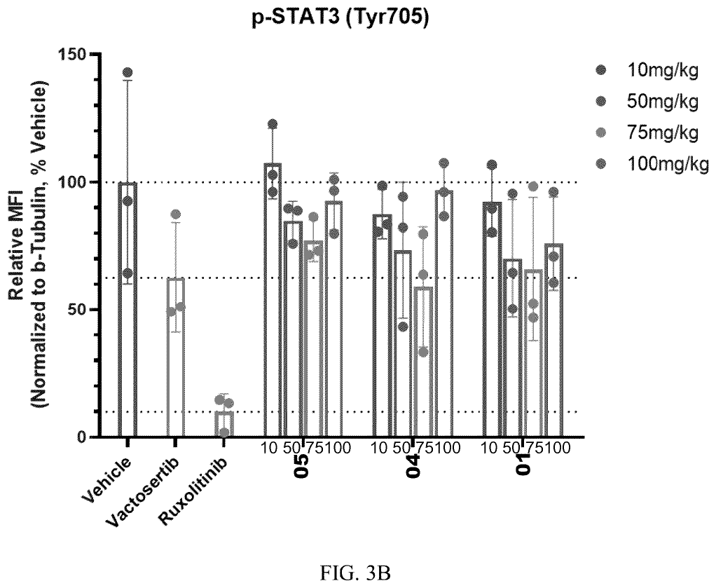

All compounds tested reduced the phopho-SMAD-2 levels in a dose-dependent fashion (FIG. 3A). Compound No. 04 showed the strongest potency, with 6% p-SMAD2 levels remaining compared to average vehicle, translating to a 94% inhibition of the biomarker p-SMAD2 when dosed with seventy-five milligrams per kilogram. The percent of p-SMAD-2 (Ser 465/467) compared to the average vehicle treated mouse is reported in Table 5A.

TABLE 5A
Average % p-SMAD2 (Ser 465/467) of vehicle

| Compound | 10 | 50 | 75 | 100 |
|---|---|---|---|---|
| No. | mg/kg | mg/kg | mg/kg | mg/kg |
| Vactosertib | | | 32 | |
| Ruxolitinib | | | 120 | |
| 05 | 136 | 74 | 11 | 13 |
| 04 | 69 | 13 | 6 | 7 |
| 01 | 97 | 33 | 13 | 8 |

The p-STAT3 levels were determined from the same tumor samples. The JAK signaling inhibitor ruxolitinib showed an average of 90% inhibition of the phosphorylation of STAT3 in tumor samples, given at 75 milligrams per kilogram (FIG. 3B, Table 5B). Vactosertib, a clinical stage ALK5 inhibitor, showed 63% of p-STAT3 levels compared to the vehicle control group. The test compounds were comparable or showed less inhibition of p-STAT3 than vactosertib (FIG. 3B, Table 5B), demonstrating their specificity.

TABLE 5B
Average % p-STAT3 (Tyr 705) of vehicle

| Compound | 10 | 50 | 75 | 100 |
|---|---|---|---|---|
| No. | mg/kg | mg/kg | mg/kg | mg/kg |
| Vactosertib | | | 63 | |
| Ruxolitinib | | | 10 | |
| 05 | 107 | 85 | 77 | 93 |
| 04 | 88 | 73 | 59 | 97 |
| 01 | 92 | 70 | 66 | 76 |

### Example 8. Longitudinal PK/PD Analysis of p-SMAD2 in A549 Xenograft Mouse Model

The ability of compounds to suppress TGF-beta signaling over time was demonstrated using a xenograft study carried out in a manner similar to that described in Example 7. Accordingly, a longitudinal, twenty-two-hour, one-dose study was performed using Compound 04 and the commercially available ALK5 inhibitor vactosertib in an A549 xenograft mouse model. In this study, p-SMAD2 (Ser465/Ser467) and a housekeeping gene (GAPDH) were measured at ten timepoints from zero (established using vehicle-treated animals) up to twenty-two hours post dose for a single drug dose of fifty milligrams per kilogram per subject (three subjects per dosing group).

For this study, xenografts were prepared and implanted in mice as follows. At five weeks of age, female athymic nude mice (purchased from Charles River) were injected with approximately 2.1 million A549 cells (ATCC, CCL-185). Cells were harvested and resuspended in plain RPMI media (no phenol red added) and Matrigel (Corning 356237) at a one to one ratio, and two hundred-microliters of the cell suspension were injected into the right hind flank of each mouse. Tumors were measured every three days by caliper, and as tumors reached an average of seventy-eighty millimeters cubed, mice were randomized in groups of three subjects. Each of the test compounds (vactosertib, Compound 04) was resuspended in 1-methyl-2-pyrrolidinone (Sigma, 494496) (10%) plus 20% Solutol (Sigma 42966) in water (90%). The drug suspensions were sonicated for fifteen minutes to generate a fine particle suspension before being given to the test subjects. Subjects were dosed (per oral gavage) with the suspension. A vehicle control group with three mice was used to establish the baseline and timepoint zero of phospho-SMAD-2 in the tumor xenograft. The test compounds were administered to the respective subject groups at fifty milligrams per kilogram.

Samples were obtained post administration of test compounds at 30 minutes, one hour, two hours, four hours, six hours, eight hours, twelve hours, sixteen hours and twenty-two hours. Tumors were harvested, snap frozen and stored at negative eighty degrees Celsius until further processing. Plasma was collected from all animals by collecting whole blood via cardiac puncture, followed by centrifugation in tubes containing EDTA (BD, microtainer tubes, 365974). A group of three mice receiving vehicle only served as the zero timepoint for both drug groups. The phospho SMAD-2 levels were determined using the Bio-Plex Pro™ Phospho-Smad2 (Ser465/Ser467) Set (BioRad 171V50019M). The phospho SMAD-2 levels were normalized to GAPDH (MILLIPLEX MAP GAPDH Total Magnetic Bead MAPmate™, Millipore 46-667MAG) levels from each sample. All analytes were analyzed in a multiplex fashion with the Bio-Plex Pro Cell Signaling Reagent Kit (BioRad 171304006M). Frozen tumor samples (fifteen to thirty milligrams) were lysed in 100 μl lysis buffer, processed in a bead mill tube, and centrifuged. The resulting lysate was used at 1:50 dilution for the assay according to the manufacturer's instructions. Bead suspension was analyzed using the Luminex system (MAGPIX).

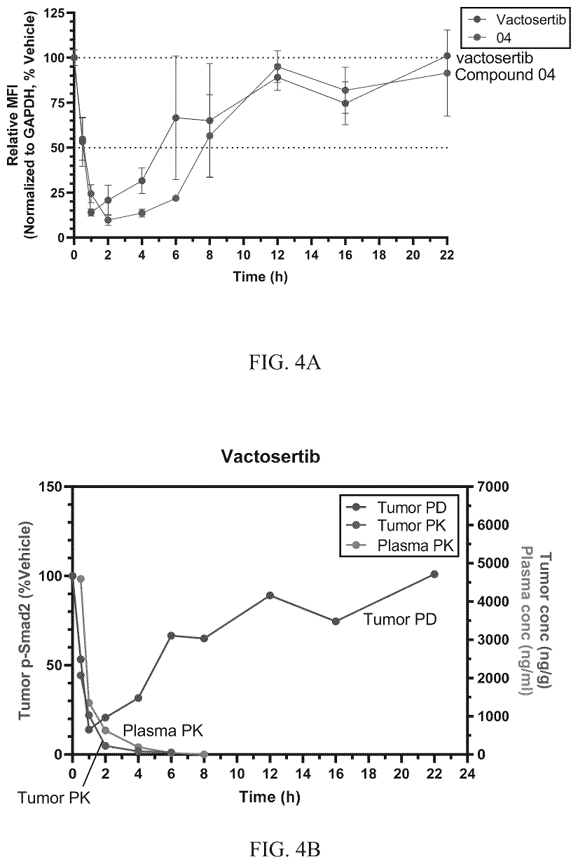

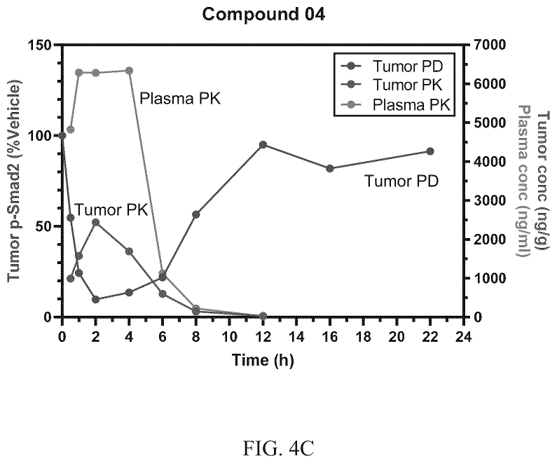

As seen in FIG. 4A, Compound 04 and vactosertib reduce the p-SMAD2 levels (normalized to GAPDH) 30 minutes post drug administration to similar levels (53% of vehicle for vactosertib and 55% of vehicle for Compound 04). However, as FIG. 4A shows, vactosertib was not able to maintain suppression of p-SMAD2 levels for an extended amount of time. As shown in FIG. 4A, vactosertib demonstrated peak suppression at one hour post dosing (14% p-SMAD2 levels normalized to GAPDH) compared to vehicle. The data in FIG. 4A and FIG. 4B show that vactosertib is quickly cleared from the system, resulting in p-SMAD2 levels rising rapidly after one hour. In contrast, Compound 04 reached its peak suppression at two hours post drug administration, achieving a suppression of p-SMAD2 levels to about 10% p-SMAD2 (normalized to GAPDH) compared to the average of the vehicle group.

FIGS. 4B and 4C show the PK/PD relationship for vactosertib (FIG. 4B) and Compound 04 (FIG. 4C). Cumulatively, the data in FIGS. 4B and 4C show that Compound 04 exhibits a longer drug tumor and plasma exposure in vivo than vactosertib, resulting in prolonged suppression of the TGF beta signaling molecule p-SMAD2 over time compared to vactosertib at comparable dosing levels.

### Example 9. Cachexia in Cancer

Cachexia is linked to chronic illness and manifests in involuntary weight loss (e.g., greater than 5% of pre-illness weight) resulting from the atrophy of skeletal muscle and adipose tissues. This condition is distinct from other conditions, like anorexia, where fat stores are depleted but muscle mass remains largely intact. Cachexia affects over half of cancer patients, resulting in poor quality of life (fatigue and weakness), and can sometimes even compromise treatment strategies in some individuals. Myostatin, a transforming growth factor-beta (TGF-beta) super-family member, has been well characterized as a negative regulator of muscle growth and development. Blocking this pathway would potentially benefit cancer patients, specifically patients with late stage disease and metastasis where cachexia is prominent.

To determine the ability of compounds to inhibit the SMAD signaling triggered by myostatin, and therefore possibly help alleviate cachexia in cancer patients, the following assay was performed. The RD cell line, a human rhabdomyosarcoma cell line, was purchased from ATCC (CCL-136) and a stably SMAD reporter plasmid (Promega, pGL4.48[luc2P/SBE/Hygro]) was introduced to create the RD SMAD Reporter (RDSR) cell line. Twenty thousand RDSR cells were seeded into a 96-well plate (Greiner bio-one 655083) in DMEM media supplemented with 10% fetal bovine serum. Cells were pre-incubated with selected compounds at various concentrations (8-point curve ranging from 10 micromolar to 610 picomolar) for one hour, then were stimulated with 50 nanograms per milliliter myostatin (R&D, 788-G8-010) for twenty-four hours. The luciferase signal was then read out with One-Glo substrate on the Envision plate reader.

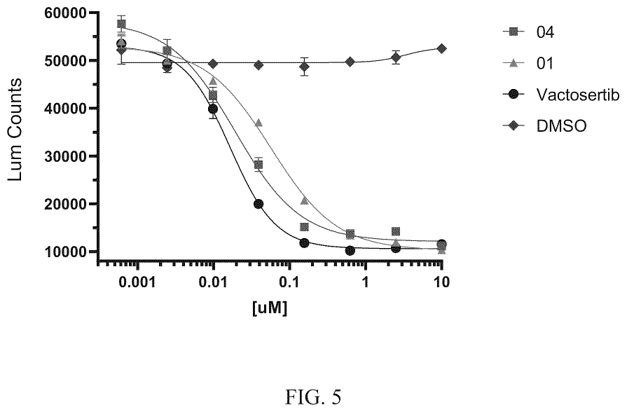

As seen in FIG. 5, SMAD signaling triggered by myostatin was inhibited with Compounds 01 and 04 with IC50s of 0.0571 micromolar and 0.0191 micromolar, respectively. Vactosertib inhibited the myostatin-induced SMAD signaling with an IC50 of 0.0164 micromolar. This data suggest that the exemplified compounds may reduce the involuntary weight loss, specifically muscle loss, in patients suffering from cachexia.

### Example 10. Longitudinal PK/PD Analysis of p-SMAD2 in A549 Xenograft Mouse Model

The study described in Example 8 was expanded to include Compound 01. Accordingly, using the same procedure for preparing test subjects and the ability of compounds tested to suppress TGF-beta signaling over time (as described in Example 8), a longitudinal, twenty-two-hour, one-dose study was performed using Compound 01, and the data were plotted alongside data obtained in Example 8 from Compound 04 and commercially available ALK5 inhibitor vactosertib, which were replotted in accordance with Example 10. For this expansion of the longitudinal study, p-SMAD2 (Ser465/Ser467) and housekeeping gene GAPDH were measured at ten timepoints from zero up to twenty-two hours post a single drug dose of fifty milligrams per kilogram per animal (three animals per group). A vehicle only treatment group of three mice was used to establish the baseline levels of p-SMAD2 in A549 xenograft tumor samples.

As in Example 8, xenografts were prepared and implanted in mice as follows. At five weeks of age, female athymic nude mice (purchased from Charles River) were injected with approximately 2.1 million A549 cells (ATCC, CCL-185). Specifically, cells were harvested and resuspended in plain RPMI media (no phenol red added) and Matrigel (Corning 356237) at a one to one ratio, and two hundred-microliters were injected into the right hind flank of each mouse. Tumors were measured every three days by caliper and as tumors reached an average of seventy-eighty millimeters cubed, mice were randomized in groups of three. As in Example 8, the compound tested was resuspended in 1-methyl-2-pyrrolidinone (Sigma, 494496) (10%) plus 20% Solutol (Sigma 42966) in water (90%) and the suspension was sonicated for fifteen minutes to generate a fine particle suspension before being administered per oral gavage to each of the subjects at fifty milligrams per kilogram per time point. A vehicle control group with three mice was used to establish the baseline and timepoint zero of phospho-SMAD-2 in the tumor xenograft.

As with the measurements obtained in Example 8, after various times post drug administration (specifically, 30 minutes, one hour, two hours, four hours, six hours, eight hours, twelve hours, sixteen hours and twenty-two hours), tumors were harvested, snap frozen and stored at negative eighty degrees Celsius until further processing. Plasma was collected from all animals by collecting whole blood via cardiac puncture, followed by centrifugation in tubes containing EDTA (BD, microtainer tubes, 365974). A group of three mice receiving vehicle only served as the zero timepoint for both drug groups. The phospho-SMAD-2 levels were determined using the Bio-Plex Pro™ Phospho-Smad2 (Ser465/Ser467) Set (BioRad 171V50019M). The phospho-SMAD-2 levels were normalized to GAPDH (MILLIPLEX MAP GAPDH Total Magnetic Bead MAPmate™, Millipore 46-667MAG) levels from each sample. All analytes were analyzed in a multiplex fashion with the Bio-Plex Pro Cell Signaling Reagent Kit (BioRad 171304006M). Briefly, frozen tumor (fifteen to thirty milligrams) was lysed in 100 μl lysis buffer, processed in bead mill tube, and centrifuged. The resulting lysate was used at 1:50 dilution for the assay according to the manufacturer's instructions. Bead suspension was analyzed using the Luminex system (MAGPIX).

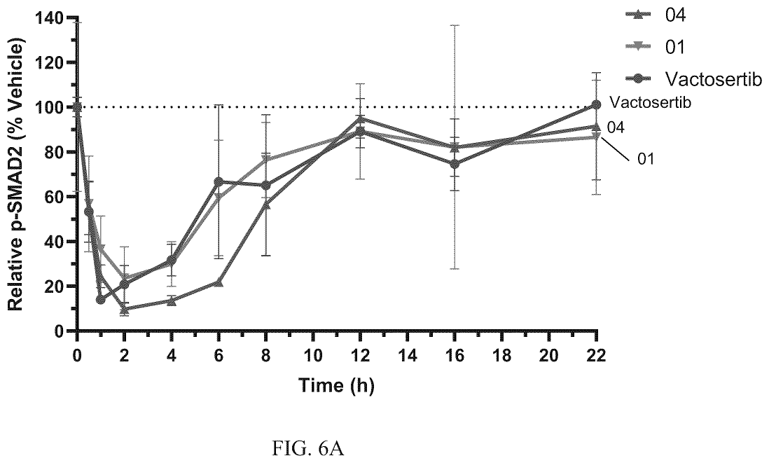

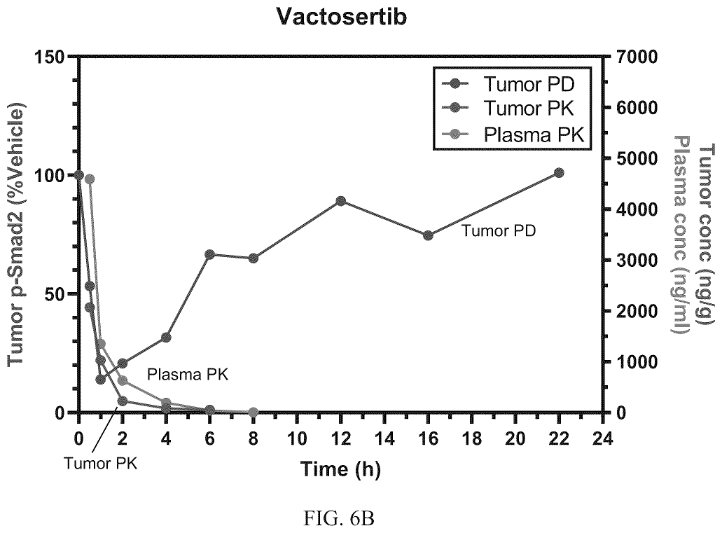

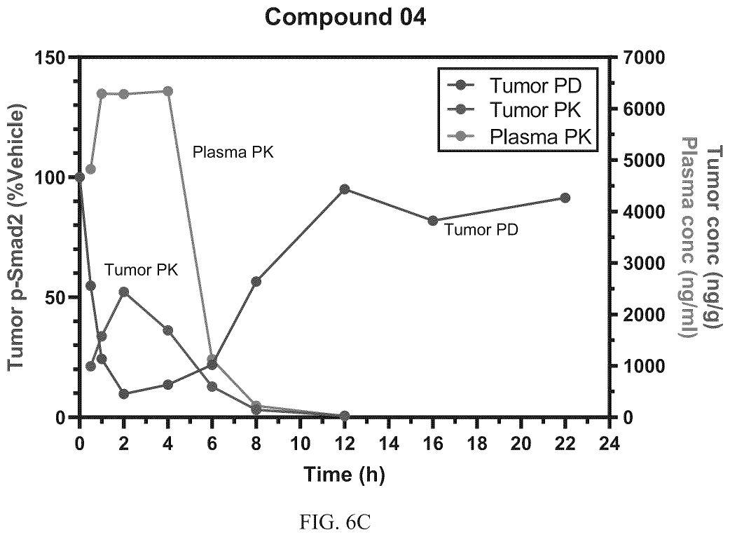

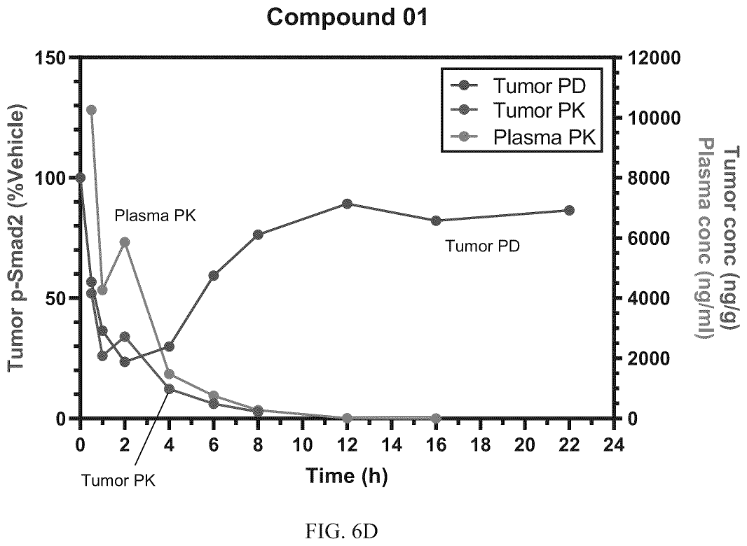

FIG. 6A shows the p-SMAD2 levels normalized to a housekeeping protein (GAPDH), represented as a percent of vehicle group treated mice. Compound 01, Compound 04 and vactosertib reduced the p-SMAD2 levels (normalized to GAPDH) after 30 minutes of drug administration to similar levels (53% of vehicle for vactosertib, 55% of vehicle for compound 04 and 57% for compound 01). However, vactosertib was not able to sustain p-SMAD2 suppression beyond one hour, and its peak suppression was reached at one hour post dosing with 14% p-SMAD2 levels (normalized to GAPDH) compared to vehicle remaining. The data in FIGS. 6A and 6B and Table 6 show that vactosertib is quickly cleared from the mice, resulting in quickly rising p-SMAD2 levels after one hour. Compound 04 reaches its peak suppression one hour later than vactosertib, at two hours post drug administration, and lower p-SMAD2 levels (normalized to GAPDH) were reached compared to vactosertib at 10% p-SMAD2 compared to the average of the vehicle group. Compound 01 also reached its peak p-SMAD2 inhibition at 2 hours post dosing, reducing the p-SMAD2 levels in the tumor tissue to 24% of vehicle.

TABLE 6
Average (averaged from 3 mice) percent p-SMAD2 levels compared to
vehicle group assayed in A549 tumor samples after indicated times
post dosing with 50 milligrams per kilogram (per oral gavage).
Each value represents the average of 3 mice per timepoint per group.
The bold values represent the peak biomarker inhibition.

| | Vactosertib | Compound 04 | Compound 01 |
|---|---|---|---|
| Time (h) | (% p-SMAD2) | (% p-SMAD2) | (% p-SMAD2) |
| 0 | 100 | 100 | 100 |
| 0.5 | 53 | 55 | 57 |
| 1 | 14 | 24 | 37 |
| 2 | 21 | 10 | 24 |
| 4 | 32 | 14 | 30 |
| 6 | 67 | 22 | 59 |
| 8 | 65 | 57 | 76 |
| 12 | 89.1 | 95.0 | 89.2 |
| 16 | 74.6 | 81.9 | 82.1 |
| 22 | 101.1 | 91.4 | 86.5 |

FIG. 6B shows the PK/PD relationship for vactosertib, FIG. 6C shows the PK/PD relationship for compound 04 and FIG. 6D shows the PK/PD relationship for compound 01. Cumulatively, the data in FIGS. 6B, 6C and 6D show that compound 04 and compound 01 exhibit a longer drug tumor and plasma exposure in vivo, resulting in prolonged suppression of the TGF beta signaling molecule p-SMAD2 over time compared to vactosertib.

### Example 11. Bleomycin-Induced Lung Fibrosis Study

The most common animal model of pulmonary fibrosis is the bleomycin-induced lung fibrosis model in rodents. This model is commonly used to investigate biology and potential therapies for fibrotic diseases affecting the lung, such as idiopathic pulmonary fibrosis (IPF). Bleomycin, a cytotoxic drug, is administered to mice or rats and causes direct cell injury triggered by DNA strand breaks, which leads to an overproduction of reactive oxygen species causing inflammation, pulmonary toxicity, activation of fibroblasts and subsequent fibrosis. Fibrosis is hallmarked by aberrant activation of lung epithelial cells and accumulation of fibroblasts and myofibroblasts with excessive production of extracellular matrix such as collagen. To test the ability of Compound 01 and Compound 04 to reduce fibrosis in vivo, a bleomycin induced lung fibrosis model in mice was utilized and endpoints such as lung hydroxyproline levels, a surrogate marker of collagen and fibrosis, and histological scoring of fibrosis were performed.

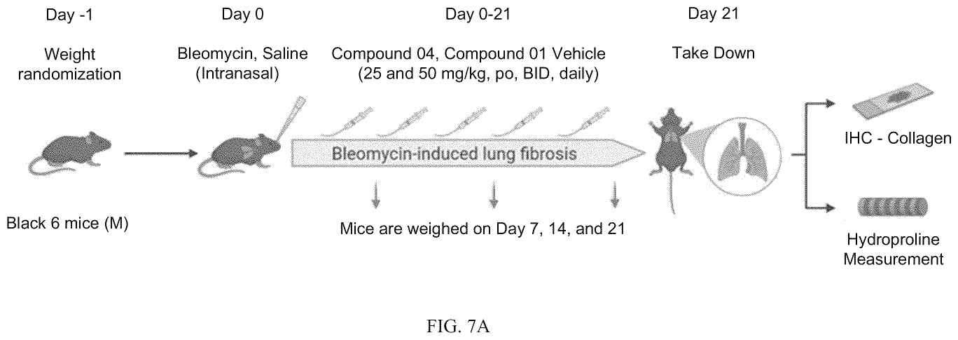

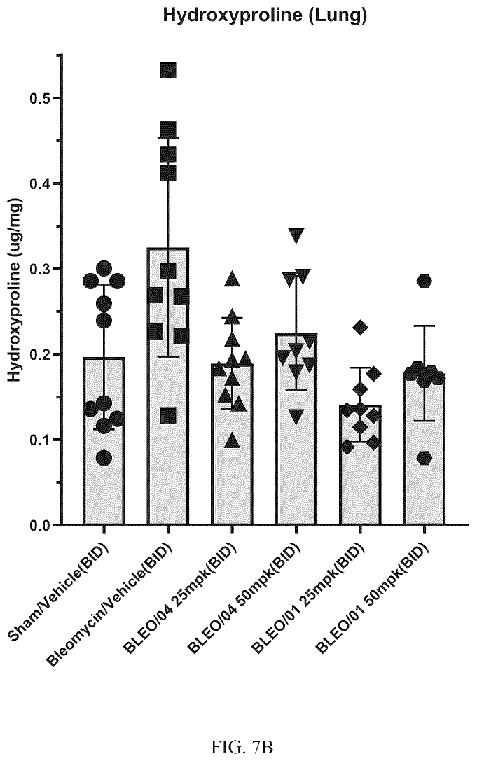

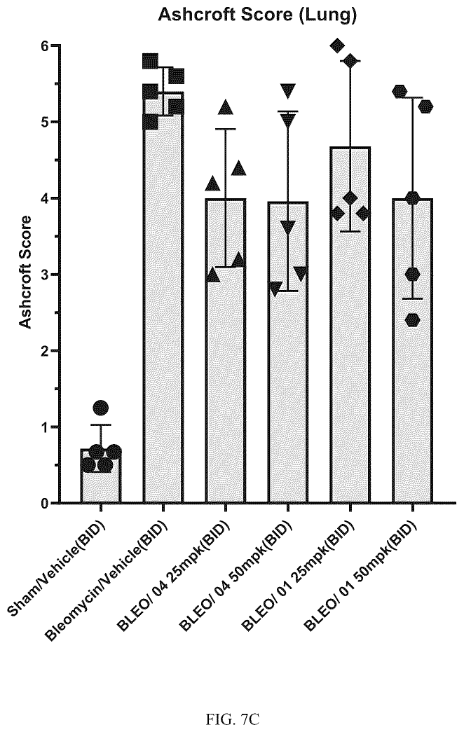

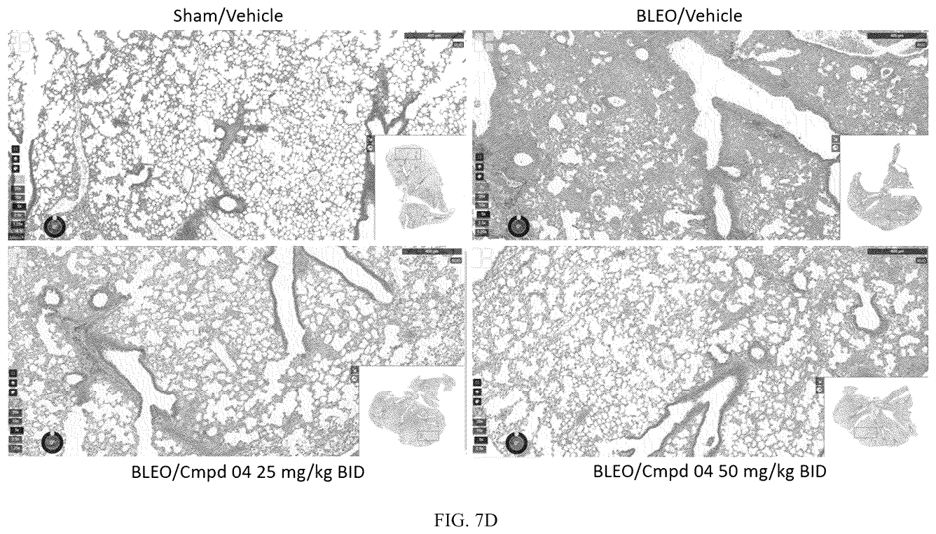

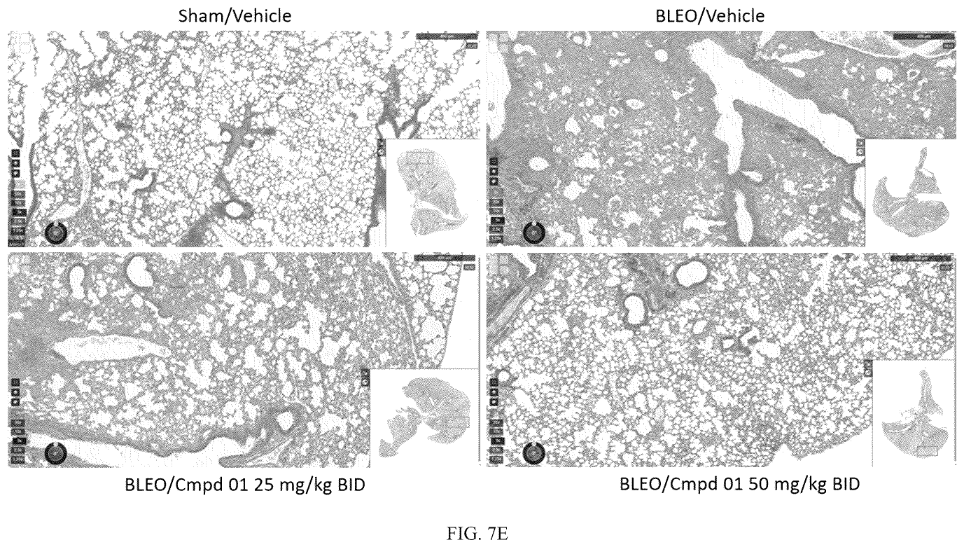

FIG. 7A shows the overall study design. For this study, male C₅₇BL/6J mice approximately 6 to 7 weeks old (purchased from Charles River) were weighed on the day prior to study day 0 to establish a baseline. All study arms contained sixteen total animals, except the sham/vehicle group, which contained 10 animals. All animals, except the sham/vehicle group were dosed intranasally with 4 international units per kilogram bleomycin in the morning of day 1 of the study. Bleomycin was prepared in a solution of 0.9 percent sodium chloride. All drugs were prepared in 10 percent Tween 20. Starting in the evening on day 1 of the study, one dose of Compound 04 or Compound 01 was given at either twenty-five or fifty milligrams per kilogram per oral gavage. For all subsequent days of the twenty-one day study, animals were dosed twice a day per oral gavage with either twenty-five or fifty milligram per kilogram of Compound 04 or Compound 01. At day twenty-one, the lungs were harvested and either snap frozen or fixed in paraformaldehyde. The left lobe of the lungs was subjected to hydroxyproline quantification, which was used as a biomarker of fibrosis and total collagen. The remaining lung tissue was fixed in paraformaldehyde to be processed for histological examination following hematoxylin-eosin (H&E) and Masson trichrome staining. The histological sections were scored using the following modified Ashcroft scale, adapted from Hübner, R-H et al., Standardized quantification of pulmonary fibrosis in histological samples, BioTechniques 2008 44: 507-17, doi: 10.2144/00011272:
Grade 0=Normal lung
Grade 1=Minimally detectable thickening of alveolar walls
Grade 2=Mild thickening of alveolar walls
Grade 3=Moderate contiguous thickening of walls with fibrous nodules
Grade 4=Thickened septae and confluent fibrotic masses totaling less than 10% of the microscopic field
Grade 5=Increased fibrosis with definite damage to lung structure and formation of fibrous bands or small fibrous masses between 10-50% of the microscopic field
Grade 6=Large contiguous fibrotic masses consolidating more than 50% of the microscopic field
Grade 7=Severe distortion of structure and large fibrous areas
Grade 8=Total fibrous obliteration of lung within the microscopic field
   The mice were weighed on days seven, fourteen, and twenty-one to monitor weight changes and mortality. Clinical observations were recorded daily.

FIG. 7B shows the amount of hydroxyproline (micrograms of hydroxyproline per milligram of lung tissue) that was measured from a portion of the lung tissue harvested from each mouse remaining on day twenty-one of the study. Using the Dunnett's multiple comparisons test, all groups show a statistically significant difference in the amount of hydroxyproline compared to the bleomycin/vehicle group except the arm receiving fifty milligrams per kilogram Compound 04 twice a day, which was not statistically significant (adjusted p-value=0.0662). The most statistically significant reduction of hydroxyproline was found in the arm of animals receiving twenty-five milligrams per kilogram of Compound 01 twice a day (adjusted p-value<0.0001). The study arm receiving twenty-five milligrams per kilogram Compound 04 and fifty milligrams per kilogram Compound 01 were calculated to be statistically significant with an adjusted p-value=0.0031 and adjusted p-value=0.0024, respectively, compared to the bleomycin/vehicle group.

FIG. 7C shows histological analysis applying the Ashcroft score of five randomly chosen animals from each treatment group. The Ashcroft score was determined by a single pathologist using the modified Ashcroft scale applied to tissue stained with Masson trichrome and H&E. The Ashcroft score is used to indicate the fibrosis severity and lung tissue structure changes due to the fibrosis. All treatment groups contained at least 2 out of the 5 total animals with a significant reduction of the Ashcroft score compared to the average Ashcroft score in the bleomycin/vehicle group. The group comparisons between study arms receiving Compound 04 and Compound 01 (all doses) compared to the bleomycin/vehicle group were not statistically significant.

FIGS. 7D and 7E show images of lung tissue stained with Masson's trichrome stain obtained from one animal from each treatment group. Each image is at 5× magnification, with the whole tissue being shown in the lower right corner, where a rectangle indicates the magnified area in relation to the entire tissue piece. Specifically, FIGS. 7D and 7E show a representative lung image from an animal treated with sham/vehicle (upper left images in FIGS. 7D and 7E, identical image), as well as a representative lung image from an animal treated with bleomycin/vehicle (upper right images in FIGS. 7D and 7E, identical image). The lower left images in FIGS. 7D and 7E are obtained from an animal that responded to Compound 04 or Compound 01, respectively, at 25 milligrams per kilogram dosed twice a day (BID). The lower right images in FIGS. 7D and 7E are obtained from an animal that responded to Compound 04 or Compound 01, respectively, at 50 milligrams per kilogram dosed twice a day (BID). The Masson's trichome stain results in a blue tissue staining when tissue contains mature collagen, a marker useful for the identification of fibrosis. As seen in the upper right images of FIGS. 7D and 7E, bleomycin/vehicle treatment resulted in a dense lung tissue with large blue patches indicating areas of severe fibrotic tissue. Treatment with Compound 04 or Compound 01 reduced the formation of fibrosis, and lung tissue obtained from animals that responded to Compound 04 or Compound 01 resembled a more healthy lung (bottom images in FIGS. 7D and 7E). These data indicate that Compounds 04 and 01 show efficacy in some animals induced to develop severe fibrosis, which could indicate the potential of these compounds to treat human conditions of fibrosis.

### Example 12. Epithelial to Mesenchymal Transition Gene Expression in A549

To evaluate whether Compounds 01 and 04 could inhibit epithelial to mesenchymal transition (EMT), an in vitro model using A549 lung fibroblasts was utilized. A549 cells were purchased from ATCC (CCL-185), and grown in RPMI media supplemented with 10 percent fetal bovine serum (FBS). On the day of the experiment, ten thousand A549 cells were seeded in eighty microliters total volume into a flat, tissue culture coated, ninety-six-well plate. Drugs, including positive control compounds vactosertib, PF-06952229 and LY3200882, were added in an eight point, one to three dilution series with the highest concentration of each drug being five micromolar, bringing the total volume of each well to ninety microliters. DMSO, which served to dissolve the 10 millimolar stock solution of each drug, was normalized across all wells After one hour of drug pretreatment, recombinant human TGF-beta1 was added to a final concentration of five nanograms per milliliter, bringing the volume to one hundred microliters. Recombinant human TGF-beta 1 was purchased from R&D Systems (7754-BH-005) and its stock solution was prepared according to manufacturer's instructions and diluted in RPMI media supplemented with 10% FBS. The ninety-six well plate with one hundred microliter volume was incubated at 37° C. at 5% CO₂ for forty-eight hours. At the end of the incubation period, gene expression levels of various EMT markers were assessed using a custom designed QuantiGene Plex Gene Expression Assay. The genes chosen were CDH1 (E-cadherin), CDH2 (N-Cadherin), SNAI1 (Snail), SNAI2 (Slug), VIM (Vimentin), SPARC, GALNT6, CTNNB1 (beta-catenin), TGFB1, and MAML3.

Briefly, cell lysate was prepared using the QuantiGene Sample Processing Kit —Cultured Cells (QS0100) in the ninety-six well plate. Cell lysates were then subjected to bead hybridization, multiple probe hybridization steps and SAPE labeling following manufacturer's instruction. The plate was read on a Luminex MAGPIX instrument. All of the genes chosen were expected to have increased expression upon induction of EMT by TGF-beta 1 stimulation, except CDH1 (E-cadherin), which is known to be downregulated as cell transition to a mesenchymal phenotype and cell adhesion is broken down

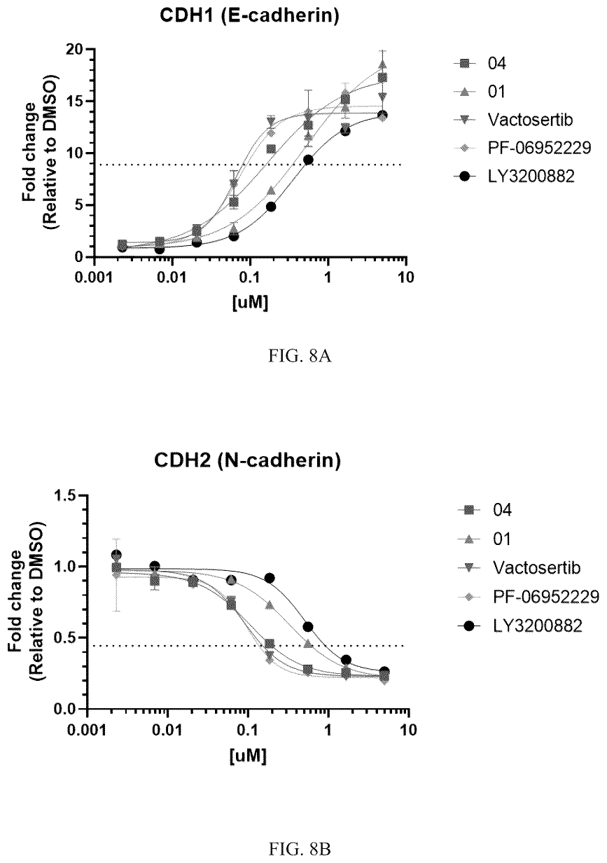

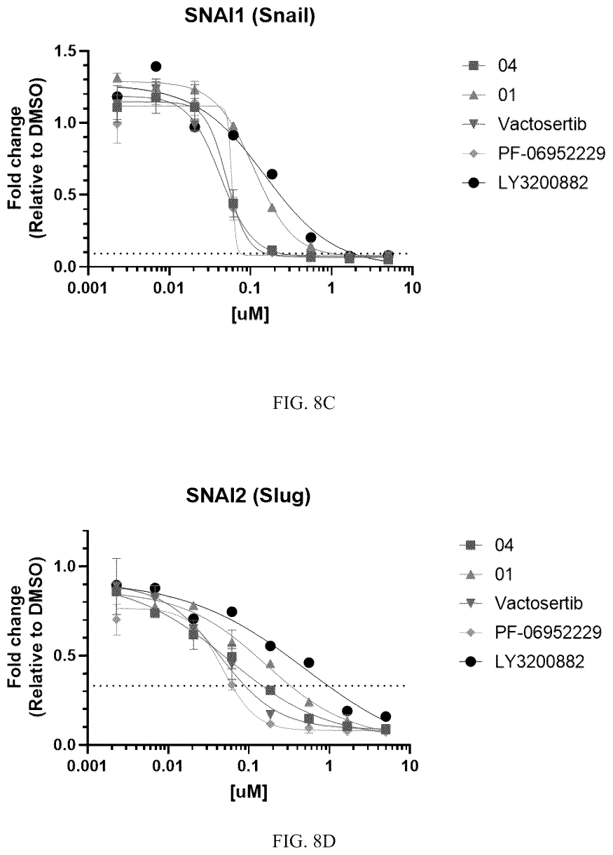

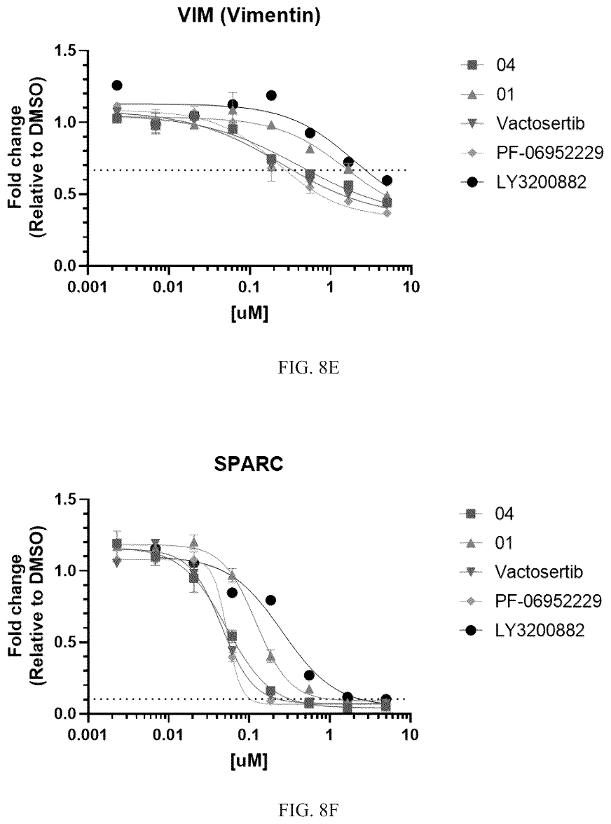

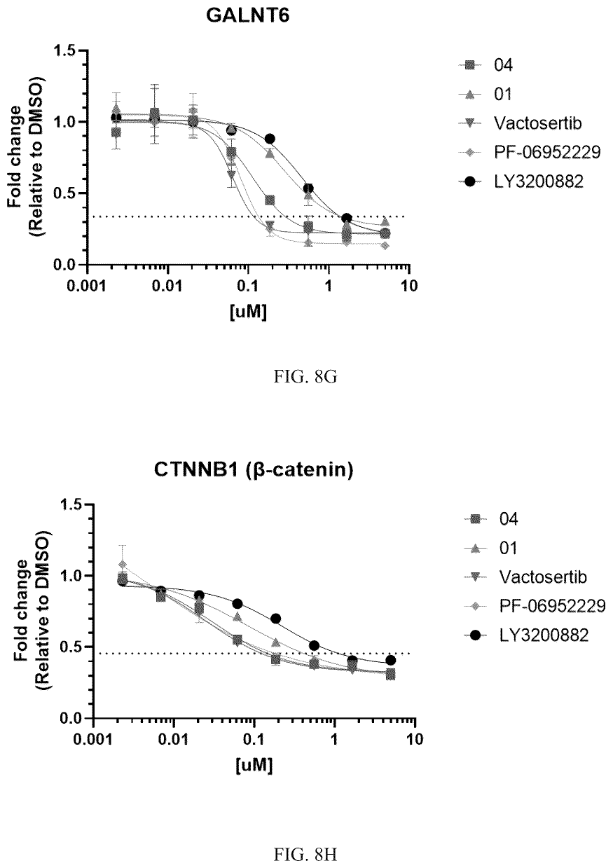

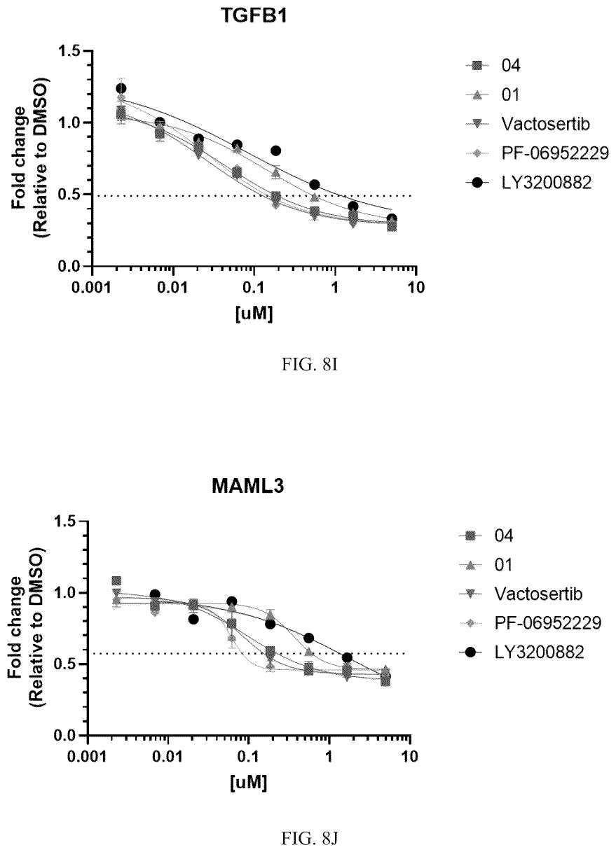

FIG. 8A shows inhibition of downregulation of CDH1 (E-Cadherin) by the positive control TGF-beta signaling inhibitors vactosertib, PF-06952229 and LY3200882, as well as Compound 01 and Compound 04. FIGS. 8B-8J show inhibition of induction of the nine genes that are induced with EMT (namely, CDH2 (N-Cadherin), SNAI1 (Snail), SNAI2 (Slug), VIM (Vimentin), SPARC, GALNT6, CTNNB1 (beta-catenin), TGFB1, and MAML3EMT) by the positive control TGF-beta signaling inhibitors vactosertib, PF-06952229 and LY3200882, as well as Compound 01 and Compound 04 As these figures indicate, the addition of Compound 01 or Compound 04 inhibited signaling triggered by TGF-beta and, as a result, blocks EMT transition in A549 cells in vitro similarly to the TGFBR1/ALK5 inhibitors vactosertib. PF-06952229 and LY3200882 employed as references in this example.

The information presented in the figures notes above indicates that Compounds 01 and 04 could potentially treat diseases where EMT contributes to disease progression, as well as complications, such as metastasis in cancer and cancer-associated tissue fibrosis, and other fibrotic diseases such as idiopathic pulmonary fibrosis.

### Example 13. Maximum Tolerated Dosage (MTD) Study

Determining the MTD is critical to calculate the therapeutic index (also referred to as therapeutic ratio), which is the ratio of the MTD to the dose required to move the biomarker or show efficacy in the same species. Evaluation of the acute and chronic maximum tolerated dose (MTD) for Compound 04 and Compound 01 was carried out by oral gavage administration of each compound to athymic nude mice according to the following procedure.

Five weeks-old female athymic nude mice (NCRNU) were purchased from Taconic Biosciences, Rensselaer NY, and acclimated for one week. Mice were approximately four months old when enrolled in the study. All compounds were resuspended in 1-methyl-2-pyrrolidinone (Sigma, 494496) (10% v/v) plus 20% Solutol (Sigma 42966) in water (90% v/v). Mice were randomized according to the baseline weight (Day 1) at the beginning of the acute and chronic MTD studies.

For the acute MTD study, mice were administered three escalating doses of each compound every other day (two animals per group). Mice were dosed by oral gavage with one hundred milligrams per kilogram on day one, five hundred milligrams per kilogram on day three, and one thousand milligrams per kilogram on day five. Body weight was measured every day, and the percent body weight change was calculated.

For the chronic MTD study, three groups, with three mice per group were dosed daily for five consecutive days, at one hundred, three hundred, or one thousand milligrams per kilogram of Compound 04 or Compound 01, and observed for five consecutive days post-dosing during recovery. Body weight was measured, and clinical observations were recorded daily for ten consecutive days. A vehicle only treatment group of three mice was included to evaluate potential toxicity of the vehicle. The percent body weight change for each day was calculated based on Day 1 measurements.

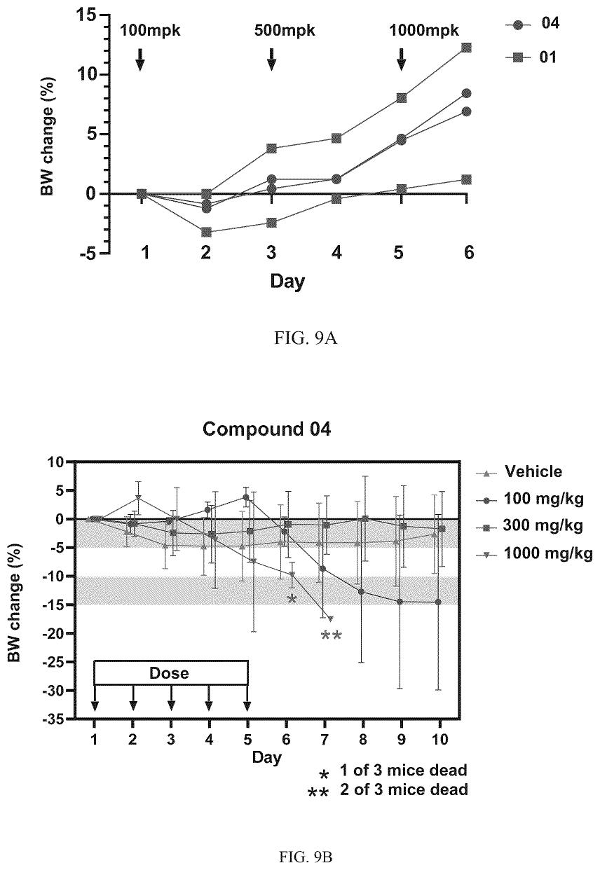

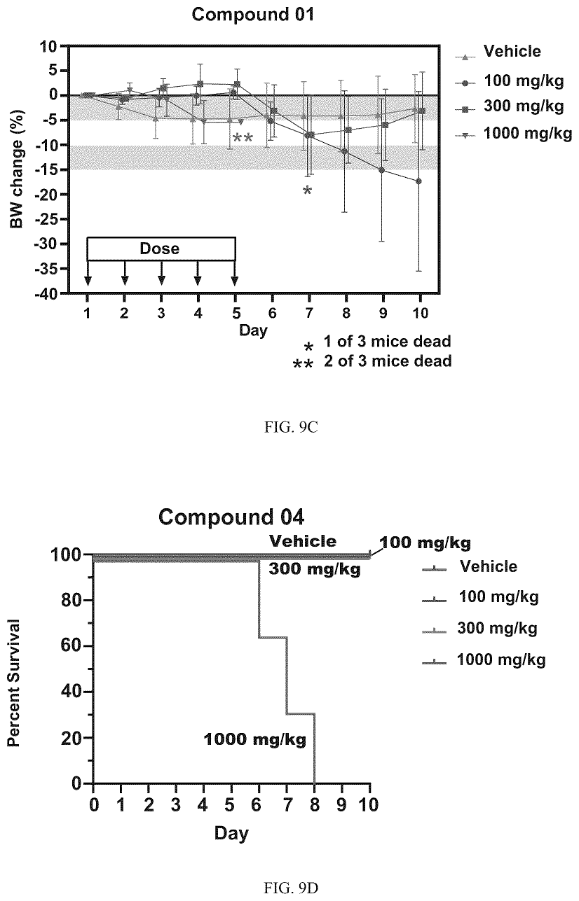

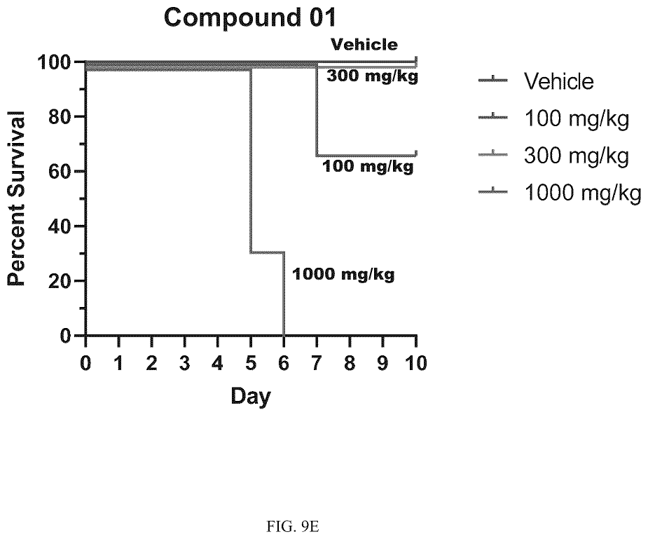

FIG. 9A shows the percent body weight change observed in the acute MTD study. Each line represents one animal dosed with either Compound 04 or Compound 01. Animals showed slight weight loss on Day 2 in both drug treatment groups after the first dose of one hundred milligrams per kilogram. However, all animals from both drug treatment groups recovered body weight throughout the course of the study, even after administration of higher drug doses. These data indicate that both compounds are well tolerated in mice at one thousand milligrams per kilogram with single dose administration.

FIGS. 9B and 9C show the percent body weight change observed in the chronic MTD study using Compound 04 (FIG. 9B) or Compound 01 (FIG. 9C). The data are presented as mean plus or minus standard deviation of the three animals in each treatment group. As these data show, Compound 04 is well tolerated at three hundred milligrams per kilogram. However, two of three animals in the one hundred milligrams per kilogram group lost more than fifteen percent body weight when treated with one hundred milligrams per kilogram of Compound 04. At one thousand milligrams per kilogram, the mice treated with Compound 04 showed signs of toxicity. At a dosage of one thousand milligrams per kilogram, all three mice died during the course of the study.

From these data, the MTD for Compound 04 was determined to be three hundred milligrams per kilogram. Based on an efficacious dose of fifty milligrams per kilogram, derived from studying p-SMAD2 inhibition in the A549 xenograft mouse model (described above), the therapeutic index of Compound 04 was estimated to be 6×(three hundred milligrams per kilogram MTD/fifty milligrams per kilogram PD efficacy).

Turning to the MTD study using Compound 01, when Compound 01 was administered to mice at one hundred milligrams per kilogram, the mice exhibited some clinical signs of distress, showing hunched posture, and one animal died on day seven and one animal exhibited more than thirty percent body weight loss. At three hundred milligrams per kilogram, however, the mice administered Compound 01 showed only mild body weight loss, with two of the three animals losing ten to fifteen percent body weight by day seven, then recovering more than 90 percent of their original body by day ten. At one thousand milligrams per kilogram, Compound 01 was not tolerated: all three of the subjects died during the course of the study due to toxicity.

From these data, the MTD for Compound 01 was determined to be less than three hundred milligrams per kilogram. Based on the efficacious dose of seventy-five milligrams per kilogram derived from studying p-SMAD2 inhibition in the A549 xenograft mouse model (described above), the therapeutic index of Compound 01 was estimated to be less than 4×(three hundred milligrams per kilogram MTD/seventy-five milligrams per kilogram PD efficacy). FIGS. 9D and 9E show the overall mortality associated with the different treatment arms of the chronic MTD study involving Compound 04 and Compound 01, respectively, indicating both compounds surpassed their MTD at one thousand milligrams per kilogram when dosed for five consecutive days.

### Example 14. Phospho-SMAD2 Assay

TGF-β is a pleiotropic cytokine involved in extremely conserved pathways relevant to cell growth, differentiation, and development. TGF-β signaling is triggered when the activated TGF-β homodimer binds to the TGF-β receptor 2 (TGFbR2), which in turn leads to the recruitment and phosphorylation of TGF-β receptor 1 (TGFbR1). Activated TGFbR1 phosphorylates the signal transduction molecules SMAD2 and SMAD3, which then bind to the common mediator SMAD4 and translocate to the nucleus where they alter gene transcription. In this assay, phospho-SMAD2 (S465/S467), a direct phosphorylation substrate of TGFbR1, was used as a readout of TGF-β pathway activity. Vactosertib (a TGFbR1 inhibitor available from MedChem Express HY-19928) was included as a positive control.

The RD SMAD reporter (RDSR) cell line was generated by stably integrating the SMAD reporter plasmid (Promega, pGL4.48[luc2P/SBE/Hygro]) into the human rhabdomyosarcoma cell line RD (ATCC, CCL-136).

RDSR cells were seeded in DMEM (ATCC30-2002)+10% FBS in 96-well plate and allowed to grow overnight at 5% CO₂ and 37° C. The next day, cells were pre-treated with Compound 04, Compound 01 or vactosertib at various concentrations, or with vehicle (DMSO), for one hour. Cells were then stimulated with recombinant human TGF-β1 (R&D Systems 7754-BH-005) at a final concentration of 100 ng/ml, in the presence of Compound 04, Compound 01, vactosertib, or DMSO for thirty minutes. A no-TGF-β1 stimulation condition was included for baseline phospho-SMAD2 level measurement.

The phospho-SMAD2 levels were determined using the Bio-Plex Pro™ Phospho-Smad2 (Ser465/Ser467) bead/antibody set (BioRad 171V50019M). Phospho-SMAD2 levels were normalized to ß-tubulin (MILLIPLEX MAP ß-Tubulin Total Magnetic Bead MAPmate™, Millipore 46-713MAG) or GAPDH (MILLIPLEX MAP GAPDH Total Magnetic Bead MAPmate™, Millipore 46-667MAG) levels from each sample. All samples were analyzed in a multiplex fashion with the Bio-Plex Pro Cell Signaling Reagent Kit (BioRad 171304006M). Briefly, at the end of treatment, RDSR cells were rinsed with ice-cold phosphate buffered saline (PBS) and lysed in fifty-five microliters of lysis buffer for thirty minutes. The resulting lysate was used for the assay according to the manufacturer's instructions. Bead suspension was analyzed using the Luminex system (MAGPIX).

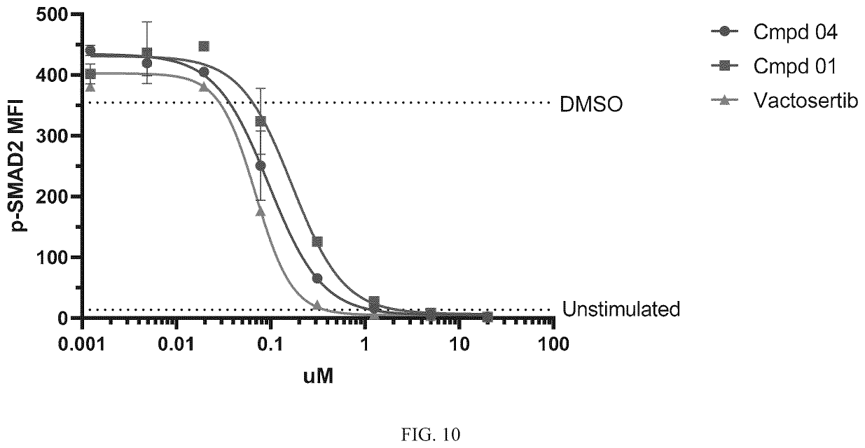

Compound 04, Compound 01 and vactosertib were tested at drug concentrations of 20 micromolar to 1.22 nanomolar using a 4-fold serial dilution. All wells contained the same DMSO concentrations. IC50 values were calculated using nonlinear regression model with GraphPad Prism software, with all compounds showing values less than 200 nM. Graphic presentation of the longitudinal results from this assay of phospho-SMAD2 are shown in FIG. 10.

### Example 15. JAK Selectivity Assay

Janus kinase (JAK) signaling inhibitors, even though approved by the FDA, have been shown to cause anemia and lymphopenia, and to suppress the immune system in some people. These properties would be undesirable in an ALK5 inhibitor to be used in non-oncology indications with repeat/chronic dosing. To demonstrate the extent of JAK signaling inhibition of Compounds 01 and 04, an in vitro cellular assay was utilized, and IC50 values calculated and compared to various JAK inhibitor molecules.

To evaluate the potency of Compound 04 and Compound 01 at inhibiting JAK signaling, a HEK blue IL-12 reporter assay was utilized. The cells were purchased from Invivogen (Catalog Nr: hkb-il12) and maintained according to vendor instructions. On the day of the experiment, cells were harvested by repeated tapping of the flask to shake loose the cells attached to the bottom of the flask. Then, cells were harvested, washed and resuspended in DMEM media supplemented with 10% fetal calf serum. 30 Microliters of cell suspension was seeded into a 384-well tissue culture-coated plate containing approximately 10,000 total cells. Compound 01, Compound 04 or a tool compound was added using the ECHO acoustic dispenser at a top concentration of 20 micromolar. Using the ECHO “Dose Response” software, the test compound was added in duplicate wells with a 3-fold dilution curve. DMSO was back-filled to normalize the DMSO across all wells. After one hour of incubation in the incubator at 37° C., the plate was removed from the incubator, and 7.5 nl of human recombinant IL-12 (50 ug/ml) was delivered with the ECHO to all wells (12.5 ng/ml final concentration of IL-12). The recombinant human IL-12 was purchased from R&D Systems (Catalog Nr: 219-IL-005). After 24 hours of incubation at 37 degrees Celsius in the incubator, the plates were removed, and 5 μl of supernatant was removed and mixed with 45 μl of Quanti-blue solution (purchased from Invivogen, rep-qbs) in a clear-bottomed, black, 384-well plate. After 30 minutes of incubation at room temperature, the optical density was measured at 620 nanometer using the Perkin Elmer Envision plate reader. A color change can be detected at this wavelength when secreted alkaline phosphatase, which is triggered by IL-12 signaling, is present in the supernatant. A lack of signal indicates that the test compound inhibited this reporter system. Table 8 lists the tool compounds included as positive controls in the assay.

TABLE 8
Tool compounds

| | | JAK Inhibition |
|---|---|---|
| Name of | Vendor/Purchase | Profile, as disclosed |
| Compound | Information | by the vendor |
| Tofacitinib | SelleckChem, | potent JAK3 |
| (CP-690550) | Catalog | inhibitor, moderate |
| | Nr: S5001 | JAK2 inhibitor |
| Baricitinib | SelleckChem, | Potent JAK 1 and |
| (INCB028050) | Catalog | JAK2 inhibitor, poor |
| | Nr: S2851 | activity against |
| | | JAK3 |
| Fedratinib | SelleckChem, | Potent JAK2 |
| (TG101348) | Catalog | inhibitor, poor |
| | Nr: S2736 | activity against |
| | | JAK1 and JAK3 |
| Ruxolitinib | SelleckChem, | potent JAK1/2 |
| (INCB018424/Jakafi) | Catalog | inhibitor (does |
| | Nr: S1378 | not inhibit JAK3) |
| Vactosertib | MedChemExpress, | Potent ALK5 |
| | Catalog | inhibitor |
| | Nr: HY-19928 | |

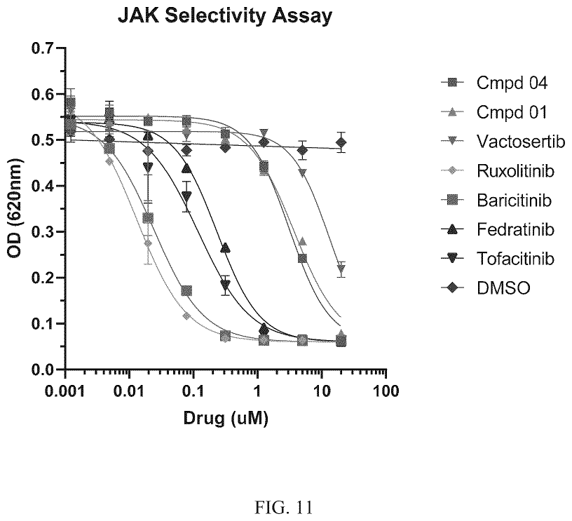

The JAK tool compounds ruxolitinib, baricitinib, fedratinib and tofacitinib showed strong potency in this assay, and the IC50 values were calculated to be under 300 nanomolar for all JAK inhibitors tested. Ruxolitinib, which is described to be the most potent JAK2 inhibitor, was also the most potent in this assay, with an IC50 value of 31 nanomolar. Compound 04 (3.7 micromolar IC50) and Compound 01 (3.8 micromolar IC50) showed weak inhibition in this assay, suggesting that the JAK signaling potency of these compounds is minimal. The ALK5 tool compound vactosertib also showed weak off target activity of 18.5 micromolar IC50. The results of this assay are presented graphically in FIG. 11. As shown in FIG. 11, Compounds 01 and 04 show similar profiles to vactosertib with regard to off-target JAK2 inhibition, distinguished from the various JAK2 inhibitors used in the study for reference.

### Example 16. In Vitro 3D Liver Fibrosis Assay

An additional fibrosis assay was carried out with selected compounds using the Visikol OpenLiver™ HepaRG™ NP 3D model directed at fibrosis. The OpenLiver™ HepaRG™ NP 3D model features the ability to recapitulate a number of liver disease pathologies. Whether evaluating the disease-inducing liability of a compound or exploring targets to exploit for therapeutic amelioration of disease state, the HepaRG™ NP 3D model is capable of recapitulating disease features such hepatocellular lipid accumulation, nonparenchymal cell phenotype shift, and collagen matrix deposition.

HepaRGs (Lonza, NSHPRG, Lot #HNS1013) and non-parenchymal (NP) cells (Stellates, Kupffers and LSECS (Lonza, Lot HUM201221)) were thawed, assessed for viability and seeded at a hepatocyte to NP cell ratio of 60:40 into an ultra-low attachment round-bottom plate (Corning, 4515). The cells were initially cultured in William's E medium supplemented with HepaRG Thaw, Plate & General Purpose Supplement (Thermo Fisher, HPRG770) and GlutaMax (Thermo Fisher, 35050061) overnight. The next day the medium was half exchanged with William's E medium supplemented with HepaRG Maintenance Supplement (Thermo Fisher, HPRG720) and GlutaMax (Thermo Fisher, 35050061). Every 2-3 days the medium was half-exchanged with fresh maintenance medium until spheroid formation, approximately seven days post-seeding. The spheroids were then cultured for an additional seven days, at which point the medium was exchanged for medium containing the treatment of interest. All cell culture occurred in a 37° C./5% CO₂ incubator.

On the day of treatment, each well was pre-treated for one hour with 1 μM vactosertib, Compound 04, or Compound 01, or DMSO alone (0.05%). TGF-β1 (PeproTech, 100-21) was reconstituted in sterile 4 mM HCl per manufacturer's guidelines to a stock concentration of 20 μg/mL. The stock was further diluted in maintenance medium to a final concentration of 100 ng/mL. Following pre-treatment, each well was treated with either 100 ng/mL of TGF-β1, or the vehicle control containing 0.5% 4 mM HCl. Spheroids were cultured for 72 hours after test compound addition.

At the 72-hour time point, spheroids, marked for labeling, were treated with a viability dye (ThermoFisher, 65-0865-14) to determine viability, fixed in neutral buffered formalin (Fisher, SF100-20), washed three times in PBS, and then stored in PBS until labeling.

Spheroids underwent simultaneous permeabilization and blocking using goat serum diluted in PBS containing Triton X100. Spheroids were then labeled using antibodies against pan-collagen. Secondary antibodies were then used to fluorescently indicate primary labeling and DAPI was added as a nuclear counterstain. All labeling was performed in the same solution used for permeabilization and blocking. Following labeling and a light dehydration in methanol, spheroids were cleared with Visikol HISTO-M™ prior to imaging. Materials used for this assay are listed in Table 10.

TABLE 10

| Reagent | Vendor | Catalog # |
|---|---|---|
| eBioscience Fixable Viability | Thermo Fisher | 65-0865-14 |
| Dye eFluor ™ 780 | | |
| Collagen Pan Polyclonal Antibody | Invitrogen | PA1-36058 |
| Goat α-rabbit AlexaFluor 488 | Invitrogen | A32731 |
| DAPI | Thermo Fisher | D3571 |
| Goat Serum | Gibco | 16210064 |
| Triton ™ X-100 | Fisher Scientific | BP151-500 |
| Visikol ® HISTO-M ™ | Visikol | HM-30 |

Labeled spheroids were imaged at 10× with 10 μm z-steps using a Molecular Devices ImageExpress Micro Confocal High-Content Imaging System. Images were acquired and saved as 16-bit 2048×2048 TIF files for further processing.

Clearing the spheroid with Visikol HISTO-M™ allows for clear images throughout the spheroid volume, therefore allowing the entire z-stack to be analyzed. The DAPI channel was utilized to determine the spheroid area of each z-slice in order to calculate spheroid volume. For pan-collagen, manual thresholding was conducted to select bright regions of collagen+ staining across the z-stacks. Thresholded areas, or integrated densities from each slice were summed and multiplied by the z-step (10 μm) to give the total volume (for pan-collagen) within the spheroid. Results for pan-collagen are reported normalized to spheroid volume.

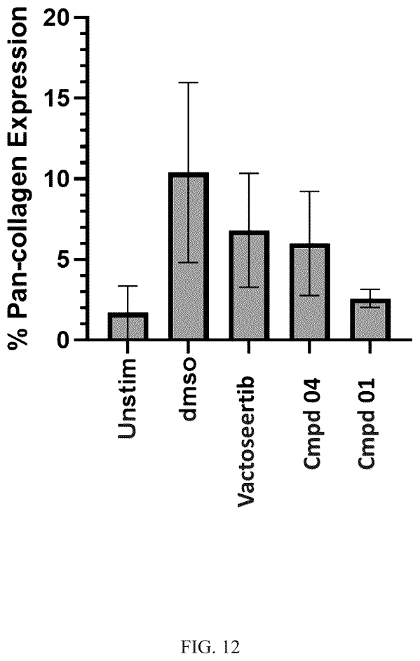

TGF-β induced pan-collagen expression as expected. Vactosertib, Compound 04, and Compound 01 inhibited TGF-β-induced pan-collagen expression to various levels, with Compound 01 being the most potent (FIG. 12). The results of this assay suggest that the tested compounds would be useful in providing treatment of fibrotic diseases.

While example embodiments have been particularly shown and described, it will be understood by those skilled in the art that various changes in form and details may be made therein without departing from the scope of the embodiments encompassed by the appended claims.

### EQUIVALENTS AND SCOPE

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or all of the group members are present in, employed in, or otherwise relevant to a given product or process.

Furthermore, the disclosure encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the disclosure, or aspects of the disclosure, is/are referred to as comprising particular elements and/or features, certain embodiments of the disclosure or aspects of the disclosure consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the disclosure, to the tenth of the unit of the lower limit of the range, unless the context clearly dictates otherwise.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

What is claimed is:

1. A compound of Formula (I): 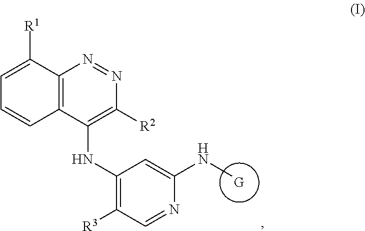
or a pharmaceutically acceptable salt thereof, wherein:
R¹ is a C₁-C₅ alkyl, C₃-C₅ carbocycle, or halogen;
R² is —H, a halogen, a C₁-C₃ alkyl optionally substituted with one or more —F, or a cyclopropyl optionally substituted with one or more —F;
R³ is —H, a halogen, a C₁-C₃ alkyl optionally substituted with one or more —F, or a cyclopropyl optionally substituted with one or more —F; and
Ring G is 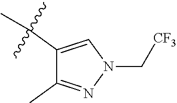 [SMILES: *c1cn(CC(F)(F)F)nc1C]
wherein 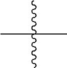
indicates the point of attachment of Ring G to the —N(H)—; or
Ring G is a C₆-C₁₀ aryl optionally substituted with:
(i) one or more halogens;
(ii) a sulfonamide;
(iii) a monocyclic, bicyclic or spirocyclic C₃-C₁₀ carbocycle which is optionally substituted with one or more C₁-C₆ alkyl or C₃-C₆ carbocycle which are optionally substituted with hydroxy or one or more halogen, wherein said carbocycle is attached to Ring G by a single bond or a methylene or ethylene linker at a position on Ring G which is meta- or para- to the —N(H)— attached to Ring G; or
(iv) a monocyclic, bicyclic, bridged or spirocyclic C₃-C₁₀ heterocycle which may contain up to 3 heteroatoms independently selected from N and O and which is optionally and independently substituted with one or more C₁-C₆ alkyl or C₃-C₆ carbocycle which are optionally substituted with hydroxy or one or more halogen, wherein said heterocycle is attached to Ring G by a single bond or a methylene or ethylene linker at a position on Ring G which is meta- or para- to the —N(H)— attached to Ring G.

2. The compound of claim 1, wherein R¹ is a C₁-C₅ alkyl or C₃-C₅ carbocycle.

3. The compound of claim 1, wherein R¹ is a halogen.

4. The compound of claim 1, wherein R¹ is methyl, cyclopropyl or chloro.

5. The compound of any one of claims 1-4, wherein R² is —H, a halogen, —CH₃, —CF₃ or cyclopropyl.

6. The compound of claim 5, wherein R² is —H.

7. The compound of any one of claims 1-6, wherein R³ is —H, a halogen, —CH₃, —CF₃ or cyclopropyl.

8. The compound of claim 7, wherein R³ is —H.

9. The compound of any one of claims 1-8, wherein Ring G is

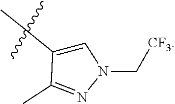 [SMILES: *c1cn(CC(F)(F)F)nc1C]

10. The compound of any one of claims 1-8, wherein Ring G is a C₆-C₁₀ aryl substituted with:
(i) one or more halogens;
(ii) a sulfonamide;
(iii) a monocyclic, bicyclic or spirocyclic C₃-C₁₀ carbocycle which is optionally substituted with one or more C₁-C₆ alkyl or C₃-C₆ carbocycle which are optionally substituted with hydroxy or one or more halogen, wherein said carbocycle is attached to Ring G by a single bond or a methylene or ethylene linker at a position on Ring G which is meta- or para- to the —N(H)— attached to Ring G; or
(iv) a monocyclic, bicyclic, bridged or spirocyclic C₃-C₁₀ heterocycle which may contain up to 3 heteroatoms independently selected from N and O and which is optionally and independently substituted with one or more C₁-C₆ alkyl or C₃-C₆ carbocycle which are optionally substituted with hydroxy or one or more halogen, wherein said heterocycle is attached to Ring G by a single bond or a methylene or ethylene linker at a position on Ring G which is meta- or para- to the —N(H)— attached to Ring G.

11. The compound of claim 10, wherein Ring G is substituted with:
i) one or more halogens;
ii) a sulfonamide;
iii) a monocyclic, bicyclic or spirocyclic C₃-C₁₀ carbocycle which is optionally substituted with one or more C₁-C₆ alkyl or C₃-C₆ carbocycle which are optionally substituted with hydroxy or one or more halogen, wherein said carbocycle is attached to Ring G by a single bond or a methylene or ethylene linker at a position on Ring G which is meta- or para- to the —N(H)— attached to Ring G;
iv) a monocyclic C₃-C₇ carbocycle which is optionally substituted with one or more C₁-C₆ alkyl or C₃-C₆ carbocycle which are optionally substituted with hydroxy or one or more halogen; or
v) a cyclohexyl which is optionally substituted with one or more C₁-C₆ alkyl or C₃-C₆ carbocycle which are optionally substituted with hydroxy or one or more halogen.

12. The compound of claim 10 or 11, wherein the carbocycle that is attached to Ring G is unsubstituted.

13. The compound of claim 10, wherein Ring G is substituted with a monocyclic, bicyclic, bridged or spirocyclic C₃-C₁₀ heterocycle which may contain up to 3 heteroatoms independently selected from N and O and which is optionally and independently substituted with one or more C₁-C₆ alkyl or C₃-C₆ carbocycle which are optionally substituted with hydroxy or one or more halogen, wherein said heterocycle is attached to Ring G by a single bond or a methylene or ethylene linker at a position on Ring G which is meta- or para- to the —N(H)— attached to Ring G.

14. The compound of claim 13, wherein Ring G is substituted with a monocyclic C₅-C₆ heterocycle which may contain up to 3 heteroatoms independently selected from N and O and which is optionally and independently substituted with one or more C₁-C₆ alkyl or C₃-C₆ carbocycle which are optionally substituted with hydroxy or one or more halogen.

15. The compound of claim 14, wherein Ring G is substituted with a piperazinyl, morpholinyl, piperidinyl or oxanyl, which is optionally and independently substituted with one or more C₁-C₆ alkyl or C₃-C₆ carbocycle which are optionally substituted with hydroxy or one or more halogen.

16. The compound of any one of claims 13-15, wherein the heterocycle that is attached to Ring G is unsubstituted or monosubstituted.

17. The compound of claim 16, wherein the heterocycle that is attached to Ring G is unsubstituted.

18. The compound of any one of claims 10-17, wherein the carbocycle or heterocycle that is attached to Ring G is optionally and independently substituted with methyl, CF₃CH₂— or HOCH₂CH₂—.

19. The compound of any one of claims 10-18, wherein the carbocycle or heterocycle attached to Ring G is attached to Ring G at a position on Ring G which is meta- to the —N(H)— attached to Ring G.

20. The compound of any one of claims 10-18, wherein the carbocycle or heterocycle attached to Ring G is attached to Ring G at a position on Ring G which is para- to the —N(H)— attached to Ring G.

21. The compound of any one of claims 1-20, wherein the C₆-C₁₀ aryl of Ring G is phenyl.

22. The compound of any one of claims 1-4, having the following structure: 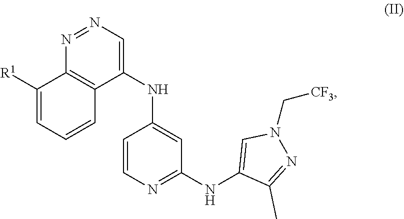
or a pharmaceutically acceptable salt thereof.

23. The compound of any one of claims 1-4, having the following structure: 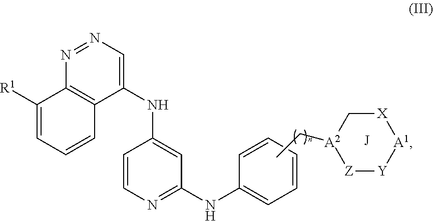
or a pharmaceutically acceptable salt thereof, wherein:
Ring J is attached to the phenylene at a position which is meta- or para- to the —N(H)— attached to the phenylene;
A¹ is —N(R⁴)—, —O— or >C(H)(R⁴);
R⁴ is —H, or a C₁-C₆ alkyl or C₃-C₆ carbocycle, each of which is optionally substituted with hydroxy or one or more halogen;
A² is >N— or >C(H)—;
Z is >CH₂; and X and Y are independently >CH₂ or >C(CH₃)₂, or X and Y are both >CH— and are bonded together through a methylene or ethylene bridge; or
Y is >CH₂ or >C(CH₃)₂, and X and Z are both >CH— and are bonded together through a methylene or ethylene bridge; and
n is 0, 1 or 2.

24. The compound of claim 23, wherein A¹ is >C(H)(R⁴).

25. The compound of claim 23, wherein A¹ is —N(R⁴)— or —O—.

26. The compound of any one of claims 23-25, wherein R⁴ is —H, or a C₁-C₆ alkyl, which is optionally substituted with hydroxy or one or more halogen.

27. The compound of claim 26, wherein R⁴ is —H, methyl, hydroxyethyl or trifluoroethyl.

28. The compound of any one of claims 23-27, wherein A² is >C(H)—.

29. The compound of any one of claims 23-28, wherein A² is >N—.

30. The compound of claim 23, wherein Ring J is:

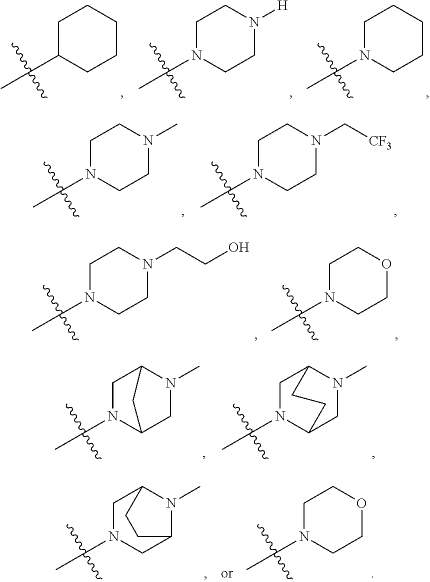

31. The compound of any one of claims 23-30, wherein Ring J is attached to the phenylene at a position which is meta- to the —N(H)— attached to the phenylene.

32. The compound of any one of claims 23-31, wherein n is 0 or 1.

33. The compound of claim 32, wherein n is 0.

34. The compound of any one of claims 1-4, 23-29 and 31, having the following structure: 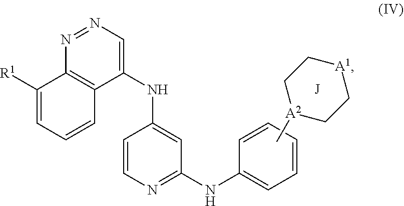
or a pharmaceutically acceptable salt thereof.

35. The compound of claim 34, wherein Ring J is:

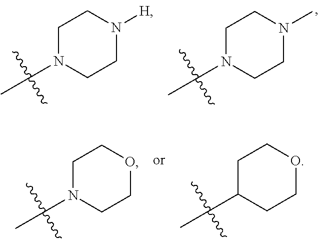

36. A compound, or a pharmaceutically acceptable salt thereof, having the structure:

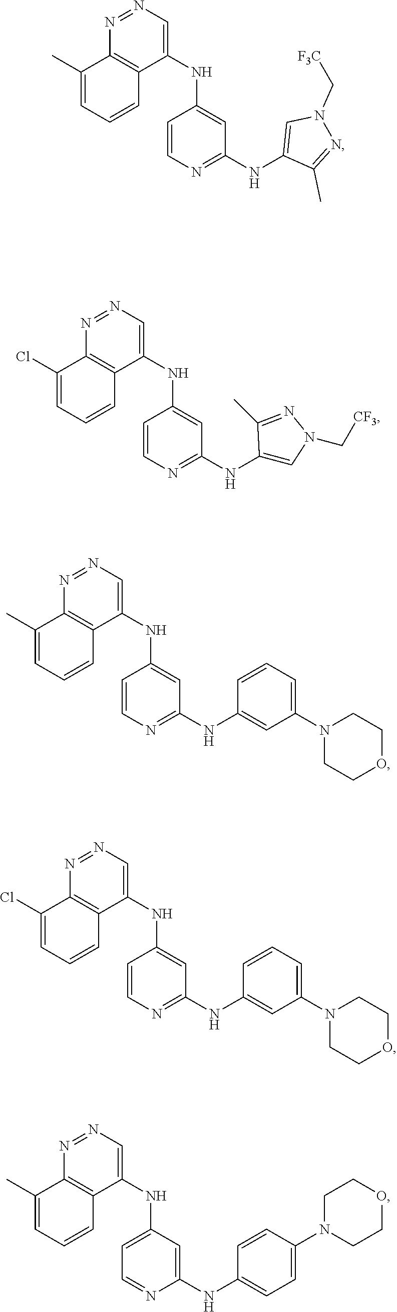 [SMILES: Cc1nn(CC(F)(F)F)cc1Nc1cc(Nc2cnnc3c(C)cccc23)ccn1 ; Cc1nn(CC(F)(F)F)cc1Nc1cc(Nc2cnnc3c(Cl)cccc23)ccn1 ; Cc1cccc2c(Nc3ccnc(Nc4cccc(N5CCOCC5)c4)c3)cnnc12 ; Clc1cccc2c(Nc3ccnc(Nc4cccc(N5CCOCC5)c4)c3)cnnc12 ; Cc1cccc2c(Nc3ccnc(Nc4ccc(N5CCOCC5)cc4)c3)cnnc12]

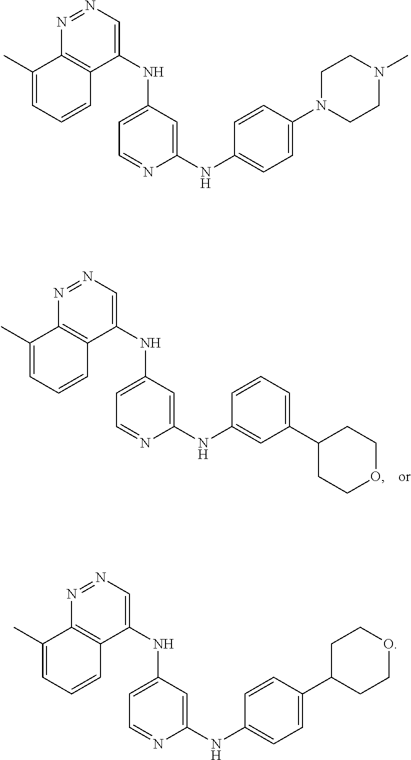

37. The compound of claim 36, wherein the compound is of the following formula: 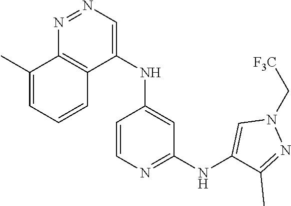 [SMILES: Cc1nn(CC(F)(F)F)cc1Nc1cc(Nc2cnnc3c(C)cccc23)ccn1]
or a pharmaceutically acceptable salt thereof.

38. The compound of claim 36, wherein the compound is of the following formula: 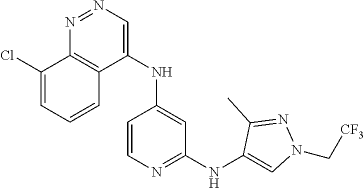 [SMILES: Cc1nn(CC(F)(F)F)cc1Nc1cc(Nc2cnnc3c(Cl)cccc23)ccn1]
or a pharmaceutically acceptable salt thereof.

39. The compound of claim 36, wherein the compound is of the following formula: 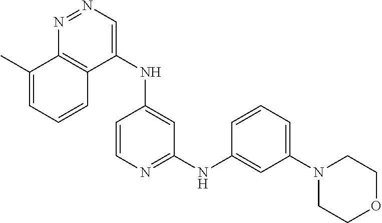 [SMILES: Cc1cccc2c(Nc3ccnc(Nc4cccc(N5CCOCC5)c4)c3)cnnc12]
or a pharmaceutically acceptable salt thereof.

40. The compound of claim 36, wherein the compound is of the following formula: 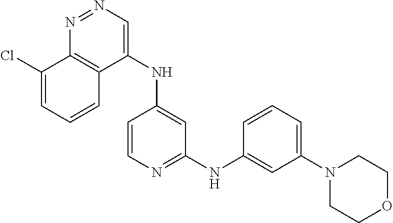 [SMILES: Clc1cccc2c(Nc3ccnc(Nc4cccc(N5CCOCC5)c4)c3)cnnc12]
or a pharmaceutically acceptable salt thereof.

41. The compound of claim 36, wherein the compound is of the following formula: 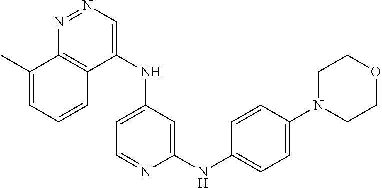 [SMILES: Cc1cccc2c(Nc3ccnc(Nc4ccc(N5CCOCC5)cc4)c3)cnnc12]
or a pharmaceutically acceptable salt thereof.

42. The compound of claim 36, wherein the compound is of the following formula: 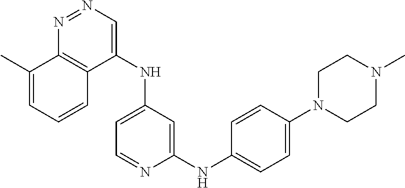
or a pharmaceutically acceptable salt thereof.

43. The compound of claim 36, wherein the compound is of the following formula: 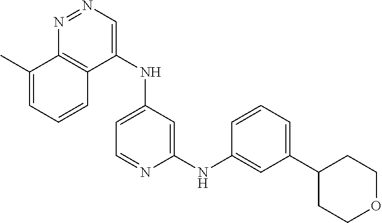 [SMILES: Cc1cccc2c(Nc3ccnc(Nc4cccc(C5CCOCC5)c4)c3)cnnc12]
or a pharmaceutically acceptable salt thereof.

44. The compound of claim 36, wherein the compound is of the following formula: 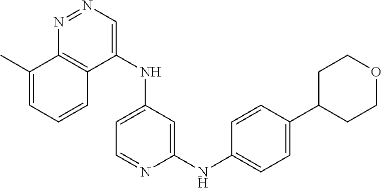 [SMILES: Cc1cccc2c(Nc3ccnc(Nc4ccc(C5CCOCC5)cc4)c3)cnnc12]
or a pharmaceutically acceptable salt thereof.

45. A pharmaceutical composition comprising a compound of any one of claims 1-44, or a pharmaceutically acceptable salt thereof, and at least one pharmaceutically acceptable excipient.

46. A pharmaceutical combination comprising a compound of any one of claims 1-44, or a pharmaceutically acceptable salt thereof, or a pharmaceutical composition of claim 45, and one or more additional therapeutic agents.

47. A method of treating a proliferative disease in a subject, the method comprising administering to the subject a compound of any one of claims 1-44, or a pharmaceutically acceptable salt thereof, or a pharmaceutical composition of claim 45.

48. The method of claim 47, wherein the proliferative disease is cancer.

49. The method of claim 48, wherein the cancer is a hematological cancer.

50. The method of claim 48, wherein the cancer comprises a solid tumor.

51. The method of claim 48, wherein the cancer is lung cancer, brain cancer, thyroid cancer, anaplastic astrocytoma, liver cancer, pancreatic cancer, skin cancer, melanoma, metastatic melanoma, colorectal cancer, breast cancer, prostate cancer, renal cancer, hepatocellular cancer, ovarian cancer, an HPV-associated cancer, multiple myeloma, myelodysplastic syndrome, a hematological cancer, or myelofibrosis.

52. The method of claim 48, wherein the cancer is non-small cell lung cancer (NSCLC), neuroblastoma, glioblastoma, anaplastic thyroid cancer (ATC), colon carcinoma, hepatocellular carcinoma (HCC), pancreatic carcinoma, anaplastic large cell lymphoma (ALCL), myelodysplastic syndrome, anaplastic astrocytoma or pancreatic ductal adenocarcinoma.

53. The method of claim 48, wherein the cancer is an adult granulosa cell tumor.

54. The method of claim 48, wherein the cancer is an HPV-associated cancer selected from cervical cancer, oropharyngeal cancer, anal cancer, vulvar/vaginal cancer, or penile cancer.

55. The method of claim 47, wherein the proliferative disease is a fibrotic condition.

56. The method of claim 55, wherein the fibrotic condition is idiopathic pulmonary fibrosis, cardiac fibrosis, a condition associated with cardiac fibrosis, valvular disease, arrhythmia, atrial fibrillation, myocardial remodeling, cardiomyopathy, dilated cardiomyopathy, ischemic cardiomyopathy, hypertrophic cardiomyopathy, restenosis, liver fibrosis, liver cirrhosis, nonalcoholic steatohepatitis, Peyronie's, Dupuytren's contracture, cystic fibrosis, beta thalassemia, actinic keratosis, hypertension, a general inflammatory disorder, dry eye, ulcer, corneal fibrosis, wet age-related macular degeneration, psoriasis, wound closure, chronic kidney disease, renal fibrosis, systemic sclerosis, or chronic Chagas' heart disease.

57. A method of inhibiting tumor growth in a subject, the method comprising administering to the subject a compound of any one of claims 1-44, or a pharmaceutically acceptable salt thereof, or a pharmaceutical composition of claim 45.

58. A method of inhibiting ALK-5 activity in vivo or in vitro, the method comprising contacting ALK-5 with a compound of any one of claims 1-44, or a pharmaceutically acceptable salt thereof, or a pharmaceutical composition of claim 45.

59. A method of treating an inflammatory disease, disorder, or condition in a subject in need thereof, comprising administering to the subject a therapeutically effective amount of a compound of any one of claims 1-44, or a pharmaceutically acceptable salt thereof, or a pharmaceutical composition of claim 45.

60. The method of claim 59, wherein the inflammatory disease, disorder, or condition is non-alcoholic fatty liver disease (NAFLD), alcoholic steatohepatitis (ASH), non-alcoholic steatohepatitis (NASH), primary biliary cholangitis (PBC), primary sclerosing cholangitis, autoimmune hepatitis, skin inflammation, or psoriasis.

61. The method of claim 59 or 60, wherein the inflammatory disease, disorder, or condition is an autoimmune disease, disorder, or condition.

62. The method of claim 61, wherein the autoimmune disease, disorder, or condition is osteoarthritis, rheumatoid arthritis, pain, inflammatory bowel disease, a respiratory disorder, or a skin disorder.

63. A method of treating a fibrotic, inflammatory or proliferative disease or condition which is susceptible to inhibition of the TGFβ signaling pathway, the method comprising administering to a subject suffering from the fibrotic, inflammatory or proliferative disease or condition a compound of any one of claims 1-44, or a pharmaceutically acceptable salt thereof, or a pharmaceutical composition of claim 45, in an amount effective to inhibit TGFβ signaling.

64. The method of any one of claims 47-57 and 59-63, wherein the subject is a human.

65. The method of any one of claims 47-57 and 59-64, wherein the proliferative disease, inflammatory disease, disorder or condition, tumor, cancer, or fibrotic, inflammatory or proliferative disease or condition expresses or has mutant forkhead box L2 (FOXL2) or FOXL2.

66. The method of any one of claims 47-57 and 59-65, further comprising administering one or more additional therapeutic agents to the subject selected from an anti-cancer agent and an immune checkpoint inhibitor.

67. The method of any one of claims 47-57 and 59-66, further comprising treating the subject with radiation therapy or surgery.

68. A method of inhibiting epithelial to mesenchymal transition (EMT) in a subject suffering from a disease or condition which is promoted by EMT, comprising administering at least one compound of any one of claims 1-44, or a pharmaceutically acceptable salt thereof, or a pharmaceutical composition of claim 45 to the subject in an amount effective to sufficiently inhibit EMT to alter the course of the disease or condition.

69. A method for enhancing the activity of one or more therapeutic agents for treating cancer in a subject in need thereof, comprising administering to the subject an effective amount of a compound of any one of claims 1-44, or a pharmaceutically acceptable salt thereof, or a pharmaceutical composition of claim 45.

70. The method of claim 69, further comprising administering to the subject one or more therapeutic agents selected from an anti-cancer agent or an immune checkpoint inhibitor.

71. The method of claim 66, 67 or 70, wherein the immune checkpoint inhibitor is a PD-1 or PD-L1 inhibitor.
